(19) United States
(12) Patent Application Publication
(10) Pub. No.: US 2024/0327931 A1
(43) Pub. Date: Oct. 3, 2024

(54) GENE EXPRESSION ASSAY FOR MEASUREMENT OF DNA MISMATCH REPAIR DEFICIENCY

(71) Applicant: Bruker Spatial Biology, Inc., Billerica, MA (US)

(72) Inventors: Patrick DANAHER, Seattle, WA (US); Sarah WARREN, Seattle, WA (US)

(21) Appl. No.: 18/743,327

(22) Filed: Jun. 14, 2024

Related U.S. Application Data
(62) Division of application No. 17/086,842, filed on Nov. 2, 2020, now Pat. No. 12,054,782.
Application No. 17/086,842 is a continuation of application No. PCT/US2019/030537, filed on May 3, 2019.
(60) Provisional application No. 62/666,870, filed on May 4, 2018.

(51) Int. Cl. C12Q 1/6886; G16B 20/20

(52) U.S. Cl. CPC C12Q 1/6886; G16B 20/20; C12Q 2600/156; C12Q 2600/158

(57) ABSTRACT

The present disclosure relates to methods using gene expression measurements to identify mismatch repair deficiency, microsatellite instability and hypermutation in a subject.

### CROSS-REFERENCE TO RELATED APPLICATIONS

This application is a division of U.S. application Ser. No. 17/086,842, now allowed, which is a continuation of International Patent Application No. PCT/US2019/030537, filed May 3, 2019, which claims priority to, and the benefit of, U.S. Provisional Application No. 62/666,870, filed May 4, 2018. The contents of each of the aforementioned patent applications are incorporated herein by reference in their entireties.

### SEQUENCE LISTING

The Sequence Listing XML associated with this application is provided electronically in XML file format and is hereby incorporated by reference into the specification. The name of the XML file containing the Sequence Listing is “NATE-038_D01US_SeqList.xml”. The XML file is 177,331 bytes in size, created on Jun. 13, 2024, and is being submitted electronically via USPTO Patent Center.

### BACKGROUND OF THE INVENTION

There are currently a variety of methods for identifying mismatch repair deficiency, microsatellite instability and hypermutation in tumor samples from a subject. Current methods rely on PCR and immunohistochemistry. These methods require a large tumor sample, are costly, and are time-intensive. Importantly, whether a subject will respond to and receive a clinical benefit from checkpoint inhibitors, e.g. drugs that target PD-1 or PD-L1, can be predicted based on the presence of mismatch repair deficiency, microsatellite instability and hypermutation. Thus, there is a need in the art for methods of identifying mismatch repair deficiency, microsatellite instability and hypermutation that are rapid, specific, and accurate, and that require smaller tumor samples. The present disclosure addresses these needs.

### SUMMARY OF THE INVENTION

The present disclosure provides a method of identifying mismatch repair deficiency in a subject comprising: a) measuring the gene expression level of at least one gene comprising MLH1, MSH2, MSH6 or PMS2 in a tumor sample from the subject; b) determining for each of the at least one gene a score Z, wherein Z=(x−μ₁)/σ₁, wherein x is the log-transformed normalized expression of the at least one gene, μ₁ is the mean of the log-transformed normalized expression of the at least one gene in non-hypermutated samples, and σ₁ is the standard deviation of the log-transformed normalized expression of the at least one gene in non-hypermutated samples; c) determining a score MLS, wherein MLS=(Zₘ+c₁)/c₂, wherein Zₘ is the minimum Z score of the at least one gene, and wherein c₁ is 0 and c₂ is 1 when one gene is used, c₁ is 0.56 and c₂ is 0.83 when two genes are used, c₁ is 0.85 and c₂ is 0.75 when three gene are used, or c₁ is 1.03 and c₂ is 0.70 when four genes are used; d) comparing the MLS score with a predetermined cutoff value, wherein the cutoff value identifies mismatch repair deficiency in a subject with at least 95% specificity; and e) producing a report identifying the presence of mismatch repair deficiency in the subject when the MLS score is equal to or greater than the predetermined cutoff value or producing a report identifying the absence of mismatch repair deficiency in the subject when the MLS score is less than the predetermined cutoff value.

The present disclosure provides a method of identifying mismatch repair deficiency in a subject comprising: a) measuring the gene expression level of at least one gene comprising MLH1, MSH2, MSH6 or PMS2 in a tumor sample from the subject; b) determining for each of the at least one gene a score Z, wherein Z=(x−μ₁)/σ₁, wherein x is the log-transformed normalized expression of the at least one gene, μ₁ is the mean of the log-transformed normalized expression of the at least one gene in non-hypermutated samples, and σ₁ is the standard deviation of the log-transformed normalized expression of the at least one gene in non-hypermutated samples; c) determining a score MLS, wherein MLS=(Zₘ+c₁)/c₂, wherein Zₘ is the minimum Z score of the at least one gene, and wherein c₁ is 0 and c₂ is 1 when one gene is used, c₁ is 0.56 and c₂ is 0.83 when two genes are used, c₁ is 0.85 and c₂ is 0.75 when three gene are used, or c₁ is 1.03 and c₂ is 0.70 when four genes are used; d) comparing the MLS score with a predetermined cutoff value, wherein the cutoff value identifies mismatch repair deficiency in a subject with at least 95% specificity; and e) identifying the presence of mismatch repair deficiency in the subject when the MLS score is equal to or greater than the predetermined cutoff value or identifying the absence of mismatch repair deficiency in the subject when the MLS score is less than the predetermined cutoff value.

The predetermined cutoff value that identifies mismatch repair deficiency in a subject can have a specificity of 99%. Alternatively, the cutoff value that identifies mismatch repair deficiency in a subject can have a specificity of 99.5%. The predetermined cutoff value can be 1.645, 2.326, or 2.576.

The at least one gene in step (a) can comprise MLH1. Alternatively, the at least one gene in step (a) can comprise each of MLH1, MSH2, MSH6 and PMS2.

The present disclosure provides a method of identifying mismatch repair deficiency in a subject comprising: a) measuring the gene expression level of at least one gene comprising EPM2AIP1, TTC30A, SMAP1, RNLS, WNT11, SFXN1, SREBF1, TYMS, EIF5AL1, or WDR76 in a tumor sample from the subject; b) determining a score HPS, wherein HPS=(y−μ₂)/σ₂, wherein y=Σᵢ₌₁¹⁰yᵢwᵢ, wherein yᵢ is the log-transformed normalized expression of the at least one gene i in the tumor sample and wᵢ is the prespecified weight for gene i, μ₂ is the mean of the linear combination of the log-transformed normalized expression of the at least one gene in non-hypermutated samples, and σ₂ is the standard deviation of the linear combination of the log-transformed normalized expression of the at least one gene in non-hypermutated samples; c) comparing the HPS score with a predetermined cutoff value, wherein the cutoff value identifies mismatch repair deficiency in a subject with at least 95% specificity; and d) producing a report identifying the presence of mismatch repair deficiency in the subject when the HPS score is equal to or greater than the predetermined cutoff value or producing a report identifying the absence of mismatch repair deficiency in the subject when the HPS score is less than the predetermined cutoff value.

The present disclosure provides a method of identifying mismatch repair deficiency in a subject comprising: a) measuring the gene expression level of at least one gene comprising EPM2AIP1, TTC30A, SMAP1, RNLS, WNT11, SFXN1, SREBF1, TYMS, EIF5AL1, or WDR76 in a tumor sample from the subject; b) determining a score HPS, wherein HPS=(y−μ₂)/σ₂, wherein y=Σᵢ₌₁¹⁰yᵢwᵢ, wherein yᵢ is the log-transformed normalized expression of the at least one gene i in the tumor sample and wᵢ is the prespecified weight for gene i, μ₂ is the mean of the linear combination of the log-transformed normalized expression of the at least one gene in non-hypermutated samples, and σ₂ is the standard deviation of the linear combination of the log-transformed normalized expression of the at least one gene in non-hypermutated samples; c) comparing the HPS score with a predetermined cutoff value, wherein the cutoff value identifies mismatch repair deficiency in a subject with at least 95% specificity; and d) identifying the presence of mismatch repair deficiency in the subject when the HPS score is equal to or greater than the predetermined cutoff value or identifying the absence of mismatch repair deficiency in the subject when the HPS score is less than the predetermined cutoff value.

The prespecified weight for gene i, wᵢ, in step (b) can be:

| Gene | Weight |
|---|---|
| EPM2AIP1 | −0.31218 |
| TTC30A | −0.19894 |
| SMAP1 | −0.1835 |
| RNLS | −0.19023 |
| WNT11 | −0.11515 |
| SFXN1 | 0.214676 |
| SREBF1 | 0.194835 |
| TYMS | 0.206972 |
| EIF5AL1 | 0.194935 |
| WDR76 | 0.188582 |

The predetermined cutoff value that identifies mismatch repair deficiency in a subject can have a specificity of 99%. Alternatively, the cutoff value that identifies mismatch repair deficiency in a subject can have a specificity of 99.5%. The cutoff value can be 1.645, 2.326, or 2.576.

The at least one gene in step (a) can comprise each of EPM2AIP1, TTC30A, SMAP1, RNLS, WNT11, SFXN1, SREBF1, TYMS, EIF5AL1, and WDR76.

The present disclosure provides a method of identifying mismatch repair deficiency in a subject comprising: a) measuring the gene expression level of at least one gene comprising MLH1, MSH2, MSH6 or PMS2 in a tumor sample from the subject; b) determining for each of the at least one gene a score Z, wherein Z=(x−μ₁)/σ₁, wherein x is the log-transformed normalized expression of the at least one gene, μ₁ is the mean of the log-transformed normalized expression of the at least one gene in non-hypermutated samples, and σ₁ is the standard deviation of the log-transformed normalized expression of the at least one gene in non-hypermutated samples; c) determining a score MLS, wherein MLS=(Zₘ+c₁)/c₂, wherein Zₘ is the minimum Z score of the at least one gene, and wherein c₁ is 0 and c₂ is 1 when one gene is used, c₁ is 0.56 and c₂ is 0.83 when two genes are used, c₁ is 0.85 and c₂ is 0.75 when three genes are used, or c₁ is 1.03 and c₂ is 0.70 when four genes are used; d) measuring the gene expression level of at least one gene comprising EPM2AIP1, TTC30A, SMAP1, RNLS, WNT11, SFXN1, SREBF1, TYMS, EIF5AL1, or WDR76 in a tumor sample from the subject; e) determining a score HPS, wherein HPS=(y−μ₂)/σ₂, wherein y=Σᵢ₌₁¹⁰yᵢwᵢ, wherein yᵢ is the log-transformed normalized expression of the at least one gene i in the tumor sample and wᵢ is the prespecified weight for gene i, μ₂ is the mean of the linear combination of the log-transformed normalized expression of the at least one gene in non-hypermutated samples, and σ₂ is the standard deviation of the linear combination of the log-transformed normalized expression of the at least one gene in non-hypermutated samples; f) determining a score MPS wherein MPS=(max(HPS,0)²+min(MLS,0)²)^{1/2}; g) comparing the MPS score with a predetermined cutoff value, wherein the cutoff value identifies mismatch repair deficiency in a subject with at least 95% specificity; and h) producing a report identifying the presence of mismatch repair deficiency in the subject when the MPS score is equal to or greater than the predetermined cutoff value or producing a report identifying the absence of mismatch repair deficiency in the subject when the MPS score is less than the predetermined cutoff value.

The present disclosure provides a method of identifying mismatch repair deficiency in a subject comprising: a) measuring the gene expression level of at least one gene comprising MLH1, MSH2, MSH6 or PMS2 in a tumor sample from the subject; b) determining for each of the at least one gene a score Z, wherein Z=(x−μ₁)/σ₁, wherein x is the log-transformed normalized expression of the at least one gene, μ₁ is the mean of the log-transformed normalized expression of the at least one gene in non-hypermutated samples, and σ₁ is the standard deviation of the log-transformed normalized expression of the at least one gene in non-hypermutated samples; c) determining a score MLS, wherein MLS=(Zₘ+c₁)/c₂, wherein Zₘ is the minimum Z score of the at least one gene, and wherein c₁ is 0 and c₂ is 1 when one gene is used, c₁ is 0.56 and c₂ is 0.83 when two genes are used, c₁ is 0.85 and c₂ is 0.75 when three genes are used, or c₁ is 1.03 and c₂ is 0.70 when four genes are used; d) measuring the gene expression level of at least one gene comprising EPM2AIP1, TTC30A, SMAP1, RNLS, WNT11, SFXN1, SREBF1, TYMS, EIF5AL1, or WDR76 in a tumor sample from the subject; e) determining a score HPS, wherein HPS=(y−μ₂)/σ₂, wherein y=Σᵢ₌₁¹⁰yᵢwᵢ, wherein yᵢ is the log-transformed normalized expression of the at least one gene i in the tumor sample and wᵢ is the prespecified weight for gene i, μ₂ is the mean of the linear combination of the log-transformed normalized expression of the at least one gene in non-hypermutated samples, and σ₂ is the standard deviation of the linear combination of the log-transformed normalized expression of the at least one gene in non-hypermutated samples; f) determining a score MPS wherein MPS=(max(HPS,0)²+min(MLS,0)²)^{1/2}; g) comparing the MPS score with a predetermined cutoff value, wherein the cutoff value identifies mismatch repair deficiency in a subject with at least 95% specificity; and h) identifying the presence of mismatch repair deficiency in the subject when the MPS score is equal to or greater than the predetermined cutoff value or identifying the absence of mismatch repair deficiency in the subject when the MPS score is less than the predetermined cutoff value.

The prespecified weight for gene i, wᵢ, in step (e) can be

| Gene | Weight |
|---|---|
| EPM2AIP1 | −0.31218 |
| TTC30A | −0.19894 |
| SMAP1 | −0.1835 |
| RNLS | −0.19023 |
| WNT11 | −0.11515 |
| SFXN1 | 0.214676 |
| SREBF1 | 0.194835 |
| TYMS | 0.206972 |
| EIF5AL1 | 0.194935 |
| WDR76 | 0.188582 |

The predetermined cutoff value that identifies mismatch repair deficiency in a subject can have a specificity of 99%. Alternatively, the cutoff value that identifies mismatch repair deficiency in a subject can have a specificity of 99.5%. The cutoff value can be 2.058, 2.699, or 2.939.

The at least one gene in step (a) can comprise MLH1. Alternatively, the at least one gene in step (a) can comprise each of MLH1, MSH2, MSH6 and PMS2.

The at least one gene in step (d) can comprise each of EPM2AIP1, TTC30A, SMAP1, RNLS, WNT11, SFXN1, SREBF1, TYMS, EIF5AL1 and WDR76.

The at least one gene in step (a) can comprise MLH1 and the at least one gene in step (d) can comprise each of EPM2AIP1, TTC30A, SMAP1, RNLS, WNT11, SFXN1, SREBF1, TYMS, EIF5AL1 and WDR76. Alternatively, the at least one gene in step (a) can comprise each of MLH1, MSH2, MSH6 and PMS2 and the at least one gene in step (d) can comprise each of EPM2AIP1, TTC30A, SMAP1, RNLS, WNT11, SFXN1, SREBF1, TYMS, EIF5AL1 and WDR76.

A subject can be diagnosed with cancer.

A report identifying mismatch repair deficiency can further identify the subject as having cancer.

A report identifying the presence of mismatch repair deficiency can further identify the subject for treatment with an anti-cancer therapy. A treatment can comprise administering to the subject immunotherapy. A treatment can comprise administering to the subject checkpoint inhibitors. A treatment can comprise administering to the subject pembrolizumab, nivolumab, atezolizumab, avelumab, durvalumab, pidilizumab, REGN2810, AMP-224, MEDI0680, PDR001, CT-001 or a combination thereof. A treatment can comprise administering to the subject a CTLA4 antibody. The CTLA4 antibody can comprise ipilimumab, tremelimumab or a combination thereof.

The methods of the present disclosure can further comprise determining a tumor inflammation signature score.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a series of graphs that shows the expression level of certain mismatch repair genes plotted against mutation load and microsatellite instability status in four different cancer types.

FIG. 2 is a series of volcano plots that shows that particular genes are positively and negatively associated with hypermutation in three different cancer types.

FIG. 3 is a series of graphs that shows the methods of the present disclosure can accurately predict microsatellite instability status in a tumor sample.

FIG. 4 is a series of box plots that shows the relationship between the expression of four mismatch repair genes and microsatellite instability in validation samples of two different cancer types.

FIG. 5 is a series of graphs that shows the performance of the methods of the present disclosure in determining microsatellite instability status in validation samples of two different cancer types.

FIG. 6 is a series of graphs showing the results of the methods of the present disclosure plotted against tumor inflammation signature score and microsatellite instability status.

### DETAILED DESCRIPTION OF THE INVENTION

The present disclosure provides methods that identify mismatch repair deficiency, hypermutation, and microsatellite instability in a subject using gene expression measurements.

The clinical benefit of checkpoint inhibitors varies widely between patients and only a small subset experience durable disease remission upon treatment. Response to checkpoint inhibition is associated with two biological axes: tumor foreignness, typically measured by tumor mutation burden or microsatellite instability (MSI), and the presence of an adaptive anti-tumor immune response, typically measured by gene expression signatures of inflammation or immunohistochemistry. Because tumor foreignness and the magnitude of the adaptive immune response in the tumor microenvironment are only weakly correlated, more accurate predictions of immunotherapy response should be possible by measuring and integrating both variables together. However, in a clinical setting, performing multiple assays is often impractical due to more tissue requirement, increased turn-around time, and cost. Here, the ability of gene expression to predict tumor MSI was investigated, and a single assay that enables measurement of tumor foreignness and tumor inflammation was developed.

DNA mismatch repair deficiency (MMRd) has been observed in most cancer types in The Cancer Genome Atlas (TCGA), and occurs in more than 5% of adrenal, rectal, colon, stomach, and uterine tumors. Tumors with this phenotype develop both point and frameshift mutations at an increased rate and are often described as hypermutated. The failure of mismatch repair (MMR) to correct replication errors at short repeated DNA sequences can lead to the phenomenon of high-level MSI (MSI-H). MSI-H cancers have distinct clinical behavior, which has led to widespread MSI testing in cancers where MSI-H is common. In colorectal cancer, the MSI-H phenotype demonstrates association with proximal tumor localization, a dense local lymphocyte infiltration, and a low frequency of distant organ metastasis. Moreover, MSI-H colorectal cancers have a better prognosis than their microsatellite-stable (MSS) counterparts. Diminished responsiveness of MSI-H colorectal cancer patients towards chemotherapy has been shown in several studies. In the era of immunotherapy, MMRd has gained greater relevance as a cause of hypermutation potentiating anti-tumor immune responses which may be augmented by checkpoint inhibition. Importantly, the frame-shift mutations that accrue in MMRd tumors lead to highly abnormal peptides that may be more immunogenic. Thus, the high pan-cancer clinical efficacy of checkpoint inhibitors in MMRd tumors may arise more from their high rate of frameshift mutations than from their total tumor mutation burden.

MMRd often arises from loss of protein expression of 1 of 4 genes essential for MMR: MLH1, MSH2, MSH6, and PMS2. Lost expression of these proteins can arise from mutations in their coding regions, either from acquired somatic mutations or from germline mutations associated with Lynch syndrome. In tumors with intact sequences for these genes, loss of protein expression can follow loss of mRNA expression. A common cause of lost mRNA expression in these genes is the CpG island methylator phenotype (CIMP), which is associated with widespread methylation across the genome and frequently silences DNA repair genes. Loss of MMR activity due to microRNA-induced downregulation of MSH2 has also been observed in colorectal tumors. MMRd can be detected by measuring either its cause or its effect. Immunohistochemistry (IHC) is used to measure loss of expression of proteins essential to the MMR machinery, and PCR and sequencing are used to measure MSI, the genomic “scarring” which occurs as a consequence of MMRd.

The biology underlying MMRd provides two opportunities for capturing MMRd with gene expression data. First, loss of expression of MMR genes may be used to detect cases of MMRd resulting from transcriptional silencing. Second, if it is assumed that MMRd and CIMP exert broad and consistent influence on the transcriptome, then a data-driven predictor of hypermutation based on RNA expression patterns may also be possible.

Various methods of the present disclosure are described in full detail herein.

In one aspect, the present disclosure provides a method of identifying mismatch repair deficiency in a subject comprising: a) measuring the gene expression level of at least one gene comprising MLH1, MSH2, MSH6 or PMS2 in a tumor sample from the subject; b) determining for each of the at least one gene a score Z, wherein Z=(x−μ₁)/σ₁, wherein x is the log-transformed normalized expression of the at least one gene, μ₁ is the mean of the log-transformed normalized expression of the at least one gene in non-hypermutated samples, and σ₁ is the standard deviation of the log-transformed normalized expression of the at least one gene in non-hypermutated samples; c) determining a score MLS, wherein MLS=(Zₘ+c₁)/c₂, wherein Zₘ is the minimum Z score of the at least one gene, and wherein c₁ is 0 and c₂ is 1 when one gene is used, c₁ is 0.56 and c₂ is 0.83 when two genes are used, c₁ is 0.85 and c₂ is 0.75 when three gene are used, or c₁ is 1.03 and c₂ is 0.70 when four genes are used; d) comparing the MLS score with a predetermined cutoff value, wherein the cutoff value identifies mismatch repair deficiency in a subject with at least 95% specificity; and e) producing a report identifying the presence of mismatch repair deficiency in the subject when the MLS score is equal to or greater than the predetermined cutoff value or producing a report identifying the absence of mismatch repair deficiency in the subject when the MLS score is less than the predetermined cutoff value.

In one aspect, the present disclosure provides a method of identifying mismatch repair deficiency in a subject comprising: a) measuring the gene expression level of at least one gene comprising MLH1, MSH2, MSH6 or PMS2 in a tumor sample from the subject; b) determining for each of the at least one gene a score Z, wherein Z=(x−μ₁)/σ₁, wherein x is the log-transformed normalized expression of the at least one gene, μ₁ is the mean of the log-transformed normalized expression of the at least one gene in non-hypermutated samples, and σ₁ is the standard deviation of the log-transformed normalized expression of the at least one gene in non-hypermutated samples; c) determining a score MLS, wherein MLS=(Zₘ+c₁)/c₂, wherein Zₘ is the minimum Z score of the at least one gene, and wherein c₁ is 0 and c₂ is 1 when one gene is used, c₁ is 0.56 and c₂ is 0.83 when two genes are used, c₁ is 0.85 and c₂ is 0.75 when three gene are used, or c₁ is 1.03 and c₂ is 0.70 when four genes are used; d) comparing the MLS score with a predetermined cutoff value, wherein the cutoff value identifies mismatch repair deficiency in a subject with at least 95% specificity; and e) identifying the presence of mismatch repair deficiency in the subject when the MLS score is equal to or greater than the predetermined cutoff value or identifying the absence of mismatch repair deficiency in the subject when the MLS score is less than the predetermined cutoff value.

In some aspects, the preceding methods can further comprise administering at least one treatment to a subject identified as having mismatch repair deficiency. A treatment can comprise anti-cancer therapy. A treatment can comprise administering to the subject immunotherapy. The at least one treatment can comprise administering to the subject at least one checkpoint inhibitor. A treatment can comprise administering to the subject pembrolizumab, nivolumab, atezolizumab, avelumab, durvalumab, pidilizumab, REGN2810, AMP-224, MEDI0680, PDR001, CT-001 or a combination thereof. A treatment can comprise administering to the subject a CTLA4 antibody. A CTLA4 antibody can comprise ipilimumab, tremelimumab or a combination thereof.

In one aspect, the present disclosure provides a method of identifying mismatch repair deficiency in a subject comprising: a) measuring the gene expression level of at least one gene comprising MLH1, MSH2, MSH6 or PMS2 in a tumor sample from the subject; b) determining for each of the at least one gene a score Z, wherein Z=(x−μ₁)/σ₁, wherein x is the log-transformed normalized expression of the at least one gene, μ₁ is the mean of the log-transformed normalized expression of the at least one gene in non-hypermutated samples, and σ₁ is the standard deviation of the log-transformed normalized expression of the at least one gene in non-hypermutated samples; c) determining a score MLS, wherein MLS=(Zₘ+c₁)/c₂, wherein Zₘ is the minimum Z score of the at least one gene, and wherein c₁ is 0 and c₂ is 1 when one gene is used, c₁ is 0.56 and c₂ is 0.83 when two genes are used, c₁ is 0.85 and c₂ is 0.75 when three gene are used, or c₁ is 1.03 and c₂ is 0.70 when four genes are used; d) comparing the MLS score with a predetermined cutoff value, wherein the cutoff value identifies mismatch repair deficiency in a subject with at least 95% specificity; and e) administering at least one treatment to the subject when the MLS score is equal to or greater than the predetermined cutoff value. A treatment can comprise anti-cancer therapy. A treatment can comprise administering to the subject immunotherapy. The at least one treatment can comprise administering to the subject at least one checkpoint inhibitor. A treatment can comprise administering to the subject pembrolizumab, nivolumab, atezolizumab, avelumab, durvalumab, pidilizumab, REGN2810, AMP-224, MEDI0680, PDR001, CT-001 or a combination thereof. A treatment can comprise administering to the subject a CTLA4 antibody. A CTLA4 antibody can comprise ipilimumab, tremelimumab or a combination thereof.

In some aspects of the preceding methods, determining μ₁ in step (b), wherein μ₁ is the mean of the log-transformed normalized expression of the at least one gene in non-hypermutated samples, comprises: 1) measuring the gene expression level of the at least one gene in a plurality of analogous, non-hypermutated tumor samples from at least one subject, wherein at least one sample in the plurality of analogous, non-hypermutated samples originates from the same tissue as the tumor sample in step (a) of the preceding method; 2) determining for each of the at least one gene the log-transformed normalized expression; and 3) determining for each of the at least one gene the mean of the log 2-transformed expression from step (2).

In some aspects of the preceding methods, determining σ₁ in step (b), wherein σ₁ is the standard deviation of the log-transformed normalized expression of the at least one gene in non-hypermutated samples, comprises: 1) measuring the gene expression level of the at least one gene in a plurality of analogous, non-hypermutated tumor samples from at least one subject, wherein at least one sample in the plurality of analogous, non-hypermutated samples originates from the same tissue as the tumor sample in step (a) of the preceding method; 2) determining for each of the at least one gene the log-transformed normalized expression; and 3) determining for each of the at least one gene the standard deviation of the log 2-transformed expression from step (2).

In some aspects of the preceding methods, measuring the gene expression of the at least one gene in a tumor sample from the subject and measuring the gene expression of the at least one gene in a plurality of analogous non-hypermutated tumor samples is performed using the same method. In some aspects of the preceding method, measuring the gene expression of the at least one gene in a tumor sample from the subject and measuring the gene expression of the at least one gene in a plurality of analogous non-hypermutated tumor samples is performed using the same apparatus. In preferred aspects of the preceding method, measuring the gene expression of the at least one gene in a tumor sample from the subject and measuring the gene expression of the at least one gene in a plurality of analogous non-hypermutated tumor samples is performed using the same method and apparatus.

In some aspects, the predetermined cutoff value in the preceding methods that identifies mismatch repair deficiency in a subject can have a specificity of 99%. In some aspects, the predetermined cutoff value in the preceding methods that identifies mismatch repair deficiency in a subject can have a specificity of at least 99%. In preferred aspects, the cutoff value that identifies mismatch repair deficiency in a subject can have a specificity of 99.5%. In preferred aspects, the cutoff value that identifies mismatch repair deficiency in a subject can have a specificity of at least 99.5%.

In some aspects, the predetermined cutoff value in the preceding methods that identifies mismatch repair deficiency in a subject can have a specificity of at least 70%, or at least 75%, or at least 80%, or at least 85%, or at least 90%, or at least 91%, or at least 92%, or at least 93%, or at least 94%, or at least 95%, or at least 96%, or at least 97% or at least 98%.

In some aspects of the preceding methods, the predetermined cutoff value of the preceding method that identifies mismatch repair deficiency in a subject can be 1.645. Alternatively, the predetermined cutoff value can be 2.326. Alternatively still, the predetermined cutoff value can be 2.576.

In some aspects, the at least one gene in step (a) of the preceding methods can comprise MLH1. Alternatively, the at least one gene in step (a) can comprise each of MLH1, MSH2, MSH6 and PMS2.

In some aspects, step (a) of the preceding methods can comprise measuring the gene expression level of at least two genes, or at least three genes or at least four genes comprising MLH1, MSH2, MSH6 or PMS2 in a tumor sample from the subject.

In some aspects, when the tumor sample is a colon adenocarcinoma (COAD), esophageal carcinoma (ESCA), stomach adenocarcinoma (STAD) or uterine corpus endometrial carcinoma (UCEC) tumor sample, σ₁ in step (b) of the preceeding methods can be:

| | MLH1 | MSH2 | MSH6 | PMS2 |
|---|---|---|---|---|
| COAD | 0.3241 | 0.4108 | 0.4198 | 0.3259 |
| ESCA | 0.5221 | 0.6602 | 0.7347 | 0.4927 |
| STAD | 0.4245 | 0.6020 | 0.4814 | 0.4314 |
| UCEC | 0.4543 | 0.7312 | 0.6158 | 0.4217 |

Table 1 shows the sequences of the at least one gene from step (a) of the preceding method.

TABLE 1
Gene sequences used in the methods of the present invention

| | GenBank | | SEQ ID |
|---|---|---|---|
| Gene | Accession No. | Sequence | NO. |
| MLH1 | NM_000249.2 | ATTGGCTGAAGGCACTTCCGTTGAGCATCTAGA | 1 |
| | | CGTTTCCTTGGCTCTTCTGGCGCCAAAATGTCGT | |
| | | TCGTGGCAGGGGTTATTCGGCGGCTGGACGAG | |
| | | ACAGTGGTGAACCGCATCGCGGCGGGGGAAGT | |
| | | TATCCAGCGGCCAGCTAATGCTATCAAAGAGAT | |
| | | GATTGAGAACTGTTTAGATGCAAAATCCACAAG | |
| | | TATTCAAGTGATTGTTAAAGAGGGAGGCCTGAA | |
| | | GTTGATTCAGATCCAAGACAATGGCACCGGGAT | |
| | | CAGGAAAGAAGATCTGGATATTGTATGTGAAA | |
| | | GGTTCACTACTAGTAAACTGCAGTCCTTTGAGG | |
| | | ATTTAGCCAGTATTTCTACCTATGGCTTTCGAG | |
| | | GTGAGGCTTTGGCCAGCATAAGCCATGTGGCTC | |
| | | ATGTTACTATTACAACGAAAACAGCTGATGGAA | |
| | | AGTGTGCATACAGAGCAAGTTACTCAGATGGA | |
| | | AAACTGAAAGCCCCTCCTAAACCATGTGCTGGC | |
| | | AATCAAGGGACCCAGATCACGGTGGAGGACCT | |
| | | TTTTTACAACATAGCCACGAGGAGAAAAGCTTT | |
| | | AAAAAATCCAAGTGAAGAATATGGGAAAATTT | |
| | | TGGAAGTTGTTGGCAGGTATTCAGTACACAATG | |
| | | CAGGCATTAGTTTCTCAGTTAAAAAACAAGGAG | |
| | | AGACAGTAGCTGATGTTAGGACACTACCCAATG | |
| | | CCTCAACCGTGGACAATATTCGCTCCATCTTTG | |
| | | GAAATGCTGTTAGTCGAGAACTGATAGAAATTG | |
| | | GATGTGAGGATAAAACCCTAGCCTTCAAAATG | |
| | | AATGGTTACATATCCAATGCAAACTACTCAGTG | |
| | | AAGAAGTGCATCTTCTTACTCTTCATCAACCAT | |
| | | CGTCTGGTAGAATCAACTTCCTTGAGAAAAGCC | |
| | | ATAGAAACAGTGTATGCAGCCTATTTGCCCAAA | |
| | | AACACACACCCATTCCTGTACCTCAGTTTAGAA | |
| | | ATCAGTCCCCAGAATGTGGATGTTAATGTGCAC | |
| | | CCCACAAAGCATGAAGTTCACTTCCTGCACGAG | |
| | | GAGAGCATCCTGGAGCGGGTGCAGCAGCACAT | |
| | | CGAGAGCAAGCTCCTGGGCTCCAATTCCTCCAG | |
| | | GATGTACTTCACCCAGACTTTGCTACCAGGACT | |
| | | TGCTGGCCCCTCTGGGGAGATGGTTAAATCCAC | |
| | | AACAAGTCTGACCTCGTCTTCTACTTCTGGAAG | |
| | | TAGTGATAAGGTCTATGCCCACCAGATGGTTCG | |
| | | TACAGATTCCCGGGAACAGAAGCTTGATGCATT | |
| | | TCTGCAGCCTCTGAGCAAACCCCTGTCCAGTCA | |
| | | GCCCCAGGCCATTGTCACAGAGGATAAGACAG | |
| | | ATATTTCTAGTGGCAGGGCTAGGCAGCAAGATG | |
| | | AGGAGATGCTTGAACTCCCAGCCCCTGCTGAAG | |
| | | TGGCTGCCAAAAATCAGAGCTTGGAGGGGGAT | |
| | | ACAACAAAGGGGACTTCAGAAATGTCAGAGAA | |
| | | GAGAGGACCTACTTCCAGCAACCCCAGAAAGA | |
| | | GACATCGGGAAGATTCTGATGTGGAAATGGTG | |
| | | GAAGATGATTCCCGAAAGGAAATGACTGCAGC | |
| | | TTGTACCCCCCGGAGAAGGATCATTAACCTCAC | |
| | | TAGTGTTTTGAGTCTCCAGGAAGAAATTAATGA | |
| | | GCAGGGACATGAGGTTCTCCGGGAGATGTTGC | |
| | | ATAACCACTCCTTCGTGGGCTGTGTGAATCCTC | |
| | | AGTGGGCCTTGGCACAGCATCAAACCAAGTTAT | |
| | | ACCTTCTCAACACCACCAAGCTTAGTGAAGAAC | |
| | | TGTTCTACCAGATACTCATTTATGATTTTGCCAA | |
| | | TTTTGGTGTTCTCAGGTTATCGGAGCCAGCACC | |
| | | GCTCTTTGACCTTGCCATGCTTGCCTTAGATAGT | |
| | | CCAGAGAGTGGCTGGACAGAGGAAGATGGTCC | |
| | | CAAAGAAGGACTTGCTGAATACATTGTTGAGTT | |
| | | TCTGAAGAAGAAGGCTGAGATGCTTGCAGACT | |
| | | ATTTCTCTTTGGAAATTGATGAGGAAGGGAACC | |
| | | TGATTGGATTACCCCTTCTGATTGACAACTATG | |
| | | TGCCCCCTTTGGAGGGACTGCCTATCTTCATTCT | |
| | | TCGACTAGCCACTGAGGTGAATTGGGACGAAG | |
| | | AAAAGGAATGTTTTGAAAGCCTCAGTAAAGAA | |
| | | TGCGCTATGTTCTATTCCATCCGGAAGCAGTAC | |
| | | ATATCTGAGGAGTCGACCCTCTCAGGCCAGCAG | |
| | | AGTGAAGTGCCTGGCTCCATTCCAAACTCCTGG | |
| | | AAGTGGACTGTGGAACACATTGTCTATAAAGCC | |
| | | TTGCGCTCACACATTCTGCCTCCTAAACATTTCA | |
| | | CAGAAGATGGAAATATCCTGCAGCTTGCTAACC | |
| | | TGCCTGATCTATACAAAGTCTTTGAGAGGTGTT | |
| | | AAATATGGTTATTTATGCACTGTGGGATGTGTT | |
| | | CTTCTTTCTCTGTATTCCGATACAAAGTGTTGTA | |
| | | TCAAAGTGTGATATACAAAGTGTACCAACATAA | |
| | | GTGTTGGTAGCACTTAAGACTTATACTTGCCTT | |
| | | CTGATAGTATTCCTTTATACACAGTGGATTGAT | |
| | | TATAAATAAATAGATGTGTCTTAACATAA | |
| MSH2 | NM_000251.1 | GGCGGGAAACAGCTTAGTGGGTGTGGGGTCGC | 2 |
| | | GCATTTTCTTCAACCAGGAGGTGAGGAGGTTTC | |
| | | GACATGGCGGTGCAGCCGAAGGAGACGCTGCA | |
| | | GTTGGAGAGCGCGGCCGAGGTCGGCTTCGTGC | |
| | | GCTTCTTTCAGGGCATGCCGGAGAAGCCGACCA | |
| | | CCACAGTGCGCCTTTTCGACCGGGGCGACTTCT | |
| | | ATACGGCGCACGGCGAGGACGCGCTGCTGGCC | |
| | | GCCCGGGAGGTGTTCAAGACCCAGGGGGTGAT | |
| | | CAAGTACATGGGGCCGGCAGGAGCAAAGAATC | |
| | | TGCAGAGTGTTGTGCTTAGTAAAATGAATTTTG | |
| | | AATCTTTTGTAAAAGATCTTCTTCTGGTTCGTCA | |
| | | GTATAGAGTTGAAGTTTATAAGAATAGAGCTGG | |
| | | AAATAAGGCATCCAAGGAGAATGATTGGTATTT | |
| | | GGCATATAAGGCTTCTCCTGGCAATCTCTCTCA | |
| | | GTTTGAAGACATTCTCTTTGGTAACAATGATAT | |
| | | GTCAGCTTCCATTGGTGTTGTGGGTGTTAAAAT | |
| | | GTCCGCAGTTGATGGCCAGAGACAGGTTGGAG | |
| | | TTGGGTATGTGGATTCCATACAGAGGAAACTAG | |
| | | GACTGTGTGAATTCCCTGATAATGATCAGTTCT | |
| | | CCAATCTTGAGGCTCTCCTCATCCAGATTGGAC | |
| | | CAAAGGAATGTGTTTTACCCGGAGGAGAGACT | |
| | | GCTGGAGACATGGGGAAACTGAGACAGATAAT | |
| | | TCAAAGAGGAGGAATTCTGATCACAGAAAGAA | |
| | | AAAAAGCTGACTTTTCCACAAAAGACATTTATC | |
| | | AGGACCTCAACCGGTTGTTGAAAGGCAAAAAG | |
| | | GGAGAGCAGATGAATAGTGCTGTATTGCCAGA | |
| | | AATGGAGAATCAGGTTGCAGTTTCATCACTGTC | |
| | | TGCGGTAATCAAGTTTTTAGAACTCTTATCAGA | |
| | | TGATTCCAACTTTGGACAGTTTGAACTGACTAC | |
| | | TTTTGACTTCAGCCAGTATATGAAATTGGATAT | |
| | | TGCAGCAGTCAGAGCCCTTAACCTTTTTCAGGG | |
| | | TTCTGTTGAAGATACCACTGGCTCTCAGTCTCT | |
| | | GGCTGCCTTGCTGAATAAGTGTAAAACCCCTCA | |
| | | AGGACAAAGACTTGTTAACCAGTGGATTAAGC | |
| | | AGCCTCTCATGGATAAGAACAGAATAGAGGAG | |
| | | AGATTGAATTTAGTGGAAGCTTTTGTAGAAGAT | |
| | | GCAGAATTGAGGCAGACTTTACAAGAAGATTT | |
| | | ACTTCGTCGATTCCCAGATCTTAACCGACTTGC | |
| | | CAAGAAGTTTCAAAGACAAGCAGCAAACTTAC | |
| | | AAGATTGTTACCGACTCTATCAGGGTATAAATC | |
| | | AACTACCTAATGTTATACAGGCTCTGGAAAAAC | |
| | | ATGAAGGAAAACACCAGAAATTATTGTTGGCA | |
| | | GTTTTTGTGACTCCTCTTACTGATCTTCGTTCTG | |
| | | ACTTCTCCAAGTTTCAGGAAATGATAGAAACAA | |
| | | CTTTAGATATGGATCAGGTGGAAAACCATGAAT | |
| | | TCCTTGTAAAACCTTCATTTGATCCTAATCTCAG | |
| | | TGAATTAAGAGAAATAATGAATGACTTGGAAA | |
| | | AGAAGATGCAGTCAACATTAATAAGTGCAGCC | |
| | | AGAGATCTTGGCTTGGACCCTGGCAAACAGATT | |
| | | AAACTGGATTCCAGTGCACAGTTTGGATATTAC | |
| | | TTTCGTGTAACCTGTAAGGAAGAAAAAGTCCTT | |
| | | CGTAACAATAAAAACTTTAGTACTGTAGATATC | |
| | | CAGAAGAATGGTGTTAAATTTACCAACAGCAA | |
| | | ATTGACTTCTTTAAATGAAGAGTATACCAAAAA | |
| | | TAAAACAGAATATGAAGAAGCCCAGGATGCCA | |
| | | TTGTTAAAGAAATTGTCAATATTTCTTCAGGCT | |
| | | ATGTAGAACCAATGCAGACACTCAATGATGTGT | |
| | | TAGCTCAGCTAGATGCTGTTGTCAGCTTTGCTC | |
| | | ACGTGTCAAATGGAGCACCTGTTCCATATGTAC | |
| | | GACCAGCCATTTTGGAGAAAGGACAAGGAAGA | |
| | | ATTATATTAAAAGCATCCAGGCATGCTTGTGTT | |
| | | GAAGTTCAAGATGAAATTGCATTTATTCCTAAT | |
| | | GACGTATACTTTGAAAAAGATAAACAGATGTTC | |
| | | CACATCATTACTGGCCCCAATATGGGAGGTAAA | |
| | | TCAACATATATTCGACAAACTGGGGTGATAGTA | |
| | | CTCATGGCCCAAATTGGGTGTTTTGTGCCATGT | |
| | | GAGTCAGCAGAAGTGTCCATTGTGGACTGCATC | |
| | | TTAGCCCGAGTAGGGGCTGGTGACAGTCAATTG | |
| | | AAAGGAGTCTCCACGTTCATGGCTGAAATGTTG | |
| | | GAAACTGCTTCTATCCTCAGGTCTGCAACCAAA | |
| | | GATTCATTAATAATCATAGATGAATTGGGAAGA | |
| | | GGAACTTCTACCTACGATGGATTTGGGTTAGCA | |
| | | TGGGCTATATCAGAATACATTGCAACAAAGATT | |
| | | GGTGCTTTTTGCATGTTTGCAACCCATTTTCATG | |
| | | AACTTACTGCCTTGGCCAATCAGATACCAACTG | |
| | | TTAATAATCTACATGTCACAGCACTCACCACTG | |
| | | AAGAGACCTTAACTATGCTTTATCAGGTGAAGA | |
| | | AAGGTGTCTGTGATCAAAGTTTTGGGATTCATG | |
| | | TTGCAGAGCTTGCTAATTTCCCTAAGCATGTAA | |
| | | TAGAGTGTGCTAAACAGAAAGCCCTGGAACTT | |
| | | GAGGAGTTTCAGTATATTGGAGAATCGCAAGG | |
| | | ATATGATATCATGGAACCAGCAGCAAAGAAGT | |
| | | GCTATCTGGAAAGAGAGCAAGGTGAAAAAATT | |
| | | ATTCAGGAGTTCCTGTCCAAGGTGAAACAAATG | |
| | | CCCTTTACTGAAATGTCAGAAGAAAACATCACA | |
| | | ATAAAGTTAAAACAGCTAAAAGCTGAAGTAAT | |
| | | AGCAAAGAATAATAGCTTTGTAAATGAAATCAT | |
| | | TTCACGAATAAAAGTTACTACGTGAAAAATCCC | |
| | | AGTAATGGAATGAAGGTAATATTGATAAGCTAT | |
| | | TGTCTGTAATAGTTTTATATTGTTTTATATTAAC | |
| | | CCTTTTTCCATAGTGTTAACTGTCAGTGCCCATG | |
| | | GGCTATCAACTTAATAAGATATTTAGTAATATT | |
| | | TTACTTTGAGGACATTTTCAAAGATTTTTATTTT | |
| | | GAAAAATGAGAGCTGTAACTGAGGACTGTTTG | |
| | | CAATTGACATAGGCAATAATAAGTGATGTGCTG | |
| | | AATTTTATAAATAAAATCATGTAGTTTGTGG | |
| MSH6 | NM_000179.2 | GGCGAGGCGCCTGTTGATTGGCCACTGGGGCCC | 3 |
| | | GGGTTCCTCCGGCGGAGCGCGCCTCCCCCCAGA | |
| | | TTTCCCGCCAGCAGGAGCCGCGCGGTAGATGCG | |
| | | GTGCTTTTAGGAGCTCCGTCCGACAGAACGGTT | |
| | | GGGCCTTGCCGGCTGTCGGTATGTCGCGACAGA | |
| | | GCACCCTGTACAGCTTCTTCCCCAAGTCTCCGG | |
| | | CGCTGAGTGATGCCAACAAGGCCTCGGCCAGG | |
| | | GCCTCACGCGAAGGCGGCCGTGCCGCCGCTGCC | |
| | | CCCGGGGCCTCTCCTTCCCCAGGCGGGGATGCG | |
| | | GCCTGGAGCGAGGCTGGGCCTGGGCCCAGGCC | |
| | | CTTGGCGCGCTCCGCGTCACCGCCCAAGGCGAA | |
| | | GAACCTCAACGGAGGGCTGCGGAGATCGGTAG | |
| | | CGCCTGCTGCCCCCACCAGTTGTGACTTCTCAC | |
| | | CAGGAGATTTGGTTTGGGCCAAGATGGAGGGTT | |
| | | ACCCCTGGTGGCCTTGTCTGGTTTACAACCACC | |
| | | CCTTTGATGGAACATTCATCCGCGAGAAAGGGA | |
| | | AATCAGTCCGTGTTCATGTACAGTTTTTTGATG | |
| | | ACAGCCCAACAAGGGGCTGGGTTAGCAAAAGG | |
| | | CTTTTAAAGCCATATACAGGTTCAAAATCAAAG | |
| | | GAAGCCCAGAAGGGAGGTCATTTTTACAGTGC | |
| | | AAAGCCTGAAATACTGAGAGCAATGCAACGTG | |
| | | CAGATGAAGCCTTAAATAAAGACAAGATTAAG | |
| | | AGGCTTGAATTGGCAGTTTGTGATGAGCCCTCA | |
| | | GAGCCAGAAGAGGAAGAAGAGATGGAGGTAG | |
| | | GCACAACTTACGTAACAGATAAGAGTGAAGAA | |
| | | GATAATGAAATTGAGAGTGAAGAGGAAGTACA | |
| | | GCCTAAGACACAAGGATCTAGGCGAAGTAGCC | |
| | | GCCAAATAAAAAAACGAAGGGTCATATCAGAT | |
| | | TCTGAGAGTGACATTGGTGGCTCTGATGTGGAA | |
| | | TTTAAGCCAGACACTAAGGAGGAAGGAAGCAG | |
| | | TGATGAAATAAGCAGTGGAGTGGGGGATAGTG | |
| | | AGAGTGAAGGCCTGAACAGCCCTGTCAAAGTT | |
| | | GCTCGAAAGCGGAAGAGAATGGTGACTGGAAA | |
| | | TGGCTCTCTTAAAAGGAAAAGCTCTAGGAAGG | |
| | | AAACGCCCTCAGCCACCAAACAAGCAACTAGC | |
| | | ATTTCATCAGAAACCAAGAATACTTTGAGAGCT | |
| | | TTCTCTGCCCCTCAAAATTCTGAATCCCAAGCC | |
| | | CACGTTAGTGGAGGTGGTGATGACAGTAGTCGC | |
| | | CCTACTGTTTGGTATCATGAAACTTTAGAATGG | |
| | | CTTAAGGAGGAAAAGAGAAGAGATGAGCACAG | |
| | | GAGGAGGCCTGATCACCCCGATTTTGATGCATC | |
| | | TACACTCTATGTGCCTGAGGATTTCCTCAATTCT | |
| | | TGTACTCCTGGGATGAGGAAGTGGTGGCAGATT | |
| | | AAGTCTCAGAACTTTGATCTTGTCATCTGTTAC | |
| | | AAGGTGGGGAAATTTTATGAGCTGTACCACATG | |
| | | GATGCTCTTATTGGAGTCAGTGAACTGGGGCTG | |
| | | GTATTCATGAAAGGCAACTGGGCCCATTCTGGC | |
| | | TTTCCTGAAATTGCATTTGGCCGTTATTCAGATT | |
| | | CCCTGGTGCAGAAGGGCTATAAAGTAGCACGA | |
| | | GTGGAACAGACTGAGACTCCAGAAATGATGGA | |
| | | GGCACGATGTAGAAAGATGGCACATATATCCA | |
| | | AGTATGATAGAGTGGTGAGGAGGGAGATCTGT | |
| | | AGGATCATTACCAAGGGTACACAGACTTACAGT | |
| | | GTGCTGGAAGGTGATCCCTCTGAGAACTACAGT | |
| | | AAGTATCTTCTTAGCCTCAAAGAAAAAGAGGA | |
| | | AGATTCTTCTGGCCATACTCGTGCATATGGTGT | |
| | | GTGCTTTGTTGATACTTCACTGGGAAAGTTTTTC | |
| | | ATAGGTCAGTTTTCAGATGATCGCCATTGTTCG | |
| | | AGATTTAGGACTCTAGTGGCACACTATCCCCCA | |
| | | GTACAAGTTTTATTTGAAAAAGGAAATCTCTCA | |
| | | AAGGAAACTAAAACAATTCTAAAGAGTTCATT | |
| | | GTCCTGTTCTCTTCAGGAAGGTCTGATACCCGG | |
| | | CTCCCAGTTTTGGGATGCATCCAAAACTTTGAG | |
| | | AACTCTCCTTGAGGAAGAATATTTTAGGGAAAA | |
| | | GCTAAGTGATGGCATTGGGGTGATGTTACCCCA | |
| | | GGTGCTTAAAGGTATGACTTCAGAGTCTGATTC | |
| | | CATTGGGTTGACACCAGGAGAGAAAAGTGAAT | |
| | | TGGCCCTCTCTGCTCTAGGTGGTTGTGTCTTCTA | |
| | | CCTCAAAAAATGCCTTATTGATCAGGAGCTTTT | |
| | | ATCAATGGCTAATTTTGAAGAATATATTCCCTT | |
| | | GGATTCTGACACAGTCAGCACTACAAGATCTGG | |
| | | TGCTATCTTCACCAAAGCCTATCAACGAATGGT | |
| | | GCTAGATGCAGTGACATTAAACAACTTGGAGAT | |
| | | TTTTCTGAATGGAACAAATGGTTCTACTGAAGG | |
| | | AACCCTACTAGAGAGGGTTGATACTTGCCATAC | |
| | | TCCTTTTGGTAAGCGGCTCCTAAAGCAATGGCT | |
| | | TTGTGCCCCACTCTGTAACCATTATGCTATTAAT | |
| | | GATCGTCTAGATGCCATAGAAGACCTCATGGTT | |
| | | GTGCCTGACAAAATCTCCGAAGTTGTAGAGCTT | |
| | | CTAAAGAAGCTTCCAGATCTTGAGAGGCTACTC | |
| | | AGTAAAATTCATAATGTTGGGTCTCCCCTGAAG | |
| | | AGTCAGAACCACCCAGACAGCAGGGCTATAAT | |
| | | GTATGAAGAAACTACATACAGCAAGAAGAAGA | |
| | | TTATTGATTTTCTTTCTGCTCTGGAAGGATTCAA | |
| | | AGTAATGTGTAAAATTATAGGGATCATGGAAG | |
| | | AAGTTGCTGATGGTTTTAAGTCTAAAATCCTTA | |
| | | AGCAGGTCATCTCTCTGCAGACAAAAAATCCTG | |
| | | AAGGTCGTTTTCCTGATTTGACTGTAGAATTGA | |
| | | ACCGATGGGATACAGCCTTTGACCATGAAAAG | |
| | | GCTCGAAAGACTGGACTTATTACTCCCAAAGCA | |
| | | GGCTTTGACTCTGATTATGACCAAGCTCTTGCT | |
| | | GACATAAGAGAAAATGAACAGAGCCTCCTGGA | |
| | | ATACCTAGAGAAACAGCGCAACAGAATTGGCT | |
| | | GTAGGACCATAGTCTATTGGGGGATTGGTAGGA | |
| | | ACCGTTACCAGCTGGAAATTCCTGAGAATTTCA | |
| | | CCACTCGCAATTTGCCAGAAGAATACGAGTTGA | |
| | | AATCTACCAAGAAGGGCTGTAAACGATACTGG | |
| | | ACCAAAACTATTGAAAAGAAGTTGGCTAATCTC | |
| | | ATAAATGCTGAAGAACGGAGGGATGTATCATT | |
| | | GAAGGACTGCATGCGGCGACTGTTCTATAACTT | |
| | | TGATAAAAATTACAAGGACTGGCAGTCTGCTGT | |
| | | AGAGTGTATCGCAGTGTTGGATGTTTTACTGTG | |
| | | CCTGGCTAACTATAGTCGAGGGGGTGATGGTCC | |
| | | TATGTGTCGCCCAGTAATTCTGTTGCCGGAAGA | |
| | | TACCCCCCCCTTCTTAGAGCTTAAAGGATCACG | |
| | | CCATCCTTGCATTACGAAGACTTTTTTTGGAGA | |
| | | TGATTTTATTCCTAATGACATTCTAATAGGCTGT | |
| | | GAGGAAGAGGAGCAGGAAAATGGCAAAGCCTA | |
| | | TTGTGTGCTTGTTACTGGACCAAATATGGGGGG | |
| | | CAAGTCTACGCTTATGAGACAGGCTGGCTTATT | |
| | | AGCTGTAATGGCCCAGATGGGTTGTTACGTCCC | |
| | | TGCTGAAGTGTGCAGGCTCACACCAATTGATAG | |
| | | AGTGTTTACTAGACTTGGTGCCTCAGACAGAAT | |
| | | AATGTCAGGTGAAAGTACATTTTTTGTTGAATT | |
| | | AAGTGAAACTGCCAGCATACTCATGCATGCAAC | |
| | | AGCACATTCTCTGGTGCTTGTGGATGAATTAGG | |
| | | AAGAGGTACTGCAACATTTGATGGGACGGCAA | |
| | | TAGCAAATGCAGTTGTTAAAGAACTTGCTGAGA | |
| | | CTATAAAATGTCGTACATTATTTTCAACTCACT | |
| | | ACCATTCATTAGTAGAAGATTATTCTCAAAATG | |
| | | TTGCTGTGCGCCTAGGACATATGGCATGCATGG | |
| | | TAGAAAATGAATGTGAAGACCCCAGCCAGGAG | |
| | | ACTATTACGTTCCTCTATAAATTCATTAAGGGA | |
| | | GCTTGTCCTAAAAGCTATGGCTTTAATGCAGCA | |
| | | AGGCTTGCTAATCTCCCAGAGGAAGTTATTCAA | |
| | | AAGGGACATAGAAAAGCAAGAGAATTTGAGAA | |
| | | GATGAATCAGTCACTACGATTATTTCGGGAAGT | |
| | | TTGCCTGGCTAGTGAAAGGTCAACTGTAGATGC | |
| | | TGAAGCTGTCCATAAATTGCTGACTTTGATTAA | |
| | | GGAATTATAGACTGACTACATTGGAAGCTTTGA | |
| | | GTTGACTTCTGACAAAGGTGGTAAATTCAGACA | |
| | | ACATTATGATCTAATAAACTTTATTTTTTAAAA | |
| | | ATGAAAAAAAAAAAAAAAAAAAAAAAAAAAA | |
| | | AAAAAAAAAAAAAAAAAAAAAAAAAAAAAAA | |
| | | AAAAAAAAAAAAAAAAAAAAAAAAAAAAAAA | |
| | | AAAAAAAAAAAAAAAAAA | |
| PMS2 | NM_000535.6 | AGCCAATGGGAGTTCAGGAGGCGGAGCGCCTG | 4 |
| | | TGGGAGCCCTGGAGGGAACTTTCCCAGTCCCCG | |
| | | AGGCGGATCGGGTGTTGCATCCATGGAGCGAG | |
| | | CTGAGAGCTCGAGTACAGAACCTGCTAAGGCC | |
| | | ATCAAACCTATTGATCGGAAGTCAGTCCATCAG | |
| | | ATTTGCTCTGGGCAGGTGGTACTGAGTCTAAGC | |
| | | ACTGCGGTAAAGGAGTTAGTAGAAAACAGTCT | |
| | | GGATGCTGGTGCCACTAATATTGATCTAAAGCT | |
| | | TAAGGACTATGGAGTGGATCTTATTGAAGTTTC | |
| | | AGACAATGGATGTGGGGTAGAAGAAGAAAACT | |
| | | TCGAAGGCTTAACTCTGAAACATCACACATCTA | |
| | | AGATTCAAGAGTTTGCCGACCTAACTCAGGTTG | |
| | | AAACTTTTGGCTTTCGGGGGGAAGCTCTGAGCT | |
| | | CACTTTGTGCACTGAGCGATGTCACCATTTCTA | |
| | | CCTGCCACGCATCGGCGAAGGTTGGAACTCGAC | |
| | | TGATGTTTGATCACAATGGGAAAATTATCCAGA | |
| | | AAACCCCCTACCCCCGCCCCAGAGGGACCACA | |
| | | GTCAGCGTGCAGCAGTTATTTTCCACACTACCT | |
| | | GTGCGCCATAAGGAATTTCAAAGGAATATTAA | |
| | | GAAGGAGTATGCCAAAATGGTCCAGGTCTTAC | |
| | | ATGCATACTGTATCATTTCAGCAGGCATCCGTG | |
| | | TAAGTTGCACCAATCAGCTTGGACAAGGAAAA | |
| | | CGACAGCCTGTGGTATGCACAGGTGGAAGCCC | |
| | | CAGCATAAAGGAAAATATCGGCTCTGTGTTTGG | |
| | | GCAGAAGCAGTTGCAAAGCCTCATTCCTTTTGT | |
| | | TCAGCTGCCCCCTAGTGACTCCGTGTGTGAAGA | |
| | | GTACGGTTTGAGCTGTTCCGATGCTCTGCATAA | |
| | | TCTTTTTTACATCTCAGGTTTCATTTCACAATGC | |
| | | ACGCATGGAGTTGGAAGGAGTTCAACAGACAG | |
| | | ACAGTTTTTCTTTATCAACCGGCGGCCTTGTGA | |
| | | CCCAGCAAAGGTCTGCAGACTCGTGAATGAGG | |
| | | TCTACCACATGTATAATCGACACCAGTATCCAT | |
| | | TTGTTGTTCTTAACATTTCTGTTGATTCAGAATG | |
| | | CGTTGATATCAATGTTACTCCAGATAAAAGGCA | |
| | | AATTTTGCTACAAGAGGAAAAGCTTTTGTTGGC | |
| | | AGTTTTAAAGACCTCTTTGATAGGAATGTTTGA | |
| | | TAGTGATGTCAACAAGCTAAATGTCAGTCAGCA | |
| | | GCCACTGCTGGATGTTGAAGGTAACTTAATAAA | |
| | | AATGCATGCAGCGGATTTGGAAAAGCCCATGG | |
| | | TAGAAAAGCAGGATCAATCCCCTTCATTAAGGA | |
| | | CTGGAGAAGAAAAAAAAGACGTGTCCATTTCC | |
| | | AGACTGCGAGAGGCCTTTTCTCTTCGTCACACA | |
| | | ACAGAGAACAAGCCTCACAGCCCAAAGACTCC | |
| | | AGAACCAAGAAGGAGCCCTCTAGGACAGAAAA | |
| | | GGGGTATGCTGTCTTCTAGCACTTCAGGTGCCA | |
| | | TCTCTGACAAAGGCGTCCTGAGACCTCAGAAAG | |
| | | AGGCAGTGAGTTCCAGTCACGGACCCAGTGAC | |
| | | CCTACGGACAGAGCGGAGGTGGAGAAGGACTC | |
| | | GGGGCACGGCAGCACTTCCGTGGATTCTGAGG | |
| | | GGTTCAGCATCCCAGACACGGGCAGTCACTGCA | |
| | | GCAGCGAGTATGCGGCCAGCTCCCCAGGGGAC | |
| | | AGGGGCTCGCAGGAACATGTGGACTCTCAGGA | |
| | | GAAAGCGCCTAAAACTGACGACTCTTTTTCAGA | |
| | | TGTGGACTGCCATTCAAACCAGGAAGATACCG | |
| | | GATGTAAATTTCGAGTTTTGCCTCAGCCAACTA | |
| | | ATCTCGCAACCCCAAACACAAAGCGTTTTAAAA | |
| | | AAGAAGAAATTCTTTCCAGTTCTGACATTTGTC | |
| | | AAAAGTTAGTAAATACTCAGGACATGTCAGCCT | |
| | | CTCAGGTTGATGTAGCTGTGAAAATTAATAAGA | |
| | | AAGTTGTGCCCCTGGACTTTTCTATGAGTTCTTT | |
| | | AGCTAAACGAATAAAGCAGTTACATCATGAAG | |
| | | CACAGCAAAGTGAAGGGGAACAGAATTACAGG | |
| | | AAGTTTAGGGCAAAGATTTGTCCTGGAGAAAAT | |
| | | CAAGCAGCCGAAGATGAACTAAGAAAAGAGAT | |
| | | AAGTAAAACGATGTTTGCAGAAATGGAAATCA | |
| | | TTGGTCAGTTTAACCTGGGATTTATAATAACCA | |
| | | AACTGAATGAGGATATCTTCATAGTGGACCAGC | |
| | | ATGCCACGGACGAGAAGTATAACTTCGAGATG | |
| | | CTGCAGCAGCACACCGTGCTCCAGGGGCAGAG | |
| | | GCTCATAGCACCTCAGACTCTCAACTTAACTGC | |
| | | TGTTAATGAAGCTGTTCTGATAGAAAATCTGGA | |
| | | AATATTTAGAAAGAATGGCTTTGATTTTGTTAT | |
| | | CGATGAAAATGCTCCAGTCACTGAAAGGGCTA | |
| | | AACTGATTTCCTTGCCAACTAGTAAAAACTGGA | |
| | | CCTTCGGACCCCAGGACGTCGATGAACTGATCT | |
| | | TCATGCTGAGCGACAGCCCTGGGGTCATGTGCC | |
| | | GGCCTTCCCGAGTCAAGCAGATGTTTGCCTCCA | |
| | | GAGCCTGCCGGAAGTCGGTGATGATTGGGACT | |
| | | GCTCTTAACACAAGCGAGATGAAGAAACTGAT | |
| | | CACCCACATGGGGGAGATGGACCACCCCTGGA | |
| | | ACTGTCCCCATGGAAGGCCAACCATGAGACAC | |
| | | ATCGCCAACCTGGGTGTCATTTCTCAGAACTGA | |
| | | CCGTAGTCACTGTATGGAATAATTGGTTTTATC | |
| | | GCAGATTTTTATGTTTTGAAAGACAGAGTCTTC | |
| | | ACTAACCTTTTTTGTTTTAAAATGAACCTGCTAC | |
| | | TTAAAAAAAATACACATCACACCCATTTAAAAG | |
| | | TGATCTTGAGAACCTTTTCAAACCAGATGGAGC | |
| | | ATTGCTTGCAAATTTTTTTTCTCTATGTTTGCAT | |
| | | GCGCTCGTGTGTGTGTGTCCAGGCAAGAACACA | |
| | | TTTTATAAAAATAAGAACACTTGGGCTGGGCAT | |
| | | GGTGGCTCATGCCTGTGATCGCAGCACTTTGGG | |
| | | AGGCCGAGGCCGGCGGATCACCTGAGATCAGA | |
| | | AGTTCGAGACCAGCCTGACCAACATGGAGAAA | |
| | | CCCTGCCTCTACTAAAAATACAAAATTAGCCAG | |
| | | GTGTGCTGGCGCATGCCTGTAATCCCCGCTACC | |
| | | CAGGAGGCTGAGGCAGGAGAATCGCTTGAACC | |
| | | CGGGAGACGGAGGTTGCAGTGAACCGAGATTG | |
| | | CGCCACTGCGCTCCAGCCTGGGTGAGATAGAAC | |
| | | AAGACTGTGTCTCAAAAAACAAAACAAAACAA | |
| | | AACAAAAAAAAAAAAACCAAACCACTTTGGAA | |
| | | GTTACTCAGGCCTCTGCTCTGGCTGGACATAGT | |
| | | TTAGTCTATAACTTTCAACCCTTAATGATAATTA | |
| | | AATTCATCTTTGTTTAATTTCATAAATTTAAAAG | |
| | | TAGGGTCCTTTTCAGTTAGTGATTCTCAGCCCTG | |
| | | ATTCACATTAAATTTTTAAACACGGGGGATTCT | |
| | | CTGCCCGGCTGGAAGAAAATGACTGGATGGGA | |
| | | CAGGGGTCACTATTTGAAACATTCCTCTGTGCG | |
| | | GCCAAGGTCGCAAAATGCTGTCCTCGCAGGGG | |
| | | AACAAAAAGAGTTTGATTTCCCATAATTTGATG | |
| | | CTGTGATTTGGTTTCCTCAGGATGTGAACTGTA | |
| | | GAACATTCCAGTTACTGGCCTTGAATGGTTCTG | |
| | | GGAATATAAGAATCCCTGTCTGTCTTTTCAAAT | |
| | | AGTTTTCATGGAACCTTGTCCTGTTTGAACTTGG | |
| | | CTGAAAATGGAAGTAAAGATGCCCTCTTGGGG | |
| | | GCCCAGAGATGACAGATGTGGCTCCCCCTGCTG | |
| | | CCCCCACCCCTTCTCCAGACTGTGGGCGGCTCC | |
| | | CCTTCCTGCTTTAGAATCCCTCAGATGGAGGAG | |
| | | GCAGTACAGTAGTCACTGTGCCATCGTGTCTGG | |
| | | CACTGTGCTGGCGTGGTCTGCAGGATCCCACTT | |
| | | ATGAACTCTCCAGATTGGGAGCTGTGGCAGGAT | |
| | | AACAGCCCCCAAGACAGCTGTGTCCTAATCCCC | |
| | | AGAACCTGTGACCACGCTGCCTCACGTGGCAGA | |
| | | AGGGACTCGGCAGGTGTGATTGAGTGAAGGAT | |
| | | CTTTTTTTTTTTTTTCTTTGAGATGAAGTTTCGCT | |
| | | CTTGTTGCCCAGGCTGGAGTTCAATAGCATGAT | |
| | | CTCAGCTCACTGCAGCCTCTGCCTCCCAGGTTC | |
| | | AAGTGATTCTCCCACCTCAGCCTCCCGAGTAGC | |
| | | TGGGATTACAGGTGTCCAGAACCATACTGGCTA | |
| | | ATTTTTGTATTTTTAGTAGAGACAGGGTTTCACC | |
| | | ATGTTGACCAGGCTGGTCTCGAACTCCTGACCT | |
| | | CAGGTGATCCGACCGCCTCGGCCTCCCAAAGTG | |
| | | CTGGGATTACAGGTGTGAGCCATCATGCCTGGC | |
| | | TGAGTTAAGGATCTTGCAACAGAGAGATTATCC | |
| | | TGGATTGTCTGGGTGGGCCCAGTCCATTGGGTG | |
| | | AGTCCTTCAAAGGTGGAGACCTTTCCCTGCTGG | |
| | | CCAGAGAGAGGCTGTCTTGCTGGTTTTGGAGAT | |
| | | GGAAGGAGGTACCACTAGTCAAGGATTGCAAG | |
| | | CAGTCTCTAGAACAGGGATTCCAACACTCCGGA | |
| | | CACAGACCAGTAGTGGTCCATGGCCTATTAGGA | |
| | | AGTGGGGTGCACAGCAGGTTAGGGGCCGGCAA | |
| | | GCCAGCGAAGCTTCATCTGTATTTATAGCCACT | |
| | | CCCCGTCGCTGGCGTTACCACCCGAGCTCCGCC | |
| | | TCCTGTCACATCAGCGGTGGGCATTAGATTCTC | |
| | | ATAGCAGCACGAGCCCTATTGTGAACTGCACAC | |
| | | ACGAGGGATGTAGGTTGCACGCTCCTTATGAGA | |
| | | ATCTGATGCCTGATGATCTGTCACTGTCTCCCGT | |
| | | CACCCCCAGATGGGGCTGTCTAGTTGCAGGAAA | |
| | | ACAAGCTCAGGGCTCCCACTGAGTCTCTGTGAT | |
| | | GGTGAGTTGTAGAATTATTTAATTATATGTTAC | |
| | | AATGTAATAATAGTAGAAATAAAGTGCACAAT | |
| | | AAATGCAATGCACTTGAATCGTCCTGAAACCAT | |
| | | CCCTCCCCGACCCCAATCCATGGAAAAATTGTG | |
| | | TTCCGCGAAACCGGTCTCTGGTGCCAAAAAGGT | |
| | | TGGGGACCGCTTCTGGAAAAGCTGGAAAAGGC | |
| | | AAGAAAACGCATTCTCTCCCTCAGCCTCTGGAA | |
| | | GGAACCAGCACTGTGGGACTAATTTACATACTG | |
| | | TAGGGTAATAAATTTGTGTTGCTTCGAACCACT | |
| | | AAAAAAAAA | |
| EPM2AIP1 | NM_014805.3 | GCTTGCGCGTTAGAGATCGCTGTCCGCTCTTCC | 5 |
| | | TATTGGTTCGTTTTTAGGAGCTCGGGGAATACG | |
| | | AAATATCCAGCCAATAGGAGCAGAGATGCCGG | |
| | | AACCGGGCTTGTGTGCCTCTGCTGAGGTGATCT | |
| | | GGCGCAGAGCGGAGGAGGTGCTTGGCGCTTCT | |
| | | CAGGCTCCTCCTCTCCCCTTGCGGCCTTTCTAAC | |
| | | GTTGGCCCTGCTCTTGTGGCCTCCCGCAGAATG | |
| | | TGGATGACGCCCAAAAGAAGCAAGATGGAAGT | |
| | | CGACGAGGCTCTAGTTTTCCGGCCCGAGTGGAC | |
| | | CCAGCGTTATTTGGTGGTGGAGCCTCCGGAGGG | |
| | | CGATGGGGCCCTGTGCCTGGTCTGTCGCCGCCT | |
| | | CATCGTAGCTACCCGCGAACGCGACGTCAGGC | |
| | | GCCACTACGAGGCTGAGCACGAATACTACGAG | |
| | | CGGTATGTGGCGGACGGCGAGCGCGCGGCCCT | |
| | | GGTGGAGCGTCTGCGTCAGGGCGACTTGCCCGT | |
| | | GGCCTCCTTCACTCCTGAAGAGAGAGCTGCTCG | |
| | | TGCAGGCCTCGGGCTCTGCCGCCTCTTGGCCTT | |
| | | GAAGGGTCGCGGCTGGGGTGAGGGGGACTTTG | |
| | | TATACCAGTGCATGGAGGTGTTGCTGAGAGAG | |
| | | GTACTGCCCGAGCATGTAAGCGTCCTGCAAGGC | |
| | | GTTGACTTATCTCCAGATATCACAAGGCAGAGG | |
| | | ATCCTGAGCATTGACAGGAATCTACGCAACCAG | |
| | | CTTTTTAACCGAGCCAGGGACTTTAAAGCCTAT | |
| | | TCTCTTGCCTTGGACGACCAGGCTTTTGTGGCCT | |
| | | ATGAGAACTACCTCCTGGTCTTTATCCGCGGTG | |
| | | TAGGCCCTGAGTTGGAGGTGCAAGAAGATCTTC | |
| | | TGACCATAATCAACCTGACTCATCATTTCAGTG | |
| | | TTGGTGCGCTCATGTCGGCAATCCTAGAGTCCC | |
| | | TGCAGACAGCAGGGCTTAGCTTGCAGAGAATG | |
| | | GTTGGACTGACCACGACCCATACTTTGAGGATG | |
| | | ATTGGTGAGAACTCAGGACTCGTCTCATACATG | |
| | | AGAGAAAAGGCCGTAAGCCCCAACTGTTGGAA | |
| | | TGTCATTCATTATTCAGGATTTCTTCACTTGGAA | |
| | | CTGTTGAGCTCCTATGATGTAGATGTTAATCAG | |
| | | ATCATAAATACCATATCCGAATGGATAGTTTTG | |
| | | ATTAAGACCAGAGGCGTTAGGCGACCTGAATTT | |
| | | CAGACTTTACTAACGGAATCTGAATCAGAGCAT | |
| | | GGTGAAAGGGTTAATGGACGATGTCTGAACAA | |
| | | TTGGCTTAGGAGAGGGAAAACTTTAAAACTAAT | |
| | | ATTCTCTCTAAGAAAAGAAATGGAAGCGTTCTT | |
| | | GGTTTCAGTAGGGGCAACAACAGTCCACTTCTC | |
| | | AGACAAACAATGGCTTTGTGACTTTGGCTTCTT | |
| | | GGTGGACATTATGGAACACCTTCGAGAACTCAG | |
| | | TGAAGAATTACGAGTTAGTAAAGTCTTTGCTGC | |
| | | TGCTGCCTTTGACCATATTTGTACTTTCGAAGTT | |
| | | AAGCTGAATTTATTTCAAAGACATATTGAGGAA | |
| | | AAAAATCTAACAGACTTTCCTGCCCTCAGAGAA | |
| | | GTTGTTGATGAGCTAAAACAGCAAAATAAGGA | |
| | | AGATGAAAAAATATTTGATCCTGATAGGTATCA | |
| | | AATGGTGATCTGTCGTCTCCAAAAAGAATTTGA | |
| | | GAGACATTTTAAGGACCTCAGGTTCATTAAAAA | |
| | | GGACTTAGAACTTTTTTCAAATCCATTTAACTTT | |
| | | AAACCTGAATATGCACCTATTTCAGTGAGGGTG | |
| | | GAGCTAACAAAACTTCAGGCAAACACTAATCTT | |
| | | TGGAATGAATACAGAATCAAAGACTTGGGGCA | |
| | | GTTTTATGCTGGATTGTCTGCTGAATCCTACCCA | |
| | | ATTATCAAAGGGGTTGCCTGTAAGGTGGCATCC | |
| | | TTGTTTGATAGTAACCAAATCTGTGAAAAGGCT | |
| | | TTTTCATATTTGACTCGAAACCAACACACTTTG | |
| | | AGTCAGCCATTAACAGATGAGCATCTCCAAGCC | |
| | | CTGTTTCGGGTTGCCACAACTGAAATGGAGCCC | |
| | | GGTTGGGATGACCTTGTGAGAGAAAGAAATGA | |
| | | ATCTAATCCATAAGGCTTTGTAGTACAAGATTG | |
| | | AAAAACTCAACAAGAATTTAATTCTAAAAGCA | |
| | | AAAATTGGTTTGAGTTTTCAAGTTTACTAATTTG | |
| | | GATTGTGAGAAAGTACCAAGTACCAGCCGTCC | |
| | | AAACTGATCACAATTAAAATTCTGACAGTTGCC | |
| | | TTTTTTTTCATCTCAAATGGCAGCATGGGACTG | |
| | | AAACATGAGAATGCCACCTTTTTTAAAACTTAG | |
| | | TTTAGTGACAAAGTCATTGTCTTTTATGATATA | |
| | | GTTAATTTTAAAGAGATTTAGTATTAATGTGAG | |
| | | TTGAATTTGCAGTCTGTTTTTTAGGTGTTCTGAA | |
| | | GATAAATGCCAAAAATTTCAGCTCTTATTTTAA | |
| | | TGGAGTGTTAAAATTCTGATTCATATAGTCTTA | |
| | | AATTATCAACTCCTTAAATGTGCTTTTGAACCA | |
| | | ATTTGCAGAAGCTCACATAGCAAGTTCATAAGT | |
| | | TTCCAAAAAGGAAGCCCATACATAACAGTGGA | |
| | | GGTGTTTTGTCTAACCATCAAAATGTTTGAGAC | |
| | | TTTTTTTTAAACATTTCTGAGTTCGAAGGTAATA | |
| | | CTGACAGATTTCTTCCCTCTTCCCTCCCCATCAC | |
| | | CCACCTCAGTGATAACACATTACTGATAGAGGA | |
| | | AGTCATTAGAATCATTTTTAAGTTTCAGATATA | |
| | | GGAGACTTCATGCAATTTGGAGATAAGACTAAT | |
| | | TATTGGGGGTTTTCCTTGGATTTTTTTTTTAATA | |
| | | ACTGGGGGCTATTTTATCAGCTTGCCTATTAAA | |
| | | GGACTATGGTAAGTATAGAATCTTAATGGTTGC | |
| | | CAGTTAGTAATTCTTTTTTTTTTTTTTTTTACTGT | |
| | | AGACACAAGTTTGGCCCTATCAAAAACGATGA | |
| | | GGAAAAAAGATTGCACTCCAGGATTAGGAGGT | |
| | | GTGAGATATTTTAGCTTTTTTGTCTTATCTGCGT | |
| | | GGGTATTGCTGCTTTATTTTAAAAAATCCTGCCT | |
| | | AAAGTAAACACTTTGTTTTAAAATGATACAGTA | |
| | | TCAGATTTTGTTAGATGCTAGAAATGGATTTAT | |
| | | TCTAAAATTTGGAACTGTCGTACACATTCTATA | |
| | | TGTAAGATAGCACACAAGTAGAAATATTTAAA | |
| | | AGCAGTCTTATTCACAGATTGCAGTAATTCTGT | |
| | | ATTTCTACTAAGATAATCTGCTTTGTGCCAAAA | |
| | | CAGTAATTTCCAAACTTCTGTTCACCATGAAAA | |
| | | GGCAATCTTAAAGTTCATTATGTAAAACTAATT | |
| | | ATAAACAGGACCCAATTTATATTCATAGATCCT | |
| | | CTCAAGTATTATACAATTTAAAAACTCTTGTTC | |
| | | CAAAGTCCTGTCTTAACTATTGAAACACCTTAA | |
| | | TCTGTGGTTACTAATCCAGCAAATTCAAGGAAC | |
| | | CAGGCTATGACTAAGAATTTAGGTGGAATTGAT | |
| | | GTCTGGGCAATTAAAATAAATGGCATAAGAGC | |
| | | TTAAAAACCAAAGTIGTGCCAGTGGCTTTCAAC | |
| | | TAGAGGCAGTAACCTGTCATTCCAGAGGATGCT | |
| | | GAGAAATGTGTAGGGGCACTTTTTTGGTTGTCA | |
| | | TATTTACTAGGGGCTTCTGTTGGCATTTAAGCCT | |
| | | AAAGACACTCACCCCTGCAGTGCATGGGACAG | |
| | | CCTGGCACAATGAAGAATTAGCCCTCCCAAAAT | |
| | | GTAGATTATTTTATTTCAAGGGATAGGGCAGAT | |
| | | TACCATTAGAAGCAAAATTAAAAGTACAAGCT | |
| | | GGGCAAACTGACAGAATACTAGATAGGAGAGA | |
| | | CTAATTCCAACCTTCTAAATTTGGCTAGTAAAG | |
| | | TGCAATAAAGGCATTGATAAGTTCTGTTAGCTC | |
| | | ACCATAGCACTTGTAAATCAGGAATTAATAATT | |
| | | GAATCAGATTTAAGGGCTCTGTCCTGTTATACA | |
| | | TATTTAAGGCAGAAAAAAAGTTACATGTCGATT | |
| | | AGGTACTTATCAAGAATGGTCAAGCTGAGATTT | |
| | | TGGTTAATAGAGTAAGCTTACATATCTAGAGAA | |
| | | ACAACATAGTGGAAAACCGAAAAAAAAAAACA | |
| | | GAAAAATCTACCGGTAATTTCCCAATAGCTTTG | |
| | | AATATTCACAGCAGAGCTTTATTACTTGAGAGA | |
| | | AAGACTGGAAGACCTGAAAGCCACTTCTGCTTT | |
| | | CTAACCCCAGTTCCTTAAATATTGAAATCTTGT | |
| | | ACATTTTGTGAAATTCCAGTATGTTTTGCTTAAG | |
| | | GTGTTAATAAAATTAGTTTGCATCATGTAGTCA | |
| | | TTGAGTGAGGGGGAGATATAAGCCAAGGATTT | |
| | | TAAATTGACCCTTAGCTATAGAGAATTTGCTAT | |
| | | AAGCTAGTCTTGTTTGTAAAAAAAAAAAAAAA | |
| | | AAAAGAAAAAGAAAAAAGTGTATTTTACTGTTT | |
| | | TCTGTATTAAGTAATTCTGTAACTGCATGGCAG | |
| | | TCTTTTTTTTTTTAAATAAATATAGTTGTTACTG | |
| | | GTCCTGTTGTAGCAGTGAATATAGTTAAAATAC | |
| | | GTACATTAAAAAAAAAATTATTAGGTCCTTACC | |
| | | AGTTACTGTCCTATAGCTCATTCCTACTAGTTTT | |
| | | CTTGACAGATTTGTATTCCCAGTGTCCCGTATTG | |
| | | CCACTCAAATTGCTCTACTATGCTAAGTCCTTGT | |
| | | TAATAGTCTTACCCTCCTTGAAACACTTGAACA | |
| | | CTTGATGACTTTAGCTTTGAGGAGATACCATCT | |
| | | CCAGGTGTGCTTTCTTAGTCTTTGCAGGCACCTC | |
| | | TTCCCTTCAATATCTGTTCTTCGTATTTTTAAAA | |
| | | AAATTTGTTTTAGACTGCCTTGTTCTGTGTCAGC | |
| | | TCGCTAGCTGATCTCATTTCCTTCCATGGTTTCC | |
| | | TTACCATTTATATGCAAATGACTGTCAGATTCA | |
| | | TATCTCCTTTCTAGATCTTCCCTAATTGATGTAT | |
| | | CTAATTGCTAACAAATGCTCTTTGCTGTCTCAG | |
| | | GCACTACATGTCATTGATCTTGCCCCCAATCCT | |
| | | GCTCCTCCTCTCATGTTTCCTCTTTGACTAAATG | |
| | | GCATTACCACTACCAACCATTCATTTGTCCTTTT | |
| | | TACCAATTCTCCAATGCTGCCATTTTAATTCAG | |
| | | GCCATCAACCTACCTAAATTATAGCAACAGCCT | |
| | | CCTTATTAGTCTCCCTGTTTTTTATTTTTATTCCT | |
| | | TTCTACACTACAACCAAATTGCTCCAAAAGACT | |
| | | TACTGATCATGTCACTGCATTGCTTTCACCATTG | |
| | | CTCTTAGGGTACAATACAAATTTATCTTCATCTT | |
| | | TAAGGTCTCAGTATGCCACTTCATCTAGGAAAC | |
| | | CTTCATTGATGCCCTCTAGATTAGGTGCCCTTAC | |
| | | TATCCATTCCCTATACACCCTGTTCTTTCCCAGA | |
| | | CATACACTTGGCACACTTTATTGTTACTGCTTAT | |
| | | TGATCACTGCTAGACTGTAAGCTTTGTAAGGGC | |
| | | AGGGACCATATAAGCCTTGTTCACTGTTATATC | |
| | | TCTAGTGCTTAGCACAATGCCTGGCATTTCAAT | |
| | | AAATGTTTGGACAAACGAATATTTGTGTAGTGT | |
| | | TTTACAATTTTTGAAGCTCTTTCACAGTCTTATT | |
| | | TGACCTTCACAGTCATTCTGCCTTAGACTGTCC | |
| | | ATTGGGTAACTTTTATCCACATATTACTAATTG | |
| | | AAAAATGAAGACAAGTTCTTTGTAACTAGGGA | |
| | | CCTCGTTGTATTCTCAGAATTTAGTGTAGTGCTT | |
| | | AGCATGTGACTTAAATATGTATTATGTGACTGT | |
| | | TAAACAAATTGTGGTTTTCTCTGTTGTATGAAA | |
| | | GGAGAGAAGGATAACAAATTGCGGTTTTCCCTG | |
| | | GTAAACACAGTAAGTAGTAAACTCAGGATTCA | |
| | | AAACCAAATATACACACCAAATCCACTATGTAA | |
| | | TATTAAGTTTGCATATCCATGTATAGAATCTTAT | |
| | | TTTTTTTTACCCTTTGTAAACAGTGTCATATATA | |
| | | TATATATATTTTTTTTTTTTTTTTAAATTTCCAAA | |
| | | GGAACCTACATATAGAGGGAAAAGATTAGACA | |
| | | ACTACTTAGTGAACTAAAACAATATGTTTTTAC | |
| | | TAAATGTTACATTTAGTATTGGAAAAAGATAAT | |
| | | GCCGCCTAAGAGTTAATAATCATTTTTCCTTTTG | |
| | | TAGGCATCAACACTAGGAGAAAATGGCATGCT | |
| | | ATTTACTTGCTACTTTCCTTTACAGATGATTTTT | |
| | | GGCTCTTCTGGGATTTAAAAGTAAGTAAATTTA | |
| | | ACAAAGTAGAAGACTGACTCAGCCCTTCTGGTC | |
| | | ACTATATATTCAGTTCACTTGTTTTTACACCTGC | |
| | | AGAATGTCCTTATCACCCAAAGGGAGATGACCC | |
| | | AAAAGTGACATCTAGTTAATGTATACTTCTAAA | |
| | | GTTTGCTGTATTCCTTTGCCTTCTTGTTCCCATG | |
| | | CCTCTCTGAACTTAATTTCTGGGTAACTGAGGC | |
| | | TTTTCAGGCTTAGGTGGGAAAGCCACACCCTTA | |
| | | GTCTGTTTCCTTAAGCCATTTTGACCAATTTATG | |
| | | GGATTAACTAGTATAATCTTAGTTGGAGTTTTA | |
| | | GTCTGAGGCATATTAAGTCATTCAGAGATCTTA | |
| | | ACAGTAGGTGTCATAGTCATCCAGTGATTTGGT | |
| | | GCTTGCTGCAAAACTGGCTTTTTTTTTTTTTTTTT | |
| | | TTTTTGAGGCGGGGTCTCACTTTGTCACCCAGG | |
| | | CTGGAGTGCAGAGGTACAATCTCAGCTCAATGC | |
| | | AATCTCTGCCTTCCTGGCTCAAGCAATTCTCCC | |
| | | ACTGCAGCCTCCTAAGTAGCTGGGAATACAGGT | |
| | | ATACACGAGTACACCCAGCTAATTTTTGTATTT | |
| | | TTATGTGGAGACAGGGTCTTGCTGTATTCCCCA | |
| | | GGCTAGTCTCGAATTCCTGGACTCAAGCAGTCC | |
| | | GCCCGCCTCGGGCTCCCAAAATGTTGGTGTTAT | |
| | | ACGTGTGAGCCTCTGCACCCGGCGGCAAAACTG | |
| | | GCTTTTAATCAACCTTTTGGCTAAAGGATTTCTC | |
| | | TTTTTATTTATTTGTAAAAGGATTTCCCATTTTT | |
| | | ATCTTTCTTTTTGATATTAAAATGTTGCCTCATC | |
| | | CTACCCAGTAAGTACTTGAATTTGAATTCTCTTC | |
| | | CTTTTCATTTTTGCCTGCAAACTGACCAGTCTTT | |
| | | TCTGAGTTCATCTCTTCTGTACGTTTTGTCAAGT | |
| | | GCAGTGAACAGCAACTACAAAATATTTTGTTTT | |
| | | TCTGTCTTTTTCTTTAGTAAAGGGTAGATGATCT | |
| | | GCCTTTCAGGTTATCTCAAGGGGCAGTTTCACC | |
| | | TTTCCATAATATAAATTACCCTTGTGTAAGTTAT | |
| | | TTCTTCCATCTTCTGATAGCAATTTCCTGAATGC | |
| | | CTGCCAGCTAACCATTAAGCCAGTGTTCAGTAT | |
| | | TTTAGCATTTTAAAAAACAAGGGACCAATTTCT | |
| | | GTGTCAGCATGGGCTAGCTTGCCATTGAATAAC | |
| | | AAAGGCAAAATCTCACTGTCTCACACAACTTTT | |
| | | CTATTGCAACTTGCCTAGGGACTTTGGTTTAGA | |
| | | TCATAGGTTGGCCATGATCAAACTATGGTCCAT | |
| | | GGGCAAAATCTGTCTAGCTCCTTATTTATCTAA | |
| | | ATAAAGTTTTACTGGAATATA | |
| TTC30A | NM_152275.3 | GCGGCGCCACAGGAACGATGCATGCCGGGACC | 6 |
| | | GGGAAGATTCAGTCTCTGAACGGCCCGGAGTA | |
| | | GTCGTCTTTCCCCTTCTGACTGCCGCCACGCTGC | |
| | | AGTCCAGAATATTTGAAGATCAAACCGAACTTG | |
| | | AGAGACTAACGAGAACGGTCCCTTTTTATTCCT | |
| | | AACAGATTCCTTCCGTGGCAAAGTAACCCGTCG | |
| | | TCTTCCGTTTCCGGTTGCCCGGTTGCCCTGTTGC | |
| | | CGTGGTAACCGCACGCATAACAGCCGTGGTGGT | |
| | | TATGGCTGGTCTGAGCGGCGCGCAGATCCCCGA | |
| | | CGGGGAGTTTACCGCGCTAGTGTACCGGCTCAT | |
| | | CCGCGATGCCCGCTACGCCGAGGCGGTGCAGCT | |
| | | GCTGGGCCGAGAACTGCAGCGGAGCCCCAGGA | |
| | | GCCGTGCCGGCCTGTCGCTGCTAGGCTACTGCT | |
| | | ACTACCGCCTGCAGGAGTTCGCGCTGGCGGCCG | |
| | | AGTGCTATGAGCAGCTGGGCCAGCTGCACCCG | |
| | | GAACTGGAGCAGTACCGCCTGTACCAGGCCCA | |
| | | GGCCCTGTACAAGGCCTGCCTTTATCCGGAGGC | |
| | | CACTCGGGTCGCCTTCCTTCTCCTGGATAACCC | |
| | | CGCCTACCACAGCCGGGTCCTCCGCCTGCAAGC | |
| | | TGCCATCAAGTATAGCGAGGGCGATCTGCCAG | |
| | | GGTCCAGGAGCCTGGTGGAGCAGCTGCTGAGT | |
| | | GGGGAAGGGGGAGAAGAAAGTGGAGGCGACA | |
| | | ATGAGACCGATGGCCAGGTCAACCTGGGTTGTT | |
| | | TGCTCTACAAGGAGGGACAGTATGAAGCTGCA | |
| | | TGCTCCAAGTTTTCTGCCACACTGCAGGCCTCG | |
| | | GGCTACCAGCCTGACCTTTCCTACAACCTGGCT | |
| | | TTGGCCTATTACAGCAGCCGACAGTATGCCTCA | |
| | | GCACTGAAGCATATCGCTGAGATTATTGAGCGT | |
| | | GGCATCCGCCAGCATCCTGAGCTAGGTGTGGGC | |
| | | ATGACCACCGAGGGCTTTGATGTTCGCAGTGTT | |
| | | GGCAACACCTTAGTTCTCCATCAGACTGCTCTG | |
| | | GTGGAAGCCTTCAACCTTAAGGCAGCCATAGA | |
| | | ATACCAACTGAGAAACTATGAGGTAGCTCAAG | |
| | | AAACCCTCACCGACATGCCACCCAGGGCAGAG | |
| | | GAAGAGTTGGACCCTGTGACCCTGCACAACCA | |
| | | GGCACTAATGAACATGGATGCCAGGCCTACAG | |
| | | AAGGGTTTGAAAAGCTACAGTTTTTGCTCCAAC | |
| | | AGAATCCCTTTCCTCCAGAGACTTTTGGCAACC | |
| | | TGTTGCTGCTCTACTGTAAATATGAGTATTTTGA | |
| | | CCTGGCAGCAGATGTCCTGGCAGAAAATGCCC | |
| | | ATTTGACGTATAAGTTCCTCACACCCTATCTCTA | |
| | | TGACTTCTTAGATGCCCTGATCACTTGCCAGAC | |
| | | AGCTCCTGAAGAGGCTTTCATTAAGCTTGATGG | |
| | | GCTAGCAGGGATGCTGACTGAGCAGCTTCGGA | |
| | | GACTCACCAAGCAAGTACAGGAAGCAAGACAC | |
| | | AACAGAGATGATGAAGCTATCAAAAAGGCAGT | |
| | | GAATGAATATGATGAAACCATGGAGAAATACA | |
| | | TTCCTGTGTTGATGGCTCAGGCAAAAATCTACT | |
| | | GGAATCTTGAAAATTATCCAATGGTGGAAAAG | |
| | | GTCTTCCGCAAATCTGTGGAATTCTGTAACGAC | |
| | | CATGATGTGTGGAAGTTGAATGTGGCTCATGTT | |
| | | CTGTTCATGCAGGAAAACAAATACAAAGAAGC | |
| | | CATTGGTTTCTATGAACCCATAGTCAAGAAGCA | |
| | | TTATGATAACATCCTGAATGTCAGTGCTATTGT | |
| | | ACTGGCTAATCTCTGTGTTTCCTATATTATGACA | |
| | | AGTCAAAATGAAGAAGCAGAGGAGTTGATGAG | |
| | | GAAGATTGAAAAGGAGGAAGAGCAGCTCTCTT | |
| | | ATGATGACCCAAATCGGAAAATGTACCATCTCT | |
| | | GCATTGTGAATTTGGTGATAGGAACTCTTTATT | |
| | | GTGCCAAAGGAAACTATGAGTTTGGTATTTCTC | |
| | | GAGTTATCAAAAGCTTGGAGCCTTATAATAAAA | |
| | | AGCTGGGAACAGATACCTGGTATTATGCCAAA | |
| | | AGATGCTTCCTGTCCTTGTTAGAAAACATGTCA | |
| | | AAACACATGATAGTCATTCATGACAGTGTTATT | |
| | | CAAGAATGTGTCCAGTTTTTAGGACACTGTGAA | |
| | | CTTTATGGCACAAACATACCTGCTGTTATTGAA | |
| | | CAACCCCTCGAAGAAGAAAGAATGCATGTTGG | |
| | | GAAGAATACAGTCACAGATGAGTCCAGACAAT | |
| | | TGAAAGCTTTGATTTATGAGATTATAGGATGGA | |
| | | ATAAGTAGTTATGACTGATAGTGGCTTTTTTCA | |
| | | AAATGGCTTTCTTACGTACCACACTTTTTTTTAT | |
| | | CTGTATTTAGCCTTGGCATCTTTATATTTGTCTT | |
| | | ATTTTGAATCTTATCCACTTTGTAAGAACAAGT | |
| | | TTATGTTTGAGCAACTTTTTCATTTAATCCAGAA | |
| | | GGGTAGGGACTATGCAGTGTAAGCTGCATCACT | |
| | | TCTGCTTTCTTCCTACTAGTGACAATCATCTGGT | |
| | | CTTGCCCTCAAGCAACAATTGCTAGAGTAACAT | |
| | | CTTTGTATAAGCAAGTAACCCCAGATAGAGTTG | |
| | | ACGTTTCAGCTTTGGGCTGTCAAAAGGGTATGT | |
| | | CATGGACCAAAGCACTGTTAGTACGGGTATGTT | |
| | | TGCATTTGGTCACTGATATGTAAATGACTGCTA | |
| | | GCCCACGGCTGGACCACTTCTCAATCAGCAAAT | |
| | | AAAGCCATGTCTATTTTGCTATCTCAGCATAGA | |
| | | CTATGCTGTCTGATAAATCTAATTCTTAACTCTA | |
| | | TTTCTCCAGTTTTTTAGTCCTTTAACTTTCTGGA | |
| | | TTGCAACGAAGTCTAGTTTAGACCTCTAAGCCC | |
| | | TTTTAGAAGTACAAGTATAATGGGAATTTCTTT | |
| | | TCTTGGTTCTTTTCAGGTTATGAGGTTTGGTCAG | |
| | | TGACAAAATTTTTTTTCATAATTTGGTTGATTGG | |
| | | TTGCTTCTTAAGTTTTATAATAAACGTTTTTCTT | |
| | | CATGTTCTATTTTTGATTTTACAGAAATGATTTT | |
| | | GCCTCCTTGTGGATACTGACATATATTAAGTGT | |
| | | GGAAGCTTATTAATATTTTTGGTTTTTTAAAAAC | |
| | | TGAAATTTTTAATTTTTACTTTTTAATTTTTTAG | |
| | | GAAAAAATAAGCACTGAACTGAGAATGAGAAG | |
| | | AATAAAAGTATGAGTTCCATACCTTCTAATTTT | |
| | | AGGCTGTCAGAAATTCCTTTATTCTTTGGGATTT | |
| | | CACAATCATTTGAACTATCAGAAGCCTTTACAA | |
| | | TTACTTTTAGCTGTAACATCCGATTCTGTATAAG | |
| | | CCACATAGAAAAAAGTTGCCTTTCTTTTTTTATG | |
| | | ACCTGGATATATAAGCAAATCAGCTAGGAAAT | |
| | | ATATAATTGTATTTTATATTAATGTTTTCTAGGA | |
| | | TTTTGGCTTACAGTAAATGTTAACCCCTATGGT | |
| | | AAGTGATTGTTATTGTTGGATGTTATACTGATT | |
| | | ATTAATAAGAAATTTGGATTTTTGCCTTTTTACC | |
| | | TGGAATTTTTGCTTACAGCCGTAGCTATGAATA | |
| | | TATATAGGGTGGTCCCCAGTCTCTGTTATGGTT | |
| | | GCGCATAAATTAATAATTTTATAAGTATTTAGA | |
| | | AATGGTATAATTCTCTTAACTTCCTCTTTCAGTT | |
| | | TTTGTACTAATGTTTGTTTTTGTTCGGGAAGAGG | |
| | | AGATTTGCTTTTAATCCTTCCAAAAAATGATGA | |
| | | ACCACCGTTCCATTCAGTAGTTTGACAAGCTGT | |
| | | TATAATGTGTATTTTTTCCTCAATTATTCTTGAA | |
| | | ATATTTAGAGCCTCTCCTGCTTCTAACATGAAG | |
| | | GCCTTTAGATGCCAGTCTGCCAGAAATCTGGAA | |
| | | ACAGAGGAACCGGTGAAGTGAAGATGTAATGG | |
| | | AGATTTAGCTAATGATGTACTTCACAATCCACC | |
| | | TTGGATCTCCTGCATGTCCAAATCTCAGTAGTT | |
| | | AATCAAGTGTCTGCTGCCATTAACAGAACAGAA | |
| | | GTAATGGATAACAGAATGGAAATAAGAGATGC | |
| | | CAGAACTACTTCCATAACTAACTCACCAAATCA | |
| | | AATCATCAGTCCTCATATTCTTGTTTTATTTAAT | |
| | | ACAAGGAGAAGAGGCCATGCACTTTCCAAAAG | |
| | | GTCAAAGCCACATAGAATAGGAAGGCAATCTC | |
| | | TAGTTTAAAGCTTTCTCTTGGAGTGTTTTCTCCC | |
| | | CCTGTCTTCAAAGGGTCTACTTGAGAGATAGTG | |
| | | GTGTTTACTGCTGCAGCATGTATCACAAGATAA | |
| | | GAAATGAAAAATCAATCTTTCTTACCACCCTGT | |
| | | TCTCTTTCCCTTTTTTATCTTTTCCCTTTTGTCAA | |
| | | TTATAGAATTATAGGGACATTTTTCTCTGATAG | |
| | | CTGGAAGTTGAACCTCAACCAGGTATAAAAGA | |
| | | TGCATAACAACCTTTTAGCAGTAAGTGTCAAGT | |
| | | GAGTGAGCACTATGATTATCAAGGTGACTTTGG | |
| | | AAACCTTTTAAAAATGCATTTTTGCAAAACAAG | |
| | | ATAACATATATTGATAAAAAGTGACTCTCAGAT | |
| | | TGGTAATGCCAGAAAAAATTTTAAGAGGACTC | |
| | | ACCAAAAGTACTAGATCTATGTAAGTTGTAGAA | |
| | | TAGAGTGAAGTTTTTTTATATATTTGTGGTAGCC | |
| | | TCCATCTTTTAAACTTTTTGAACTCAGTAGAAA | |
| | | AACAGACTGAAATTTTAAAGACATGCAGTATTT | |
| | | GTATCATTTTAAATTCTGTAACACTGGGAATTA | |
| | | AATATACTCAACTTTAGAGGAAAAAAAAAAAA | |
| | | AAAAAA | |
| SMAP1 | NM_001044305.2 | GACCCAGTCCCCCTCCCCCTCCCCTCGCCGGCT | 7 |
| | | AGGGTGGTGCGTGCCGGCAGGCCGGTCAAGGA | |
| | | GGCGGGACACGTCGGCGCTACCACCGCCACCG | |
| | | CCGCCGCCGCCCCTCCTCCCGTTCCAGCTGCCG | |
| | | CTGCCGCTTCCTGGGCTGAGTCCGCCCGCGGTC | |
| | | CCGGCGGCGCCAGGTGCGTTCACTCTGCCCGGC | |
| | | TCCAGCCAGCGTCCGCCGCCGCCGTAGCTGCCC | |
| | | CAGGCTCCCCGCCCCGCTGCCGAGATGGCGACG | |
| | | CGCTCCTGTCGGGAGAAGGCTCAGAAGCTGAA | |
| | | CGAGCAGCACCAGCTCATCCTATCCAAGCTTCT | |
| | | GAGGGAGGAGGACAACAAGTACTGCGCCGACT | |
| | | GCGAGGCCAAAGGTCCTCGATGGGCTTCCTGGA | |
| | | ATATTGGTGTGTTTATTTGCATCAGATGTGCTG | |
| | | GAATTCATAGAAATCTTGGGGTTCATATATCCA | |
| | | GGGTCAAATCAGTCAACCTAGACCAATGGACA | |
| | | GCAGAACAGATACAGTGCATGCAAGATATGGG | |
| | | AAATACTAAAGCAAGACTACTCTATGAAGCCA | |
| | | ATCTTCCAGAGAACTTTCGAAGACCACAGACAG | |
| | | ATCAAGCAGTGGAATTTTTCATCAGAGATAAAT | |
| | | ATGAAAAGAAGAAATACTACGATAAAAATGCC | |
| | | ATAGCTATTACAAATATTTCCTCCTCTGATGCTC | |
| | | CTCTTCAGCCTTTGGTATCCTCTCCTTCTCTGCA | |
| | | AGCTGCTGTTGACAAAAATAAATTGGAGAAAG | |
| | | AAAAGGAAAAAAAAAAGGAAGAGAAAAAGAG | |
| | | AGAAAAGGAGCCAGAAAAGCCGGCAAAACCAC | |
| | | TTACAGCTGAAAAGCTGCAGAAGAAAGATCAG | |
| | | CAACTGGAGCCTAAAAAAAGTACCAGCCCTAA | |
| | | AAAAGCTGCGGAGCCCACTGTGGATCTTTTAGG | |
| | | ACTTGATGGCCCTGCTGTGGCACCAGTGACCAA | |
| | | CGGGAACACAACGGTGCCACCCCTGAACGATG | |
| | | ATCTGGACATCTTTGGACCGATGATTTCTAATC | |
| | | CCTTACCTGCAACTGTCATGCCCCCAGCTCAGG | |
| | | GGACACCCTCTGCACCAGCAGCTGCAACCCTGT | |
| | | CTACAGTAACATCTGGGGATCTAGATTTATTCA | |
| | | CTGAGCAAACTACAAAATCAGAAGAAGTGGCA | |
| | | AAGAAACAACTTTCCAAAGACTCCATCTTATCT | |
| | | CTGTATGGCACAGGAACCATTCAACAGCAAAG | |
| | | TACTCCTGGTGTATTTATGGGACCCACAAATAT | |
| | | ACCATTTACCTCACAAGCACCAGCTGCATTTCA | |
| | | GGGCTTTCCATCGATGGGCGTGCCTGTGCCTGC | |
| | | AGCTCCTGGCCTTATAGGAAATGTGATGGGACA | |
| | | GAGTCCAAGCATGATGGTGGGCATGCCCATGCC | |
| | | CAATGGGTTTATGGGAAATGCACAAACTGGTGT | |
| | | GATGCCACTTCCTCAGAACGTTGTTGGCCCCCA | |
| | | AGGAGGAATGGTGGGACAAATGGGTGCACCCC | |
| | | AGAGTAAGTTTGGCCTGCCGCAAGCTCAGCAGC | |
| | | CCCAGTGGAGCCTCTCACAGATGAATCAGCAG | |
| | | ATGGCTGGCATGAGTATCAGTAGTGCAACCCCT | |
| | | ACTGCAGGTTTTGGCCAGCCCTCCAGCACAACA | |
| | | GCAGGATGGTCTGGAAGCTCATCAGGTCAGACT | |
| | | CTCAGCACACAACTGTGGAAATGAAAACTGCA | |
| | | ATACAAGTTTCATCCAGAACTACCACCTGACAT | |
| | | TCCTTGCTGAAACGCATCTAGTTCCCCTGTTTAT | |
| | | TCATATGCATATTTTTTTTCTTTTTACCCATTTGT | |
| | | TCATATTAAGAATGATCTGATTGACCGTGTTGG | |
| | | TCTGTACTGATTCAATTTGATGTGGTGAAAAGC | |
| | | AGGTTGATAAATCATTTTATGTCAAGGGCAGCT | |
| | | TTGCTCATATTTCCCATGATTTCATGTACTGCAT | |
| | | TATTTGAGAAGCTGCTCAACTTGCAAAATCAGT | |
| | | TTTCCTCTCAATAAAATTATAGCTCTAATGTTTG | |
| | | CATATAAGGGAAGTAGTTATCATGTTAGTAATA | |
| | | CCTCTAATAGTATAAACCCCACCCCAAAATTAG | |
| | | CCAGTAATCCTGTAGGAAGGTACTGTATGATCA | |
| | | AATGTTTAATCATATAAATAGAATGTAAATGTC | |
| | | TCACTGAGCACTGTTTTCTAGTGTATCAAAATG | |
| | | CTCTTATTTCATCATTCACTTCACTGTGCTGTTG | |
| | | TTATGATGTGCTTAACAGGGAACGTGATTAGTG | |
| | | AAAGGAAGATAAACGTGGATGTTACTCCAAAA | |
| | | CTTCGTTTAATGAATGCTTAAAGAATTCAAATT | |
| | | TTATCTGCCTCTCTTGTAATTTGGATCTCTTCTT | |
| | | AATGTACATAGTGCTAACATGAAGACCTTTTTC | |
| | | TGCACTATATGCAAACAGGGTAACTAACTAAA | |
| | | ACAAAGCCACTTTCAATCTTCAATCCTTGAAGG | |
| | | TATATCTAGGTTTATGACAGTAATTGTGTTTAC | |
| | | ATTTTATGGTGCCTAGTATTGACAAAATGTTAT | |
| | | TTCCCTACATTAAACATGACTCCATAGACCTTTT | |
| | | CATTTGTGGGTTTTTATTTCCTATGATGTATACT | |
| | | GCCACTAACCTTCCAAAAATTACTTAGTATTGC | |
| | | AAAGTCAGGAATCATCAGGAACGTTTAGCTGA | |
| | | CAAAATACTTGTCTGTTTTAAAAACCTGTTCAA | |
| | | GTCTACCAACCTGTTCAAGTCTACCAATTATAA | |
| | | GGGCAAATTGGAGAAAAAGAAAAAATATATAC | |
| | | TCAAGAGTGGTATCTTGCAGTATCGGCACTGTA | |
| | | CAAAAAAATCTTCCAATTTAGTTGTTGTAGAGA | |
| | | AAACATGCAGAACAAATGAAGACAAAACATAC | |
| | | ATTTTGTACCAACCATCCAATTAGCTTATGTTA | |
| | | ACTGACAAGCTCCATTTAAACAGATGTCCATCA | |
| | | GATGACAAGAAAGGCTGCTGTACTGAAGTAAA | |
| | | ACAAACAATACCTGAATGCTCTGTAGCCTAAAC | |
| | | TCCAAACATCCTCTTCCATATGGATCCACTGGC | |
| | | TGGACAAACTGCACCAGTTGCTGCTTCAATTTA | |
| | | TACCTCAATTTTCACTGTGTCCAGGTGGTACTTT | |
| | | GGCTCGTTGGCTAGATTAACCTTCTCTGTCCGA | |
| | | GTGTGCCACACGAGAACCTGAAGGGGAAGGAA | |
| | | ATAGCTTGGGTAGCGCACTCTTCATGGTGACAC | |
| | | TCGAGGTCGGGCAGCACAAGTGTAATGAATAC | |
| | | CTTAGTGCAGTTATTTGCTTTCGGTTCCAGTTCT | |
| | | TCGACTGTTGTTATCTGTTTGAGAAAGTCAGAT | |
| | | TCTTGCATCCCTGGCTGGGATCCACGACGCTTA | |
| | | AATACAGCTTTTGGATTGGACAAAATGACTTGA | |
| | | AGACTTACAGCAAATCCTTTGTGAAAAATAAAA | |
| | | AAAAAAAAGAGACTTTAAAAAAAAAAAAAAA | |
| RNLS | NM_001031709.2 | AAAGCTCAGGGCCCAGGTCGGCCCAGGGAGCA | 8 |
| | | CGGAACCAAAGAGCGCTAGCGCCGGTTCGGCC | |
| | | GCCTTTCCAGAAAGCCCGGGCCGAACGGCCCC | |
| | | GCCGCAGAGACTCAGCGCGGATCGCTGCTCCCT | |
| | | CTCGCCATGGCGCAGGTGCTGATCGTGGGCGCC | |
| | | GGGATGACAGGAAGCTTGTGCGCTGCGCTGCTG | |
| | | AGGAGGCAGACGTCCGGTCCCTTGTACCTTGCT | |
| | | GTGTGGGACAAGGCTGAGGACTCAGGGGGAAG | |
| | | AATGACTACAGCCTGCAGTCCTCATAATCCTCA | |
| | | GTGCACAGCTGACTTGGGTGCTCAGTACATCAC | |
| | | CTGCACTCCTCATTATGCCAAAAAACACCAACG | |
| | | TTTTTATGATGAACTGTTAGCCTATGGCGTTTTG | |
| | | AGGCCTCTAAGCTCGCCTATTGAAGGAATGGTG | |
| | | ATGAAAGAAGGAGACTGTAACTTTGTGGCACCT | |
| | | CAAGGAATTTCTTCAATTATTAAGCATTACTTG | |
| | | AAAGAATCAGGTGCAGAAGTCTACTTCAGACA | |
| | | TCGTGTGACACAGATCAACCTAAGAGATGACA | |
| | | AATGGGAAGTATCCAAACAAACAGGCTCCCCT | |
| | | GAGCAGTTTGATCTTATTGTTCTCACAATGCCA | |
| | | GTTCCTGAGATTCTGCAGCTTCAAGGTGACATC | |
| | | ACCACCTTAATTAGTGAATGCCAAAGGCAGCA | |
| | | ACTGGAGGCTGTGAGCTACTCCTCTCGATATGC | |
| | | TCTGGGCCTCTTTTATGAAGCTGGTACGAAGAT | |
| | | TGATGTCCCTTGGGCTGGGCAGTACATCACCAG | |
| | | TAATCCCTGCATACGCTTCGTCTCCATTGATAAT | |
| | | AAGAAGCGCAATATAGAGTCATCAGAAATTGG | |
| | | GCCTTCCCTCGTGATTCACACCACTGTCCCATTT | |
| | | GGAGTTACATACTTGGAACACAGCATTGAGGAT | |
| | | GTGCAAGAGTTAGTCTTCCAGCAGCTGGAAAAC | |
| | | ATTTTGCCGGGTTTGCCTCAGCCAATTGCTACC | |
| | | AAATGCCAAAAATGGAGACATTCACAGGTTAC | |
| | | AAATGCTGCTGCCAACTGTCCTGGCCAAATGAC | |
| | | TCTGCATCACAAACCTTTCCTTGCATGTGGAGG | |
| | | GGATGGATTTACTCAGTCCAACTTTGATGGCTG | |
| | | CATCACTTCTGCCCTATGTGTTCTGGAAGCTTTA | |
| | | AAGAATTATATTTAGTGCCTATATCCTTATTCTC | |
| | | TACATGTGTATTGGGTTTTTATTTTCACAATTTT | |
| | | CTGTTATTGATTATTTTGTTTTCTATTTTGCTAA | |
| | | GAAAAATTACTGGAAAATTGTTCTTCACTTATT | |
| | | ATCATTTTTCATGTGGAGTATAAAATCAATTTT | |
| | | GTAATTTTGATAGTTACAACCCATGCTAGAATG | |
| | | GAAATTCCTCACACCTTGCACCTTCCCTACTTTT | |
| | | CTGAATTGCTATGACTACTCCTTGTTGGAGGAA | |
| | | AAGTGGTACTTAAAAAATAACAAACGACTCTCT | |
| | | CAAAAAAATTACATTAAATCACAATAACAGTTT | |
| | | GTGTGCCAAAAACTTGATTATCCTTATGAAAAT | |
| | | TTCAATTCTGAATAAAGAATAATCACATTATCA | |
| | | AAGCCCCATCTTAAGTCTTCGGATGTGTCCTTG | |
| | | AATCAATATTTTTGCAAATTATACAAAACAAGA | |
| | | TTTTTCCAAAATGTAGGTAACAGAGTGTAATTC | |
| | | TTATTTCTCATTTATCCCCCAAGTTATTAAGTGA | |
| | | TCCTGAATTGTAGGTCATATATGTCATCATCTTA | |
| | | GTGTGGAGGGCAACTTGACTGATAAAGAGACC | |
| | | TTCCTTCAGATTTTCAGAAAGTATAAGATTCCA | |
| | | CATGATTTTCCCAGCCACACAGTACTTTTTAACT | |
| | | TTCAAACAAATTCCAGTCCTAATATGAAAGATA | |
| | | AAAATTAAATAGAAACAGAGAGAAAGTATATC | |
| | | GATCCTTACCTTTTGCTATATTTTATAGCTGTTG | |
| | | CTGTTACTTTATGGGTTCTCCAGTATGTGCTGTG | |
| | | GCATTTAGACTGTGTCGAGTTTAATGAATTTAA | |
| | | CACAACAAAAAATTTACTGAACCAGAAAATAG | |
| | | ATGCACTTAAAATAGTTCAATATTTGCCAAGTT | |
| | | GGTGGTTCAGCATATCACCCACATGCTTCAGTG | |
| | | ACCTGACCCCACGACTTGCTAGCTGGAGAGAA | |
| | | ATCAATCTCCAGCCTTCCAAACCAGCTACCTGT | |
| | | TGCTAATTTGAAAAGCAAAATGATGAGTTCTAT | |
| | | TTCAGCATTTTGAAAGGAGAAAAATCATTGCAG | |
| | | CCTCTCAAACTAACAAAAGTTCAACAAAAGACT | |
| | | TCTTACTGTAATAGTGTTTAAAGTTTCACACTTA | |
| | | CATGTCCACTGTCATACATACACATACACAGGC | |
| | | ACAGGCAGAACTTGCTTCTATAGCTGCAAAGTG | |
| | | GGTTTTATGACCCTATAGCATATTATTATATGTT | |
| | | TCCTCTTAGCAATAAATTGGTGAAAAACTTAAA | |
| | | TGCCAAAAAA | |
| WNT11 | XM_011545241.2 | CCGGGCCTTTGCCGACATGCGCTGGAACTGCTC | 9 |
| | | CTCCATTGAGCTCGCCCCCAACTATTTGCTTGA | |
| | | CCTGGAGAGAGGACACCAGCCACTGGCCTAGG | |
| | | GCCCACCCTGATCCGGTATGACCTCGTCTCAGC | |
| | | CCCATTACATCTGCAAAGACCCCACTTCGTCAT | |
| | | AAGATTATGCTCACAGGGACCCGGGAGTCGGC | |
| | | CTTCGTGTATGCGCTGTCGGCCGCCGCCATCAG | |
| | | CCACGCCATCGCCCGGGCCTGCACCTCCGGCGA | |
| | | CCTGCCCGGCTGCTCCTGCGGCCCCGTCCCAGG | |
| | | TGAGCCACCCGGGCCCGGGAACCGCTGGGGAG | |
| | | GATGTGCGGACAACCTCAGCTACGGGCTCCTCA | |
| | | TGGGGGCCAAGTTTTCCGATGCTCCTATGAAGG | |
| | | TGAAAAAAACAGGATCCCAAGCCAATAAACTG | |
| | | ATGCGTCTACACAACAGTGAAGTGGGGAGACA | |
| | | GGCTCTGCGCGCCTCTCTGGAAATGAAGTGTAA | |
| | | GTGCCATGGGGTGTCTGGCTCCTGCTCCATCCG | |
| | | CACCTGCTGGAAGGGGCTGCAGGAGCTGCAGG | |
| | | ATGTGGCTGCTGACCTCAAGACCCGATACCTGT | |
| | | CGGCCACCAAGGTAGTGCACCGACCCATGGGC | |
| | | ACCCGCAAGCACCTGGTGCCCAAGGACCTGGA | |
| | | TATCCGGCCTGTGAAGGACTCGGAACTCGTCTA | |
| | | TCTGCAGAGCTCACCTGACTTCTGCATGAAGAA | |
| | | TGAGAAGGTGGGCTCCCACGGGACACAAGACA | |
| | | GGCAGTGCAACAAGACATCCAACGGAAGCGAC | |
| | | AGCTGCGACCTTATGTGCTGCGGGCGTGGCTAC | |
| | | AACCCCTACACAGACCGCGTGGTCGAGCGGTG | |
| | | CCACTGTAAGTACCACTGGTGCTGCTACGTCAC | |
| | | CTGCCGCAGGTGTGAGCGTACCGTGGAGCGCTA | |
| | | TGTCTGCAAGTGAGGCCCTGCCCTCCGCCCCAC | |
| | | GCAGGAGCGAGGACTCTGCTCAAGGACCCTCA | |
| | | GCAACTGGGGCCAGGGGCCTGGAGACACTCCA | |
| | | TGGAGCTCTGCTTGTGAATTCCAGATGCCAGGC | |
| | | ATGGGAGGCGGCTTGTGCTTTGCCTTCACTTGG | |
| | | AAGCCACCAGGAACAGAAGGTCTGGCCACCCT | |
| | | GGAAGGAGGGCAGGACATCAAAGGAAACCGAC | |
| | | AAGATTAAAAATAACTTGGCAGCCTGAGGCTCT | |
| | | GGAGTGCCCACAGGCTGGTGTAAGGAGCGGGG | |
| | | CTTGGGATCGGTGAGACTGATACAGACTTGACC | |
| | | TTTCAGGGCCACAGAGACCAGCCTCCGGGAAG | |
| | | GGGTCTGCCCGCCTTCTTCAGAATGTTCTGCGG | |
| | | GACCCCCTGGCCCACCCTGGGGTCTGAGCCTGC | |
| | | TGGGCCCACCACATGGAATCACTAGCTTGGGTT | |
| | | GTAAATGTTTTCTTTTGTTTTTTGCTTTTTCTTCC | |
| | | TTTGGGATGTGGAAGCTACAGAAATATTTATAA | |
| | | AACATAGCTTTTTCTTTGGGGTGGCACTTCTCA | |
| | | ATTCCTCTTTATATATTTTATATATATAAATATA | |
| | | TATGTATATATATAATGATCTCTATTTTAAAACT | |
| | | AGCTTTTTAAGCAGCTGTATGAAATAAATGCTG | |
| | | AGTGAGCCCCAGCCCGCCCCTGCA | |
| SFXN1 | NM_001322977.1 | CGGACGCGCGCTCACAGGCGCGCGCGAGGACG | 10 |
| | | CGCTCCGGGGACGCGCGAGGACGCCGTGGCGG | |
| | | GAGAAGCGTTTCCGGTGGCGGCGGAGGCTGCA | |
| | | CTGAGCGGGACCTGCGAGCAGCGCGGGCGGCA | |
| | | GCCCGGGGGAAGCGGTGAGTCGCGGGCGGCAG | |
| | | GCCCAGCCAGTCCGGGACCATGTCTGGAGAACT | |
| | | ACCACCAAACATTAACATCAAGGAACCTCGAT | |
| | | GGGATCAAAGCACTTTCATTGGACGAGCCAATC | |
| | | ATTTCTTCACTGTAACTGACCCCAGGAACATTC | |
| | | TGTTAACCAACGAACAACTCGAGAGTGCGAGA | |
| | | AAAATAGTACATGATTACAGGCAAGGAATTGTT | |
| | | CCTCCTGGTCTTACAGAAAATGAATTGTGGAGA | |
| | | GCAAAGTACATCTATGATTCAGCTTTTCATCCT | |
| | | GACACTGGTGAGAAGATGATTTTGATAGGAAG | |
| | | AATGTCAGCCCAGGTTCCCATGAACATGACCAT | |
| | | CACAGGTTGTATGATGACGTTTTACAGGACTAC | |
| | | GCCGGCTGTGCTGTTCTGGCAGTGGATTAACCA | |
| | | GTCCTTCAATGCCGTCGTCAATTACACCAACAG | |
| | | AAGTGGAGACGCACCCCTCACTGTCAATGAGTT | |
| | | GGGAACAGCTTACGTTTCTGCAACAACTGGTGC | |
| | | CGTAGCAACAGCTCTAGGACTCAATGCATTGAC | |
| | | CAAGCATGTCTCACCACTGATAGGACGTTTTGT | |
| | | TCCCTTTGCTGCCGTAGCTGCTGCTAATTGCATT | |
| | | AATATTCCATTAATGAGGCAAAGGGAACTCAA | |
| | | AGTTGGCATTCCCGTCACGGATGAGAATGGGA | |
| | | ACCGCTTGGGGGAGTCGGCGAACGCTGCGAAA | |
| | | CAAGCCATCACGCAAGTTGTCGTGTCCAGGATT | |
| | | CTCATGGCAGCCCCTGGCATGGCCATCCCTCCA | |
| | | TTCATTATGAACACTTTGGAAAAGAAAGCCTTT | |
| | | TTGAAGAGGTTCCCATGGATGAGTGCACCCATT | |
| | | CAAGTTGGGTTAGTTGGCTTCTGTTTGGTGTTTG | |
| | | CTACACCCCTGTGTTGTGCCCTGTTTCCTCAGAA | |
| | | AAGTTCCATGTCTGTGACAAGCTTGGAGGCCGA | |
| | | GTTGCAAGCTAAGATCCAAGAGAGCCATCCTG | |
| | | AATTGCGACGCGTGTACTTCAATAAGGGATTGT | |
| | | AAAGCAGGGAGGAAACCTCTGCAGCTCATTCT | |
| | | GCCACTGCAAAGCTGGTGTAGCCATGCTGGTGA | |
| | | GAAAAATCCTGTTCAACCTGGGTTCTCCCAGTT | |
| | | ACGGAAACCTTTTAAAGATCCACATTAGCCTTT | |
| | | TAGAATAAAGCTGCTACTTTAACAGAGCACCTG | |
| | | GCGTGGGCCAAGTGCCTGATACTCCCTTACACT | |
| | | GAATCATGTTATGATTTATAGAAATACCTTTCC | |
| | | TGTAGCTTTTATAGTCATTGTTTTTCAAAGACGA | |
| | | TATACCAGCCCTCACCCAGGTTTTAAAAAAGCA | |
| | | CTGGTAGGCATAGAATAGGTGCTCAGTATATGG | |
| | | TCAGTAAATGTTCTATTGATTATCAATCAGTGA | |
| | | AAAAAGAAATCTGTTTAAAATACTGAATTTTCA | |
| | | TCTCACTCCCATTGCAAATCAAGGAGATCTCAG | |
| | | CAGTGAACTGGGAAAATACAAAAGCTCTGGGC | |
| | | TAATCTATAAAAACTTACCCTGAAATATTAAGG | |
| | | GCAGTTTGCTTCTAGTTTGGGGATTGCGCTAGC | |
| | | CCAATGAAGGTGATGAAGCTTTTGGATTTGGAG | |
| | | GGTAAAAGCTCCTTCACACCCCTTCCAAAAGTC | |
| | | AGTCACAGACCACTGCAACATGCCTTCCCTGCT | |
| | | GGATCATTATATACATTCAGATTGTGAGTGGAT | |
| | | TGCCTTGGTTGACTTTTAATTTATTGTTTTTTGTT | |
| | | CTTATAAAGATGATAATCTTACCTTGCAGTTAT | |
| | | TGACTTTATATTCAATTATTTACATCAAATAATG | |
| | | AAATAACTGAAATGTACAAATGTCAAATTTTGG | |
| | | AAGTATATTCAATACCAATGCTGTATGAGTGGG | |
| | | CTGAATCCAGTTCATTGTTTTTTTTTTGGTAAGA | |
| | | AGTGAGACTACAGTTCCAGCTACCTACATGTCT | |
| | | TTTCTTGTCATCCTTATAGATCTCTTTGGCTTTC | |
| | | AGAAAGATACAGTGATAATGTGTGTATGAATC | |
| | | AGTCACAATGAATTTTACTTGAATATTGTATGT | |
| | | TGCATTCCACTTCATTTGAAAATAATGAAACCA | |
| | | TGTACCACTGTTTACATCATCTGTAGTGATTTCA | |
| | | TAGATAATATATTTAATATGACAGATTATGTTT | |
| | | CAACTCTGTAGATGTTTAACGTCATAGACAGTT | |
| | | GGCCCTCTGTATCCGTGAGCTCTATATCTGTGA | |
| | | ATTCAACCAAGTTTGGATGGAAAATTTTTTTTTT | |
| | | TTTTTTTTTTTTGAGACGGAGTCTCGCTCTGTCA | |
| | | CCCAGGCTGGAGTGCAGTGGCGTAGTCTCGGCT | |
| | | CACTGCAAGCTCCGCCTCCCGGGTTCACGCCGT | |
| | | TCTCCTGCCTCAGCCTCTCTGAGAAGCTGGGAC | |
| | | TACAGGCGCCCGCCACCACGCCCGGCTAATTTT | |
| | | TTTGTATTTTTAGTAGAGACGGGGTTTCACTGT | |
| | | GGTCTCGATCTCCTGACCTCGTGATCCGCCCGC | |
| | | CTTGGCCTCCCAAGGTGCTGGGATTACAAGCGT | |
| | | GAGCCACCGCACCCGGCCTGAAAATATTTTCTA | |
| | | AAAAGATAAAAAATATACATAACGATGAAAAA | |
| | | TAATACAAATTTAAAAACCAATACAGTATAACA | |
| | | ACTATTTACATAGTGCTTACATTGTATTAGGTGT | |
| | | TATAAGCAATCTAGAGATGATTTAGCAAGTATA | |
| | | CAGGAGGATGTGCCTAGGTTATATGCAAATACT | |
| | | GTGCCATTTTATATCAGGAACTTGAGCATCTGC | |
| | | AGATATTGGTATCGGAGGGCGGTCCTGGAACC | |
| | | AAGCATCCACGGATACTGAGGGGTGACATTTCA | |
| | | TGAAGTGTAGATCATTGTATTCAGAGATTGTAA | |
| | | ATGAAAAAAATATAGAAACTATTTAGTTTTGGT | |
| | | AGATTTTTTTTCTGACAATGTGACCAGACTGAA | |
| | | TTTCCTCATAAAGAAAAAATGGCGTGCCTTGTG | |
| | | TCTGTGTTTCTCTTTTCTCTGAAAGGATTAATAG | |
| | | ATCTGAAGCTTTGGGCCACTCAGAGCCTTCCTT | |
| | | GATGCTGCCAGAGTCTTCTTATTTAGATTTTCTG | |
| | | TCTTAAACCATTGGAAGCAAAACGGTTTTCCCA | |
| | | TGACATTCTGGCCTTGGACAGATTCTGTTGTCCT | |
| | | CGACGCGTCTCTTTATAAAGTGGTAAAAGCCTG | |
| | | AAATTCAGGGCAGCTCTCCATGAGGTGCTGAAG | |
| | | GGCTCTTTTCATAAGAAGCTAAGGCACTGCTGC | |
| | | CTGCCCCAGGTGTCCCGCTCCTCTCAGAGTCCT | |
| | | CCCCCTACCAGGTAGTGTGTAGCTCCATTTCAG | |
| | | AATGTTAACCTCCAGTGAAGAGCTAATGACTGG | |
| | | TTAGAAGATTGACAAACTAACCAAAATTTTACA | |
| | | CACTCCGGTTATGTGTGTGAAAGGTTATAAAAG | |
| | | GAATGGCCGGGTGCGGTGGCTCACCCCTGTAAT | |
| | | CCCAGCACTTTGGGAGGCCGAGGCGGGTGGAT | |
| | | CACCTGAGGTCAGGAGTTTGAGACCAGCCTGGC | |
| | | CAACATGGAGAAACCCCGCCTCTACTAAAAAT | |
| | | ACAAAAAATTAGCCAGGCATGGAGGCACATGC | |
| | | CTATAATCCCAGCTACTCGGGAGGCTGAGGTAG | |
| | | GAGAATCGCTTGAATCCGGGAGCTGGAGGTTG | |
| | | CAGTGAGCCAAGATCGCACCATTGCACTCCAGC | |
| | | CTGGGCAACAAGAGCGAAACTCCATCTCAAAA | |
| | | AAAAAAAAAAGAGATTATAAAAGGGATGATGA | |
| | | ACATGGAGCTGCATCTTTTTAAACGTTGTTTTTT | |
| | | GATGCTTCAGACTCTTAATGCTTTTATATAAAG | |
| | | CTATCAACTGTATGTTGATCACAGTTTATAAGA | |
| | | AAGAACAAATCAAGATTGGCAATCCTTGCCGAT | |
| | | CTTTTAGAAATACCTTTTCTGGAGAAAAAAAAA | |
| | | TCCACATGAAGTGCAATAAGCTTATAAAGCTAA | |
| | | GTAGTTATTAATATTTCTATTAACATGATACAA | |
| | | AGGATGATGATTGTAAGTGTTTACTGACTGGCA | |
| | | GCTTTTATTTCAGTATTAGCACAGCGTCTTGCCA | |
| | | GTGTTGGAGGCCATGTATTATTTCAGTTCAACT | |
| | | GGATGAAATGTTAAATAAACTCAGAATGAAAA | |
| | | TAAA | |
| SREBF1 | NM_001005291.1 | AGCAGAGCTGCGGCCGGGGGAACCCAGTTTCC | 11 |
| | | GAGGAACTTTTCGCCGGCGCCGGGCCGCCTCTG | |
| | | AGGCCAGGGCAGGACACGAACGCGCGGAGCGG | |
| | | CGGCGGCGACTGAGAGCCGGGGCCGCGGCGGC | |
| | | GCTCCCTAGGAAGGGCCGTACGAGGCGGCGGG | |
| | | CCCGGCGGGCCTCCCGGAGGAGGCGGCTGCGC | |
| | | CATGGACGAGCCACCCTTCAGCGAGGCGGCTTT | |
| | | GGAGCAGGCGCTGGGCGAGCCGTGCGATCTGG | |
| | | ACGCGGCGCTGCTGACCGACATCGAAGGTGAA | |
| | | GTCGGCGCGGGGAGGGGTAGGGCCAACGGCCT | |
| | | GGACGCCCCAAGGGCGGGCGCAGATCGCGGAG | |
| | | CCATGGATTGCACTTTCGAAGACATGCTTCAGC | |
| | | TTATCAACAACCAAGACAGTGACTTCCCTGGCC | |
| | | TATTTGACCCACCCTATGCTGGGAGTGGGGCAG | |
| | | GGGGCACAGACCCTGCCAGCCCCGATACCAGC | |
| | | TCCCCAGGCAGCTTGTCTCCACCTCCTGCCACA | |
| | | TTGAGCTCCTCTCTTGAAGCCTTCCTGAGCGGG | |
| | | CCGCAGGCAGCGCCCTCACCCCTGTCCCCTCCC | |
| | | CAGCCTGCACCCACTCCATTGAAGATGTACCCG | |
| | | TCCATGCCCGCTTTCTCCCCTGGGCCTGGTATCA | |
| | | AGGAAGAGTCAGTGCCACTGAGCATCCTGCAG | |
| | | ACCCCCACCCCACAGCCCCTGCCAGGGGCCCTC | |
| | | CTGCCACAGAGCTTCCCAGCCCCAGCCCCACCG | |
| | | CAGTTCAGCTCCACCCCTGTGTTAGGCTACCCC | |
| | | AGCCCTCCGGGAGGCTTCTCTACAGGAAGCCCT | |
| | | CCCGGGAACACCCAGCAGCCGCTGCCTGGCCTG | |
| | | CCACTGGCTTCCCCGCCAGGGGTCCCGCCCGTC | |
| | | TCCTTGCACACCCAGGTCCAGAGTGTGGTCCCC | |
| | | CAGCAGCTACTGACAGTCACAGCTGCCCCCACG | |
| | | GCAGCCCCTGTAACGACCACTGTGACCTCGCAG | |
| | | ATCCAGCAGGTCCCGGTCCTGCTGCAGCCCCAC | |
| | | TTCATCAAGGCAGACTCGCTGCTTCTGACAGCC | |
| | | ATGAAGACAGACGGAGCCACTGTGAAGGCGGC | |
| | | AGGTCTCAGTCCCCTGGTCTCTGGCACCACTGT | |
| | | GCAGACAGGGCCTTTGCCGACCCTGGTGAGTGG | |
| | | CGGAACCATCTTGGCAACAGTCCCACTGGTCGT | |
| | | AGATGCGGAGAAGCTGCCTATCAACCGGCTCG | |
| | | CAGCTGGCAGCAAGGCCCCGGCCTCTGCCCAG | |
| | | AGCCGTGGAGAGAAGCGCACAGCCCACAACGC | |
| | | CATTGAGAAGCGCTACCGCTCCTCCATCAATGA | |
| | | CAAAATCATTGAGCTCAAGGATCTGGTGGTGGG | |
| | | CACTGAGGCAAAGCTGAATAAATCTGCTGTCTT | |
| | | GCGCAAGGCCATCGACTACATTCGCTTTCTGCA | |
| | | ACACAGCAACCAGAAACTCAAGCAGGAGAACC | |
| | | TAAGTCTGCGCACTGCTGTCCACAAAAGCAAAT | |
| | | CTCTGAAGGATCTGGTGTCGGCCTGTGGCAGTG | |
| | | GAGGGAACACAGACGTGCTCATGGAGGGCGTG | |
| | | AAGACTGAGGTGGAGGACACACTGACCCCACC | |
| | | CCCCTCGGATGCTGGCTCACCTTTCCAGAGCAG | |
| | | CCCCTTGTCCCTTGGCAGCAGGGGCAGTGGCAG | |
| | | CGGTGGCAGTGGCAGTGACTCGGAGCCTGACA | |
| | | GCCCAGTCTTTGAGGACAGCAAGGCAAAGCCA | |
| | | GAGCAGCGGCCGTCTCTGCACAGCCGGGGCAT | |
| | | GCTGGACCGCTCCCGCCTGGCCCTGTGCACGCT | |
| | | CGTCTTCCTCTGCCTGTCCTGCAACCCCTTGGCC | |
| | | TCCTTGCTGGGGGCCCGGGGGCTTCCCAGCCCC | |
| | | TCAGATACCACCAGCGTCTACCATAGCCCTGGG | |
| | | CGCAACGTGCTGGGCACCGAGAGCAGAGATGG | |
| | | CCCTGGCTGGGCCCAGTGGCTGCTGCCCCCAGT | |
| | | GGTCTGGCTGCTCAATGGGCTGTTGGTGCTCGT | |
| | | CTCCTTGGTGCTTCTCTTTGTCTACGGTGAGCCA | |
| | | GTCACACGGCCCCACTCAGGCCCCGCCGTGTAC | |
| | | TTCTGGAGGCATCGCAAGCAGGCTGACCTGGAC | |
| | | CTGGCCCGGGGAGACTTTGCCCAGGCTGCCCAG | |
| | | CAGCTGTGGCTGGCCCTGCGGGCACTGGGCCGG | |
| | | CCCCTGCCCACCTCCCACCTGGACCTGGCTTGT | |
| | | AGCCTCCTCTGGAACCTCATCCGTCACCTGCTG | |
| | | CAGCGTCTCTGGGTGGGCCGCTGGCTGGCAGGC | |
| | | CGGGCAGGGGGCCTGCAGCAGGACTGTGCTCT | |
| | | GCGAGTGGATGCTAGCGCCAGCGCCCGAGACG | |
| | | CAGCCCTGGTCTACCATAAGCTGCACCAGCTGC | |
| | | ACACCATGGGGAAGCACACAGGCGGGCACCTC | |
| | | ACTGCCACCAACCTGGCGCTGAGTGCCCTGAAC | |
| | | CTGGCAGAGTGTGCAGGGGATGCCGTGTCTGTG | |
| | | GCGACGCTGGCCGAGATCTATGTGGCGGCTGCA | |
| | | TTGAGAGTGAAGACCAGTCTCCCACGGGCCTTG | |
| | | CATTTTCTGACACGCTTCTTCCTGAGCAGTGCCC | |
| | | GCCAGGCCTGCCTGGCACAGAGTGGCTCAGTGC | |
| | | CTCCTGCCATGCAGTGGCTCTGCCACCCCGTGG | |
| | | GCCACCGTTTCTTCGTGGATGGGGACTGGTCCG | |
| | | TGCTCAGTACCCCATGGGAGAGCCTGTACAGCT | |
| | | TGGCCGGGAACCCAGTGGACCCCCTGGCCCAG | |
| | | GTGACTCAGCTATTCCGGGAACATCTCTTAGAG | |
| | | CGAGCACTGAACTGTGTGACCCAGCCCAACCCC | |
| | | AGCCCTGGGTCAGCTGATGGGGACAAGGAATT | |
| | | CTCGGATGCCCTCGGGTACCTGCAGCTGCTGAA | |
| | | CAGCTGTTCTGATGCTGCGGGGGCTCCTGCCTA | |
| | | CAGCTTCTCCATCAGTTCCAGCATGGCCACCAC | |
| | | CACCGGCGTAGACCCGGTGGCCAAGTGGTGGG | |
| | | CCTCTCTGACAGCTGTGGTGATCCACTGGCTGC | |
| | | GGCGGGATGAGGAGGCGGCTGAGCGGCTGTGC | |
| | | CCGCTGGTGGAGCACCTGCCCCGGGTGCTGCAG | |
| | | GAGTCTGAGAGACCCCTGCCCAGGGCAGCTCTG | |
| | | CACTCCTTCAAGGCTGCCCGGGCCCTGCTGGGC | |
| | | TGTGCCAAGGCAGAGTCTGGTCCAGCCAGCCTG | |
| | | ACCATCTGTGAGAAGGCCAGTGGGTACCTGCA | |
| | | GGACAGCCTGGCTACCACACCAGCCAGCAGCT | |
| | | CCATTGACAAGGCCGTGCAGCTGTTCCTGTGTG | |
| | | ACCTGCTTCTTGTGGTGCGCACCAGCCTGTGGC | |
| | | GGCAGCAGCAGCCCCCGGCCCCGGCCCCAGCA | |
| | | GCCCAGGGCACCAGCAGCAGGCCCCAGGCTTC | |
| | | CGCCCTTGAGCTGCGTGGCTTCCAACGGGACCT | |
| | | GAGCAGCCTGAGGCGGCTGGCACAGAGCTTCC | |
| | | GGCCCGCCATGCGGAGGGTGTTCCTACATGAGG | |
| | | CCACGGCCCGGCTGATGGCGGGGGCCAGCCCC | |
| | | ACACGGACACACCAGCTCCTCGACCGCAGTCTG | |
| | | AGGCGGCGGGCAGGCCCCGGTGGCAAAGGAGG | |
| | | CGCGGTGGCGGAGCTGGAGCCGCGGCCCACGC | |
| | | GGCGGGAGCACGCGGAGGCCTTGCTGCTGGCC | |
| | | TCCTGCTACCTGCCCCCCGGCTTCCTGTCGGCG | |
| | | CCCGGGCAGCGCGTGGGCATGCTGGCTGAGGC | |
| | | GGCGCGCACACTCGAGAAGCTTGGCGATCGCC | |
| | | GGCTGCTGCACGACTGTCAGCAGATGCTCATGC | |
| | | GCCTGGGCGGTGGGACCACTGTCACTTCCAGCT | |
| | | AGACCCCGTGTCCCCGGCCTCAGCACCCCTGTC | |
| | | TCTAGCCACTTTGGTCCCGTGCAGCTTCTGTCCT | |
| | | GCGTCGAAGCTTTGAAGGCCGAAGGCAGTGCA | |
| | | AGAGACTCTGGCCTCCACAGTTCGACCTGCGGC | |
| | | TGCTGTGTGCCTTCGCGGTGGAAGGCCCGAGGG | |
| | | GCGCGATCTTGACCCTAAGACCGGCGGCCATGA | |
| | | TGGTGCTGACCTCTGGTGGCCGATCGGGGCACT | |
| | | GCAGGGGCCGAGCCATTTTGGGGGGCCCCCCTC | |
| | | CTTGCTCTGCAGGCACCTTAGTGGCTTTTTTCCT | |
| | | CCTGTGTACAGGGAAGAGAGGGGTACATTTCCC | |
| | | TGTGCTGACGGAAGCCAACTTGGCTTTCCCGGA | |
| | | CTGCAAGCAGGGCTCTGCCCCAGAGGCCTCTCT | |
| | | CTCCGTCGTGGGAGAGAGACGTGTACATAGTGT | |
| | | AGGTCAGCGTGCTTAGCCTCCTGACCTGAGGCT | |
| | | CCTGTGCTACTTTGCCTTTTGCAAACTTTATTTT | |
| | | CATAGATTGAGAAGTTTTGTACAGAGAATTAAA | |
| | | AATGAAATTATTTATAATCTGGAAAAAA | |
| TYMS | NM_001071.1 | GGGGGGGGGGGGACCACTTGGCCTGCCTCCGT | 12 |
| | | CCCGCCGCGCCACTTGGCCTGCCTCCGTCCCGC | |
| | | CGCGCCACTTCGCCTGCCTCCGTCCCCCGCCCG | |
| | | CCGCGCCATGCCTGTGGCCGGCTCGGAGCTGCC | |
| | | GCGCCGGCCCTTGCCCCCCGCCGCACAGGAGCG | |
| | | GGACGCCGAGCCGCGTCCGCCGCACGGGGAGC | |
| | | TGCAGTACCTGGGGCAGATCCAACACATCCTCC | |
| | | GCTGCGGCGTCAGGAAGGACGACCGCACGGGC | |
| | | ACCGGCACCCTGTCGGTATTCGGCATGCAGGCG | |
| | | CGCTACAGCCTGAGAGATGAATTCCCTCTGCTG | |
| | | ACAACCAAACGTGTGTTCTGGAAGGGTGTTTTG | |
| | | GAGGAGTTGCTGTGGTTTATCAAGGGATCCACA | |
| | | AATGCTAAAGAGCTGTCTTCCAAGGGAGTGAA | |
| | | AATCTGGGATGCCAATGGATCCCGAGACTTTTT | |
| | | GGACAGCCTGGGATTCTCCACCAGAGAAGAAG | |
| | | GGGACTTGGGCCCAGTTTATGGCTTCCAGTGGA | |
| | | GGCATTTTGGGGCAGAATACAGAGATATGGAA | |
| | | TCAGATTATTCAGGACAGGGAGTTGACCAACTG | |
| | | CAAAGAGTGATTGACACCATCAAAACCAACCC | |
| | | TGACGACAGAAGAATCATCATGTGCGCTTGGA | |
| | | ATCCAAGAGATCTTCCTCTGATGGCGCTGCCTC | |
| | | CATGCCATGCCCTCTGCCAGTTCTATGTGGTGA | |
| | | ACAGTGAGCTGTCCTGCCAGCTGTACCAGAGAT | |
| | | CGGGAGACATGGGCCTCGGTGTGCCTTTCAACA | |
| | | TCGCCAGCTACGCCCTGCTCACGTACATGATTG | |
| | | CGCACATCACGGGCCTGAAGCCAGGTGACTTTA | |
| | | TACACACTTTGGGAGATGCACATATTTACCTGA | |
| | | ATCACATCGAGCCACTGAAAATTCAGCTTCAGC | |
| | | GAGAACCCAGACCTTTCCCAAAGCTCAGGATTC | |
| | | TTCGAAAAGTTGAGAAAATTGATGACTTCAAAG | |
| | | CTGAAGACTTTCAGATTGAAGGGTACAATCCGC | |
| | | ATCCAACTATTAAAATGGAAATGGCTGTTTAGG | |
| | | GTGCTTTCAAAGGAGCTTGAAGGATATTGTCAG | |
| | | TCTTTAGGGGTTGGGCTGGATGCCGAGGTAAAA | |
| | | GTTCTTTTTGCTCTAAAAGAAAAAGGAACTAGG | |
| | | TCAAAAATCTGTCCGTGACCTATCAGTTATTAA | |
| | | TTTTTAAGGATGTTGCCACTGGCAAATGTAACT | |
| | | GTGCCAGTTCTTTCCATAATAAAAGGCTTTGAG | |
| | | TTAACTCACTGAGGGTATCTGACAATGCTGAGG | |
| | | TTATGAACAAAGTGAGGAGAATGAAATGTATG | |
| | | TGCTCTTAGCAAAAACATGTATGTGCATTTCAA | |
| | | TCCCACGTACTTATAAAGAAGGTTGGTGAATTT | |
| | | CACAAGCTATTTTTGGAATATTTTTAGAATATTT | |
| | | TAAGAATTTCACAAGCTATTCCCTCAAATCTGA | |
| | | GGGAGCTGAGTAACACCATCGATCATGATGTA | |
| | | GAGTGTGGTTATGAACTTTATAGTTGTTTTATAT | |
| | | GTTGCTATAATAAAGAAGTGTTCTGC | |
| EIF5AL1 | NM_001099692.1 | GGGGTCGAGTCAGTGCCGTTTGCGCCAGTTGGA | 13 |
| | | ATCGAAGCCTCTTAAAATGGCAGATGATTTGGA | |
| | | CTTCGAGACAGGAGATGCAGGGGCCTCAGCCA | |
| | | CCTTCCCAATGCAGTGCTCAGCATTACGTAAGA | |
| | | ATGGCTTTGTGGTGCTCAAAGGCTGGCCATGTA | |
| | | AGATCGTGGAGATGTCTGCTTCGAAGACTGGCA | |
| | | AGCACGGCCACGCCAAGGTCCATCTGGTTGGTA | |
| | | TTGACATCTTTACTGGGAAGAAATATGAAGATA | |
| | | TCTGCCCGTCAACTCATAATATGGATGTCCCCA | |
| | | ACATCAAAAGGAATGACTTCCAGCTGATTGGCA | |
| | | TCCAGGATGGGTACCTATCACTGCTCCAGGACA | |
| | | GCGGGGAGGTACCAGAGGACCTTCGTCTCCCTG | |
| | | AGGGAGACCTTGGCAAGGAGATTGAGCAGAAG | |
| | | TACGACTGTGGAGAAGAGATCCTGATCACGGT | |
| | | GCTGTCTGCCATGACAGAGGAGGCAGCTGTTGC | |
| | | AATCAAGGCCATGGCAAAATAACTGGCTCCCA | |
| | | AGGTGGCAGTGGTGGCAGCAGTGATCCTCCGA | |
| | | ACCTGCAGAGGCCCCCTCCCCCAGCCTGGCCTG | |
| | | GCTCTGGCCTGGTCCTAGGTTGGACTCCTCCTA | |
| | | CACAATTTATTTGACGTTTTATTTTGGTTTTCCC | |
| | | CACCCCCTCAATCTGTCAGGGAGCCCCTGCCCT | |
| | | TCACCTAGCTCCCTTGGCCAGGAGCGAGCGAAG | |
| | | CCATGGCCTTGGTGAAGCTGCCCTCCTCTTCTCC | |
| | | CCTCACACTACAGCCCTGGTGGGGGAGAAGGG | |
| | | GGTGGGTGCTGCTTGTGGTTTAGTCTTTTTTTTT | |
| | | TTTTTAAATTCAATCTGGAATCAGAAAGCGGTG | |
| | | GATTCTGGCAAATGGTCCTTGTGCCCTCCCCAC | |
| | | TCATCCTTGGTCTGGTCCCCTGTTGCCCATAGCC | |
| | | CTTTACCCTGAGCACCACCCAACAGACTGGGGA | |
| | | CCAGCCCCCTCGCCTGCCTGTGTCTCTCCCCAA | |
| | | ACCCCTTTAGATGGGGAGGGAAGAAGAGGAGA | |
| | | GGGGAGGGGACCTGCCCCCTCCTCAGGCATCTG | |
| | | GGAAGGGCCTGCCCCCATGGGCTTTACCCTTCC | |
| | | CTGCGGGCTCTCTCCCCGACACATTTGTTAAAA | |
| | | TCAAACCTGAATAAAACTACAAGTTTAATATGA | |
| | | AAAAAAAAAAAAAAGAAAGAAAGACGTGTAA | |
| | | AATGCCAAGAACTCTAGGAAACAGGGACAAAA | |
| | | ACACTTCAAAGAGAAAGTTCATGCACTTGTTTC | |
| | | TGACCACCCAGGGCACCCTTCAGCACACGCTGT | |
| | | CTGGAGTGGCCTGAAGCAAGGAGTGTCTTGTGA | |
| | | GGTGCAGAGGATGCAATGGGAGCAGGGTCCTG | |
| | | TCCCCACCCTAAAGGAGTTCACAGTTTAACGCA | |
| | | AATGAGAAGCCAGTGAGGACATCACTACTCCT | |
| | | GCTGTGAACTTGGGAACTAGAAACACAAAACC | |
| | | TGAGTCTGGAGGGAAGCTAAGGAAGCATTCTG | |
| | | CTCTGGAGTAGACATGAGTGCGTGTGAAGCTTC | |
| | | TGATCTCCCATGAGAGCAATGGGGACATGGGG | |
| | | CAGAATCTAAAACCCATGACTGAAAGCACCAA | |
| | | ATTGCTAAAATGGCAATAAAGAGACATGAGGC | |
| | | CAAGATGGAGAAGAAGGAACCCAGGACGAGG | |
| | | GTCAGCCTCACATTTGGGGCTCATTTCCCTCAG | |
| | | TTTCCTCACTGAATTTCAGAAGGGACTAACTGA | |
| | | GATGCAAAGAAGCAGAGCAGCTTTTGCACCAT | |
| | | GTGGAGGACTAGATGGAAAACAAGTAGACTGA | |
| | | GGGTCTGCTAGTGAAGGTGACCCCTACTGAAGT | |
| | | CCACTGGCTTTGGTTGGGACCCAGAAGAGTCAC | |
| | | ACGCCAGGAATAGAGGTGGACAGGAAACACCC | |
| | | TGACTTTTGTAGGGACTGAACCTCACTGATAAC | |
| | | CTCAATTGCGGATGGTATGGAGGGTGTCTAGGT | |
| | | GTGCTAGGACCCCTGCCCATTCCCCAGAAATAG | |
| | | ACTCCCATCTTTTCTACAGCAAGATAACGTGCT | |
| | | AGTAGGCCTCAATTCATTGCTAAATATTTTTAA | |
| | | CGAGTGTCTTACATTTAGCCAAAAAGACTAGTC | |
| | | ATGTGGCAGGAAAAATACAATGTCATATGACC | |
| | | AAAAGCTAAAAGACTGTGAAAATGAATCCAGA | |
| | | GGTGACCCAAGCATTGAATTTAACAATGCCAGT | |
| | | ACCTGGACCTCCGCTTGCCCCTAAAACATTACA | |
| | | ATCAAGAATGTAGGAAGGGAAAGGAAACACGA | |
| | | AGATTAATCAAGCAGGAAGGACAAGCTCAGTT | |
| | | TTGCACCCACTGAATTTGCCACAAATATTGTGG | |
| | | AAAATATTCTCGGGGACATTGCAGTTGTCTACT | |
| | | TTGGTTGGCACATGGTTCATACAACAGTGTTTG | |
| | | TGTCAGTGAACATCTTACTCTTCCTCGGCAGTCT | |
| | | TTCTTTGCCCAGAGATTTCGCAATGACTGTTGA | |
| | | CCTTCATCATCACCTTTTGGACTTTGGCTTGCAC | |
| | | TTTAGCTTCTGTAGATCTCCATGATGTAAAGAA | |
| | | GTATTTTAGGTCCATTTTAATTCCTGCAAAGGA | |
| | | TAAAATCCTTCTATTTGTGTGCATATAAGTGGA | |
| | | CCTGAGCCCTTGGTTAGGGTGTAGAGAGGAGA | |
| | | AGGGGAGAAACCTGAGGGCCAGAAGCTGTTCT | |
| | | TTCCCTTAAAAGGGCAAACTCATTTCCACACTA | |
| | | TGGGGACTCTGACAGATAGCATACCTTCCTGTC | |
| | | TATGGCTATTGGACCTGCAGGCTTTCCCCTGTA | |
| | | AATCCGTGTTCTGTCATTGACATTTTGTGACTGT | |
| | | AAGACAGACTTGAGATAAGACATCTAGAAAAC | |
| | | AATAATTGAACAATGATGTGAATATATTTCACA | |
| | | CAACTGAACTGTACATTTCAACAAGGTTAAGAT | |
| | | GGTAATTATCACGTTATACATTTTTTACCGCAG | |
| | | GTTAAAATGTTTCACAGGTTGAAAGGAAAGCA | |
| | | ACTACCTTCAGTTCTCTGAGTTCAAGAATTTGT | |
| | | AACATTTCACCCCCTGCTCCTTCCTGATCTTCTG | |
| | | TGGAGCATCTTTTTTCCATCCATGCTCTACTCAG | |
| | | AGCCCACTTTCCCTTCCCTGACACCAGCTTCACT | |
| | | GAGGCTGGTTGGAACCTAACACAAAACATTCTC | |
| | | AGTAATGACTGAATTCCCACAAAGAATTCCATA | |
| | | TAGACTGCATATGAGTTGAATCTTCTAAGACAT | |
| | | GAAATATTTGTTCTCTTCTTGGCTAATATGCAAT | |
| | | GCAAATCCTGTTGCAGATGTACGTCATATACCT | |
| | | CTGAAATTCCTGATGTATTCAATGAAATAACAT | |
| | | CTTTAAAGTTCTGTGTAGAATGTTTTTTTTCTGA | |
| | | TTTCTTCACATACGATAGAAAAAAAAACCCAAA | |
| | | AAAACATGTACTAGGATTTCAATAGAAGCAAT | |
| | | GGGTGATCTAAAAAGATGAAAGAGCAACCGCA | |
| | | TGCGCCCTACAGCTACCGCTAGATTTTATGGGG | |
| | | AAAGCAGCTGGCCCAGTTTGCAGCTAGGAGAA | |
| | | ATGTCAAACACATGAAGAAATGAGAAGCAAAG | |
| | | AAAAACCATGAGGCATGAACATTTCATGGCAA | |
| | | TCACGATGTCCTGGTTTGTGAGATAATGGGATA | |
| | | GAGGAGTAGAAAACAAGGAGAAAGATGAGAA | |
| | | GGTACAAAGTGGTTCAAGTCAAACAGCTCAACT | |
| | | GAACTTTTCTTAATGGAATATTTAAAAAGTGGT | |
| | | ACATTAAAAAACTTCCCCCAGTTCACATCAAAA | |
| | | ATTCTCTCTTCAGGACTAAGTTGGGTAGAGACT | |
| | | GTTCAATGTGCCTAGATATCTTCAGAACTTATA | |
| | | TATTTTCTGTTTTCTACGTATGTTGAAGGGCAGT | |
| | | GCCAAATGATGTGTAATTATCTAGGTTGTAAAA | |
| | | ATAAAACATACTCCCCCTTCCCTTGAGGATAAA | |
| | | AAAAAAAAAAAA | |
| WDR76 | NM_024908.3 | CTGCTCTGGCGCTGCGGCCGCTGGGGATCTGAG | 14 |
| | | TGGGCTCCGCCCCGCCTCGGACCCGCCCCTCCC | |
| | | GGCCTCCCGCCGCAATCTTGGCGGGAAGGCGCC | |
| | | GGCCGCTAAGAAGCCGAAAGATGTCCAGGTCG | |
| | | GGCGCGGCGGCTGAGAAGGCGGACTCCAGACA | |
| | | GCGACCCCAGATGAAGGTAAATGAATATAAAG | |
| | | AAAATCAAAACATCGCTTATGTGTCTCTGAGAC | |
| | | CAGCACAGACTACAGTTTTAATAAAAACAGCTA | |
| | | AGGTCTATCTTGCCCCCTTTTCACTCAGTAATTA | |
| | | CCAGCTAGACCAGCTTATGTGCCCCAAATCCCT | |
| | | ATCAGAAAAGAATTCTAACAATGAAGTGGCGT | |
| | | GTAAGAAGACTAAAATAAAGAAAACTTGCAGA | |
| | | AGGATTATACCTCCAAAGATGAAAAACACATCT | |
| | | TCCAAGGCAGAATCCACGCTGCAAAATTCATCC | |
| | | TCAGCTGTTCATACTGAAAGTAACAAGCTACAA | |
| | | CCCAAGAGAACGGCAGATGCGATGAATCTCAG | |
| | | TGTTGATGTGGAAAGTAGTCAGGATGGAGACA | |
| | | GTGATGAAGATACCACACCATCCCTGGATTTTT | |
| | | CGGGATTGTCACCCTACGAAAGGAAGAGACTG | |
| | | AAGAACATATCAGAAAACGCAGACTTTTTTGCT | |
| | | TCTCTTCAGTTGTCTGAGTCTGCTGCAAGACTCC | |
| | | GTGAAATGATAGAGAAGAGACAGCCTCCTAAA | |
| | | TCCAAAAGAAAGAAGCCTAAGAGAGAAAATGG | |
| | | GATTGGATGTAGAAGGTCAATGCGATTACTAAA | |
| | | AGTTGATCCTTCGGGAGTTTCATTACCAGCAGC | |
| | | TCCAACACCGCCGACATTAGTAGCAGATGAAA | |
| | | CTCCTTTGTTACCTCCTGGGCCTTTAGAAATGAC | |
| | | TTCTGAAAATCAAGAAGACAACAATGAACGAT | |
| | | TTAAAGGATTTCTGCACACATGGGCAGGAATGA | |
| | | GCAAGCCAAGTAGTAAGAACACTGAGAAGGGA | |
| | | TTATCTAGCATTAAAAGCTACAAAGCCAATTTA | |
| | | AATGGCATGGTCATTAGTGAAGATACCGTTTAC | |
| | | AAAGTTACCACAGGCCCAATATTCTCTATGGCT | |
| | | CTCCATCCATCAGAAACTAGAACTTTGGTAGCA | |
| | | GTTGGGGCCAAATTTGGGCAAGTTGGACTTTGT | |
| | | GATTTGACCCAGCAACCTAAAGAAGATGGAGT | |
| | | TTATGTTTTTCATCCCCATAGTCAGCCAGTTAGC | |
| | | TGTCTTTACTTCTCACCCGCCAATCCGGCCCAC | |
| | | ATACTGTCACTGAGCTATGATGGCACGTTACGC | |
| | | TGTGGGGATTTTTCCAGGGCTATTTTTGAAGAG | |
| | | GTGTATAGAAATGAAAGAAGTAGCTTTTCCTCC | |
| | | TTCGACTTCTTGGCAGAAGATGCCTCCACTTTA | |
| | | ATAGTAGGACACTGGGATGGAAATATGTCACT | |
| | | GGTGGATAGACGGACACCTGGAACTTCTTATGA | |
| | | GAAACTTACCAGTTCTTCTATGGGAAAAATAAG | |
| | | AACTGTTCATGTCCACCCAGTGCATAGACAGTA | |
| | | TTTTATCACTGCCGGATTGAGGGATACTCATAT | |
| | | TTATGATGCAAGGCGATTGAATTCCAGGAGAA | |
| | | GTCAGCCTTTGATTTCTTTGACTGAACATACAA | |
| | | AGAGCATTGCTTCCGCCTATTTTTCACCTCTTAC | |
| | | TGGTAACAGAGTGGTGACCACATGTGCTGATTG | |
| | | TAATCTGAGAATTTTTGACAGCAGCTGTATATC | |
| | | TTCTAAGATTCCGCTCCTCACCACCATCAGGCA | |
| | | CAACACTTTCACTGGGCGATGGCTGACCAGGTT | |
| | | CCAAGCCATGTGGGATCCTAAACAAGAAGACT | |
| | | GTGTCATAGTTGGCAGCATGGCCCATCCACGAC | |
| | | GGGTAGAAATCTTCCATGAGACAGGAAAGAGG | |
| | | GTGCATTCGTTTGGTGGAGAATACCTTGTCTCT | |
| | | GTGTGTTCCATCAATGCCATGCACCCAACTCGG | |
| | | TATATTTTGGCTGGAGGTAATTCCAGCGGGAAG | |
| | | ATACATGTTTTTATGAATGAAAAAAGCTGCTGA | |
| | | GTTTTTGGTTTAGGAACATCAATTTGTTCAAATT | |
| | | GACCACTGTCTAAGGAGCCTAGTAATCGGCGTG | |
| | | CCTTAGTGTGTTTATGTGGTAATGTGTTACATTT | |
| | | AGCAATTATAACATTGTTTTATTAATAAGACTA | |
| | | TAAGAAGAGTGTACTTTTAGTAAGGGAGAAGT | |
| | | CTTGGAGGGTTGCTTCTGCAGGACGGGGAGGG | |
| | | AATTTGAGGGGAGGCTGAGGTGCCGTCAGGAC | |
| | | TTTTTTTTTTTTTTTTTTTTTGAGATGGAGTTTTG | |
| | | CTCTTGTTGCCCAGGCTGGAGTGCAATAGCGCG | |
| | | ATCTTGGCTCACCGCAACCTCCGCCTCCCAGGT | |
| | | TCAAGCGATTCTCCTGCCTCAGACTCCTAAGTA | |
| | | GCTGGGATTACAGGCACCTGCCACCACGCCTGG | |
| | | CTATTTTTTTGTATTTTTAGTAGAGATGGGGTTT | |
| | | CATCATGTTGGCCAGGCTGGTCTCGAGCTCCTG | |
| | | ACCTCAGGTGATCTGCCCGCCTCGGCCTCCAAA | |
| | | AGTGCTGGAATTACAGGCGTGAGCCACCATGCC | |
| | | TGGCCATCAGAACTTGTAATCAAGACAGTATGT | |
| | | TGAGAAATTCTAACATTATAAATTACAAAGCTT | |
| | | TGACTATTAAAGTTTTTGTGATCTAATGATACA | |
| | | GTTTTGATTCTATAGTAATTTGTGGCTTATTTTA | |
| | | TAGTTTATAATGAATACTTATTTCTAGACTCATA | |
| | | CACTGGAAGGGGACCCGGAAAGGTAATGTAAC | |
| | | TCAGTGATTTTAAAACTTGATTTTTTTAACTGAG | |
| | | AACTTTTTTTGCCCCCTGCCTGTAGGTTAAGTCT | |
| | | TACGTGAAATGCCAAGATAATTGCTGAGCAGCT | |
| | | TTGGTTACCCAGGGCGGGGTCTGGGTCTGTCTG | |
| | | TACTTTGCCTTTACTCTAGATGGCTCCTGAGAC | |
| | | ACAGGCAGGACTCCCAAGCACCGGGTTGGGAT | |
| | | CTGCCCTGGTCCCGGCATTCCAGTATAAGATTG | |
| | | CCTCAGACCTGTGTTTTTCAGACTGGGTTTTTGC | |
| | | TCTTCACATGAAATCAAGTTAGATGACAATGAC | |
| | | TGGTGTTGAAAAAAATGAAAAGGAAAGAATTT | |
| | | GTAAAGAACAGAAAATATATTTGAGTAAGTATT | |
| | | GTTTGGTAAAACTTAGTTACATATGCATATATA | |
| | | TTTGTTAGGTATATATGTTTATGTGTATTCTGAT | |
| | | GTAAAATATATATATATATATATTTTATTACTAT | |
| | | AGTACCATGGGTAATGGATAAAGAAGTTAAAG | |
| | | CTACTGCTTAGAATGAAGAAGGCCCCAGGCTTA | |
| | | CCTGTCCCGATCTTTAAACTGTCCGAAGGAAAT | |
| | | TCAATAGCCTGTTAAGTGAATACCTTCATTCTT | |
| | | ACTTGTATTTGGGGGAATATTATGAAATACTCA | |
| | | CCACTTTTGGTATTTTATGAAAATGTTTTCTTTT | |
| | | CAGAAGTTATGGTAATTTCAATGTGTTTGTTGTT | |
| | | GGGAGGGGAGCTGCCAAATCAGTTACTAATATT | |
| | | ACTGTGTGACATCTATCCAACTTTTTTCATTATT | |
| | | CTTCATTGCCAAATACTGAAAGACTTGTAAATG | |
| | | GCTTTGGCAATATGTTTGAATTCTAAGAGGAAA | |
| | | TATTTTCCCATAATTGTATATCAGAGAAATATA | |
| | | GTGATATACAATTTCCTTGAAAACCAATTTCTA | |
| | | AATAATTTTCTTCTCTGTAATCTAAGTGTAAAA | |
| | | AGGTTTAGTTTTTTAATAGGTTTAGGTGTTTATA | |
| | | AGCAATAGTTCTCTATTTTCTAGTTGATATAAGT | |
| | | AGAAGAATTGACAAGTGAGATGGAAATGTTAA | |
| | | TTTATAAAGGGAAAGAAAAGCTAGGTGAGGTT | |
| | | GAGTTATAATTAAACTGTTCAGGAAACATCGTA | |
| | | AAGGCTTTAGGCTCCCTTTTTCATTTCTATACCA | |
| | | ATTAATCTCATGGGTTCTAGAGTGGTTAGTTCT | |
| | | ACGGGAATTGTTTTTGTTTTTGTTTTTAAAGATG | |
| | | CTGAAAACTACTCTCAATCAAATTAGTACCATC | |
| | | ATTTAAGCTTTGAATACTTGGCAGTAATTGCCT | |
| | | GGGCTCGTCAATAAATGTTAGCAAATTCTTGAT | |
| | | GTTCAAAAAAAAAA | |

In one aspect, the present disclosure provides a method of identifying mismatch repair deficiency in a subject comprising: a) measuring the gene expression level of at least one gene comprising EPM2AIP1, TTC30A, SMAP1, RNLS, WNT11, SFXN1, SREBF1, TYMS, EIF5AL1, or WDR76 in a tumor sample from the subject; b) determining a score HPS, wherein HPS=(y−μ₂)/σ₂, wherein y=Σᵢ₌₁¹⁰yᵢwᵢ, wherein yᵢ is the log-transformed normalized expression of the at least one gene i in the tumor sample and wᵢ is the prespecified weight for gene i, μ₂ is the mean of the linear combination of the log-transformed normalized expression of the at least one gene in non-hypermutated samples, and σ₂ is the standard deviation of the linear combination of the log-transformed normalized expression of the at least one gene in non-hypermutated samples; c) comparing the HPS score with a predetermined cutoff value, wherein the cutoff value identifies mismatch repair deficiency in a subject with at least 95% specificity; and d) producing a report identifying the presence of mismatch repair deficiency in the subject when the HPS score is equal to or greater than the predetermined cutoff value or producing a report identifying the absence of mismatch repair deficiency in the subject when the HPS score is less than the predetermined cutoff value.

In one aspect, the present disclosure provides a method of identifying mismatch repair deficiency in a subject comprising: a) measuring the gene expression level of at least one gene comprising EPM2AIP1, TTC30A, SMAP1, RNLS, WNT11, SFXN1, SREBF1, TYMS, EIF5AL1, or WDR76 in a tumor sample from the subject; b) determining a score HPS, wherein HPS=(y−μ₂)/σ₂, wherein y=Σᵢ₌₁¹⁰yᵢwᵢ, wherein yᵢ is the log-transformed normalized expression of the at least one gene i in the tumor sample and wᵢ is the prespecified weight for gene i, μ₂ is the mean of the linear combination of the log-transformed normalized expression of the at least one gene in non-hypermutated samples, and σ₂ is the standard deviation of the linear combination of the log-transformed normalized expression of the at least one gene in non-hypermutated samples; c) comparing the HPS score with a predetermined cutoff value, wherein the cutoff value identifies mismatch repair deficiency in a subject with at least 95% specificity; and d) identifying the presence of mismatch repair deficiency in the subject when the HPS score is equal to or greater than the predetermined cutoff value or identifying the absence of mismatch repair deficiency in the subject when the HPS score is less than the predetermined cutoff value.

In some aspects, the preceding methods can further comprise administering at least one treatment to a subject identified as having mismatch repair deficiency. A treatment can comprise anti-cancer therapy. A treatment can comprise administering to the subject immunotherapy. The at least one treatment can comprise administering to the subject at least one checkpoint inhibitor. A treatment can comprise administering to the subject pembrolizumab, nivolumab, atezolizumab, avelumab, durvalumab, pidilizumab, REGN2810, AMP-224, MEDI0680, PDR001, CT-001 or a combination thereof. A treatment can comprise administering to the subject a CTLA4 antibody. A CTLA4 antibody can comprise ipilimumab, tremelimumab or a combination thereof.

In one aspect, the present disclosure provides a method of identifying mismatch repair deficiency in a subject comprising: a) measuring the gene expression level of at least one gene comprising EPM2AIP1, TTC30A, SMAP1, RNLS, WNT11, SFXN1, SREBF1, TYMS, EIF5AL1, or WDR76 in a tumor sample from the subject; b) determining a score HPS, wherein HPS=(y−μ₂)/σ₂, wherein y=Σᵢ₌₁¹⁰yᵢwᵢ, wherein yᵢ is the log-transformed normalized expression of the at least one gene i in the tumor sample and wᵢ is the prespecified weight for gene i, μ₂ is the mean of the linear combination of the log-transformed normalized expression of the at least one gene in non-hypermutated samples, and σ₂ is the standard deviation of the linear combination of the log-transformed normalized expression of the at least one gene in non-hypermutated samples; c) comparing the HPS score with a predetermined cutoff value, wherein the cutoff value identifies mismatch repair deficiency in a subject with at least 95% specificity; and d) administering at least one treatment to the subject when the HPS score is equal to or greater than the predetermined cutoff value. A treatment can comprise anti-cancer therapy. A treatment can comprise administering to the subject immunotherapy. The at least one treatment can comprise administering to the subject at least one checkpoint inhibitor. A treatment can comprise administering to the subject pembrolizumab, nivolumab, atezolizumab, avelumab, durvalumab, pidilizumab, REGN2810, AMP-224, MEDI0680, PDR001, CT-001 or a combination thereof. A treatment can comprise administering to the subject a CTLA4 antibody. A CTLA4 antibody can comprise ipilimumab, tremelimumab or a combination thereof.

In some aspects of the preceding methods, determining μ₂ in step (b), wherein μ₂ is the mean of the linear combination of the log-transformed normalized expression of the at least one gene in non-hypermutated samples, comprises: 1) measuring the gene expression level of the at least one gene in a plurality of analogous, non-hypermutated tumor samples from at least one subject, wherein at least one sample in the plurality of analogous, non-hypermutated samples originates from the same tissue as the tumor sample in step (a) of the preceding methods; 2) determining z, wherein z=Σᵢ₌₁¹⁰zᵢwᵢ, wherein zᵢ is the log-transformed normalized expression of the at least one gene i from step (1) and wᵢ is the prespecified weight for gene i; and 3) determining for each of the at least one gene the mean of z from step (2).

In some aspects of the preceding methods, determining σ₂ in step (b), wherein σ₂ is the standard deviation of the linear combination of the log-transformed normalized expression of the at least one gene in non-hypermutated samples, comprises: 1) measuring the gene expression level of the at least one gene in a plurality of analogous, non-hypermutated tumor samples from at least one subject, wherein at least one sample in the plurality of analogous, non-hypermutated samples originates from the same tissue as the tumor sample in step (a) of the preceding methods; 2) determining z, wherein z=Σᵢ₌₁¹⁰zᵢwᵢ, wherein zᵢ is the log-transformed normalized expression of the at least one gene i from step (1) and wᵢ is the prespecified weight for gene i; and 3) determining for each of the at least one gene the standard deviation of z from step (2).

In some aspects of the preceding methods, measuring the gene expression of the at least one gene in a tumor sample from the subject and measuring the gene expression of the at least one gene in a plurality of analogous non-hypermutated tumor samples is performed using the same method. In some aspects of the preceding methods, measuring the gene expression of the at least one gene in a tumor sample from the subject and measuring the gene expression of the at least one gene in a plurality of analogous non-hypermutated tumor samples is performed using the same apparatus. In preferred aspects of the preceding methods, measuring the gene expression of the at least one gene in a tumor sample from the subject and measuring the gene expression of the at least one gene in a plurality of analogous non-hypermutated tumor samples is performed using the same method and apparatus.

In some aspects, the prespecified weight for gene i, wᵢ, in step (b) of the preceding methods can be:

| Gene | Weight |
|---|---|
| EPM2AIP1 | −0.31218 |
| TTC30A | −0.19894 |
| SMAP1 | −0.1835 |
| RNLS | −0.19023 |
| WNT11 | −0.11515 |
| SFXN1 | 0.214676 |
| SREBF1 | 0.194835 |
| TYMS | 0.206972 |
| EIF5AL1 | 0.194935 |
| WDR76 | 0.188582 |

In some aspects, the predetermined cutoff value in the preceding methods that identifies mismatch repair deficiency in a subject can have a specificity of 99%. In some aspects, the predetermined cutoff value in the preceding methods that identifies mismatch repair deficiency in a subject can have a specificity of at least 99%. In preferred aspects, the cutoff value that identifies mismatch repair deficiency in a subject can have a specificity of 99.5%. In preferred aspects, the cutoff value that identifies mismatch repair deficiency in a subject can have a specificity of at least 99.5%.

In some aspects, the predetermined cutoff value in the preceding methods that identifies mismatch repair deficiency in a subject can have a specificity of at least 70%, or at least 75%, or at least 80%, or at least 85%, or at least 90%, or at least 91%, or at least 92%, or at least 93%, or at least 94%, or at least 95%, or at least 96%, or at least 97% or at least 98%.

In some aspects, the predetermined cutoff value of the preceding methods that identifies mismatch repair deficiency in a subject can be 1.645. Alternatively, the predetermined cutoff value can be 2.326. Alternatively still, the predetermined cutoff value can be 2.576.

The at least one gene in step (a) of the preceding methods can comprise each of EPM2AIP1, TTC30A, SMAP1, RNLS, WNT11, SFXN1, SREBF1, TYMS, EIF5AL1, and WDR76.

In some aspects, step (a) of the preceding methods can comprise measuring the gene expression level of at least two genes, or at least three genes, or at least four genes, or at least five genes, or at least six genes, or at least seven genes, or at least eight genes, or at least nine genes or at least 10 genes comprising EPM2AIP1, TTC30A, SMAP1, RNLS, WNT11, SFXN1, SREBF1, TYMS, EIF5AL1, and WDR76.

In some aspects, when the tumor sample is a colon adenocarcinoma (COAD), esophageal carcinoma (ESCA), stomach adenocarcinoma (STAD) or uterine corpus endometrial carcinoma (UCEC) tumor sample, σ₂, the standard deviation of the linear combination of the log transformed gene expression of the at least one gene in non-hypermutated samples, in step (b) of the preceding methods can be:

| Tumor | |
|---|---|
| Type | σ₂ |
| COAD | 0.6604 |
| ESCA | 0.7617 |
| STAD | 0.8153 |
| UCEC | 0.7027 |

Table 1 shows the sequences of the at least one gene from step (a) of the preceding methods.

In one aspect, the present disclosure provides a method of identifying mismatch repair deficiency in a subject comprising: a) measuring the gene expression level of at least one gene comprising MLH1, MSH2, MSH6 or PMS2 in a tumor sample from the subject; b) determining for each of the at least one gene a score Z, wherein Z=(x−μ₁)/σ₁, wherein x is the log-transformed normalized expression of the at least one gene, μ₁ is the mean of the log-transformed normalized expression of the at least one gene in non-hypermutated samples, and σ₁ is the standard deviation of the log-transformed normalized expression of the at least one gene in non-hypermutated samples; c) determining a score MLS, wherein MLS=(Zₘ+c₁)/c₂, wherein Zₘ is the minimum Z score of the at least one gene, and wherein c₁ is 0 and c₂ is 1 when one gene is used, c₁ is 0.56 and c₂ is 0.83 when two genes are used, c₁ is 0.85 and c₂ is 0.75 when three genes are used, or c₁ is 1.03 and c₂ is 0.70 when four genes are used; d) measuring the gene expression level of at least one gene comprising EPM2AIP1, TTC30A, SMAP1, RNLS, WNT11, SFXN1, SREBF1, TYMS, EIF5AL1, or WDR76 in a tumor sample from the subject; e) determining a score HPS, wherein HPS=(y−μ₂)/σ₂, wherein y=Σᵢ₌₁¹⁰yᵢwᵢ, wherein yᵢ is the log-transformed normalized expression of the at least one gene i in the tumor sample and wᵢ is the prespecified weight for gene i, μ₂ is the mean of the linear combination of the log-transformed normalized expression of the at least one gene in non-hypermutated samples, and σ₂ is the standard deviation of the linear combination of the log-transformed normalized expression of the at least one gene in non-hypermutated samples; f) determining a score MPS wherein MPS=(max(HPS,0)²+min(MLS,0)²)^{1/2}; g) comparing the MPS score with a predetermined cutoff value, wherein the cutoff value identifies mismatch repair deficiency in a subject with at least 95% specificity; and h) producing a report identifying the presence of mismatch repair deficiency in the subject when the MPS score is equal to or greater than the predetermined cutoff value or producing a report identifying the absence of mismatch repair deficiency in the subject when the MPS score is less than the predetermined cutoff value.

In one aspect, the present disclosure provides a method of identifying mismatch repair deficiency in a subject comprising: a) measuring the gene expression level of at least one gene comprising MLH1, MSH2, MSH6 or PMS2 in a tumor sample from the subject; b) determining for each of the at least one gene a score Z, wherein Z=(x−μ₁)/σ₁, wherein x is the log-transformed normalized expression of the at least one gene, μ₁ is the mean of the log-transformed normalized expression of the at least one gene in non-hypermutated samples, and σ₁ is the standard deviation of the log-transformed normalized expression of the at least one gene in non-hypermutated samples; c) determining a score MLS, wherein MLS=(Zₘ+c₁)/c₂, wherein Zₘ is the minimum Z score of the at least one gene, and wherein c₁ is 0 and c₂ is 1 when one gene is used, c₁ is 0.56 and c₂ is 0.83 when two genes are used, c₁ is 0.85 and c₂ is 0.75 when three genes are used, or c₁ is 1.03 and c₂ is 0.70 when four genes are used; d) measuring the gene expression level of at least one gene comprising EPM2AIP1, TTC30A, SMAP1, RNLS, WNT11, SFXN1, SREBF1, TYMS, EIF5AL1, or WDR76 in a tumor sample from the subject; e) determining a score HPS, wherein HPS=(y−μ₂)/σ₂, wherein y=Σᵢ₌₁¹⁰yᵢwᵢ, wherein yᵢ is the log-transformed normalized expression of the at least one gene i in the tumor sample and wᵢ is the prespecified weight for gene i, μ₂ is the mean of the linear combination of the log-transformed normalized expression of the at least one gene in non-hypermutated samples, and σ₂ is the standard deviation of the linear combination of the log-transformed normalized expression of the at least one gene in non-hypermutated samples; f) determining a score MPS wherein MPS=(max(HPS,0)²+min(MLS,0)²)^{1/2}; g) comparing the MPS score with a predetermined cutoff value, wherein the cutoff value identifies mismatch repair deficiency in a subject with at least 95% specificity; and h) identifying the presence of mismatch repair deficiency in the subject when the MPS score is equal to or greater than the predetermined cutoff value or identifying the absence of mismatch repair deficiency in the subject when the MPS score is less than the predetermined cutoff value.

In some aspects, the preceding methods can further comprise administering at least one treatment to a subject identified as having mismatch repair deficiency. A treatment can comprise anti-cancer therapy. A treatment can comprise administering to the subject immunotherapy. The at least one treatment can comprise administering to the subject at least one checkpoint inhibitor. A treatment can comprise administering to the subject pembrolizumab, nivolumab, atezolizumab, avelumab, durvalumab, pidilizumab, REGN2810, AMP-224, MEDI0680, PDR001, CT-001 or a combination thereof. A treatment can comprise administering to the subject a CTLA4 antibody. A CTLA4 antibody can comprise ipilimumab, tremelimumab or a combination thereof.

In one aspect, the present disclosure provides a method of identifying mismatch repair deficiency in a subject comprising: a) measuring the gene expression level of at least one gene comprising MLH1, MSH2, MSH6 or PMS2 in a tumor sample from the subject; b) determining for each of the at least one gene a score Z, wherein Z=(x−μ₁)/σ₁, wherein x is the log-transformed normalized expression of the at least one gene, μ₁ is the mean of the log-transformed normalized expression of the at least one gene in non-hypermutated samples, and σ₁ is the standard deviation of the log-transformed normalized expression of the at least one gene in non-hypermutated samples; c) determining a score MLS, wherein MLS=(Zₘ+c₁)/c₂, wherein Zₘ is the minimum Z score of the at least one gene, and wherein c₁ is 0 and c₂ is 1 when one gene is used, c₁ is 0.56 and c₂ is 0.83 when two genes are used, c₁ is 0.85 and c₂ is 0.75 when three genes are used, or c₁ is 1.03 and c₂ is 0.70 when four genes are used; d) measuring the gene expression level of at least one gene comprising EPM2AIP1, TTC30A, SMAP1, RNLS, WNT11, SFXN1, SREBF1, TYMS, EIF5AL1, or WDR76 in a tumor sample from the subject; e) determining a score HPS, wherein HPS=(y−μ₂)/σ₂, wherein y=Σᵢ₌₁¹⁰yᵢwᵢ, wherein yᵢ is the log-transformed normalized expression of the at least one gene i in the tumor sample and wᵢ is the prespecified weight for gene i, μ₂ is the mean of the linear combination of the log-transformed normalized expression of the at least one gene in non-hypermutated samples, and σ₂ is the standard deviation of the linear combination of the log-transformed normalized expression of the at least one gene in non-hypermutated samples; f) determining a score MPS wherein MPS=(max(HPS,0)²+min(MLS,0)²)^{1/2}; g) comparing the MPS score with a predetermined cutoff value, wherein the cutoff value identifies mismatch repair deficiency in a subject with at least 95% specificity; and h) administering at least one treatment to the subject when the MPS score is equal to or greater than the predetermined cutoff value. A treatment can comprise anti-cancer therapy. A treatment can comprise administering to the subject immunotherapy. The at least one treatment can comprise administering to the subject at least one checkpoint inhibitor. A treatment can comprise administering to the subject pembrolizumab, nivolumab, atezolizumab, avelumab, durvalumab, pidilizumab, REGN2810, AMP-224, MEDI0680, PDR001, CT-001 or a combination thereof. A treatment can comprise administering to the subject a CTLA4 antibody. A CTLA4 antibody can comprise ipilimumab, tremelimumab or a combination thereof.

In some aspects of the preceding methods, determining μ₁ in step (b), wherein μ₁ is the mean of the log-transformed normalized expression of the at least one gene in non-hypermutated samples, comprises: 1) measuring the gene expression level of the at least one gene in a plurality of analogous, non-hypermutated tumor samples from at least one subject, wherein at least one sample in the plurality of analogous, non-hypermutated samples originates from the same tissue as the tumor sample in step (a) of the preceding methods; 2) determining for each of the at least one gene the log-transformed normalized expression; and 3) determining for each of the at least one gene the mean of the log 2-transformed expression from step (2).

In some aspects of the preceding methods, determining σ₁ in step (b), wherein σ₁ is the standard deviation of the log-transformed normalized expression of the at least one gene in non-hypermutated samples, comprises: 1) measuring the gene expression level of the at least one gene in a plurality of analogous, non-hypermutated tumor samples from at least one subject, wherein at least one sample in the plurality of analogous, non-hypermutated samples originates from the same tissue as the tumor sample in step (a) of the preceding methods; 2) determining for each of the at least one gene the log-transformed normalized expression; and 3) determining for each of the at least one gene the standard deviation of the log 2-transformed expression from step (2).

In some aspects of the preceding methods, determining μ₂ in step (e), wherein μ₂ is the mean of the linear combination of the log-transformed normalized expression of the at least one gene in non-hypermutated samples, comprises: 1) measuring the gene expression level of the at least one gene in a plurality of analogous, non-hypermutated tumor samples from at least one subject, wherein at least one sample in the plurality of analogous, non-hypermutated samples originates from the same tissue as the tumor sample in step (a) of the preceding methods; 2) determining z, wherein z=Σᵢ₌₁¹⁰zᵢwᵢ, wherein zᵢ is the log-transformed normalized expression of the at least one gene i from step (1) and wᵢ is the prespecified weight for gene i; and 3) determining for each of the at least one gene the mean of z from step (2).

In some aspects of the preceding methods, determining σ₂ in step (e), wherein σ₂ is the standard deviation of the linear combination of the log-transformed normalized expression of the at least one gene in non-hypermutated samples, comprises: 1) measuring the gene expression level of the at least one gene in a plurality of analogous, non-hypermutated tumor samples from at least one subject, wherein at least one sample in the plurality of analogous, non-hypermutated samples originates from the same tissue as the tumor sample in step (a) of the preceding methods; 2) determining z, wherein z=Σᵢ₌₁¹⁰zᵢwᵢ, wherein zᵢ is the log-transformed normalized expression of the at least one gene i from step (1) and wᵢ is the prespecified weight for gene i; and 3) determining for each of the at least one gene the standard deviation of z from step (2).

In some aspects of the preceding methods, measuring the gene expression of the at least one gene in a tumor sample from the subject and measuring the gene expression of the at least one gene in a plurality of analogous non-hypermutated tumor samples is performed using the same method. In some aspects of the preceding methods, measuring the gene expression of the at least one gene in a tumor sample from the subject and measuring the gene expression of the at least one gene in a plurality of analogous non-hypermutated tumor samples is performed using the same apparatus. In preferred aspects of the preceding methods, measuring the gene expression of the at least one gene in a tumor sample from the subject and measuring the gene expression of the at least one gene in a plurality of analogous non-hypermutated tumor samples is performed using the same method and apparatus.

In some aspects, the prespecified weight for gene i, wᵢ, in step (e) of the preceding methods can be:

| Gene | Weight |
|---|---|
| EPM2AIP1 | −0.31218 |
| TTC30A | −0.19894 |
| SMAP1 | −0.1835 |
| RNLS | −0.19023 |
| WNT11 | −0.11515 |
| SFXN1 | 0.214676 |
| SREBF1 | 0.194835 |
| TYMS | 0.206972 |
| EIF5AL1 | 0.194935 |
| WDR76 | 0.188582 |

In some aspects, the predetermined cutoff value in the preceding methods that identifies mismatch repair deficiency in a subject can have a specificity of 99%. In some aspects, the predetermined cutoff value in the preceding methods that identifies mismatch repair deficiency in a subject can have a specificity of at least 99%. In preferred aspects, the cutoff value that identifies mismatch repair deficiency in a subject can have a specificity of 99.5%. In preferred aspects, the cutoff value that identifies mismatch repair deficiency in a subject can have a specificity of at least 99.5%.

In some aspects, the predetermined cutoff value in the preceding methods that identifies mismatch repair deficiency in a subject can have a specificity of at least 70%, or at least 75%, or at least 80%, or at least 85%, or at least 90%, or at least 91%, or at least 92%, or at least 93%, or at least 94%, or at least 95%, or at least 96%, or at least 97% or at least 98%.

In some aspects, the predetermined cutoff value of the preceding methods that identifies mismatch repair deficiency in a subject can be 2.058. Alternatively, the predetermined cutoff value can be 2.699. Alternatively still, the predetermined cutoff value can be 2.939.

The at least one gene in step (a) of the preceding methods can comprise MLH1. Alternatively, the at least one gene in step (a) can comprise each of MLH1, MSH2, MSH6 and PMS2.

The at least one gene in step (d) of the preceding can comprise each of EPM2AIP1, TTC30A, SMAP1, RNLS, WNT11, SFXN1, SREBF1, TYMS, EIF5AL1 and WDR76.

The at least one gene in step (a) of the preceding can comprise MLH1 and the at least one gene in step (d) of the preceding can comprise each of EPM2AIP1, TTC30A, SMAP1, RNLS, WNT11, SFXN1, SREBF1, TYMS, EIF5AL1 and WDR76. Alternatively, the at least one gene in step (a) of the preceding can comprise each of MLH1, MSH2, MSH6 and PMS2 and the at least one gene in step (d) of the preceding can comprise each of EPM2AIP1, TTC30A, SMAP1, RNLS, WNT11, SFXN1, SREBF1, TYMS, EIF5AL1 and WDR76.

In some aspects, step (a) of the preceding methods can comprise measuring the gene expression level of at least two genes, or at least three genes or at least four genes comprising MLH1, MSH2, MSH6 or PMS2 in a tumor sample from the subject.

In some aspects, step (d) of the preceding methods can comprise measuring the gene expression level of at least two genes, or at least three genes, or at least four genes, or at least five genes, or at least six genes, or at least seven genes, or at least eight genes, or at least nine genes or at least 10 genes comprising EPM2AIP1, TTC30A, SMAP1, RNLS, WNT11, SFXN1, SREBF1, TYMS, EIF5AL1, and WDR76.

In some aspects, when the tumor sample is a colon adenocarcinoma (COAD), esophageal carcinoma (ESCA), stomach adenocarcinoma (STAD) or uterine corpus endometrial carcinoma (UCEC) tumor sample, σ₁, the standard deviation of the expression of the at least one gene in non-hypermutated samples, in step (b) of the preceding methods can be

| | MLH1 | MSH2 | MSH6 | PMS2 |
|---|---|---|---|---|
| COAD | 0.3241 | 0.4108 | 0.4198 | 0.3259 |
| ESCA | 0.5221 | 0.6602 | 0.7347 | 0.4927 |
| STAD | 0.4245 | 0.6020 | 0.4814 | 0.4314 |
| UCEC | 0.4543 | 0.7312 | 0.6158 | 0.4217 |

In some aspects, when the tumor sample is a colon adenocarcinoma (COAD), esophageal carcinoma (ESCA), stomach adenocarcinoma (STAD) or uterine corpus endometrial carcinoma (UCEC) tumor sample, σ₂, the standard deviation of the linear combination of the log transformed gene expression of the at least one gene in non-hypermutated samples, in step (e) of the preceding methods can be

| Tumor | |
|---|---|
| Type | σ₂ |
| COAD | 0.6604 |
| ESCA | 0.7617 |
| STAD | 0.8153 |
| UCEC | 0.7027 |

Table 1 shows the sequences of the at least one gene from step (a) and the at least one gene from step (d) of the preceding methods.

In some aspects, a subject can be diagnosed with cancer.

In some aspects, a report of the preceding methods identifying mismatch repair deficiency can further identify the subject as having cancer. In some aspects of the methods of the present disclosure, identifying mismatch repair deficiency in a subject can further identify the subject as having cancer.

In some aspects, a report of the preceding method that identifies the presence of mismatch repair deficiency in a subject can further identify the subject for treatment with an anti-cancer therapy. In some aspects of the methods of the present disclosure, identifying the presence of mismatch repair deficiency in a subject can further identify the subject for treatment with anti-cancer therapy.

In some aspects, a treatment with an anti-cancer therapy can comprise administering a treatment to a subject identified as having mismatch repair deficiency. A treatment can comprise administering to the subject immunotherapy. A treatment can also comprise administering to the subject checkpoint inhibitors. A treatment can comprise administering to the subject pembrolizumab, nivolumab, atezolizumab, avelumab, durvalumab, pidilizumab, REGN2810, AMP-224, MEDI0680, PDR001, CT-001 or a combination thereof. A treatment can comprise administering to the subject a CTLA4 antibody. The CTLA4 antibody can comprise ipilimumab, tremelimumab or a combination thereof.

In aspects of the methods of the present disclosure, gene expression is measured using methods known in the art. In preferred aspects, the methods are enzyme free methods e.g. US2003/0013091, US2007/0166708, US2010/0015607, US2010/0261026, US2010/0262374, US2010/0112710, US2010/0047924, and US2014/0371088, each of which is incorporated herein by reference in its entirety. Preferably, nCounter® probes, systems, and methods from NanoString Technologies®, as described in US2003/0013091, US2007/0166708, US2010/0015607, US2010/0261026, US2010/0262374, US2010/0112710, US2010/0047924, US2014/0371088, US2014/0017688, and US2011/0086774) are a preferred means for measuring gene expression. nCounter® probes, systems, and methods from NanoString Technologies® allow simultaneous multiplexed identification a plurality (800 or more) distinct target proteins and/or target nucleic acids. Each of the above-mentioned patent publications is incorporated herein by reference in its entirety. The above-mentioned nCounter® probes, systems, and methods from NanoString Technologies® can be combined with any aspect or embodiment described herein.

In one aspect, the present disclosure provides a method of determining a tumor inflammation signature score in a subject comprising: a) measuring the raw RNA level of at least one gene comprising CCL5, CD27, CD274, CD276, CD8A, CMKRLR1, CXCL9, CXCR6, HLA-DQA1, HLA-DRB1, HLA-E, IDO1, LAG3, NKG7, PDCD1LG2, PSMB10, STAT1 and TIGIT; b) measuring the raw RNA level of at least one gene comprising ABCF1, C14ORF102, G6PD, OAZ1, POLR2A, SDHA, STK11IP, TBC1D10B, TBP, UBB and ZBTB34; c) normalizing the measured raw RNA level of the at least one gene from step (a) using the measured raw RNA levels of the at least one gene from step (b); and d) generating a tumor inflammation signature score (TIS) wherein TIS=Σᵢ₌₁¹⁰qᵢwᵢ, wherein qᵢ is the normalized raw RNA level of the at least one gene i from step (c), and wᵢ is a prespecified weight for gene i.

A more detailed description for determining a tumor inflammation signature score in a subject is disclosed in PCT/US2015/064445 (WO2016/094377), which is incorporated by reference in its entirety. See also Ayers M et al. The Journal of clinical investigation. 2017 Aug. 1; 127(8):2930-40.

In some aspects, the prespecified weight for gene i, wᵢ, in step (d) of the preceding method can be

| Gene | Weight |
|---|---|
| CCL5 | 0.008346 |
| CD27 | 0.072293 |
| CD274 | 0.042853 |
| CD276 | −0.0239 |
| CD8A | 0.031021 |
| CMKRLR1 | 0.151253 |
| CXCL9 | 0.074135 |
| CXCR6 | 0.004313 |
| HLA-DQA1 | 0.020091 |
| HLA-DRB1 | 0.058806 |
| HLA-E | 0.07175 |
| IDO1 | 0.060679 |
| LAG3 | 0.123895 |
| NKG7 | 0.075524 |
| PDCDILG2 | 0.003734 |
| PSMB10 | 0.032999 |
| STAT1 | 0.250229 |
| TIGIT | 0.084767 |

In alternative aspects of the preceding method, step (a) comprises measuring the raw RNA level of at least two genes, or at least three genes, or at least four genes, or at least five genes, or at least six genes, or at least seven genes, or at least eight genes, or at least nine genes, or at least 10 genes, or at least 11 genes, or at least 12 genes, or at least 13 genes, or at least 14 genes, or at least 15 genes, or at least 16 genes, at least 17 genes comprising CCL5, CD27, CD274, CD276, CD8A, CMKRLR1, CXCL9, CXCR6, HLA-DQA1, HLA-DRB1, HLA-E, IDO1, LAG3, NKG7, PDCDILG2, PSMB10, STAT1 and TIGIT. In a preferred aspect, step (a) comprises measuring the raw RNA level of at least 18 genes comprising each of CCL5, CD27, CD274, CD276, CD8A, CMKRLR1, CXCL9, CXCR6, HLA-DQA1, HLA-DRB1, HLA-E, IDO1, LAG3, NKG7, PDCDILG2, PSMB10, STAT1 and TIGIT.

In alternative aspects of the preceding method, step (b) comprises measuring the raw RNA level of at least two genes, or at least three genes, or at least four genes, or at least five genes, or at least six genes, or at least seven genes, or at least eight genes, or at least nine genes, or at least 10 genes or at least 11 genes comprising ABCF1, C14ORF102, G6PD, OAZ1, POLR2A, SDHA, STK11IP, TBC1D10B, TBP, UBB and ZBTB34. In a preferred aspect, step (b) comprises measuring the raw RNA level of at least 11 genes comprising each of ABCF1, C14ORF102, G6PD, OAZ1, POLR2A, SDHA, STK11IP, TBC1D10B, TBP, UBB and ZBTB34.

Table 2 shows the sequences of the at least one gene from step (a) and the at least one gene from step (b) of the preceding method.

TABLE 2
Sequences of genes measured for determining tumor inflammation
signature score

| | Gen Bank | | SEQ |
|---|---|---|---|
| Gene | Accession No. | Sequence | ID No. |
| CCL5 | NM_002985.2 | GCTGCAGAGGATTCCTGCAGAGGATCAAGACAG | 15 |
| | | CACGTGGACCTCGCACAGCCTCTCCCACAGGTA | |
| | | CCATGAAGGTCTCCGCGGCAGCCCTCGCTGTCAT | |
| | | CCTCATTGCTACTGCCCTCTGCGCTCCTGCATCT | |
| | | GCCTCCCCATATTCCTCGGACACCACACCCTGCT | |
| | | GCTTTGCCTACATTGCCCGCCCACTGCCCCGTGC | |
| | | CCACATCAAGGAGTATTTCTACACCAGTGGCAA | |
| | | GTGCTCCAACCCAGCAGTCGTCTTTGTCACCCGA | |
| | | AAGAACCGCCAAGTGTGTGCCAACCCAGAGAAG | |
| | | AAATGGGTTCGGGAGTACATCAACTCTTTGGAG | |
| | | ATGAGCTAGGATGGAGAGTCCTTGAACCTGAAC | |
| | | TTACACAAATTTGCCTGTTTCTGCTTGCTCTTGTC | |
| | | CTAGCTTGGGAGGCTTCCCCTCACTATCCTACCC | |
| | | CACCCGCTCCTTGAAGGGCCCAGATTCTACCACA | |
| | | CAGCAGCAGTTACAAAAACCTTCCCCAGGCTGG | |
| | | ACGTGGTGGCTCACGCCTGTAATCCCAGCACTTT | |
| | | GGGAGGCCAAGGTGGGTGGATCACTTGAGGTCA | |
| | | GGAGTTCGAGACCAGCCTGGCCAACATGATGAA | |
| | | ACCCCATCTCTACTAAAAATACAAAAAATTAGC | |
| | | CGGGCGTGGTAGCGGGCGCCTGTAGTCCCAGCT | |
| | | ACTCGGGAGGCTGAGGCAGGAGAATGGCGTGAA | |
| | | CCCGGGAGGCGGAGCTTGCAGTGAGCCGAGATC | |
| | | GCGCCACTGCACTCCAGCCTGGGCGACAGAGCG | |
| | | AGACTCCGTCTCAAAAAAAAAAAAAAAAAAAA | |
| | | AAATACAAAAATTAGCCGGGCGTGGTGGCCCAC | |
| | | GCCTGTAATCCCAGCTACTCGGGAGGCTAAGGC | |
| | | AGGAAAATTGTTTGAACCCAGGAGGTGGAGGCT | |
| | | GCAGTGAGCTGAGATTGTGCCACTTCACTCCAGC | |
| | | CTGGGTGACAAAGTGAGACTCCGTCACAACAAC | |
| | | AACAACAAAAAGCTTCCCCAACTAAAGCCTAGA | |
| | | AGAGCTTCTGAGGCGCTGCTTTGTCAAAAGGAA | |
| | | GTCTCTAGGTTCTGAGCTCTGGCTTTGCCTTGGC | |
| | | TTTGCCAGGGCTCTGTGACCAGGAAGGAAGTCA | |
| | | GCATGCCTCTAGAGGCAAGGAGGGGAGGAACAC | |
| | | TGCACTCTTAAGCTTCCGCCGTCTCAACCCCTCA | |
| | | CAGGAGCTTACTGGCAAACATGAAAAATCGGCT | |
| | | TACCATTAAAGTTCTCAATGCAACCATAAAAAA | |
| | | AAAA | |
| CD27 | NM_001242.4 | CGGAAGGGGAAGGGGGTGGAGGTTGCTGCTATG | 16 |
| | | AGAGAGAAAAAAAAAACAGCCACAATAGAGAT | |
| | | TCTGCCTTCAAAGGTTGGCTTGCCACCTGAAGCA | |
| | | GCCACTGCCCAGGGGGTGCAAAGAAGAGACAGC | |
| | | AGCGCCCAGCTTGGAGGTGCTAACTCCAGAGGC | |
| | | CAGCATCAGCAACTGGGCACAGAAAGGAGCCGC | |
| | | CTGGGCAGGGACCATGGCACGGCCACATCCCTG | |
| | | GTGGCTGTGCGTTCTGGGGACCCTGGTGGGGCTC | |
| | | TCAGCTACTCCAGCCCCCAAGAGCTGCCCAGAG | |
| | | AGGCACTACTGGGCTCAGGGAAAGCTGTGCTGC | |
| | | CAGATGTGTGAGCCAGGAACATTCCTCGTGAAG | |
| | | GACTGTGACCAGCATAGAAAGGCTGCTCAGTGT | |
| | | GATCCTTGCATACCGGGGGTCTCCTTCTCTCCTG | |
| | | ACCACCACACCCGGCCCCACTGTGAGAGCTGTC | |
| | | GGCACTGTAACTCTGGTCTTCTCGTTCGCAACTG | |
| | | CACCATCACTGCCAATGCTGAGTGTGCCTGTCGC | |
| | | AATGGCTGGCAGTGCAGGGACAAGGAGTGCACC | |
| | | GAGTGTGATCCTCTTCCAAACCCTTCGCTGACCG | |
| | | CTCGGTCGTCTCAGGCCCTGAGCCCACACCCTCA | |
| | | GCCCACCCACTTACCTTATGTCAGTGAGATGCTG | |
| | | GAGGCCAGGACAGCTGGGCACATGCAGACTCTG | |
| | | GCTGACTTCAGGCAGCTGCCTGCCCGGACTCTCT | |
| | | CTACCCACTGGCCACCCCAAAGATCCCTGTGCA | |
| | | GCTCCGATTTTATTCGCATCCTTGTGATCTTCTCT | |
| | | GGAATGTTCCTTGTTTTCACCCTGGCCGGGGCCC | |
| | | TGTTCCTCCATCAACGAAGGAAATATAGATCAA | |
| | | ACAAAGGAGAAAGTCCTGTGGAGCCTGCAGAGC | |
| | | CTTGTCGTTACAGCTGCCCCAGGGAGGAGGAGG | |
| | | GCAGCACCATCCCCATCCAGGAGGATTACCGAA | |
| | | AACCGGAGCCTGCCTGCTCCCCCTGAGCCAGCA | |
| | | CCTGCGGGAGCTGCACTACAGCCCTGGCCTCCA | |
| | | CCCCCACCCCGCCGACCATCCAAGGGAGAGTGA | |
| | | GACCTGGCAGCCACAACTGCAGTCCCATCCTCTT | |
| | | GTCAGGGCCCTTTCCTGTGTACACGTGACAGAGT | |
| | | GCCTTTTCGAGACTGGCAGGGACGAGGACAAAT | |
| | | ATGGATGAGGTGGAGAGTGGGAAGCAGGAGCC | |
| | | CAGCCAGCTGCGCCTGCGCTGCAGGAGGGCGGG | |
| | | GGCTCTGGTTGTAAAACACACTTCCTGCTGCGAA | |
| | | AGACCCACATGCTACAAGACGGGCAAAATAAAG | |
| | | TGACAGATGACCACCCTGCA | |
| CD274 | NM_014143.3 | GGCGCAACGCTGAGCAGCTGGCGCGTCCCGCGC | 17 |
| | | GGCCCCAGTTCTGCGCAGCTTCCCGAGGCTCCGC | |
| | | ACCAGCCGCGCTTCTGTCCGCCTGCAGGGCATTC | |
| | | CAGAAAGATGAGGATATTTGCTGTCTTTATATTC | |
| | | ATGACCTACTGGCATTTGCTGAACGCATTTACTG | |
| | | TCACGGTTCCCAAGGACCTATATGTGGTAGAGT | |
| | | ATGGTAGCAATATGACAATTGAATGCAAATTCC | |
| | | CAGTAGAAAAACAATTAGACCTGGCTGCACTAA | |
| | | TTGTCTATTGGGAAATGGAGGATAAGAACATTA | |
| | | TTCAATTTGTGCATGGAGAGGAAGACCTGAAGG | |
| | | TTCAGCATAGTAGCTACAGACAGAGGGCCCGGC | |
| | | TGTTGAAGGACCAGCTCTCCCTGGGAAATGCTG | |
| | | CACTTCAGATCACAGATGTGAAATTGCAGGATG | |
| | | CAGGGGTGTACCGCTGCATGATCAGCTATGGTG | |
| | | GTGCCGACTACAAGCGAATTACTGTGAAAGTCA | |
| | | ATGCCCCATACAACAAAATCAACCAAAGAATTT | |
| | | TGGTTGTGGATCCAGTCACCTCTGAACATGAACT | |
| | | GACATGTCAGGCTGAGGGCTACCCCAAGGCCGA | |
| | | AGTCATCTGGACAAGCAGTGACCATCAAGTCCT | |
| | | GAGTGGTAAGACCACCACCACCAATTCCAAGAG | |
| | | AGAGGAGAAGCTTTTCAATGTGACCAGCACACT | |
| | | GAGAATCAACACAACAACTAATGAGATTTTCTA | |
| | | CTGCACTTTTAGGAGATTAGATCCTGAGGAAAA | |
| | | CCATACAGCTGAATTGGTCATCCCAGAACTACCT | |
| | | CTGGCACATCCTCCAAATGAAAGGACTCACTTG | |
| | | GTAATTCTGGGAGCCATCTTATTATGCCTTGGTG | |
| | | TAGCACTGACATTCATCTTCCGTTTAAGAAAAGG | |
| | | GAGAATGATGGATGTGAAAAAATGTGGCATCCA | |
| | | AGATACAAACTCAAAGAAGCAAAGTGATACACA | |
| | | TTTGGAGGAGACGTAATCCAGCATTGGAACTTCT | |
| | | GATCTTCAAGCAGGGATTCTCAACCTGTGGTTTA | |
| | | GGGGTTCATCGGGGCTGAGCGTGACAAGAGGAA | |
| | | GGAATGGGCCCGTGGGATGCAGGCAATGTGGGA | |
| | | CTTAAAAGGCCCAAGCACTGAAAATGGAACCTG | |
| | | GCGAAAGCAGAGGAGGAGAATGAAGAAAGATG | |
| | | GAGTCAAACAGGGAGCCTGGAGGGAGACCTTGA | |
| | | TACTTTCAAATGCCTGAGGGGCTCATCGACGCCT | |
| | | GTGACAGGGAGAAAGGATACTTCTGAACAAGGA | |
| | | GCCTCCAAGCAAATCATCCATTGCTCATCCTAGG | |
| | | AAGACGGGTTGAGAATCCCTAATTTGAGGGTCA | |
| | | GTTCCTGCAGAAGTGCCCTTTGCCTCCACTCAAT | |
| | | GCCTCAATTTGTTTTCTGCATGACTGAGAGTCTC | |
| | | AGTGTTGGAACGGGACAGTATTTATGTATGAGTT | |
| | | TTTCCTATTTATTTTGAGTCTGTGAGGTCTTCTTG | |
| | | TCATGTGAGTGTGGTTGTGAATGATTTCTTTTGA | |
| | | AGATATATTGTAGTAGATGTTACAATTTTGTCGC | |
| | | CAAACTAAACTTGCTGCTTAATGATTTGCTCACA | |
| | | TCTAGTAAAACATGGAGTATTTGTAAGGTGCTTG | |
| | | GTCTCCTCTATAACTACAAGTATACATTGGAAGC | |
| | | ATAAAGATCAAACCGTTGGTTGCATAGGATGTC | |
| | | ACCTTTATTTAACCCATTAATACTCTGGTTGACC | |
| | | TAATCTTATTCTCAGACCTCAAGTGTCTGTGCAG | |
| | | TATCTGTTCCATTTAAATATCAGCTTTACAATTA | |
| | | TGTGGTAGCCTACACACATAATCTCATTTCATCG | |
| | | CTGTAACCACCCTGTTGTGATAACCACTATTATT | |
| | | TTACCCATCGTACAGCTGAGGAAGCAAACAGAT | |
| | | TAAGTAACTTGCCCAAACCAGTAAATAGCAGAC | |
| | | CTCAGACTGCCACCCACTGTCCTTTTATAATACA | |
| | | ATTTACAGCTATATTTTACTTTAAGCAATTCTTTT | |
| | | ATTCAAAAACCATTTATTAAGTGCCCTTGCAATA | |
| | | TCAATCGCTGTGCCAGGCATTGAATCTACAGATG | |
| | | TGAGCAAGACAAAGTACCTGTCCTCAAGGAGCT | |
| | | CATAGTATAATGAGGAGATTAACAAGAAAATGT | |
| | | ATTATTACAATTTAGTCCAGTGTCATAGCATAAG | |
| | | GATGATGCGAGGGGAAAACCCGAGCAGTGTTGC | |
| | | CAAGAGGAGGAAATAGGCCAATGTGGTCTGGGA | |
| | | CGGTTGGATATACTTAAACATCTTAATAATCAGA | |
| | | GTAATTTTCATTTACAAAGAGAGGTCGGTACTTA | |
| | | AAATAACCCTGAAAAATAACACTGGAATTCCTT | |
| | | TTCTAGCATTATATTTATTCCTGATTTGCCTTTGC | |
| | | CATATAATCTAATGCTTGTTTATATAGTGTCTGG | |
| | | TATTGTTTAACAGTTCTGTCTTTTCTATTTAAATG | |
| | | CCACTAAATTTTAAATTCATACCTTTCCATGATT | |
| | | CAAAATTCAAAAGATCCCATGGGAGATGGTTGG | |
| | | AAAATCTCCACTTCATCCTCCAAGCCATTCAAGT | |
| | | TTCCTTTCCAGAAGCAACTGCTACTGCCTTTCAT | |
| | | TCATATGTTCTTCTAAAGATAGTCTACATTTGGA | |
| | | AATGTATGTTAAAAGCACGTATTTTTAAAATTTT | |
| | | TTTCCTAAATAGTAACACATTGTATGTCTGCTGT | |
| | | GTACTTTGCTATTTTTATTTATTTTAGTGTTTCTT | |
| | | ATATAGCAGATGGAATGAATTTGAAGTTCCCAG | |
| | | GGCTGAGGATCCATGCCTTCTTTGTTTCTAAGTT | |
| | | ATCTTTCCCATAGCTTTTCATTATCTTTCATATGA | |
| | | TCCAGTATATGTTAAATATGTCCTACATATACAT | |
| | | TTAGACAACCACCATTTGTTAAGTATTTGCTCTA | |
| | | GGACAGAGTTTGGATTTGTTTATGTTTGCTCAAA | |
| | | AGGAGACCCATGGGCTCTCCAGGGTGCACTGAG | |
| | | TCAATCTAGTCCTAAAAAGCAATCTTATTATTAA | |
| | | CTCTGTATGACAGAATCATGTCTGGAACTTTTGT | |
| | | TTTCTGCTTTCTGTCAAGTATAAACTTCACTTTGA | |
| | | TGCTGTACTTGCAAAATCACATTTTCTTTCTGGA | |
| | | AATTCCGGCAGTGTACCTTGACTGCTAGCTACCC | |
| | | TGTGCCAGAAAAGCCTCATTCGTTGTGCTTGAAC | |
| | | CCTTGAATGCCACCAGCTGTCATCACTACACAGC | |
| | | CCTCCTAAGAGGCTTCCTGGAGGTTTCGAGATTC | |
| | | AGATGCCCTGGGAGATCCCAGAGTTTCCTTTCCC | |
| | | TCTTGGCCATATTCTGGTGTCAATGACAAGGAGT | |
| | | ACCTTGGCTTTGCCACATGTCAAGGCTGAAGAA | |
| | | ACAGTGTCTCCAACAGAGCTCCTTGTGTTATCTG | |
| | | TTTGTACATGTGCATTTGTACAGTAATTGGTGTG | |
| | | ACAGTGTTCTTTGTGTGAATTACAGGCAAGAATT | |
| | | GTGGCTGAGCAAGGCACATAGTCTACTCAGTCT | |
| | | ATTCCTAAGTCCTAACTCCTCCTTGTGGTGTTGG | |
| | | ATTTGTAAGGCACTTTATCCCTTTTGTCTCATGTT | |
| | | TCATCGTAAATGGCATAGGCAGAGATGATACCT | |
| | | AATTCTGCATTTGATTGTCACTTTTTGTACCTGCA | |
| | | TTAATTTAATAAAATATTCTTATTTATTTTGTTAC | |
| | | TTGGTACACCAGCATGTCCATTTTCTTGTTTATTT | |
| | | TGTGTTTAATAAAATGTTCAGTTTAACATCCCAG | |
| | | TGGAGAAAGTTAAAAAA | |
| CD276 | NM_001024736.1 | CCGGCCTCAGGGACGCACCGGAGCCGCCTTTCC | 18 |
| | | GGGCCTCAGGCGGATTCTCCGGCGCGGCCCGCC | |
| | | CCGCCCCTCGGACTCCCCGGGCCGCCCCCGGCCC | |
| | | CCATTCGGGCCGGGCCTCGCTGCGGCGGCGACT | |
| | | GAGCCAGGCTGGGCCGCGTCCCTGAGTCCCAGA | |
| | | GTCGGCGCGGCGCGGCAGGGGCAGCCTTCCACC | |
| | | ACGGGGAGCCCAGCTGTCAGCCGCCTCACAGGA | |
| | | AGATGCTGCGTCGGCGGGGCAGCCCTGGCATGG | |
| | | GTGTGCATGTGGGTGCAGCCCTGGGAGCACTGT | |
| | | GGTTCTGCCTCACAGGAGCCCTGGAGGTCCAGG | |
| | | TCCCTGAAGACCCAGTGGTGGCACTGGTGGGCA | |
| | | CCGATGCCACCCTGTGCTGCTCCTTCTCCCCTGA | |
| | | GCCTGGCTTCAGCCTGGCACAGCTCAACCTCATC | |
| | | TGGCAGCTGACAGATACCAAACAGCTGGTGCAC | |
| | | AGCTTTGCTGAGGGCCAGGACCAGGGCAGCGCC | |
| | | TATGCCAACCGCACGGCCCTCTTCCCGGACCTGC | |
| | | TGGCACAGGGCAACGCATCCCTGAGGCTGCAGC | |
| | | GCGTGCGTGTGGCGGACGAGGGCAGCTTCACCT | |
| | | GCTTCGTGAGCATCCGGGATTTCGGCAGCGCTGC | |
| | | CGTCAGCCTGCAGGTGGCCGCTCCCTACTCGAA | |
| | | GCCCAGCATGACCCTGGAGCCCAACAAGGACCT | |
| | | GCGGCCAGGGGACACGGTGACCATCACGTGCTC | |
| | | CAGCTACCAGGGCTACCCTGAGGCTGAGGTGTT | |
| | | CTGGCAGGATGGGCAGGGTGTGCCCCTGACTGG | |
| | | CAACGTGACCACGTCGCAGATGGCCAACGAGCA | |
| | | GGGCTTGTTTGATGTGCACAGCATCCTGCGGGTG | |
| | | GTGCTGGGTGCAAATGGCACCTACAGCTGCCTG | |
| | | GTGCGCAACCCCGTGCTGCAGCAGGATGCGCAC | |
| | | AGCTCTGTCACCATCACACCCCAGAGAAGCCCC | |
| | | ACAGGAGCCGTGGAGGTCCAGGTCCCTGAGGAC | |
| | | CCGGTGGTGGCCCTAGTGGGCACCGATGCCACC | |
| | | CTGCGCTGCTCCTTCTCCCCCGAGCCTGGCTTCA | |
| | | GCCTGGCACAGCTCAACCTCATCTGGCAGCTGA | |
| | | CAGACACCAAACAGCTGGTGCACAGTTTCACCG | |
| | | AAGGCCGGGACCAGGGCAGCGCCTATGCCAACC | |
| | | GCACGGCCCTCTTCCCGGACCTGCTGGCACAAG | |
| | | GCAATGCATCCCTGAGGCTGCAGCGCGTGCGTG | |
| | | TGGCGGACGAGGGCAGCTTCACCTGCTTCGTGA | |
| | | GCATCCGGGATTTCGGCAGCGCTGCCGTCAGCCT | |
| | | GCAGGTGGCCGCTCCCTACTCGAAGCCCAGCAT | |
| | | GACCCTGGAGCCCAACAAGGACCTGCGGCCAGG | |
| | | GGACACGGTGACCATCACGTGCTCCAGCTACCG | |
| | | GGGCTACCCTGAGGCTGAGGTGTTCTGGCAGGA | |
| | | TGGGCAGGGTGTGCCCCTGACTGGCAACGTGAC | |
| | | CACGTCGCAGATGGCCAACGAGCAGGGCTTGTT | |
| | | TGATGTGCACAGCGTCCTGCGGGTGGTGCTGGG | |
| | | TGCGAATGGCACCTACAGCTGCCTGGTGCGCAA | |
| | | CCCCGTGCTGCAGCAGGATGCGCACGGCTCTGT | |
| | | CACCATCACAGGGCAGCCTATGACATTCCCCCC | |
| | | AGAGGCCCTGTGGGTGACCGTGGGGCTGTCTGT | |
| | | CTGTCTCATTGCACTGCTGGTGGCCCTGGCTTTC | |
| | | GTGTGCTGGAGAAAGATCAAACAGAGCTGTGAG | |
| | | GAGGAGAATGCAGGAGCTGAGGACCAGGATGG | |
| | | GGAGGGAGAAGGCTCCAAGACAGCCCTGCAGCC | |
| | | TCTGAAACACTCTGACAGCAAAGAAGATGATGG | |
| | | ACAAGAAATAGCCTGACCATGAGGACCAGGGAG | |
| | | CTGCTACCCCTCCCTACAGCTCCTACCCTCTGGC | |
| | | TGCAATGGGGCTGCACTGTGAGCCCTGCCCCCA | |
| | | ACAGATGCATCCTGCTCTGACAGGTGGGCTCCTT | |
| | | CTCCAAAGGATGCGATACACAGACCACTGTGCA | |
| | | GCCTTATTTCTCCAATGGACATGATTCCCAAGTC | |
| | | ATCCTGCTGCCTTTTTTCTTATAGACACAATGAA | |
| | | CAGACCACCCACAACCTTAGTTCTCTAAGTCATC | |
| | | CTGCCTGCTGCCTTATTTCACAGTACATACATTT | |
| | | CTTAGGGACACAGTACACTGACCACATCACCAC | |
| | | CCTCTTCTTCCAGTGCTGCGTGGACCATCTGGCT | |
| | | GCCTTTTTTCTCCAAAAGATGCAATATTCAGACT | |
| | | GACTGACCCCCTGCCTTATTTCACCAAAGACACG | |
| | | ATGCATAGTCACCCCGGCCTTGTTTCTCCAATGG | |
| | | CCGTGATACACTAGTGATCATGTTCAGCCCTGCT | |
| | | TCCACCTGCATAGAATCTTTTCTTCTCAGACAGG | |
| | | GACAGTGCGGCCTCAACATCTCCTGGAGTCTAG | |
| | | AAGCTGTTTCCTTTCCCCTCCTTCCTCCTCTTGCT | |
| | | CTAGCCTTAATACTGGCCTTTTCCCTCCCTGCCC | |
| | | CAAGTGAAGACAGGGCACTCTGCGCCCACCACA | |
| | | TGCACAGCTGTGCATGGAGACCTGCAGGTGCAC | |
| | | GTGCTGGAACACGTGTGGTTCCCCCCTGGCCCAG | |
| | | CCTCCTCTGCAGTGCCCCTCTCCCCTGCCCATCC | |
| | | TCCCCACGGAAGCATGTGCTGGTCACACTGGTTC | |
| | | TCCAGGGGTCTGTGATGGGGCCCCTGGGGGTCA | |
| | | GCTTCTGTCCCTCTGCCTTCTCACCTCTTTGTTCC | |
| | | TTTCTTTTCATGTATCCATTCAGTTGATGTTTATT | |
| | | GAGCAACTACAGATGTCAGCACTGTGTTAGGTG | |
| | | CTGGGGGCCCTGCGTGGGAAGATAAAGTTCCTC | |
| | | CCTCAAGGACTCCCCATCCAGCTGGGAGACAGA | |
| | | CAACTAACTACACTGCACCCTGCGGTTTGCAGG | |
| | | GGGCTCCTGCCTGGCTCCCTGCTCCACACCTCCT | |
| | | CTGTGGCTCAAGGCTTCCTGGATACCTCACCCCC | |
| | | ATCCCACCCATAATTCTTACCCAGAGCATGGGGT | |
| | | TGGGGCGGAAACCTGGAGAGAGGGACATAGCCC | |
| | | CTCGCCACGGCTAGAGAATCTGGTGGTGTCCAA | |
| | | AATGTCTGTCCAGGTGTGGGCAGGTGGGCAGGC | |
| | | ACCAAGGCCCTCTGGACCTTTCATAGCAGCAGA | |
| | | AAAGGCAGAGCCTGGGGCAGGGCAGGGCCAGG | |
| | | AATGCTTTGGGGACACCGAGGGGACTGCCCCCC | |
| | | ACCCCCACCATGGTGCTATTCTGGGGCTGGGGC | |
| | | AGTCTTTTCCTGGCTTGCCTCTGGCCAGCTCCTG | |
| | | GCCTCTGGTAGAGTGAGACTTCAGACGTTCTGAT | |
| | | GCCTTCCGGATGTCATCTCTCCCTGCCCCAGGAA | |
| | | TGGAAGATGTGAGGACTTCTAATTTAAATGTGG | |
| | | GACTCGGAGGGATTTTGTAAACTGGGGGTATAT | |
| | | TTTGGGGAAAATAAATGTCTTTGTAAAAAGCTTA | |
| | | AAAAAAAAAAAAAAAAA | |
| CD8A | NM_001768.5 | CGAAAAGGAGGGTGACTCTCCTCGGCGGGGGCT | 19 |
| | | TCGGGTGACATCACATCCTCCAAATGCGAAATC | |
| | | AGGCTCCGGGCCGGCCGAAGGGCGCAACTTTCC | |
| | | CCCCTCGGCGCCCCACCGGCTCCCGCGCGCCTCC | |
| | | CCTCGCGCCCGAGCTTCGAGCCAAGCAGCGTCC | |
| | | TGGGGAGCGCGTCATGGCCTTACCAGTGACCGC | |
| | | CTTGCTCCTGCCGCTGGCCTTGCTGCTCCACGCC | |
| | | GCCAGGCCGAGCCAGTTCCGGGTGTCGCCGCTG | |
| | | GATCGGACCTGGAACCTGGGCGAGACAGTGGAG | |
| | | CTGAAGTGCCAGGTGCTGCTGTCCAACCCGACG | |
| | | TCGGGCTGCTCGTGGCTCTTCCAGCCGCGCGGCG | |
| | | CCGCCGCCAGTCCCACCTTCCTCCTATACCTCTC | |
| | | CCAAAACAAGCCCAAGGCGGCCGAGGGGCTGG | |
| | | ACACCCAGCGGTTCTCGGGCAAGAGGTTGGGGG | |
| | | ACACCTTCGTCCTCACCCTGAGCGACTTCCGCCG | |
| | | AGAGAACGAGGGCTACTATTTCTGCTCGGCCCT | |
| | | GAGCAACTCCATCATGTACTTCAGCCACTTCGTG | |
| | | CCGGTCTTCCTGCCAGCGAAGCCCACCACGACG | |
| | | CCAGCGCCGCGACCACCAACACCGGCGCCCACC | |
| | | ATCGCGTCGCAGCCCCTGTCCCTGCGCCCAGAG | |
| | | GCGTGCCGGCCAGCGGCGGGGGGCGCAGTGCAC | |
| | | ACGAGGGGGCTGGACTTCGCCTGTGATATCTAC | |
| | | ATCTGGGCGCCCTTGGCCGGGACTTGTGGGGTCC | |
| | | TTCTCCTGTCACTGGTTATCACCCTTTACTGCAA | |
| | | CCACAGGAACCGAAGACGTGTTTGCAAATGTCC | |
| | | CCGGCCTGTGGTCAAATCGGGAGACAAGCCCAG | |
| | | CCTTTCGGCGAGATACGTCTAACCCTGTGCAACA | |
| | | GCCACTACATTACTTCAAACTGAGATCCTTCCTT | |
| | | TTGAGGGAGCAAGTCCTTCCCTTTCATTTTTTCC | |
| | | AGTCTTCCTCCCTGTGTATTCATTCTCATGATTAT | |
| | | TATTTTAGTGGGGGCGGGGTGGGAAAGATTACT | |
| | | TTTTCTTTATGTGTTTGACGGGAAACAAAACTAG | |
| | | GTAAAATCTACAGTACACCACAAGGGTCACAAT | |
| | | ACTGTTGTGCGCACATCGCGGTAGGGCGTGGAA | |
| | | AGGGGCAGGCCAGAGCTACCCGCAGAGTTCTCA | |
| | | GAATCATGCTGAGAGAGCTGGAGGCACCCATGC | |
| | | CATCTCAACCTCTTCCCCGCCCGTTTTACAAAGG | |
| | | GGGAGGCTAAAGCCCAGAGACAGCTTGATCAAA | |
| | | GGCACACAGCAAGTCAGGGTTGGAGCAGTAGCT | |
| | | GGAGGGACCTTGTCTCCCAGCTCAGGGCTCTTTC | |
| | | CTCCACACCATTCAGGTCTTTCTTTCCGAGGCCC | |
| | | CTGTCTCAGGGTGAGGTGCTTGAGTCTCCAACGG | |
| | | CAAGGGAACAAGTACTTCTTGATACCTGGGATA | |
| | | CTGTGCCCAGAGCCTCGAGGAGGTAATGAATTA | |
| | | AAGAAGAGAACTGCCTTTGGCAGAGTTCTATAA | |
| | | TGTAAACAATATCAGACTTTTTTTTTTTATAATC | |
| | | AAGCCTAAAATTGTATAGACCTAAAATAAAATG | |
| | | AAGTGGTGAGCTTAACCCTGGAAAATGAATCCC | |
| | | TCTATCTCTAAAGAAAATCTCTGTGAAACCCCTA | |
| | | TGTGGAGGCGGAATTGCTCTCCCAGCCCTTGCAT | |
| | | TGCAGAGGGGCCCATGAAAGAGGACAGGCTACC | |
| | | CCTTTACAAATAGAATTTGAGCATCAGTGAGGTT | |
| | | AAACTAAGGCCCTCTTGAATCTCTGAATTTGAGA | |
| | | TACAAACATGTTCCTGGGATCACTGATGACTTTT | |
| | | TATACTTTGTAAAGACAATTGTTGGAGAGCCCCT | |
| | | CACACAGCCCTGGCCTCTGCTCAACTAGCAGAT | |
| | | ACAGGGATGAGGCAGACCTGACTCTCTTAAGGA | |
| | | GGCTGAGAGCCCAAACTGCTGTCCCAAACATGC | |
| | | ACTTCCTTGCTTAAGGTATGGTACAAGCAATGCC | |
| | | TGCCCATTGGAGAGAAAAAACTTAAGTAGATAA | |
| | | GGAAATAAGAACCACTCATAATTCTTCACCTTAG | |
| | | GAATAATCTCCTGTTAATATGGTGTACATTCTTC | |
| | | CTGATTATTTTCTACACATACATGTAAAATATGT | |
| | | CTTTCTTTTTTAAATAGGGTTGTACTATGCTGTTA | |
| | | TGAGTGGCTTTAATGAATAAACATTTGTAGCATC | |
| | | CTCTTTAATGGGTAAACAGCATCCGAAAAAAAA | |
| | | AAAAAAAAAAAAAAAAAAAAAAAAAAAAAAAA | |
| | | AAAAAAAAAAAAAAAAAAAAAAAAAAAAAAAA | |
| | | AAAAAAAAAAAAAAAAAAAAAAAAAAAAAAAA | |
| | | AAAAAAAAAAAAAAAAAAAA | |
| CMKRLR1 | NM_004072.1 | GAATTCGGCACGAGTCAGGGAAGCAGCCCCGGC | 20 |
| | | GGCCAGCAGGGAGCTCAGGACAGAGCAGGCTCC | |
| | | CTGGGAAGCCTCCGGGTGATAGGGGTGTTCCAG | |
| | | CTGCGGCGCTCTGGGGGTTCAGAGGGGGATCTT | |
| | | GAATGAACAAATGAATGAACTGCTTTCTGGGCA | |
| | | AACAGCCACAGCCAGAGGAGCCTGTGATTGGCA | |
| | | GAAAGAAGCCAGGGTGTGCAAGTCTCCCCAACA | |
| | | GCCTCGAGTGGCCTGCAGTCACAGGGAACCCTC | |
| | | AGGAAGACCTTCCGGGCAGAGACCAGAGGGAA | |
| | | GCCCATCTCTCCAGCAGAACTGCTTGGATTTTTC | |
| | | TACCAGGAGGCTCAGGGCTCTGCAACAATGATA | |
| | | GCAGAAGCTGATGGCATCTAGAGATCTAGGCTG | |
| | | GGACTAGCACAGCATCACTTCTACCACTTTCTGT | |
| | | TGGTCACAGCAACTCACCATGCCAGTGCAGATT | |
| | | CAAGGGGAGGAGAAATAGAGTCCACTTCTTGAT | |
| | | GGGAGGCGTGACATAGAATGGAGGATGAAGATT | |
| | | ACAACACTTCCATCAGTTACGGTGATGAATACCC | |
| | | TGATTATTTAGACTCCATTGTGGTTTTGGAGGAC | |
| | | TTATCCCCCTTGGAAGCCAGGGTGACCAGGATCT | |
| | | TCCTGGTGGTGGTCTACAGCATCGTCTGCTTCCT | |
| | | CGGGATTCTGGGCAATGGTCTGGTGATCATCATT | |
| | | GCCACCTTCAAGATGAAGAAGACAGTGAACATG | |
| | | GTCTGGTTCCTCAACCTGGCAGTGGCAGATTTCC | |
| | | TGTTCAACGTCTTCCTCCCAATCCATATCACCTA | |
| | | TGCCGCCATGGACTACCACTGGGTTTTCGGGACA | |
| | | GCCATGTGCAAGATCAGCAACTTCCTTCTCATCC | |
| | | ACAACATGTTCACCAGCGTCTTCCTGCTGACCAT | |
| | | CATCAGCTCTGACCGCTGCATCTCTGTGCTCCTC | |
| | | CCTGTCTGGTCCCAGAACCACCGCAGCGTTCGCC | |
| | | TGGCTTACATGGCCTGCATGGTCATCTGGGTCCT | |
| | | GGCTTTCTTCTTGAGTTCCCCATCTCTCGTCTTCC | |
| | | GGGACACAGCCAACCTGCATGGGAAAATATCCT | |
| | | GCTTCAACAACTTCAGCCTGTCCACACCTGGGTC | |
| | | TTCCTCGTGGCCCACTCACTCCCAAATGGACCCT | |
| | | GTGGGGTATAGCCGGCACATGGTGGTGACTGTC | |
| | | ACCCGCTTCCTCTGTGGCTTCCTGGTCCCAGTCC | |
| | | TCATCATCACAGCTTGCTACCTCACCATCGTGTG | |
| | | CAAACTGCAGCGCAACCGCCTGGCCAAGACCAA | |
| | | GAAGCCCTTCAAGATTATTGTGACCATCATCATT | |
| | | ACCTTCTTCCTCTGCTGGTGCCCCTACCACACAC | |
| | | TCAACCTCCTAGAGCTCCACCACACTGCCATGCC | |
| | | TGGCTCTGTCTTCAGCCTGGGTTTGCCCCTGGCC | |
| | | ACTGCCCTTGCCATTGCCAACAGCTGCATGAACC | |
| | | CCATTCTGTATGTTTTCATGGGTCAGGACTTCAA | |
| | | GAAGTTCAAGGTGGCCCTCTTCTCTCGCCTGGTC | |
| | | AATGCTCTAAGTGAAGATACAGGCCACTCTTCCT | |
| | | ACCCCAGCCATAGAAGCTTTACCAAGATGTCAT | |
| | | CAATGAATGAGAGGACTTCTATGAATGAGAGGG | |
| | | AGACCGGCATGCTTTGATCCTCACTGTGGAACCC | |
| | | CTCAATGGACTCTCTCAACCCAGGGACACCCAA | |
| | | GGATATGTCTTCTGAAGATCAAGGCAAGAACCT | |
| | | CTTTAGCATCCACCAATTTTCACTGCATTTTGCA | |
| | | TGGGATGAACAGTGTTTTATGCTGGGAATCTAG | |
| | | GGCCTGGAACCCCTTTCTTCTAGTGGACAGAACA | |
| | | TGCTGTGTTCCATACAGCCTTGGACTAGCAATTT | |
| | | ATGCTTCTTGGGAGGCCAGCCTTGACTGACTCAA | |
| | | AGCAAAAAAGGAAGAATTC | |
| CXCL9 | NM_002416.1 | ATCCAATACAGGAGTGACTTGGAACTCCATTCTA | 21 |
| | | TCACTATGAAGAAAAGTGGTGTTCTTTTCCTCTT | |
| | | GGGCATCATCTTGCTGGTTCTGATTGGAGTGCAA | |
| | | GGAACCCCAGTAGTGAGAAAGGGTCGCTGTTCC | |
| | | TGCATCAGCACCAACCAAGGGACTATCCACCTA | |
| | | CAATCCTTGAAAGACCTTAAACAATTTGCCCCAA | |
| | | GCCCTTCCTGCGAGAAAATTGAAATCATTGCTAC | |
| | | ACTGAAGAATGGAGTTCAAACATGTCTAAACCC | |
| | | AGATTCAGCAGATGTGAAGGAACTGATTAAAAA | |
| | | GTGGGAGAAACAGGTCAGCCAAAAGAAAAAGC | |
| | | AAAAGAATGGGAAAAAACATCAAAAAAAGAAA | |
| | | GTTCTGAAAGTTCGAAAATCTCAACGTTCTCGTC | |
| | | AAAAGAAGACTACATAAGAGACCACTTCACCAA | |
| | | TAAGTATTCTGTGTTAAAAATGTTCTATTTTAAT | |
| | | TATACCGCTATCATTCCAAAGGAGGATGGCATA | |
| | | TAATACAAAGGCTTATTAATTTGACTAGAAAATT | |
| | | TAAAACATTACTCTGAAATTGTAACTAAAGTTAG | |
| | | AAAGTTGATTTTAAGAATCCAAACGTTAAGAAT | |
| | | TGTTAAAGGCTATGATTGTCTTTGTTCTTCTACC | |
| | | ACCCACCAGTTGAATTTCATCATGCTTAAGGCCA | |
| | | TGATTTTAGCAATACCCATGTCTACACAGATGTT | |
| | | CACCCAACCACATCCCACTCACAACAGCTGCCT | |
| | | GGAAGAGCAGCCCTAGGCTTCCACGTACTGCAG | |
| | | CCTCCAGAGAGTATCTGAGGCACATGTCAGCAA | |
| | | GTCCTAAGCCTGTTAGCATGCTGGTGAGCCAAG | |
| | | CAGTTTGAAATTGAGCTGGACCTCACCAAGCTG | |
| | | CTGTGGCCATCAACCTCTGTATTTGAATCAGCCT | |
| | | ACAGGCCTCACACACAATGTGTCTGAGAGATTC | |
| | | ATGCTGATTGTTATTGGGTATCACCACTGGAGAT | |
| | | CACCAGTGTGTGGCTTTCAGAGCCTCCTTTCTGG | |
| | | CTTTGGAAGCCATGTGATTCCATCTTGCCCGCTC | |
| | | AGGCTGACCACTTTATTTCTTTTTGTTCCCCTTTG | |
| | | CTTCATTCAAGTCAGCTCTTCTCCATCCTACCAC | |
| | | AATGCAGTGCCTTTCTTCTCTCCAGTGCACCTGT | |
| | | CATATGCTCTGATTTATCTGAGTCAACTCCTTTCT | |
| | | CATCTTGTCCCCAACACCCCACAGAAGTGCTTTC | |
| | | TTCTCCCAATTCATCCTCACTCAGTCCAGCTTAG | |
| | | TTCAAGTCCTGCCTCTTAAATAAACCTTTTTGGA | |
| | | CACACAAATTATCTTAAAACTCCTGTTTCACTTG | |
| | | GTTCAGTACCACATGGGTGAACACTCAATGGTT | |
| | | AACTAATTCTTGGGTGTTTATCCTATCTCTCCAA | |
| | | CCAGATTGTCAGCTCCTTGAGGGCAAGAGCCAC | |
| | | AGTATATTTCCCTGTTTCTTCCACAGTGCCTAAT | |
| | | AATACTGTGGAACTAGGTTTTAATAATTTTTTAA | |
| | | TTGATGTTGTTATGGGCAGGATGGCAACCAGAC | |
| | | CATTGTCTCAGAGCAGGTGCTGGCTCTTTCCTGG | |
| | | CTACTCCATGTTGGCTAGCCTCTGGTAACCTCTT | |
| | | ACTTATTATCTTCAGGACACTCACTACAGGGACC | |
| | | AGGGATGATGCAACATCCTTGTCTTTTTATGACA | |
| | | GGATGTTTGCTCAGCTTCTCCAACAATAAGAAGC | |
| | | ACGTGGTAAAACACTTGCGGATATTCTGGACTGT | |
| | | TTTTAAAAAATATACAGTTTACCGAAAATCATAT | |
| | | AATCTTACAATGAAAAGGACTTTATAGATCAGC | |
| | | CAGTGACCAACCTTTTCCCAACCATACAAAAATT | |
| | | CCTTTTCCCGAAGGAAAAGGGCTTTCTCAATAAG | |
| | | CCTCAGCTTTCTAAGATCTAACAAGATAGCCACC | |
| | | GAGATCCTTATCGAAACTCATTTTAGGCAAATAT | |
| | | GAGTTTTATTGTCCGTTTACTTGTTTCAGAGTTTG | |
| | | TATTGTGATTATCAATTACCACACCATCTCCCAT | |
| | | GAAGAAAGGGAACGGTGAAGTACTAAGCGCTA | |
| | | GAGGAAGCAGCCAAGTCGGTTAGTGGAAGCATG | |
| | | ATTGGTGCCCAGTTAGCCTCTGCAGGATGTGGA | |
| | | AACCTCCTTCCAGGGGAGGTTCAGTGAATTGTGT | |
| | | AGGAGAGGTTGTCTGTGGCCAGAATTTAAACCT | |
| | | ATACTCACTTTCCCAAATTGAATCACTGCTCACA | |
| | | CTGCTGATGATTTAGAGTGCTGTCCGGTGGAGAT | |
| | | CCCACCCGAACGTCTTATCTAATCATGAAACTCC | |
| | | CTAGTTCCTTCATGTAACTTCCCTGAAAAATCTA | |
| | | AGTGTTTCATAAATTTGAGAGTCTGTGACCCACT | |
| | | TACCTTGCATCTCACAGGTAGACAGTATATAACT | |
| | | AACAACCAAAGACTACATATTGTCACTGACACA | |
| | | CACGTTATAATCATTTATCATATATATACATACA | |
| | | TGCATACACTCTCAAAGCAAATAATTTTTCACTT | |
| | | CAAAACAGTATTGACTTGTATACCTTGTAATTTG | |
| | | AAATATTTTCTTTGTTAAAATAGAATGGTATCAA | |
| | | TAAATAGACCATTAATCAG | |
| CXCR6 | NM_006564.1 | GCAGACCTTGCTTCATGAGCAAGCTCATCTCTGG | 22 |
| | | AACAAACTGGCAAAGCATCTCTGCTGGTGTTCAT | |
| | | CAGAACAGACACCATGGCAGAGCATGATTACCA | |
| | | TGAAGACTATGGGTTCAGCAGTTTCAATGACAG | |
| | | CAGCCAGGAGGAGCATCAAGACTTCCTGCAGTT | |
| | | CAGCAAGGTCTTTCTGCCCTGCATGTACCTGGTG | |
| | | GTGTTTGTCTGTGGTCTGGTGGGGAACTCTCTGG | |
| | | TGCTGGTCATATCCATCTTCTACCATAAGTTGCA | |
| | | GAGCCTGACGGATGTGTTCCTGGTGAACCTACCC | |
| | | CTGGCTGACCTGGTGTTTGTCTGCACTCTGCCCT | |
| | | TCTGGGCCTATGCAGGCATCCATGAATGGGTGTT | |
| | | TGGCCAGGTCATGTGCAAGAGCCTACTGGGCAT | |
| | | CTACACTATTAACTTCTACACGTCCATGCTCATC | |
| | | CTCACCTGCATCACTGTGGATCGTTTCATTGTAG | |
| | | TGGTTAAGGCCACCAAGGCCTACAACCAGCAAG | |
| | | CCAAGAGGATGACCTGGGGCAAGGTCACCAGCT | |
| | | TGCTCATCTGGGTGATATCCCTGCTGGTTTCCTT | |
| | | GCCCCAAATTATCTATGGCAATGTCTTTAATCTC | |
| | | GACAAGCTCATATGTGGTTACCATGACGAGGCA | |
| | | ATTTCCACTGTGGTTCTTGCCACCCAGATGACAC | |
| | | TGGGGTTCTTCTTGCCACTGCTCACCATGATTGT | |
| | | CTGCTATTCAGTCATAATCAAAACACTGCTTCAT | |
| | | GCTGGAGGCTTCCAGAAGCACAGATCTCTAAAG | |
| | | ATCATCTTCCTGGTGATGGCTGTGTTCCTGCTGA | |
| | | CCCAGATGCCCTTCAACCTCATGAAGTTCATCCG | |
| | | CAGCACACACTGGGAATACTATGCCATGACCAG | |
| | | CTTTCACTACACCATCATGGTGACAGAGGCCATC | |
| | | GCATACCTGAGGGCCTGCCTTAACCCTGTGCTCT | |
| | | ATGCCTTTGTCAGCCTGAAGTTTCGAAAGAACTT | |
| | | CTGGAAACTTGTGAAGGACATTGGTTGCCTCCCT | |
| | | TACCTTGGGGTCTCACATCAATGGAAATCTTCTG | |
| | | AGGACAATTCCAAGACTTTTTCTGCCTCCCACAA | |
| | | TGTGGAGGCCACCAGCATGTTCCAGTTATAGGC | |
| | | CTTGCCAGGGTTTCGAGAAGCTGCTCTGGAATTT | |
| | | GCAAGTCATGGCTGTGCCCTCTTGATGTGGTGAG | |
| | | GCAGGCTTTGTTTATAGCTTGCGCATTCTCATGG | |
| | | AGAAGTTATCAGACACTCTGGCTGGTTTGGAAT | |
| | | GCTTCTTCTCAGGCATGAACATGTACTGTTCTCT | |
| | | TCTTGAACACTCATGCTGAAAGCCCAAGTAGGG | |
| | | GGTCTAAAATTTTTAAGGACTTTCCTTCCTCCAT | |
| | | CTCCAAGAATGCTGAAACCAAGGGGGATGACAT | |
| | | GTGACTCCTATGATCTCAGGTTCTCCTTGATTGG | |
| | | GACTGGGGCTGAAGGTTGAAGAGGTGAGCACGG | |
| | | CCAACAAAGCTGTTGATGGTAGGTGGCACACTG | |
| | | GGTGCCCAAGCTCAGAAGGCTCTTCTGACTACTG | |
| | | GGCAAAGAGTGTAGATCAGAGCAGCAGTGAAA | |
| | | ACAAGTGCTGGCACCACCAGGCACCTCACAGAA | |
| | | ATGAGATCAGGCTCTGCCTCACCTTGGGGCTTGA | |
| | | CTTTTGTATAGGTAGATGTTCAGATTGCTTTGAT | |
| | | TAATCCAGAATAACTAGCACCAGGGACTATGAA | |
| | | TGGGCAAAACTGAATTATAAGAGGCTGATAATT | |
| | | CCAGTGGTCCATGGAATGCTTGAAAAATGTGCA | |
| | | AAACAGCGTTTAAGACTGTAATGAATCTAAGCA | |
| | | GCATTTCTGAAGTGGACTCTTTGGTGGCTTTGCA | |
| | | TTTTAAAAATGAAATTTTCCAATGTCTGCCACAC | |
| | | AAACGTATGTAAATGTATATACCCACACACATA | |
| | | CACACATATGTCATATATTACTAGCATATGAGTT | |
| | | TCATAGCTAAGAAATAAAACTGTTAAAGTCTCC | |
| | | AAACT | |
| HLA- | NM_002122.3 | ACAATTACTCTACAGCTCAGAACACCAACTGCT | 23 |
| DQA1 | | GAGGCTGCCTTGGGAAGAGGATGATCCTAAACA | |
| | | AAGCTCTGCTGCTGGGGGCCCTCGCTCTGACCAC | |
| | | CGTGATGAGCCCCTGTGGAGGTGAAGACATTGT | |
| | | GGCTGACCACGTTGCCTCTTGTGGTGTAAACTTG | |
| | | TACCAGTTTTACGGTCCCTCTGGCCAGTACACCC | |
| | | ATGAATTTGATGGAGATGAGCAGTTCTACGTGG | |
| | | ACCTGGAGAGGAAGGAGACTGCCTGGCGGTGGC | |
| | | CTGAGTTCAGCAAATTTGGAGGTTTTGACCCGCA | |
| | | GGGTGCACTGAGAAACATGGCTGTGGCAAAACA | |
| | | CAACTTGAACATCATGATTAAACGCTACAACTCT | |
| | | ACCGCTGCTACCAATGAGGTTCCTGAGGTCACA | |
| | | GTGTTTTCCAAGTCTCCCGTGACACTGGGTCAGC | |
| | | CCAACACCCTCATTTGTCTTGTGGACAACATCTT | |
| | | TCCTCCTGTGGTCAACATCACATGGCTGAGCAAT | |
| | | GGGCAGTCAGTCACAGAAGGTGTTTCTGAGACC | |
| | | AGCTTCCTCTCCAAGAGTGATCATTCCTTCTTCA | |
| | | AGATCAGTTACCTCACCTTCCTCCCTTCTGCTGA | |
| | | TGAGATTTATGACTGCAAGGTGGAGCACTGGGG | |
| | | CCTGGACCAGCCTCTTCTGAAACACTGGGAGCCT | |
| | | GAGATTCCAGCCCCTATGTCAGAGCTCACAGAG | |
| | | ACTGTGGTCTGTGCCCTGGGGTTGTCTGTGGGCC | |
| | | TCATGGGCATTGTGGTGGGCACTGTCTTCATCAT | |
| | | CCAAGGCCTGCGTTCAGTTGGTGCTTCCAGACAC | |
| | | CAAGGGCCATTGTGAATCCCATCCTGGAAGGGA | |
| | | AGGTGCATCGCCATCTACAGGAGCAGAAGAATG | |
| | | GACTTGCTAAATGACCTAGCACTATTCTCTGGCC | |
| | | CGATTTATCATATCCCTTTTCTCCTCCAAATATTT | |
| | | CTCCTCTCACCTTTTCTCTGGGACTTAAGCTGCT | |
| | | ATATCCCCTCAGAGCTCACAAATGCCTTTACATT | |
| | | CTTTCCCTGACCTCCTGATTTTTTTTTTCTTTTCTC | |
| | | AAATGTTACCTACAAAGACATGCCTGGGGTAAG | |
| | | CCACCCGGCTACCTAATTCCTCAGTAACCTCCAT | |
| | | CTAAAATCTCCAAGGAAGCAATAAATTCCTTTTA | |
| | | TGAGATCTATGTCAAATTTTTCCATCTTTCATCC | |
| | | AGGGCTGACTGAAACTATGGCTAATAATTGGGG | |
| | | TACTCTTATGTTTCAATCCAATTTAACCTCATTTC | |
| | | CCAGATCATTTTTCATGTCCAGTAACACAGAAGC | |
| | | CACCAAGTACAGTATAGCCTGATAATATGTTGAT | |
| | | TTCTTAGCTGACATTAATATTTCTTGCTTCCTTGT | |
| | | GTTCCCACCCTTGGCACTGCCACCCACCCCTCAA | |
| | | TTCAGGCAACAATGAAATTAATGGATACCGTCT | |
| | | GCCCTTGGCCCAGAATTGTTATAGCAAAAATTTT | |
| | | AGAACCAAAAAATAAGTCTGTACTAATTTCAAT | |
| | | GTGGCTTTTAAAAGTATGACAGAGAAATAAGTT | |
| | | AGGATAAAGGAAATTTGAATCTCA | |
| HLA- | NM_002124.1 | TAGTTCTCCCTGAGTGAGACTTGCCTGCTTCTCT | 24 |
| DRB1 | | GGCCCCTGGTCCTGTCCTGTTCTCCAGCATGGTG | |
| | | TGTCTGAAGCTCCCTGGAGGCTCCTGCATGACAG | |
| | | CGCTGACAGTGACACTGATGGTGCTGAGCTCCC | |
| | | CACTGGCTTTGGCTGGGGACACCCGACCACGTTT | |
| | | CTTGTGGCAGCTTAAGTTTGAATGTCATTTCTTC | |
| | | AATGGGACGGAGCGGGTGCGGTTGCTGGAAAGA | |
| | | TGCATCTATAACCAAGAGGAGTCCGTGCGCTTC | |
| | | GACAGCGACGTGGGGGAGTACCGGGCGGTGACG | |
| | | GAGCTGGGGCGGCCTGATGCCGAGTACTGGAAC | |
| | | AGCCAGAAGGACCTCCTGGAGCAGAGGCGGGCC | |
| | | GCGGTGGACACCTACTGCAGACACAACTACGGG | |
| | | GTTGGTGAGAGCTTCACAGTGCAGCGGCGAGTT | |
| | | GAGCCTAAGGTGACTGTGTATCCTTCAAAGACC | |
| | | CAGCCCCTGCAGCACCACAACCTCCTGGTCTGCT | |
| | | CTGTGAGTGGTTTCTATCCAGGCAGCATTGAAGT | |
| | | CAGGTGGTTCCGGAACGGCCAGGAAGAGAAGGC | |
| | | TGGGGTGGTGTCCACAGGCCTGATCCAGAATGG | |
| | | AGATTGGACCTTCCAGACCCTGGTGATGCTGGA | |
| | | AACAGTTCCTCGGAGTGGAGAGGTTTACACCTG | |
| | | CCAAGTGGAGCACCCAAGTGTGACGAGCCCTCT | |
| | | CACAGTGGAATGGAGAGCACGGTCTGAATCTGC | |
| | | ACAGAGCAAGATGCTGAGTGGAGTCGGGGGCTT | |
| | | CGTGCTGGGCCTGCTCTTCCTTGGGGCCGGGCTG | |
| | | TTCATCTACTTCAGGAATCAGAAAGGACACTCTG | |
| | | GACTTCAGCCAACAGGATTCCTGAGCTGAAATG | |
| | | CAGATGACCACATTCAAGGAAGAACCTTCTGTC | |
| | | CCAGCTTTGCAGAATGAAAAGCTTTCCTGCTTGG | |
| | | CAGTTATTCTTCCACAAGAGAGGGCTTTCTCAGG | |
| | | ACCTGGTTGCTACTGGTTCGGCAACTGCAGAAA | |
| | | ATGTCCTCCCTTGTGGCTTCCTCAGCTCCTGCCCT | |
| | | TGGCCTGAAGTCCCAGCATTGATGACAGCGCCT | |
| | | CATCTTCAACTTTTGTGCTCCCCTTTGCCTAAACC | |
| | | GTATGGCCTCCCGTGCATCTGTACTCACCCTGTA | |
| | | CGACAAACACATTACATTATTAAATGTTTCTCAA | |
| | | AGATGGAGTT | |
| HLA-E | NM_005516.4 | CGGACTCAAGAAGTTCTCAGGACTCAGAGGCTG | 25 |
| | | GGATCATGGTAGATGGAACCCTCCTTTTACTCCT | |
| | | CTCGGAGGCCCTGGCCCTTACCCAGACCTGGGC | |
| | | GGGCTCCCACTCCTTGAAGTATTTCCACACTTCC | |
| | | GTGTCCCGGCCCGGCCGCGGGGAGCCCCGCTTC | |
| | | ATCTCTGTGGGCTACGTGGACGACACCCAGTTCG | |
| | | TGCGCTTCGACAACGACGCCGCGAGTCCGAGGA | |
| | | TGGTGCCGCGGGCGCCGTGGATGGAGCAGGAGG | |
| | | GGTCAGAGTATTGGGACCGGGAGACACGGAGCG | |
| | | CCAGGGACACCGCACAGATTTTCCGAGTGAACC | |
| | | TGCGGACGCTGCGCGGCTACTACAATCAGAGCG | |
| | | AGGCCGGGTCTCACACCCTGCAGTGGATGCATG | |
| | | GCTGCGAGCTGGGGCCCGACGGGCGCTTCCTCC | |
| | | GCGGGTATGAACAGTTCGCCTACGACGGCAAGG | |
| | | ATTATCTCACCCTGAATGAGGACCTGCGCTCCTG | |
| | | GACCGCGGTGGACACGGCGGCTCAGATCTCCGA | |
| | | GCAAAAGTCAAATGATGCCTCTGAGGCGGAGCA | |
| | | CCAGAGAGCCTACCTGGAAGACACATGCGTGGA | |
| | | GTGGCTCCACAAATACCTGGAGAAGGGGAAGGA | |
| | | GACGCTGCTTCACCTGGAGCCCCCAAAGACACA | |
| | | CGTGACTCACCACCCCATCTCTGACCATGAGGCC | |
| | | ACCCTGAGGTGCTGGGCCCTGGGCTTCTACCCTG | |
| | | CGGAGATCACACTGACCTGGCAGCAGGATGGGG | |
| | | AGGGCCATACCCAGGACACGGAGCTCGTGGAGA | |
| | | CCAGGCCTGCAGGGGATGGAACCTTCCAGAAGT | |
| | | GGGCAGCTGTGGTGGTGCCTTCTGGAGAGGAGC | |
| | | AGAGATACACGTGCCATGTGCAGCATGAGGGGC | |
| | | TACCCGAGCCCGTCACCCTGAGATGGAAGCCGG | |
| | | CTTCCCAGCCCACCATCCCCATCGTGGGCATCAT | |
| | | TGCTGGCCTGGTTCTCCTTGGATCTGTGGTCTCT | |
| | | GGAGCTGTGGTTGCTGCTGTGATATGGAGGAAG | |
| | | AAGAGCTCAGGTGGAAAAGGAGGGAGCTACTCT | |
| | | AAGGCTGAGTGGAGCGACAGTGCCCAGGGGTCT | |
| | | GAGTCTCACAGCTTGTAAAGCCTGAGACAGCTG | |
| | | CCTTGTGTGCGACTGAGATGCACAGCTGCCTTGT | |
| | | GTGCGACTGAGATGCAGGATTTCCTCACGCCTCC | |
| | | CCTATGTGTCTTAGGGGACTCTGGCTTCTCTTTTT | |
| | | GCAAGGGCCTCTGAATCTGTCTGTGTCCCTGTTA | |
| | | GCACAATGTGAGGAGGTAGAGAAACAGTCCACC | |
| | | TCTGTGTCTACCATGACCCCCTTCCTCACACTGA | |
| | | CCTGTGTTCCTTCCCTGTTCTCTTTTCTATTAAAA | |
| | | ATAAGAACCTGGGCAGAGTGCGGCAGCTCATGC | |
| | | CTGTAATCCCAGCACTTAGGGAGGCCGAGGAGG | |
| | | GCAGATCACGAGGTCAGGAGATCGAAACCATCC | |
| | | TGGCTAACACGGTGAAACCCCGTCTCTACTAAA | |
| | | AAATACAAAAAATTAGCTGGGCGCAGAGGCACG | |
| | | GGCCTGTAGTCCCAGCTACTCAGGAGGCGGAGG | |
| | | CAGGAGAATGGCGTCAACCCGGGAGGCGGAGGT | |
| | | TGCAGTGAGCCAGGATTGTGCGACTGCACTCCA | |
| | | GCCTGGGTGACAGGGTGAAACGCCATCTCAAAA | |
| | | AATAAAAATTGAAAAATAAAAAAAAAAAAAAA | |
| | | AAA | |
| IDO1 | NM_002164.3 | AATTTCTCACTGCCCCTGTGATAAACTGTGGTCA | 26 |
| | | CTGGCTGTGGCAGCAACTATTATAAGATGCTCTG | |
| | | AAAACTCTTCAGACACTGAGGGGCACCAGAGGA | |
| | | GCAGACTACAAGAATGGCACACGCTATGGAAAA | |
| | | CTCCTGGACAATCAGTAAAGAGTACCATATTGA | |
| | | TGAAGAAGTGGGCTTTGCTCTGCCAAATCCACA | |
| | | GGAAAATCTACCTGATTTTTATAATGACTGGATG | |
| | | TTCATTGCTAAACATCTGCCTGATCTCATAGAGT | |
| | | CTGGCCAGCTTCGAGAAAGAGTTGAGAAGTTAA | |
| | | ACATGCTCAGCATTGATCATCTCACAGACCACA | |
| | | AGTCACAGCGCCTTGCACGTCTAGTTCTGGGATG | |
| | | CATCACCATGGCATATGTGTGGGGCAAAGGTCA | |
| | | TGGAGATGTCCGTAAGGTCTTGCCAAGAAATAT | |
| | | TGCTGTTCCTTACTGCCAACTCTCCAAGAAACTG | |
| | | GAACTGCCTCCTATTTTGGTTTATGCAGACTGTG | |
| | | TCTTGGCAAACTGGAAGAAAAAGGATCCTAATA | |
| | | AGCCCCTGACTTATGAGAACATGGACGTTTTGTT | |
| | | CTCATTTCGTGATGGAGACTGCAGTAAAGGATTC | |
| | | TTCCTGGTCTCTCTATTGGTGGAAATAGCAGCTG | |
| | | CTTCTGCAATCAAAGTAATTCCTACTGTATTCAA | |
| | | GGCAATGCAAATGCAAGAACGGGACACTTTGCT | |
| | | AAAGGCGCTGTTGGAAATAGCTTCTTGCTTGGA | |
| | | GAAAGCCCTTCAAGTGTTTCACCAAATCCACGAT | |
| | | CATGTGAACCCAAAAGCATTTTTCAGTGTTCTTC | |
| | | GCATATATTTGTCTGGCTGGAAAGGCAACCCCC | |
| | | AGCTATCAGACGGTCTGGTGTATGAAGGGTTCT | |
| | | GGGAAGACCCAAAGGAGTTTGCAGGGGGCAGTG | |
| | | CAGGCCAAAGCAGCGTCTTTCAGTGCTTTGACGT | |
| | | CCTGCTGGGCATCCAGCAGACTGCTGGTGGAGG | |
| | | ACATGCTGCTCAGTTCCTCCAGGACATGAGAAG | |
| | | ATATATGCCACCAGCTCACAGGAACTTCCTGTGC | |
| | | TCATTAGAGTCAAATCCCTCAGTCCGTGAGTTTG | |
| | | TCCTTTCAAAAGGTGATGCTGGCCTGCGGGAAG | |
| | | CTTATGACGCCTGTGTGAAAGCTCTGGTCTCCCT | |
| | | GAGGAGCTACCATCTGCAAATCGTGACTAAGTA | |
| | | CATCCTGATTCCTGCAAGCCAGCAGCCAAAGGA | |
| | | GAATAAGACCTCTGAAGACCCTTCAAAACTGGA | |
| | | AGCCAAAGGAACTGGAGGCACTGATTTAATGAA | |
| | | TTTCCTGAAGACTGTAAGAAGTACAACTGAGAA | |
| | | ATCCCTTTTGAAGGAAGGTTAATGTAACCCAAC | |
| | | AAGAGCACATTTTATCATAGCAGAGACATCTGT | |
| | | ATGCATTCCTGTCATTACCCATTGTAACAGAGCC | |
| | | ACAAACTAATACTATGCAATGTTTTACCAATAAT | |
| | | GCAATACAAAAGACCTCAAAATACCTGTGCATT | |
| | | TCTTGTAGGAAAACAACAAAAGGTAATTATGTG | |
| | | TAATTATACTAGAAGTTTTGTAATCTGTATCTTA | |
| | | TCATTGGAATAAAATGACATTCAATAAATAAAA | |
| | | AAAAAAAAAAAAAAAAAAAAAAAAAAAAAAAA | |
| | | AAAAAAAAAAAAAAAAAAAAAAAAAAAAAAAA | |
| | | AAAAAAAAAAAAAAAAAAAA | |
| LAG3 | NM_002286.5 | ACAGGGGTGAAGGCCCAGAGACCAGCAGAACG | 27 |
| | | GCATCCCAGCCACGACGGCCACTTTGCTCTGTCT | |
| | | GCTCTCCGCCACGGCCCTGCTCTGTTCCCTGGGA | |
| | | CACCCCCGCCCCCACCTCCTCAGGCTGCCTGATC | |
| | | TGCCCAGCTTTCCAGCTTTCCTCTGGATTCCGGC | |
| | | CTCTGGTCATCCCTCCCCACCCTCTCTCCAAGGC | |
| | | CCTCTCCTGGTCTCCCTTCTTCTAGAACCCCTTCC | |
| | | TCCACCTCCCTCTCTGCAGAACTTCTCCTTTACCC | |
| | | CCCACCCCCCACCACTGCCCCCTTTCCTTTTCTG | |
| | | ACCTCCTTTTGGAGGGCTCAGCGCTGCCCAGACC | |
| | | ATAGGAGAGATGTGGGAGGCTCAGTTCCTGGGC | |
| | | TTGCTGTTTCTGCAGCCGCTTTGGGTGGCTCCAG | |
| | | TGAAGCCTCTCCAGCCAGGGGCTGAGGTCCCGG | |
| | | TGGTGTGGGCCCAGGAGGGGGCTCCTGCCCAGC | |
| | | TCCCCTGCAGCCCCACAATCCCCCTCCAGGATCT | |
| | | CAGCCTTCTGCGAAGAGCAGGGGTCACTTGGCA | |
| | | GCATCAGCCAGACAGTGGCCCGCCCGCTGCCGC | |
| | | CCCCGGCCATCCCCTGGCCCCCGGCCCTCACCCG | |
| | | GCGGCGCCCTCCTCCTGGGGGCCCAGGCCCCGC | |
| | | CGCTACACGGTGCTGAGCGTGGGTCCCGGAGGC | |
| | | CTGCGCAGCGGGAGGCTGCCCCTGCAGCCCCGC | |
| | | GTCCAGCTGGATGAGCGCGGCCGGCAGCGCGGG | |
| | | GACTTCTCGCTATGGCTGCGCCCAGCCCGGCGCG | |
| | | CGGACGCCGGCGAGTACCGCGCCGCGGTGCACC | |
| | | TCAGGGACCGCGCCCTCTCCTGCCGCCTCCGTCT | |
| | | GCGCCTGGGCCAGGCCTCGATGACTGCCAGCCC | |
| | | CCCAGGATCTCTCAGAGCCTCCGACTGGGTCATT | |
| | | TTGAACTGCTCCTTCAGCCGCCCTGACCGCCCAG | |
| | | CCTCTGTGCATTGGTTCCGGAACCGGGGCCAGG | |
| | | GCCGAGTCCCTGTCCGGGAGTCCCCCCATCACCA | |
| | | CTTAGCGGAAAGCTTCCTCTTCCTGCCCCAAGTC | |
| | | AGCCCCATGGACTCTGGGCCCTGGGGCTGCATC | |
| | | CTCACCTACAGAGATGGCTTCAACGTCTCCATCA | |
| | | TGTATAACCTCACTGTTCTGGGTCTGGAGCCCCC | |
| | | AACTCCCTTGACAGTGTACGCTGGAGCAGGTTCC | |
| | | AGGGTGGGGCTGCCCTGCCGCCTGCCTGCTGGT | |
| | | GTGGGGACCCGGTCTTTCCTCACTGCCAAGTGGA | |
| | | CTCCTCCTGGGGGAGGCCCTGACCTCCTGGTGAC | |
| | | TGGAGACAATGGCGACTTTACCCTTCGACTAGA | |
| | | GGATGTGAGCCAGGCCCAGGCTGGGACCTACAC | |
| | | CTGCCATATCCATCTGCAGGAACAGCAGCTCAA | |
| | | TGCCACTGTCACATTGGCAATCATCACAGTGACT | |
| | | CCCAAATCCTTTGGGTCACCTGGATCCCTGGGGA | |
| | | AGCTGCTTTGTGAGGTGACTCCAGTATCTGGACA | |
| | | AGAACGCTTTGTGTGGAGCTCTCTGGACACCCCA | |
| | | TCCCAGAGGAGTTTCTCAGGACCTTGGCTGGAG | |
| | | GCACAGGAGGCCCAGCTCCTTTCCCAGCCTTGGC | |
| | | AATGCCAGCTGTACCAGGGGGAGAGGCTTCTTG | |
| | | GAGCAGCAGTGTACTTCACAGAGCTGTCTAGCC | |
| | | CAGGTGCCCAACGCTCTGGGAGAGCCCCAGGTG | |
| | | CCCTCCCAGCAGGCCACCTCCTGCTGTTTCTCAT | |
| | | CCTTGGTGTCCTTTCTCTGCTCCTTTTGGTGACTG | |
| | | GAGCCTTTGGCTTTCACCTTTGGAGAAGACAGTG | |
| | | GCGACCAAGACGATTTTCTGCCTTAGAGCAAGG | |
| | | GATTCACCCTCCGCAGGCTCAGAGCAAGATAGA | |
| | | GGAGCTGGAGCAAGAACCGGAGCCGGAGCCGG | |
| | | AGCCGGAACCGGAGCCCGAGCCCGAGCCCGAGC | |
| | | CGGAGCAGCTCTGACCTGGAGCTGAGGCAGCCA | |
| | | GCAGATCTCAGCAGCCCAGTCCAAATAAACTCC | |
| | | CTGTCAGCAGCAAAAA | |
| NKG7 | NM_005601.3 | TCATGTGACAAAGCGCAGGACCCCTCACTGCCC | 28 |
| | | CAACTGCTTGCTGTTCTCTCTTTCTTGGGCTCTAA | |
| | | GGACCCAGGAGTCTGGGTGCACAGCCTCCTTCTC | |
| | | TCTGAGATTCAAGAGTCTGATCAGCAGCCTCTTC | |
| | | CTCCTCCAGGACCCAGAAGCCCTGAGCTTATCCC | |
| | | CATGGAGCTCTGCCGGTCCCTGGCCCTGCTGGGG | |
| | | GGCTCCCTGGGCCTGATGTTCTGCCTGATTGCTT | |
| | | TGAGCACCGATTTCTGGTTTGAGGCTGTGGGTCC | |
| | | CACCCACTCAGCTCACTCGGGCCTCTGGCCAACA | |
| | | GGGCATGGGGACATCATATCAGGCTACATCCAC | |
| | | GTGACGCAGACCTTCAGCATTATGGCTGTTCTGT | |
| | | GGGCCCTGGTGTCCGTGAGCTTCCTGGTCCTGTC | |
| | | CTGCTTCCCCTCACTGTTCCCCCCAGGCCACGGC | |
| | | CCGCTTGTCTCAACCACCGCAGCCTTTGCTGCAG | |
| | | CCATCTCCATGGTGGTGGCCATGGCGGTGTACAC | |
| | | CAGCGAGCGGTGGGACCAGCCTCCACACCCCCA | |
| | | GATCCAGACCTTCTTCTCCTGGTCCTTCTACCTG | |
| | | GGCTGGGTCTCAGCTATCCTCTTGCTCTGTACAG | |
| | | GTGCCCTGAGCCTGGGTGCTCACTGTGGCGGTCC | |
| | | CCGTCCTGGCTATGAAACCTTGTGAGCAGAAGG | |
| | | CAAGAGCGGCAAGATGAGTTTTGAGCGTTGTAT | |
| | | TCCAAAGGCCTCATCTGGAGCCTCGGGAAAGTC | |
| | | TGGTCCCACATCTGCCCGCCCTTCCAGCCCTTCC | |
| | | CCAGCCCCTCCTCTTGTTTCTTCATTCATTCAACA | |
| | | AAATTTGGCTGGAA | |
| PDCD1LG2 | NM_025239.3 | GCAAACCTTAAGCTGAATGAACAACTTTTCTTCT | 29 |
| | | CTTGAATATATCTTAACGCCAAATTTTGAGTGCT | |
| | | TTTTTGTTACCCATCCTCATATGTCCCAGCTAGA | |
| | | AAGAATCCTGGGTTGGAGCTACTGCATGTTGATT | |
| | | GTTTTGTTTTTCCTTTTGGCTGTTCATTTTGGTGG | |
| | | CTACTATAAGGAAATCTAACACAAACAGCAACT | |
| | | GTTTTTTGTTGTTTACTTTTGCATCTTTACTTGTG | |
| | | GAGCTGTGGCAAGTCCTCATATCAAATACAGAA | |
| | | CATGATCTTCCTCCTGCTAATGTTGAGCCTGGAA | |
| | | TTGCAGCTTCACCAGATAGCAGCTTTATTCACAG | |
| | | TGACAGTCCCTAAGGAACTGTACATAATAGAGC | |
| | | ATGGCAGCAATGTGACCCTGGAATGCAACTTTG | |
| | | ACACTGGAAGTCATGTGAACCTTGGAGCAATAA | |
| | | CAGCCAGTTTGCAAAAGGTGGAAAATGATACAT | |
| | | CCCCACACCGTGAAAGAGCCACTTTGCTGGAGG | |
| | | AGCAGCTGCCCCTAGGGAAGGCCTCGTTCCACA | |
| | | TACCTCAAGTCCAAGTGAGGGACGAAGGACAGT | |
| | | ACCAATGCATAATCATCTATGGGGTCGCCTGGG | |
| | | ACTACAAGTACCTGACTCTGAAAGTCAAAGCTT | |
| | | CCTACAGGAAAATAAACACTCACATCCTAAAGG | |
| | | TTCCAGAAACAGATGAGGTAGAGCTCACCTGCC | |
| | | AGGCTACAGGTTATCCTCTGGCAGAAGTATCCTG | |
| | | GCCAAACGTCAGCGTTCCTGCCAACACCAGCCA | |
| | | CTCCAGGACCCCTGAAGGCCTCTACCAGGTCAC | |
| | | CAGTGTTCTGCGCCTAAAGCCACCCCCTGGCAG | |
| | | AAACTTCAGCTGTGTGTTCTGGAATACTCACGTG | |
| | | AGGGAACTTACTTTGGCCAGCATTGACCTTCAAA | |
| | | GTCAGATGGAACCCAGGACCCATCCAACTTGGC | |
| | | TGCTTCACATTTTCATCCCCTTCTGCATCATTGCT | |
| | | TTCATTTTCATAGCCACAGTGATAGCCCTAAGAA | |
| | | AACAACTCTGTCAAAAGCTGTATTCTTCAAAAG | |
| | | ACACAACAAAAAGACCTGTCACCACAACAAAGA | |
| | | GGGAAGTGAACAGTGCTATCTGAACCTGTGGTC | |
| | | TTGGGAGCCAGGGTGACCTGATATGACATCTAA | |
| | | AGAAGCTTCTGGACTCTGAACAAGAATTCGGTG | |
| | | GCCTGCAGAGCTTGCCATTTGCACTTTTCAAATG | |
| | | CCTTTGGATGACCCAGCACTTTAATCTGAAACCT | |
| | | GCAACAAGACTAGCCAACACCTGGCCATGAAAC | |
| | | TTGCCCCTTCACTGATCTGGACTCACCTCTGGAG | |
| | | CCTATGGCTTTAAGCAAGCACTACTGCACTTTAC | |
| | | AGAATTACCCCACTGGATCCTGGACCCACAGAA | |
| | | TTCCTTCAGGATCCTTCTTGCTGCCAGACTGAAA | |
| | | GCAAAAGGAATTATTTCCCCTCAAGTTTTCTAAG | |
| | | TGATTTCCAAAAGCAGAGGTGTGTGGAAATTTC | |
| | | CAGTAACAGAAACAGATGGGTTGCCAATAGAGT | |
| | | TATTTTTTATCTATAGCTTCCTCTGGGTACTAGA | |
| | | AGAGGCTATTGAGACTATGAGCTCACAGACAGG | |
| | | GCTTCGCACAAACTCAAATCATAATTGACATGTT | |
| | | TTATGGATTACTGGAATCTTGATAGCATAATGAA | |
| | | GTTGTTCTAATTAACAGAGAGCATTTAAATATAC | |
| | | ACTAAGTGCACAAATTGTGGAGTAAAGTCATCA | |
| | | AGCTCTGTTTTTGAGGTCTAAGTCACAAAGCATT | |
| | | TGTTTTAACCTGTAATGGCACCATGTTTAATGGT | |
| | | GGTTTTTTTTTTGAACTACATCTTTCCTTTAAAAA | |
| | | TTATTGGTTTCTTTTTATTTGTTTTTACCTTAGAA | |
| | | ATCAATTATATACAGTCAAAAATATTTGATATGC | |
| | | TCATACGTTGTATCTGCAGCAATTTCAGATAAGT | |
| | | AGCTAAAATGGCCAAAGCCCCAAACTAAGCCTC | |
| | | CTTTTCTGGCCCTCAATATGACTTTAAATTTGAC | |
| | | TTTTCAGTGCCTCAGTTTGCACATCTGTAATACA | |
| | | GCAATGCTAAGTAGTCAAGGCCTTTGATAATTG | |
| | | GCACTATGGAAATCCTGCAAGATCCCACTACAT | |
| | | ATGTGTGGAGCAGAAGGGTAACTCGGCTACAGT | |
| | | AACAGCTTAATTTTGTTAAATTTGTTCTTTATACT | |
| | | GGAGCCATGAAGCTCAGAGCATTAGCTGACCCT | |
| | | TGAACTATTCAAATGGGCACATTAGCTAGTATA | |
| | | ACAGACTTACATAGGTGGGCCTAAAGCAAGCTC | |
| | | CTTAACTGAGCAAAATTTGGGGCTTATGAGAAT | |
| | | GAAAGGGTGTGAAATTGACTAACAGACAAATCA | |
| | | TACATCTCAGTTTCTCAATTCTCATGTAAATCAG | |
| | | AGAATGCCTTTAAAGAATAAAACTCAATTGTTAT | |
| | | TCTTCAACGTTCTTTATATATTCTACTTTTGGGTA | |
| PSMB10 | NM_002801.2 | AGACGTGAAGCCTAGCAGAGGACTTTTTAGCTG | 30 |
| | | CTCACTGGCCCCGCTTGTCTGGCCGACTCATCCG | |
| | | CCCGCGACCCCTAATCCCCTCTGCCTGCCCCAAG | |
| | | ATGCTGAAGCCAGCCCTGGAGCCCCGAGGGGGC | |
| | | TTCTCCTTCGAGAACTGCCAAAGAAATGCATCAT | |
| | | TGGAACGCGTCCTCCCGGGGCTCAAGGTCCCTC | |
| | | ACGCACGCAAGACCGGGACCACCATCGCGGGCC | |
| | | TGGTGTTCCAAGACGGGGTCATTCTGGGCGCCG | |
| | | ATACGCGAGCCACTAACGATTCGGTCGTGGCGG | |
| | | ACAAGAGCTGCGAGAAGATCCACTTCATCGCCC | |
| | | CCAAAATCTACTGCTGTGGGGCTGGAGTAGCCG | |
| | | CGGACGCCGAGATGACCACACGGATGGTGGCGT | |
| | | CCAAGATGGAGCTACACGCGTTATCTACGGGCC | |
| | | GCGAGCCCCGCGTGGCCACGGTCACTCGCATCC | |
| | | TGCGCCAGACGCTCTTCAGGTACCAGGGCCACG | |
| | | TGGGTGCATCGCTGATCGTGGGCGGCGTAGACC | |
| | | TGACTGGACCGCAGCTCTACGGCGTGCATCCCC | |
| | | ATGGCTCCTACAGCCGTCTGCCCTTCACAGCCCT | |
| | | GGGCTCTGGTCAGGACGCGGCCCTGGCGGTGCT | |
| | | AGAAGACCGGTTCCAGCCGAACATGACGCTGGA | |
| | | GGCTGCTCAGGGGCTGCTGGTGGAAGCCGTCAC | |
| | | CGCCGGGATCTTGGGTGACCTGGGCTCCGGGGG | |
| | | CAATGTGGACGCATGTGTGATCACAAAGACTGG | |
| | | CGCCAAGCTGCTGCGGACACTGAGCTCACCCAC | |
| | | AGAGCCCGTGAAGAGGTCTGGCCGCTACCACTT | |
| | | TGTGCCTGGAACCACAGCTGTCCTGACCCAGAC | |
| | | AGTGAAGCCACTAACCCTGGAGCTAGTGGAGGA | |
| | | AACTGTGCAGGCTATGGAGGTGGAGTAAGCTGA | |
| | | GGCTTAGAGCTTGGAACAAGGGGGAATAAACCC | |
| | | AGAAAATACAGTTAAACAAAAAAAAAAAAAAA | |
| | | AAAAAAAAAAAAAAAA | |
| STAT1 | NM_007315.2 | AGCGGGGCGGGGCGCCAGCGCTGCCTTTTCTCCT | 31 |
| | | GCCGGGTAGTTTCGCTTTCCTGCGCAGAGTCTGC | |
| | | GGAGGGGCTCGGCTGCACCGGGGGGATCGCGCC | |
| | | TGGCAGACCCCAGACCGAGCAGAGGCGACCCAG | |
| | | CGCGCTCGGGAGAGGCTGCACCGCCGCGCCCCC | |
| | | GCCTAGCCCTTCCGGATCCTGCGCGCAGAAAAG | |
| | | TTTCATTTGCTGTATGCCATCCTCGAGAGCTGTC | |
| | | TAGGTTAACGTTCGCACTCTGTGTATATAACCTC | |
| | | GACAGTCTTGGCACCTAACGTGCTGTGCGTAGCT | |
| | | GCTCCTTTGGTTGAATCCCCAGGCCCTTGTTGGG | |
| | | GCACAAGGTGGCAGGATGTCTCAGTGGTACGAA | |
| | | CTTCAGCAGCTTGACTCAAAATTCCTGGAGCAG | |
| | | GTTCACCAGCTTTATGATGACAGTTTTCCCATGG | |
| | | AAATCAGACAGTACCTGGCACAGTGGTTAGAAA | |
| | | AGCAAGACTGGGAGCACGCTGCCAATGATGTTT | |
| | | CATTTGCCACCATCCGTTTTCATGACCTCCTGTC | |
| | | ACAGCTGGATGATCAATATAGTCGCTTTTCTTTG | |
| | | GAGAATAACTTCTTGCTACAGCATAACATAAGG | |
| | | AAAAGCAAGCGTAATCTTCAGGATAATTTTCAG | |
| | | GAAGACCCAATCCAGATGTCTATGATCATTTACA | |
| | | GCTGTCTGAAGGAAGAAAGGAAAATTCTGGAAA | |
| | | ACGCCCAGAGATTTAATCAGGCTCAGTCGGGGA | |
| | | ATATTCAGAGCACAGTGATGTTAGACAAACAGA | |
| | | AAGAGCTTGACAGTAAAGTCAGAAATGTGAAGG | |
| | | ACAAGGTTATGTGTATAGAGCATGAAATCAAGA | |
| | | GCCTGGAAGATTTACAAGATGAATATGACTTCA | |
| | | AATGCAAAACCTTGCAGAACAGAGAACACGAGA | |
| | | CCAATGGTGTGGCAAAGAGTGATCAGAAACAAG | |
| | | AACAGCTGTTACTCAAGAAGATGTATTTAATGCT | |
| | | TGACAATAAGAGAAAGGAAGTAGTTCACAAAAT | |
| | | AATAGAGTTGCTGAATGTCACTGAACTTACCCA | |
| | | GAATGCCCTGATTAATGATGAACTAGTGGAGTG | |
| | | GAAGCGGAGACAGCAGAGCGCCTGTATTGGGGG | |
| | | GCCGCCCAATGCTTGCTTGGATCAGCTGCAGAA | |
| | | CTGGTTCACTATAGTTGCGGAGAGTCTGCAGCA | |
| | | AGTTCGGCAGCAGCTTAAAAAGTTGGAGGAATT | |
| | | GGAACAGAAATACACCTACGAACATGACCCTAT | |
| | | CACAAAAAACAAACAAGTGTTATGGGACCGCAC | |
| | | CTTCAGTCTTTTCCAGCAGCTCATTCAGAGCTCG | |
| | | TTTGTGGTGGAAAGACAGCCCTGCATGCCAACG | |
| | | CACCCTCAGAGGCCGCTGGTCTTGAAGACAGGG | |
| | | GTCCAGTTCACTGTGAAGTTGAGACTGTTGGTGA | |
| | | AATTGCAAGAGCTGAATTATAATTTGAAAGTCA | |
| | | AAGTCTTATTTGATAAAGATGTGAATGAGAGAA | |
| | | ATACAGTAAAAGGATTTAGGAAGTTCAACATTT | |
| | | TGGGCACGCACACAAAAGTGATGAACATGGAGG | |
| | | AGTCCACCAATGGCAGTCTGGCGGCTGAATTTC | |
| | | GGCACCTGCAATTGAAAGAACAGAAAAATGCTG | |
| | | GCACCAGAACGAATGAGGGTCCTCTCATCGTTA | |
| | | CTGAAGAGCTTCACTCCCTTAGTTTTGAAACCCA | |
| | | ATTGTGCCAGCCTGGTTTGGTAATTGACCTCGAG | |
| | | ACGACCTCTCTGCCCGTTGTGGTGATCTCCAACG | |
| | | TCAGCCAGCTCCCGAGCGGTTGGGCCTCCATCCT | |
| | | TTGGTACAACATGCTGGTGGCGGAACCCAGGAA | |
| | | TCTGTCCTTCTTCCTGACTCCACCATGTGCACGA | |
| | | TGGGCTCAGCTTTCAGAAGTGCTGAGTTGGCAGT | |
| | | TTTCTTCTGTCACCAAAAGAGGTCTCAATGTGGA | |
| | | CCAGCTGAACATGTTGGGAGAGAAGCTTCTTGG | |
| | | TCCTAACGCCAGCCCCGATGGTCTCATTCCGTGG | |
| | | ACGAGGTTTTGTAAGGAAAATATAAATGATAAA | |
| | | AATTTTCCCTTCTGGCTTTGGATTGAAAGCATCC | |
| | | TAGAACTCATTAAAAAACACCTGCTCCCTCTCTG | |
| | | GAATGATGGGTGCATCATGGGCTTCATCAGCAA | |
| | | GGAGCGAGAGCGTGCCCTGTTGAAGGACCAGCA | |
| | | GCCGGGGACCTTCCTGCTGCGGTTCAGTGAGAG | |
| | | CTCCCGGGAAGGGGCCATCACATTCACATGGGT | |
| | | GGAGCGGTCCCAGAACGGAGGCGAACCTGACTT | |
| | | CCATGCGGTTGAACCCTACACGAAGAAAGAACT | |
| | | TTCTGCTGTTACTTTCCCTGACATCATTCGCAATT | |
| | | ACAAAGTCATGGCTGCTGAGAATATTCCTGAGA | |
| | | ATCCCCTGAAGTATCTGTATCCAAATATTGACAA | |
| | | AGACCATGCCTTTGGAAAGTATTACTCCAGGCC | |
| | | AAAGGAAGCACCAGAGCCAATGGAACTTGATGG | |
| | | CCCTAAAGGAACTGGATATATCAAGACTGAGTT | |
| | | GATTTCTGTGTCTGAAGTTCACCCTTCTAGACTT | |
| | | CAGACCACAGACAACCTGCTCCCCATGTCTCCTG | |
| | | AGGAGTTTGACGAGGTGTCTCGGATAGTGGGCT | |
| | | CTGTAGAATTCGACAGTATGATGAACACAGTAT | |
| | | AGAGCATGAATTTTTTTCATCTTCTCTGGCGACA | |
| | | GTTTTCCTTCTCATCTGTGATTCCCTCCTGCTACT | |
| | | CTGTTCCTTCACATCCTGTGTTTCTAGGGAAATG | |
| | | AAAGAAAGGCCAGCAAATTCGCTGCAACCTGTT | |
| | | GATAGCAAGTGAATTTTTCTCTAACTCAGAAACA | |
| | | TCAGTTACTCTGAAGGGCATCATGCATCTTACTG | |
| | | AAGGTAAAATTGAAAGGCATTCTCTGAAGAGTG | |
| | | GGTTTCACAAGTGAAAAACATCCAGATACACCC | |
| | | AAAGTATCAGGACGAGAATGAGGGTCCTTTGGG | |
| | | AAAGGAGAAGTTAAGCAACATCTAGCAAATGTT | |
| | | ATGCATAAAGTCAGTGCCCAACTGTTATAGGTTG | |
| | | TTGGATAAATCAGTGGTTATTTAGGGAACTGCTT | |
| | | GACGTAGGAACGGTAAATTTCTGTGGGAGAATT | |
| | | CTTACATGTTTTCTTTGCTTTAAGTGTAACTGGC | |
| | | AGTTTTCCATTGGTTTACCTGTGAAATAGTTCAA | |
| | | AGCCAAGTTTATATACAATTATATCAGTCCTCTT | |
| | | TCAAAGGTAGCCATCATGGATCTGGTAGGGGGA | |
| | | AAATGTGTATTTTATTACATCTTTCACATTGGCT | |
| | | ATTTAAAGACAAAGACAAATTCTGTTTCTTGAGA | |
| | | AGAGAATATTAGCTTTACTGTTTGTTATGGCTTA | |
| | | ATGACACTAGCTAATATCAATAGAAGGATGTAC | |
| | | ATTTCCAAATTCACAAGTTGTGTTTGATATCCAA | |
| | | AGCTGAATACATTCTGCTTTCATCTTGGTCACAT | |
| | | ACAATTATTTTTACAGTTCTCCCAAGGGAGTTAG | |
| | | GCTATTCACAACCACTCATTCAAAAGTTGAAATT | |
| | | AACCATAGATGTAGATAAACTCAGAAATTTAAT | |
| | | TCATGTTTCTTAAATGGGCTACTTTGTCCTTTTTG | |
| | | TTATTAGGGTGGTATTTAGTCTATTAGCCACAAA | |
| | | ATTGGGAAAGGAGTAGAAAAAGCAGTAACTGAC | |
| | | AACTTGAATAATACACCAGAGATAATATGAGAA | |
| | | TCAGATCATTTCAAAACTCATTTCCTATGTAACT | |
| | | GCATTGAGAACTGCATATGTTTCGCTGATATATG | |
| | | TGTTTTTCACATTTGCGAATGGTTCCATTCTCTCT | |
| | | CCTGTACTTTTTCCAGACACTTTTTTGAGTGGAT | |
| | | GATGTTTCGTGAAGTATACTGTATTTTTACCTTTT | |
| | | TCCTTCCTTATCACTGACACAAAAAGTAGATTAA | |
| | | GAGATGGGTTTGACAAGGTTCTTCCCTTTTACAT | |
| | | ACTGCTGTCTATGTGGCTGTATCTTGTTTTTCCAC | |
| | | TACTGCTACCACAACTATATTATCATGCAAATGC | |
| | | TGTATTCTTCTTTGGTGGAGATAAAGATTTCTTG | |
| | | AGTTTTGTTTTAAAATTAAAGCTAAAGTATCTGT | |
| | | ATTGCATTAAATATAATATGCACACAGTGCTTTC | |
| | | CGTGGCACTGCATACAATCTGAGGCCTCCTCTCT | |
| | | CAGTTTTTATATAGATGGCGAGAACCTAAGTTTC | |
| | | AGTTGATTTTACAATTGAAATGACTAAAAAACA | |
| | | AAGAAGACAACATTAAAACAATATTGTTTCTA | |
| TIGIT | NM_173799.2 | ACATCTGCTTCCTGTAGGCCCTCTGGGCAGAAGC | 32 |
| | | ATGCGCTGGTGTCTCCTCCTGATCTGGGCCCAGG | |
| | | GGCTGAGGCAGGCTCCCCTCGCCTCAGGAATGA | |
| | | TGACAGGCACAATAGAAACAACGGGGAACATTT | |
| | | CTGCAGAGAAAGGTGGCTCTATCATCTTACAAT | |
| | | GTCACCTCTCCTCCACCACGGCACAAGTGACCCA | |
| | | GGTCAACTGGGAGCAGCAGGACCAGCTTCTGGC | |
| | | CATTTGTAATGCTGACTTGGGGTGGCACATCTCC | |
| | | CCATCCTTCAAGGATCGAGTGGCCCCAGGTCCC | |
| | | GGCCTGGGCCTTACCCTCCAGTCGCTGACCGTGA | |
| | | ACGATACAGGGGAGTACTTCTGCATCTATCACA | |
| | | CCTACCCTGATGGGGCGTACACTGGGAGAATCT | |
| | | TCCTGGAGGTCCTAGAAAGCTCAGTGGCTGAGC | |
| | | ACGGTGCCAGGTTCCAGATTCCATTGCTTGGAGC | |
| | | CATGGCCGCGACGCTGGTGGTCATCTGCACAGC | |
| | | AGTCATCGTGGTGGTCGCGTTGACTAGAAAGAA | |
| | | GAAAGCCCTCAGAATCCATTCTGTGGAAGGTGA | |
| | | CCTCAGGAGAAAATCAGCTGGACAGGAGGAATG | |
| | | GAGCCCCAGTGCTCCCTCACCCCCAGGAAGCTG | |
| | | TGTCCAGGCAGAAGCTGCACCTGCTGGGCTCTGT | |
| | | GGAGAGCAGCGGGGAGAGGACTGTGCCGAGCT | |
| | | GCATGACTACTTCAATGTCCTGAGTTACAGAAGC | |
| | | CTGGGTAACTGCAGCTTCTTCACAGAGACTGGTT | |
| | | AGCAACCAGAGGCATCTTCTGGAAGATACACTT | |
| | | TTGTCTTTGCTATTATAGATGAATATATAAGCAG | |
| | | CTGCACTCTCCATCAGTGCTGCGTGTGTGTGTGT | |
| | | GTGTGTATGTGTGTGTGTGTTCAGTTGAGTGAAT | |
| | | AAATGTCATCCTCTTCTCCATCTTCATTTCCTTGG | |
| | | CCTTTTCGTTCTATTCCATTTTGCATTATGGCAGG | |
| | | CCTAGGGTGAGTAACGTGGATCTTGATCATAAA | |
| | | TGCAAAATTAAAAAATATCTTGACCTGGTTTTAA | |
| | | ATCTGGCAGTTTGAGCAGATCCTATGTCTCTGAG | |
| | | AGACACATTCCTCATAATGGCCAGCATTTTGGGC | |
| | | TACAAGGTTTTGTGGTTGATGATGAGGATGGCAT | |
| | | GACTGCAGAGCCATCCTCATCTCATTTTTTCACG | |
| | | TCATTTTCAGTAACTTTCACTCATTCAAAGGCAG | |
| | | GTTATAAGTAAGTCCTGGTAGCAGCCTCTATGGG | |
| | | GAGATTTGAGAGTGACTAAATCTTGGTATCTGCC | |
| | | CTCAAGAACTTACAGTTAAATGGGGAGACAATG | |
| | | TTGTCATGAAAAGGTATTATAGTAAGGAGAGAA | |
| | | GGAGACATACACAGGCCTTCAGGAAGAGACGAC | |
| | | AGTTTGGGGTGAGGTAGTTGGCATAGGCTTATCT | |
| | | GTGATGAAGTGGCCTGGGAGCACCAAGGGGATG | |
| | | TTGAGGCTAGTCTGGGAGGAGCAGGAGTTTTGT | |
| | | CTAGGGAACTTGTAGGAAATTCTTGGAGCTGAA | |
| | | AGTCCCACAAAGAAGGCCCTGGCACCAAGGGAG | |
| | | TCAGCAAACTTCAGATTTTATTCTCTGGGCAGGC | |
| | | ATTTCAAGTTTCCTTTTGCTGTGACATACTCATCC | |
| | | ATTAGACAGCCTGATACAGGCCTGTAGCCTCTTC | |
| | | CGGCCGTGTGTGCTGGGGAAGCCCCAGGAAACG | |
| | | CACATGCCCACACAGGGAGCCAAGTCGTAGCAT | |
| | | TTGGGCCTTGATCTACCTTTTCTGCATCAATACA | |
| | | CTCTTGAGCCTTTGAAAAAAGAACGTTTCCCACT | |
| | | AAAAAGAAAATGTGGATTTTTAAAATAGGGACT | |
| | | CTTCCTAGGGGAAAAAGGGGGGCTGGGAGTGAT | |
| | | AGAGGGTTTAAAAAATAAACACCTTCAAACTAA | |
| | | CTTCTTCGAACCCTTTTATTCACTCCCTGACGACT | |
| | | TTGTGCTGGGGTTGGGGTAACTGAACTGCTTATT | |
| | | TCTGTTTAATTGCATTCAGGCTGGATCTTAGAAG | |
| | | ACTTTTATCCTTCCACCATCTCTCTCAGAGGAAT | |
| | | GAGCGGGGAGGTTGGATTTACTGGTGACTGATT | |
| | | TTCTTTCATGGGCCAAGGAACTGAAAGAGAATG | |
| | | TGAAGCAAGGTTGTGTCTTGCGCATGGTTAAAA | |
| | | ATAAAGCATTGTCCTGCTTCCTAAG | |
| ABCF1 | NM_001090.2 | GCGCCAGCTTGGAGAGCCAGCCCCATCGGGGTT | 33 |
| | | CCCCGCCGCCGGAAGCGGAAATAGCACCGGGCG | |
| | | CCGCCACAGTAGCTGTAACTGCCACCGCGATGC | |
| | | CGAAGGCGCCCAAGCAGCAGCCGCCGGAGCCCG | |
| | | AGTGGATCGGGGACGGAGAGAGCACGAGCCCAT | |
| | | CAGACAAAGTGGTGAAGAAAGGGAAGAAGGAC | |
| | | AAGAAGATCAAAAAAACGTTCTTTGAAGAGCTG | |
| | | GCAGTAGAAGATAAACAGGCTGGGGAAGAAGA | |
| | | GAAAGTGCTCAAGGAGAAGGAGCAGCAGCAGC | |
| | | AGCAACAGCAACAGCAGCAAAAAAAAAAGCGA | |
| | | GATACCCGAAAAGGCAGGCGGAAGAAGGATGT | |
| | | GGATGATGATGGAGAAGAGAAAGAGCTCATGG | |
| | | AGCGTCTTAAGAAGCTCTCAGTGCCAACCAGTG | |
| | | ATGAGGAGGATGAAGTACCCGCCCCAAAACCCC | |
| | | GCGGAGGGAAGAAAACCAAGGGTGGTAATGTTT | |
| | | TTGCAGCCCTGATTCAGGATCAGAGTGAGGAAG | |
| | | AGGAGGAGGAAGAAAAACATCCTCCTAAGCCTG | |
| | | CCAAGCCGGAGAAGAATCGGATCAATAAGGCCG | |
| | | TATCTGAGGAACAGCAGCCTGCACTCAAGGGCA | |
| | | AAAAGGGAAAGGAAGAGAAGTCAAAAGGGAAG | |
| | | GCTAAGCCTCAAAATAAATTCGCTGCTCTGGAC | |
| | | AATGAAGAGGAGGATAAAGAAGAAGAAATTAT | |
| | | AAAGGAAAAGGAGCCTCCCAAACAAGGGAAGG | |
| | | AGAAGGCCAAGAAGGCAGAGCAGATGGAGTAT | |
| | | GAGCGCCAAGTGGCTTCATTAAAAGCAGCCAAT | |
| | | GCAGCTGAAAATGACTTCTCCGTGTCCCAGGCG | |
| | | GAGATGTCCTCCCGCCAAGCCATGTTAGAAAAT | |
| | | GCATCTGACATCAAGCTGGAGAAGTTCAGCATC | |
| | | TCCGCTCATGGCAAGGAGCTGTTCGTCAATGCA | |
| | | GACCTGTACATTGTAGCCGGCCGCCGCTACGGG | |
| | | CTGGTAGGACCCAATGGCAAGGGCAAGACCACA | |
| | | CTCCTCAAGCACATTGCCAACCGAGCCCTGAGC | |
| | | ATCCCTCCCAACATTGATGTGTTGCTGTGTGAGC | |
| | | AGGAGGTGGTAGCAGATGAGACACCAGCAGTCC | |
| | | AGGCTGTTCTTCGAGCTGACACCAAGCGATTGA | |
| | | AGCTGCTGGAAGAGGAGCGGCGGCTTCAGGGAC | |
| | | AGCTGGAACAAGGGGATGACACAGCTGCTGAGA | |
| | | GGCTAGAGAAGGTGTATGAGGAATTGCGGGCCA | |
| | | CTGGGGCGGCAGCTGCAGAGGCCAAAGCACGGC | |
| | | GGATCCTGGCTGGCCTGGGCTTTGACCCTGAAAT | |
| | | GCAGAATCGACCCACACAGAAGTTCTCAGGGGG | |
| | | CTGGCGCATGCGTGTCTCCCTGGCCAGGGCACTG | |
| | | TTCATGGAGCCCACACTGCTGATGCTGGATGAG | |
| | | CCCACCAACCACCTGGACCTCAACGCTGTCATCT | |
| | | GGCTTAATAACTACCTCCAGGGCTGGCGGAAGA | |
| | | CCTTGCTGATCGTCTCCCATGACCAGGGCTTCTT | |
| | | GGATGATGTCTGCACTGATATCATCCACCTCGAT | |
| | | GCCCAGCGGCTCCACTACTATAGGGGCAATTAC | |
| | | ATGACCTTCAAAAAGATGTACCAGCAGAAGCAG | |
| | | AAAGAACTGCTGAAACAGTATGAGAAGCAAGA | |
| | | GAAAAAGCTGAAGGAGCTGAAGGCAGGCGGGA | |
| | | AGTCCACCAAGCAGGCGGAAAAACAAACGAAG | |
| | | GAAGCCCTGACTCGGAAGCAGCAGAAATGCCGA | |
| | | CGGAAAAACCAAGATGAGGAATCCCAGGAGGC | |
| | | CCCTGAGCTCCTGAAGCGCCCTAAGGAGTACAC | |
| | | TGTGCGCTTCACTTTTCCAGACCCCCCACCACTC | |
| | | AGCCCTCCAGTGCTGGGTCTGCATGGTGTGACAT | |
| | | TCGGCTACCAGGGACAGAAACCACTCTTTAAGA | |
| | | ACTTGGATTTTGGCATCGACATGGATTCAAGGAT | |
| | | TTGCATTGTGGGCCCTAATGGTGTGGGGAAGAG | |
| | | TACGCTACTCCTGCTGCTGACTGGCAAGCTGACA | |
| | | CCGACCCATGGGGAAATGAGAAAGAACCACCGG | |
| | | CTGAAAATTGGCTTCTTCAACCAGCAGTATGCAG | |
| | | AGCAGCTGCGCATGGAGGAGACGCCCACTGAGT | |
| | | ACCTGCAGCGGGGCTTCAACCTGCCCTACCAGG | |
| | | ATGCCCGCAAGTGCCTGGGCCGCTTCGGCCTGG | |
| | | AGAGTCACGCCCACACCATCCAGATCTGCAAAC | |
| | | TCTCTGGTGGTCAGAAGGCGCGAGTTGTGTTTGC | |
| | | TGAGCTGGCCTGTCGGGAACCTGATGTCCTCATC | |
| | | TTGGACGAGCCAACCAATAACCTGGACATAGAG | |
| | | TCTATTGATGCTCTAGGGGAGGCCATCAATGAAT | |
| | | ACAAGGGTGCTGTGATCGTTGTCAGCCATGATG | |
| | | CCCGACTCATCACAGAAACCAATTGCCAGCTGT | |
| | | GGGTGGTGGAGGAGCAGAGTGTTAGCCAAATCG | |
| | | ATGGTGACTTTGAAGACTACAAGCGGGAGGTGT | |
| | | TGGAGGCCCTGGGTGAAGTCATGGTCAGCCGGC | |
| | | CCCGAGAGTGAGCTTTCCTTCCCAGAAGTCTCCC | |
| | | GAGAGACATATTTGTGTGGCCTAGAAGTCCTCTG | |
| | | TGGTCTCCCCTCCTCTGAAGACTGCCTCTGGCCT | |
| | | GCAGCTGACCTGGCAACCATTCAGGCACATGAA | |
| | | GGTGGAGTGTGACCTTGATGTGACCGGGATCCC | |
| | | ACTCTGATTGCATCCATTTCTCTGAAAGACTTGT | |
| | | TTGTTCTGCTTCTCTTCATATAACTGAGCTGGCCT | |
| | | TATCCTTGGCATCCCCCTAAACAAACAAGAGGT | |
| | | GACCACCTTATTGTGAGGTTCCATCCAGCCAAGT | |
| | | TTATGTGGCCTATTGTCTCAGGACTCTCATCACT | |
| | | CAGAAGCCTGCCTCTGATTTACCCTACAGCTTCA | |
| | | GGCCCAGCTGCCCCCCAGTCTTTGGGTGGTGCTG | |
| | | TTCTTTTCTGGTGGATTTAATGCTGACTCACTGG | |
| | | TACAAACAGCTGTTGAAGCTCAGAGCTGGAGGT | |
| | | GAGCTTCTGAGGCCTTTGCCATTATCCAGCCCAA | |
| | | GATTTGGTGCCTGCAGCCTCTTGTCTGGTTGAGG | |
| | | ACTTGGGGCAGGAAAGGAATGCTGCTGAACTTG | |
| | | AATTTCCCTTTACAAGGGGAAGAAATAAAGGAA | |
| | | AGGAGTTGCTGCCGACCTGTCACTGTTTGGAGAT | |
| | | TGATGGGAGTTGGAACTGTTCTCAGTCTTGATTT | |
| | | GCTTTATTCAGTTTTCTAGCAGCTTTTAATAGTCC | |
| | | CCTCTTCCCCACTAAATGGATCTTGTTTGCAGTC | |
| | | TTGCTGACAGTGTTTGCTGTTTAAGGATCATAGG | |
| | | ATTCCTTTCCCCCAACCCTTCACGCAAGGAAAAA | |
| | | GCAAAGTGATTCATACCTTCTATCTTGGAAAAAA | |
| | | AAAAAA | |
| C14ORF102 | NM_017970.3 | CCCCTTGGCCCCGCCCCACCCTGCTTTGCCCTGC | 34 |
| | | CTCTCCCTGCCCCGCCGCGCCCCAGTCCCTTGAC | |
| | | GACCCTCCTCTCTGGGCCCCGCCCCTCCCGCTTC | |
| | | GGGGTCAAGCCCCAGAGAGCGCCGCGAAAACCA | |
| | | CATTTCCCAGAGTGCACCGCGACGGCAGGGGTC | |
| | | CTCAGACCGGCGCTCGCTCGCCGGCGCCATCCCT | |
| | | ATAGAGAAGAACGGAGGTACGGCCTGTGGTCAT | |
| | | GGCGCTGTTCCCAGCCTTTGCGGGGCTTAGTGAG | |
| | | GCTCCCGATGGCGGGAGCTCCAGGAAAGAGTTA | |
| | | GACTGGCTGAGCAACCCAAGCTTTTGTGTTGGAT | |
| | | CCATAACGTCCCTGAGCCAACAAACTGAAGCAG | |
| | | CTCCAGCCCATGTTTCTGAAGGGTTACCGCTGAC | |
| | | AAGGAGTCATCTGAAATCAGAGTCTTCAGATGA | |
| | | AAGTGACACTAACAAAAAGCTCAAACAAACAAG | |
| | | TAGAAAAAAGAAGAAAGAGAAAAAGAAAAAAA | |
| | | GGAAGCATCAGCATCATAAGAAAACAAAGAGG | |
| | | AAGCATGGGCCGTCGAGTAGCAGCAGGTCTGAG | |
| | | ACAGACACCGATTCTGAAAAGGACAAACCTTCC | |
| | | AGAGGCGTTGGAGGCAGTAAAAAGGAATCTGAG | |
| | | GAACCGAATCAAGGAAATAATGCTGCAGCTGAT | |
| | | ACTGGACATCGCTTTGTTTGGCTTGAGGACATTC | |
| | | AGGCTGTGACGGGAGAAACCTTCAGAACAGATA | |
| | | AGAAACCAGATCCTGCGAACTGGGAGTACAAGT | |
| | | CTCTCTACCGAGGGGATATAGCAAGATACAAGA | |
| | | GGAAAGGAGACTCCTGCCTTGGCATTAACCCTA | |
| | | AGAAGCAGTGCATATCTTGGGAAGGGACTTCCA | |
| | | CAGAGAAGAAGCATTCACGCAAGCAGGTTGAAC | |
| | | GCTATTTTACTAAGAAGAGTGTGGGATTAATGA | |
| | | ACATCGATGGAGTTGCCATTAGCAGTAAAACTG | |
| | | AACCTCCCTCATCTGAGCCCATCTCCTTTATACC | |
| | | AGTGAAGGACTTGGAAGATGCGGCTCCTGTTAC | |
| | | AACCTGGTTGAATCCTCTGGGGATTTATGATCAG | |
| | | TCAACCACACATTGGCTACAAGGACAGGGTCCT | |
| | | CCAGAGCAGGAATCAAAGCAGCCAGACGCACA | |
| | | GCCAGACAGCGAGAGTGCGGCTCTCAAGGCCAA | |
| | | GGTGGAGGAGTTTAACAGGAGGGTGCGGGAGA | |
| | | ATCCTCGGGATACGCAGCTGTGGATGGCATTTGT | |
| | | TGCTTTTCAGGACGAGGTCATGAAAAGTCCTGG | |
| | | CCTGTATGCCATCGAGGAAGGAGAGCAGGAAAA | |
| | | GCGAAAGAGGTCCCTGAAGCTCATTCTGGAGAA | |
| | | GAAGCTGGCCATTCTGGAGCGGGCCATTGAGAG | |
| | | CAACCAGAGCAGTGTGGATCTGAAACTGGCCAA | |
| | | GCTGAAGCTCTGCACAGAGTTCTGGGAGCCCTC | |
| | | CACTCTGGTCAAAGAGTGGCAGAAACTGATATT | |
| | | TTTGCATCCCAATAATACAGCCCTTTGGCAGAAA | |
| | | TACCTTTTATTTTGCCAGAGCCAGTTTAGTACCT | |
| | | TTTCGATATCAAAAATTCACAGTCTTTATGGAAA | |
| | | ATGCTTGAGCACTTTGTCTGCTGTTAAGGACGGC | |
| | | AGCATCTTATCTCACCCTGCGTTGCCTGGCACGG | |
| | | AAGAGGCCATGTTTGCACTCTTTCTTCAGCAGTG | |
| | | CCACTTTCTGCGGCAGGCTGGCCACTCTGAGAA | |
| | | GGCCATCTCATTGTTCCAGGCCATGGTGGACTTC | |
| | | ACCTTCTTCAAACCCGACAGCGTGAAAGATCTG | |
| | | CCTACCAAAGGACAGGTGGAATTCTTTGAACCC | |
| | | TTTTGGGACAGTGGAGAGCCCCGGGCTGGGGAG | |
| | | AAGGGAGCCCGAGGCTGGAAGGCGTGGATGCAC | |
| | | CAGCAGGAACGAGGTGGCTGGGTGGTCATCAAC | |
| | | CCAGATGAGGATGACGATGAACCAGAAGAGGAT | |
| | | GACCAGGAAATAAAAGATAAGACTCTGCCCAGG | |
| | | TGGCAGATCTGGCTTGCTGCTGAGCGTTCCCGTG | |
| | | ACCAGAGGCACTGGCGGCCCTGGCGCCCTGATA | |
| | | AGACCAAGAAGCAAACCGAGGAAGACTGTGAG | |
| | | GATCCCGAGAGACAGGTGTTGTTTGATGATATTG | |
| | | GGCAATCTTTGATCAGACTTTCCAGCCATGATCT | |
| | | TCAGTTCCAGCTGGTGGAGGCCTTCCTGCAGTTC | |
| | | TTGGGTGTGCCTTCTGGCTTTACTCCTCCAGCCT | |
| | | CCTGTCTTTATCTGGCCATGGATGAGAACAGCAT | |
| | | CTTTGATAATGGACTTTATGATGAAAAGCCCTTG | |
| | | ACTTTTTTCAACCCTTTGTTTTCTGGGGCTAGCTG | |
| | | TGTTGGCCGCATGGATAGGTTGGGCTATCCTCGC | |
| | | TGGACCAGGGGTCAGAACCGAGAGGGCGAGGA | |
| | | GTTCATCCGCAATGTCTTCCACCTTGTCATGCCT | |
| | | TTATTTTCAGGCAAAGAGAAGTCCCAGCTCTGCT | |
| | | TCTCCTGGTTACAGTATGAGATTGCAAAGGTCAT | |
| | | TTGGTGCCTGCACACTAAAAACAAGAAGAGATT | |
| | | AAAGTCTCAAGGGAAGAACTGCAAAAAACTAGC | |
| | | CAAGAATCTCCTTAAGGAGCCAGAAAACTGCAA | |
| | | CAACTTTTGCCTGTGGAAGCAGTATGCACATCTG | |
| | | GAGTGGTTGCTTGGCAACACGGAGGATGCCAGA | |
| | | AAAGTTTTTGACACAGCACTTGGCATGGCAGGA | |
| | | AGCAGAGAACTGAAAGACTCTGACCTCTGTGAG | |
| | | CTCAGTCTGCTCTATGCTGAGCTGGAGGTGGAGC | |
| | | TGTCGCCAGAAGTGAGAAGGGCTGCCACAGCTC | |
| | | GAGCTGTTCACATATTAACCAAGCTGACTGAGA | |
| | | GCAGCCCCTATGGGCCCTACACTGGACAGGTGT | |
| | | TGGCTGTTCACATTTTGAAAGCGCGAAAGGCTTA | |
| | | TGAGCACGCACTGCAGGACTGTTTGGGTGACAG | |
| | | CTGTGTCTCCAATCCAGCTCCCACCGATTCCTGT | |
| | | AGCCGCCTAATTAGCCTGGCTAAATGCTTCATGC | |
| | | TCTTCCAGTATTTGACCATAGGGATTGATGCTGC | |
| | | TGTGCAGATATACGAACAGGTGTTTGCAAAACT | |
| | | GAACAGTTCTGTTTTCCCAGAAGGCTCTGGCGAG | |
| | | GGGGACAGTGCCAGCTCCCAGAGTTGGACCAGT | |
| | | GTTCTCGAAGCCATCACACTGATGCACACGAGC | |
| | | CTGCTGAGATTCCACATGAAAGTGAGTGTTTACC | |
| | | CGCTGGCCCCTCTGCGAGAGGCACTCTCACAGG | |
| | | CTTTAAAGTTGTATCCAGGCAACCAGGTTCTTTG | |
| | | GAGGTCCTATGTACAGATTCAGAATAAGTCCCA | |
| | | CAGTGCCAGCAAAACCAGGAGATTTTTTGACAC | |
| | | AATCACCAGGTCTGCCAAACCCTTGGAGCCTTG | |
| | | GTTGTTTGCAATTGAAGCTGAGAAACTGAGGAA | |
| | | GAGACTGGTGGAAACTGTCCAGAGGTTAGACGG | |
| | | TAGAGAGATCCACGCCACAATTCCTGAGACCGG | |
| | | CTTAATGCATCGGATCCAAGCCCTGTTTGAAAAT | |
| | | GCCATGCGCAGCGACAGTGGCAGCCAGTGCCCC | |
| | | TTGCTGTGGAGGATGTATTTGAACTTTCTGGTTT | |
| | | CCTTAGGAAATAAAGAAAGAAGCAAAGGTGTAT | |
| | | TCTACAAAGCACTTCAGAATTGCCCTTGGGCAA | |
| | | AGGTGTTGTACCTGGACGCCGTGGAGTATTTCCC | |
| | | CGATGAGATGCAGGAGATCCTGGACCTGATGAC | |
| | | TGAGAAGGAGCTCCGGGTGCGCCTGCCGCTGGA | |
| | | GGAGCTGGAGCTGCTGCTGGAGGATTAGAGAGC | |
| | | AGCGGGAAAACGGGCTGTGCCTGCGAGGCCAAG | |
| | | TTGCCCACCCTGCGGAGCTAGGAGGCGCGAGCA | |
| | | GAGAACGTGTGTGTTAGGAGAACTCGGCTTTTG | |
| | | AAATGTTCTTTCTCGATAGTAATAATGTGGGCTG | |
| | | CCAGCCTCTCACATCTTGCACACTTTTTGGGTGT | |
| | | GTAAATGACACAAAAGTTATTTACATATTATATA | |
| | | TGTGAATATGTGTATATATGTACATAGCCAGAG | |
| | | AGTCATGCCACGTGGTCATTAAACCGATGATGA | |
| | | TTGAGGCGTGAAAAAAAAAAAAAAAA | |
| G6PD | NM_000402.2 | AGGGACAGCCCAGAGGAGGCGTGGCCACGCTGC | 35 |
| | | CGGCGGAAGTGGAGCCCTCCGCGAGCGCGCGAG | |
| | | GCCGCCGGGGCAGGCGGGGAAACCGGACAGTA | |
| | | GGGGCGGGGCCGGGCCGGCGATGGGGATGCGG | |
| | | GAGCACTACGCGGAGCTGCACCCGTGCCCGCCG | |
| | | GAATTGGGGATGCAGAGCAGCGGCAGCGGGTAT | |
| | | GGCAGGCAGCCGGCGGGCCGGCCTCCAGCGCAG | |
| | | GTGCCCGAGAGGCAGGGGCTGGCCTGGGATGCG | |
| | | CGCGCACCTGCCCTCGCCCCGCCCCGCCCGCACG | |
| | | AGGGGTGGTGGCCGAGGCCCCGCCCCGCACGCC | |
| | | TCGCCTGAGGCGGGTCCGCTCAGCCCAGGCGCC | |
| | | CGCCCCCGCCCCCGCCGATTAAATGGGCCGGCG | |
| | | GGGCTCAGCCCCCGGAAACGGTCGTAACTTCGG | |
| | | GGCTGCGAGCGCGGAGGGCGACGACGACGAAG | |
| | | CGCAGACAGCGTCATGGCAGAGCAGGTGGCCCT | |
| | | GAGCCGGACCCAGGTGTGCGGGATCCTGCGGGA | |
| | | AGAGCTTTTCCAGGGCGATGCCTTCCATCAGTCG | |
| | | GATACACACATATTCATCATCATGGGTGCATCGG | |
| | | GTGACCTGGCCAAGAAGAAGATCTACCCCACCA | |
| | | TCTGGTGGCTGTTCCGGGATGGCCTTCTGCCCGA | |
| | | AAACACCTTCATCGTGGGCTATGCCCGTTCCCGC | |
| | | CTCACAGTGGCTGACATCCGCAAACAGAGTGAG | |
| | | CCCTTCTTCAAGGCCACCCCAGAGGAGAAGCTC | |
| | | AAGCTGGAGGACTTCTTTGCCCGCAACTCCTATG | |
| | | TGGCTGGCCAGTACGATGATGCAGCCTCCTACC | |
| | | AGCGCCTCAACAGCCACATGGATGCCCTCCACC | |
| | | TGGGGTCACAGGCCAACCGCCTCTTCTACCTGGC | |
| | | CTTGCCCCCGACCGTCTACGAGGCCGTCACCAA | |
| | | GAACATTCACGAGTCCTGCATGAGCCAGATAGG | |
| | | CTGGAACCGCATCATCGTGGAGAAGCCCTTCGG | |
| | | GAGGGACCTGCAGAGCTCTGACCGGCTGTCCAA | |
| | | CCACATCTCCTCCCTGTTCCGTGAGGACCAGATC | |
| | | TACCGCATCGACCACTACCTGGGCAAGGAGATG | |
| | | GTGCAGAACCTCATGGTGCTGAGATTTGCCAAC | |
| | | AGGATCTTCGGCCCCATCTGGAACCGGGACAAC | |
| | | ATCGCCTGCGTTATCCTCACCTTCAAGGAGCCCT | |
| | | TTGGCACTGAGGGTCGCGGGGGCTATTTCGATG | |
| | | AATTTGGGATCATCCGGGACGTGATGCAGAACC | |
| | | ACCTACTGCAGATGCTGTGTCTGGTGGCCATGGA | |
| | | GAAGCCCGCCTCCACCAACTCAGATGACGTCCG | |
| | | TGATGAGAAGGTCAAGGTGTTGAAATGCATCTC | |
| | | AGAGGTGCAGGCCAACAATGTGGTCCTGGGCCA | |
| | | GTACGTGGGGAACCCCGATGGAGAGGGCGAGGC | |
| | | CACCAAAGGGTACCTGGACGACCCCACGGTGCC | |
| | | CCGCGGGTCCACCACCGCCACTTTTGCAGCCGTC | |
| | | GTCCTCTATGTGGAGAATGAGAGGTGGGATGGG | |
| | | GTGCCCTTCATCCTGCGCTGCGGCAAGGCCCTGA | |
| | | ACGAGCGCAAGGCCGAGGTGAGGCTGCAGTTCC | |
| | | ATGATGTGGCCGGCGACATCTTCCACCAGCAGT | |
| | | GCAAGCGCAACGAGCTGGTGATCCGCGTGCAGC | |
| | | CCAACGAGGCCGTGTACACCAAGATGATGACCA | |
| | | AGAAGCCGGGCATGTTCTTCAACCCCGAGGAGT | |
| | | CGGAGCTGGACCTGACCTACGGCAACAGATACA | |
| | | AGAACGTGAAGCTCCCTGACGCCTACGAGCGCC | |
| | | TCATCCTGGACGTCTTCTGCGGGAGCCAGATGCA | |
| | | CTTCGTGCGCAGCGACGAGCTCCGTGAGGCCTG | |
| | | GCGTATTTTCACCCCACTGCTGCACCAGATTGAG | |
| | | CTGGAGAAGCCCAAGCCCATCCCCTATATTTATG | |
| | | GCAGCCGAGGCCCCACGGAGGCAGACGAGCTGA | |
| | | TGAAGAGAGTGGGTTTCCAGTATGAGGGCACCT | |
| | | ACAAGTGGGTGAACCCCCACAAGCTCTGAGCCC | |
| | | TGGGCACCCACCTCCACCCCCGCCACGGCCACC | |
| | | CTCCTTCCCGCCGCCCGACCCCGAGTCGGGAGG | |
| | | ACTCCGGGACCATTGACCTCAGCTGCACATTCCT | |
| | | GGCCCCGGGCTCTGGCCACCCTGGCCCGCCCCTC | |
| | | GCTGCTGCTACTACCCGAGCCCAGCTACATTCCT | |
| | | CAGCTGCCAAGCACTCGAGACCATCCTGGCCCC | |
| | | TCCAGACCCTGCCTGAGCCCAGGAGCTGAGTCA | |
| | | CCTCCTCCACTCACTCCAGCCCAACAGAAGGAA | |
| | | GGAGGAGGGCGCCCATTCGTCTGTCCCAGAGCT | |
| | | TATTGGCCACTGGGTCTCACTCCTGAGTGGGGCC | |
| | | AGGGTGGGAGGGAGGGACAAGGGGGAGGAAAG | |
| | | GGGCGAGCACCCACGTGAGAGAATCTGCCTGTG | |
| | | GCCTTGCCCGCCAGCCTCAGTGCCACTTGACATT | |
| | | CCTTGTCACCAGCAACATCTCGAGCCCCCTGGAT | |
| | | GTCCCCTGTCCCACCAACTCTGCACTCCATGGCC | |
| | | ACCCCGTGCCACCCGTAGGCAGCCTCTCTGCTAT | |
| | | AAGAAAAGCAGACGCAGCAGCTGGGACCCCTCC | |
| | | CAACCTCAATGCCCTGCCATTAAATCCGCAAAC | |
| | | AGCCAAAAAAAAAAAAAAAAAAAA | |
| OAZ1 | NM_004152.2 | TTTTGCGAACGGCGAGCAGCGGCGGCGGCGCGG | 36 |
| | | AGAGACGCAGCGGAGGTTTTCCTGGTTTCGGAC | |
| | | CCCAGCGGCCGGATGGTGAAATCCTCCCTGCAG | |
| | | CGGATCCTCAATAGCCACTGCTTCGCCAGAGAG | |
| | | AAGGAAGGGGATAAACCCAGCGCCACCATCCAC | |
| | | GCCAGCCGCACCATGCCGCTCCTAAGCCTGCAC | |
| | | AGCCGCGGCGGCAGCAGCAGTGAGAGTTCCAGG | |
| | | GTCTCCCTCCACTGCTGTAGTAACCCGGGTCCGG | |
| | | GGCCTCGGTGGTGCTCCTGATGCCCCTCACCCAC | |
| | | CCCTGAAGATCCCAGGTGGGCGAGGGAATAGTC | |
| | | AGAGGGATCACAATCTTTCAGCTAACTTATTCTA | |
| | | CTCCGATGATCGGCTGAATGTAACAGAGGAACT | |
| | | AACGTCCAACGACAAGACGAGGATTCTCAACGT | |
| | | CCAGTCCAGGCTCACAGACGCCAAACGCATTAA | |
| | | CTGGCGAACAGTGCTGAGTGGCGGCAGCCTCTA | |
| | | CATCGAGATCCCGGGCGGCGCGCTGCCCGAGGG | |
| | | GAGCAAGGACAGCTTTGCAGTTCTCCTGGAGTTC | |
| | | GCTGAGGAGCAGCTGCGAGCCGACCATGTCTTC | |
| | | ATTTGCTTCCACAAGAACCGCGAGGACAGAGCC | |
| | | GCCTTGCTCCGAACCTTCAGCTTTTTGGGCTTTG | |
| | | AGATTGTGAGACCGGGGCATCCCCTTGTCCCCA | |
| | | AGAGACCCGACGCTTGCTTCATGGCCTACACGTT | |
| | | CGAGAGAGAGTCTTCGGGAGAGGAGGAGGAGT | |
| | | AGGGCCGCCTCGGGGCTGGGCATCCGGCCCCTG | |
| | | GGGCCACCCCTTGTCAGCCGGGTGGGTAGGAAC | |
| | | CGTAGACTCGCTCATCTCGCCTGGGTTTGTCCGC | |
| | | ATGTTGTAATCGTGCAAATAAACGCTCACTCCGA | |
| | | ATTAGCGGTGTATTTCTTGAAGTTTAATATTGTG | |
| | | TTTGTGATACTGAAGTATTTGCTTTAATTCTAAA | |
| | | TAAAAATTTATATTTTACTTTTTTATTGCTGGTTT | |
| | | AAGATGATTCAGATTATCCTTGTACTTTGAGGAG | |
| | | AAGTTTCTTATTTGGAGTCTTTTGGAAACAGTCT | |
| | | TAGTCTTTTAACTTGGAAAGATGAGGTATTAATC | |
| | | CCCTCCATTGCTCTCCAAAAGCCAATAAAGTGAT | |
| | | TACACCCGA | |
| POLR2A | NM_000937.2 | GAGAGCGCGGCCGGGACGGTTGGAGAAGAAGG | 37 |
| | | CGGCTCCCCGGAAGGGGGAGAGACAAACTGCCG | |
| | | TAACCTCTGCCGTTCAGGAACCCGGTTACTTATT | |
| | | TATTCGTTACCCTTTTTCTTCTTCCTCCCCCAAAA | |
| | | ACCTTTTCCTTTTCCCTTCTTTTTTTTTCCTTTTTG | |
| | | GGAGCTGAAAAATTTCCGGTAAGGGAAAGAAGG | |
| | | GCTCCTTTCGCTCCTTATTTCGCCGCCTCCTTCCC | |
| | | TCCGCCACCTTCCCCTCCTCCGGCTTTTTCCTCCC | |
| | | AACTCGGGGAGGTCCTTCCCGGTGGCCGCCCTG | |
| | | ACGAGGTCTGAGCACCTAGGCGGAGGCGGCGCA | |
| | | GGCTTTTTGTAGTGAGGTTTGCGCCTGCGCAGGC | |
| | | GCCTGCCTCCGCCATGCACGGGGGTGGCCCCCC | |
| | | CTCGGGGGACAGCGCATGCCCGCTGCGCACCAT | |
| | | CAAGAGAGTCCAGTTCGGAGTCCTGAGTCCGGA | |
| | | TGAACTGAAGCGAATGTCTGTGACGGAGGGTGG | |
| | | CATCAAATACCCAGAGACGACTGAGGGAGGCCG | |
| | | CCCCAAGCTTGGGGGGCTGATGGACCCGAGGCA | |
| | | GGGGGTGATTGAGCGGACTGGCCGCTGCCAAAC | |
| | | ATGTGCAGGAAACATGACAGAGTGTCCTGGCCA | |
| | | CTTTGGCCACATTGAACTGGCCAAGCCTGTGTTT | |
| | | CACGTGGGCTTCCTGGTGAAGACAATGAAAGTT | |
| | | TTGCGCTGTGTCTGCTTCTTCTGCTCCAAACTGCT | |
| | | TGTGGACTCTAACAACCCAAAGATCAAGGATAT | |
| | | CCTGGCTAAGTCCAAGGGACAGCCCAAGAAGCG | |
| | | GCTCACACATGTCTACGACCTTTGCAAGGGCAA | |
| | | AAACATATGCGAGGGTGGGGAGGAGATGGACA | |
| | | ACAAGTTCGGTGTGGAACAACCTGAGGGTGACG | |
| | | AGGATCTGACCAAAGAAAAGGGCCATGGTGGCT | |
| | | GTGGGCGGTACCAGCCCAGGATCCGGCGTTCTG | |
| | | GCCTAGAGCTGTATGCGGAATGGAAGCACGTTA | |
| | | ATGAGGACTCTCAGGAGAAGAAGATCCTGCTGA | |
| | | GTCCAGAGCGAGTGCATGAGATCTTCAAACGCA | |
| | | TCTCAGATGAGGAGTGTTTTGTGCTGGGCATGGA | |
| | | GCCCCGCTATGCACGGCCAGAGTGGATGATTGT | |
| | | CACAGTGCTGCCTGTGCCCCCGCTCTCCGTGCGG | |
| | | CCTGCTGTTGTGATGCAGGGCTCTGCCCGTAACC | |
| | | AGGATGACCTGACTCACAAACTGGCTGACATCG | |
| | | TGAAGATCAACAATCAGCTGCGGCGCAATGAGC | |
| | | AGAACGGCGCAGCGGCCCATGTCATTGCAGAGG | |
| | | ATGTGAAGCTCCTCCAGTTCCATGTGGCCACCAT | |
| | | GGTGGACAATGAGCTGCCTGGCTTGCCCCGTGC | |
| | | CATGCAGAAGTCTGGGCGTCCCCTCAAGTCCCTG | |
| | | AAGCAGCGGTTGAAGGGCAAGGAAGGCCGGGT | |
| | | GCGAGGGAACCTGATGGGCAAAAGAGTGGACTT | |
| | | CTCGGCCCGTACTGTCATCACCCCCGACCCCAAC | |
| | | CTCTCCATTGACCAGGTTGGCGTGCCCCGCTCCA | |
| | | TTGCTGCCAACATGACCTTTGCGGAGATTGTCAC | |
| | | CCCCTTCAACATTGACAGACTTCAAGAACTAGTG | |
| | | CGCAGGGGGAACAGTCAGTACCCAGGCGCCAAG | |
| | | TACATCATCCGAGACAATGGTGATCGCATTGACT | |
| | | TGCGTTTCCACCCCAAGCCCAGTGACCTTCACCT | |
| | | GCAGACCGGCTATAAGGTGGAACGGCACATGTG | |
| | | TGATGGGGACATTGTTATCTTCAACCGGCAGCCA | |
| | | ACTCTGCACAAAATGTCCATGATGGGGCATCGG | |
| | | GTCCGCATTCTCCCATGGTCTACCTTTCGCTTGA | |
| | | ATCTTAGCGTGACAACTCCGTACAATGCAGACTT | |
| | | TGACGGGGATGAGATGAACTTGCACCTGCCACA | |
| | | GTCTCTGGAGACGCGAGCAGAGATCCAGGAGCT | |
| | | GGCCATGGTTCCTCGCATGATTGTCACCCCCCAG | |
| | | AGCAATCGGCCTGTCATGGGTATTGTGCAGGAC | |
| | | ACACTCACAGCAGTGCGCAAATTCACCAAGAGA | |
| | | GACGTCTTCCTGGAGCGGGGTGAAGTGATGAAC | |
| | | CTCCTGATGTTCCTGTCGACGTGGGATGGGAAG | |
| | | GTCCCACAGCCGGCCATCCTAAAGCCCCGGCCC | |
| | | CTGTGGACAGGCAAGCAAATCTTCTCCCTCATCA | |
| | | TACCTGGTCACATCAATTGTATCCGTACCCACAG | |
| | | CACCCATCCCGATGATGAAGACAGTGGCCCTTA | |
| | | CAAGCACATCTCTCCTGGGGACACCAAGGTGGT | |
| | | GGTGGAGAATGGGGAGCTGATCATGGGCATCCT | |
| | | GTGTAAGAAGTCTCTGGGCACGTCAGCTGGCTC | |
| | | CCTGGTCCACATCTCCTACCTAGAGATGGGTCAT | |
| | | GACATCACTCGCCTCTTCTACTCCAACATTCAGA | |
| | | CTGTCATTAACAACTGGCTCCTCATCGAGGGTCA | |
| | | TACTATTGGCATTGGGGACTCCATTGCTGATTCT | |
| | | AAGACTTACCAGGACATTCAGAACACTATTAAG | |
| | | AAGGCCAAGCAGGACGTAATAGAGGTCATCGAG | |
| | | AAGGCACACAACAATGAGCTGGAGCCCACCCCA | |
| | | GGGAACACTCTGCGGCAGACGTTTGAGAATCAG | |
| | | GTGAACCGCATTCTTAACGATGCCCGAGACAAG | |
| | | ACTGGCTCCTCTGCTCAGAAATCCCTGTCTGAAT | |
| | | ACAACAACTTCAAGTCTATGGTCGTGTCCGGAG | |
| | | CTAAAGGTTCCAAGATTAACATCTCCCAGGTCAT | |
| | | TGCTGTCGTTGGACAGCAGAACGTCGAGGGCAA | |
| | | GCGGATTCCATTTGGCTTCAAGCACCGGACTCTG | |
| | | CCTCACTTCATCAAGGATGACTACGGGCCTGAG | |
| | | AGCCGTGGCTTTGTGGAGAACTCCTACCTAGCCG | |
| | | GCCTCACACCCACTGAGTTCTTTTTCCACGCCAT | |
| | | GGGGGGTCGTGAGGGGCTCATTGACACGGCTGT | |
| | | CAAGACTGCTGAGACTGGATACATCCAGCGGCG | |
| | | GCTGATCAAGTCCATGGAGTCAGTGATGGTGAA | |
| | | GTACGACGCGACTGTGCGGAACTCCATCAACCA | |
| | | GGTGGTGCAGCTGCGCTACGGCGAAGACGGCCT | |
| | | GGCAGGCGAGAGCGTTGAGTTCCAGAACCTGGC | |
| | | TACGCTTAAGCCTTCCAACAAGGCTTTTGAGAAG | |
| | | AAGTTCCGCTTTGATTATACCAATGAGAGGGCCC | |
| | | TGCGGCGCACTCTGCAGGAGGACCTGGTGAAGG | |
| | | ACGTGCTGAGCAACGCACACATCCAGAACGAGT | |
| | | TGGAGCGGGAATTTGAGCGGATGCGGGAGGATC | |
| | | GGGAGGTGCTCAGGGTCATCTTCCCAACTGGAG | |
| | | ACAGCAAGGTCGTCCTCCCCTGTAACCTGCTGCG | |
| | | GATGATCTGGAATGCTCAGAAAATCTTCCACATC | |
| | | AACCCACGCCTTCCCTCCGACCTGCACCCCATCA | |
| | | AAGTGGTGGAGGGAGTCAAGGAATTGAGCAAG | |
| | | AAGCTGGTGATTGTGAATGGGGATGACCCACTA | |
| | | AGTCGACAGGCCCAGGAAAATGCCACGCTGCTC | |
| | | TTCAACATCCACCTGCGGTCCACGTTGTGTTCCC | |
| | | GCCGCATGGCAGAGGAGTTTCGGCTCAGTGGGG | |
| | | AGGCCTTCGACTGGCTGCTTGGGGAGATTGAGT | |
| | | CCAAGTTCAACCAAGCCATTGCGCATCCCGGGG | |
| | | AAATGGTGGGGGCTCTGGCTGCGCAGTCCCTTG | |
| | | GAGAACCTGCCACCCAGATGACCTTGAATACCT | |
| | | TCCACTATGCTGGTGTGTCTGCCAAGAATGTGAC | |
| | | GCTGGGTGTGCCCCGACTTAAGGAGCTCATCAA | |
| | | CATTTCCAAGAAGCCAAAGACTCCTTCGCTTACT | |
| | | GTCTTCCTGTTGGGCCAGTCCGCTCGAGATGCTG | |
| | | AGAGAGCCAAGGATATTCTGTGCCGTCTGGAGC | |
| | | ATACAACGTTGAGGAAGGTGACTGCCAACACAG | |
| | | CCATCTACTATGACCCCAACCCCCAGAGCACGG | |
| | | TGGTGGCAGAGGATCAGGAATGGGTGAATGTCT | |
| | | ACTATGAAATGCCTGACTTTGATGTGGCCCGAAT | |
| | | CTCCCCCTGGCTGTTGCGGGTGGAGCTGGATCGG | |
| | | AAGCACATGACTGACCGGAAGCTCACCATGGAG | |
| | | CAGATTGCTGAAAAGATCAATGCTGGTTTTGGTG | |
| | | ACGACTTGAACTGCATCTTTAATGATGACAATGC | |
| | | AGAGAAGCTGGTGCTCCGTATTCGCATCATGAA | |
| | | CAGCGATGAGAACAAGATGCAAGAGGAGGAAG | |
| | | AGGTGGTGGACAAGATGGATGATGATGTCTTCC | |
| | | TGCGCTGCATCGAGTCCAACATGCTGACAGATA | |
| | | TGACCCTGCAGGGCATCGAGCAGATCAGCAAGG | |
| | | TGTACATGCACTTGCCACAGACAGACAACAAGA | |
| | | AGAAGATCATCATCACGGAGGATGGGGAATTCA | |
| | | AGGCCCTGCAGGAGTGGATCCTGGAGACGGACG | |
| | | GCGTGAGCTTGATGCGGGTGCTGAGTGAGAAGG | |
| | | ACGTGGACCCCGTACGCACCACGTCCAATGACA | |
| | | TTGTGGAGATCTTCACGGTGCTGGGCATTGAAGC | |
| | | CGTGCGGAAGGCCCTGGAGCGGGAGCTGTACCA | |
| | | CGTCATCTCCTTTGATGGCTCCTATGTCAATTAC | |
| | | CGACACTTGGCTCTCTTGTGTGATACCATGACCT | |
| | | GTCGTGGCCACTTGATGGCCATCACCCGACACG | |
| | | GAGTCAACCGCCAGGACACAGGACCACTCATGA | |
| | | AGTGTTCCTTTGAGGAAACGGTGGACGTGCTTAT | |
| | | GGAAGCAGCCGCACACGGTGAGAGTGACCCCAT | |
| | | GAAGGGGGTCTCTGAGAATATCATGCTGGGCCA | |
| | | GCTGGCTCCGGCCGGCACTGGCTGCTTTGACCTC | |
| | | CTGCTTGATGCAGAGAAGTGCAAGTATGGCATG | |
| | | GAGATCCCCACCAATATCCCCGGCCTGGGGGCT | |
| | | GCTGGACCCACCGGCATGTTCTTTGGTTCAGCAC | |
| | | CCAGTCCCATGGGTGGAATCTCTCCTGCCATGAC | |
| | | ACCTTGGAACCAGGGTGCAACCCCTGCCTATGG | |
| | | CGCCTGGTCCCCCAGTGTTGGGAGTGGAATGAC | |
| | | CCCAGGGGCAGCCGGCTTCTCTCCCAGTGCTGCG | |
| | | TCAGATGCCAGCGGCTTCAGCCCAGGTTACTCCC | |
| | | CTGCCTGGTCTCCCACACCGGGCTCCCCGGGGTC | |
| | | CCCAGGTCCCTCAAGCCCCTACATCCCTTCACCA | |
| | | GGTGGTGCCATGTCTCCCAGCTACTCGCCAACGT | |
| | | CACCTGCCTACGAGCCCCGCTCTCCTGGGGGCTA | |
| | | CACACCCCAGAGTCCCTCTTATTCCCCCACTTCA | |
| | | CCCTCCTACTCCCCTACCTCTCCATCCTATTCTCC | |
| | | AACCAGTCCCAACTATAGTCCCACATCACCCAG | |
| | | CTATTCGCCAACGTCACCCAGCTACTCACCGACC | |
| | | TCTCCCAGCTACTCACCCACCTCTCCCAGCTACT | |
| | | CGCCCACCTCTCCCAGCTATTCGCCCACCTCTCC | |
| | | CAGCTACTCACCCACTTCCCCTAGCTATTCGCCC | |
| | | ACTTCCCCTAGCTACTCGCCAACGTCTCCCAGCT | |
| | | ACTCGCCGACATCTCCCAGCTACTCGCCAACTTC | |
| | | ACCCAGCTATTCTCCCACTTCTCCCAGCTACTCA | |
| | | CCTACCTCTCCAAGCTATTCACCCACCTCCCCCA | |
| | | GCTACTCACCCACTTCCCCAAGTTACTCACCCAC | |
| | | CAGCCCGAACTATTCTCCAACCAGTCCCAATTAC | |
| | | ACCCCAACATCACCCAGCTACAGCCCGACATCA | |
| | | CCCAGCTATTCCCCTACTAGTCCCAACTACACAC | |
| | | CTACCAGCCCTAACTACAGCCCAACCTCTCCAAG | |
| | | CTACTCTCCAACATCACCCAGCTATTCCCCGACC | |
| | | TCACCAAGTTACTCCCCTTCCAGCCCACGATACA | |
| | | CACCACAGTCTCCAACCTATACCCCAAGCTCACC | |
| | | CAGCTACAGCCCCAGTTCGCCCAGCTACAGCCC | |
| | | AACCTCACCCAAGTACACCCCAACCAGTCCTTCT | |
| | | TATAGTCCCAGCTCCCCAGAGTATACCCCAACCT | |
| | | CTCCCAAGTACTCACCTACCAGTCCCAAATATTC | |
| | | ACCCACCTCTCCCAAGTACTCGCCTACCAGTCCC | |
| | | ACCTATTCACCCACCACCCCAAAATACTCCCCAA | |
| | | CATCTCCTACTTATTCCCCAACCTCTCCAGTCTA | |
| | | CACCCCAACCTCTCCCAAGTACTCACCTACTAGC | |
| | | CCCACTTACTCGCCCACTTCCCCCAAGTACTCGC | |
| | | CCACCAGCCCCACCTACTCGCCCACCTCCCCCAA | |
| | | AGGCTCAACCTACTCTCCCACTTCCCCTGGTTAC | |
| | | TCGCCCACCAGCCCCACCTACAGTCTCACAAGCC | |
| | | CGGCTATCAGCCCGGATGACAGTGACGAGGAGA | |
| | | ACTGAGGGCACGTGGGGTGCGGCAGCGGGCTAG | |
| | | GGCCCAGGGCAGCTTGCCCGTGCTGCCGTGCAG | |
| | | TTCTTGCCTCCCTCACGGGGCGTCACCCCCAGCC | |
| | | CAGCTCCGTTGTACATAAATACCTTGTGACAGAG | |
| | | CTCCCGGTGAACTTCTGGATCCCGTTTCTGATGC | |
| | | AGATTCTTGTCTTGTTCTCCACTTGTGCTGTTAGA | |
| | | ACTCACTGGCCCAGTGGTGTTCTACCTCCTACCC | |
| | | CACCCACCCCCTGCCTGTCCCCAAATTGAAGATC | |
| | | CTTCCTTGCCTGTGGCTTGATGCGGGGGGGGTAA | |
| | | AGGGTATTTTAACTTAGGGGTAGTTCCTGCTGTG | |
| | | AGTGGTTACAGCTGATCCTCGGGAAGAACAAAG | |
| | | CTAAAGCTGCCTTTTGTCTGTTATTTTATTTTTTT | |
| | | GAAGTTTAAATAAAGTTTACTAATTTTGACC | |
| SDHA | NM_004168.1 | GACTGCGCGGCGGCAACAGCAGACATGTCGGGG | 38 |
| | | GTCCGGGGCCTGTCGCGGCTGCTGAGCGCTCGG | |
| | | CGCCTGGCGCTGGCCAAGGCGTGGCCAACAGTG | |
| | | TTGCAAACAGGAACCCGAGGTTTTCACTTCACTG | |
| | | TTGATGGGAACAAGAGGGCATCTGCTAAAGTTT | |
| | | CAGATTCCATTTCTGCTCAGTATCCAGTAGTGGA | |
| | | TCATGAATTTGATGCAGTGGTGGTAGGCGCTGG | |
| | | AGGGGCAGGCTTGCGAGCTGCATTTGGCCTTTCT | |
| | | GAGGCAGGGTTTAATACAGCATGTGTTACCAAG | |
| | | CTGTTTCCTACCAGGTCACACACTGTTGCAGCGC | |
| | | AGGGAGGAATCAATGCTGCTCTGGGGAACATGG | |
| | | AGGAGGACAACTGGAGGTGGCATTICTACGACA | |
| | | CCGTGAAGGGCTCCGACTGGCTGGGGGACCAGG | |
| | | ATGCCATCCACTACATGACGGAGCAGGCCCCCG | |
| | | CCGCCGTGGTCGAGCTAGAAAATTATGGCATGC | |
| | | CGTTTAGCAGAACTGAAGATGGGAAGATTTATC | |
| | | AGCGTGCATTTGGTGGACAGAGCCTCAAGTTTG | |
| | | GAAAGGGCGGGCAGGCCCATCGGTGCTGCTGTG | |
| | | TGGCTGATCGGACTGGCCACTCGCTATTGCACAC | |
| | | CTTATATGGACGGTCTCTGCGATATGATACCAGC | |
| | | TATTTTGTGGAGTATTTTGCCTTGGATCTCCTGAT | |
| | | GGAGAACGGGGAGTGCCGTGGTGTCATCGCACT | |
| | | GTGCATAGAGGACGGGTCCATCCATCGCATAAG | |
| | | AGCAAAGAACACTGTTGTTGCCACAGGAGGCTA | |
| | | CGGGCGCACCTACTTCAGCTGCACGTCTGCCCAC | |
| | | ACCAGCACTGGCGACGGCACGGCCATGATCACC | |
| | | AGGGCAGGCCTTCCTTGCCAGGACCTAGAGTTT | |
| | | GTTCAGTTCCACCCCACAGGCATATATGGTGCTG | |
| | | GTTGTCTCATTACGGAAGGATGTCGTGGAGAGG | |
| | | GAGGCATTCTCATTAACAGTCAAGGCGAAAGGT | |
| | | TTATGGAGCGATACGCCCCTGTCGCGAAGGACC | |
| | | TGGCGTCTAGAGATGTGGTGTCTCGGTCGATGAC | |
| | | TCTGGAGATCCGAGAAGGAAGAGGCTGTGGCCC | |
| | | TGAGAAAGATCACGTCTACCTGCAGCTGCACCA | |
| | | CCTACCTCCAGAGCAGCTGGCCACGCGCCTGCCT | |
| | | GGCATTTCAGAGACAGCCATGATCTTCGCTGGC | |
| | | GTGGACGTCACGAAGGAGCCGATCCCTGTCCTC | |
| | | CCCACCGTGCATTATAACATGGGCGGCATTCCCA | |
| | | CCAACTACAAGGGGCAGGTCCTGAGGCACGTGA | |
| | | ATGGCCAGGATCAGATTGTGCCCGGCCTGTACG | |
| | | CCTGTGGGGAGGCCGCCTGTGCCTCGGTACATG | |
| | | GTGCCAACCGCCTCGGGGCAAACTCGCTCTTGG | |
| | | ACCTGGTTGTCTTTGGTCGGGCATGTGCCCTGAG | |
| | | CATCGAAGAGTCATGCAGGCCTGGAGATAAAGT | |
| | | CCCTCCAATTAAACCAAACGCTGGGGAAGAATC | |
| | | TGTCATGAATCTTGACAAATTGAGATTTGCTGAT | |
| | | GGAAGCATAAGAACATCGGAACTGCGACTCAGC | |
| | | ATGCAGAAGTCAATGCAAAATCATGCTGCCGTG | |
| | | TTCCGTGTGGGAAGCGTGTTGCAAGAAGGTTGT | |
| | | GGGAAAATCAGCAAGCTCTATGGAGACCTAAAG | |
| | | CACCTGAAGACGTTCGACCGGGGAATGGTCTGG | |
| | | AACACAGACCTGGTGGAGACCCTGGAGCTGCAG | |
| | | AACCTGATGCTGTGTGCGCTGCAGACCATCTACG | |
| | | GAGCAGAGGCGCGGAAGGAGTCACGGGGCGCG | |
| | | CATGCCAGGGAAGACTACAAGGTGCGGATTGAT | |
| | | GAGTACGATTACTCCAAGCCCATCCAGGGGCAA | |
| | | CAGAAGAAGCCCTTTGAGGAGCACTGGAGGAAG | |
| | | CACACCCTGTCCTTTGTGGACGTTGGCACTGGGA | |
| | | AGGTCACTCTGGAATATAGACCCGTAATCGACA | |
| | | AAACTTTGAACGAGGCTGACTGTGCCACCATCC | |
| | | CGCCAGCCATTCGCTCCTACTGATGAGACAAGA | |
| | | TGTGGTGATGACAGAATCAGCTTTTGTAATTATG | |
| | | TATAATAGCTCATGCATGTGTCCATGTCATAACT | |
| | | GTCTTCATACGCTTCTGCACTCTGGGGAAGAAGG | |
| | | AGTACATTGAAGGGAGATTGGCACCTAGTGGCT | |
| | | GGGAGCTTGCCAGGAACCCAGTGGCCAGGGAGC | |
| | | GTGGCACTTACCTTTGTCCCTTGCTTCATTCTTGT | |
| | | GAGATGATAAAACTGGGCACAGCTCTTAAATAA | |
| | | AATATAAATGAG | |
| STK11IP | NM_052902.2 | GATAGGCGCCGGGCAGCTGAGCTGGTAGGAGGA | 39 |
| | | CCAGACGGGGATGTTCGGCTCCGCCCCCCAGCG | |
| | | TCCCGTGGCCATGACGACCGCTCAGAGGGACTC | |
| | | CCTGTTGTGGAAGCTCGCGGGGTTGCTGCGGGA | |
| | | GTCCGGGGATGTGGTCCTGTCTGGCTGTAGCACC | |
| | | CTGAGCCTGCTGACTCCCACACTGCAACAGCTG | |
| | | AACCACGTATTTGAGCTGCACCTGGGGCCATGG | |
| | | GGCCCTGGCCAGACAGGCTTTGTGGCTCTGCCCT | |
| | | CCCATCCTGCCGACTCCCCTGTTATTCTTCAGCTT | |
| | | CAGTTTCTCTTCGATGTGCTGCAGAAAACACTTT | |
| | | CACTCAAGCTGGTCCATGTTGCTGGTCCTGGCCC | |
| | | CACAGGGCCCATCAAGATTTTCCCCTTCAAATCC | |
| | | CTTCGGCACCTGGAGCTCCGAGGTGTTCCCCTCC | |
| | | ACTGTCTGCATGGCCTCCGAGGCATCTACTCCCA | |
| | | GCTGGAGACCCTGATTTGCAGCAGGAGCCTCCA | |
| | | GGCATTAGAGGAGCTCCTCTCAGCCTGCGGCGG | |
| | | CGACTTCTGCTCTGCCCTCCCTTGGCTGGCTCTG | |
| | | CTTTCTGCCAACTTCAGCTACAATGCACTGACCG | |
| | | CCTTAGACAGCTCCCTGCGCCTCTTGTCAGCTCT | |
| | | GCGTTTCTTGAACCTAAGCCACAATCAAGTCCAG | |
| | | GACTGTCAGGGATTCCTGATGGATTTGTGTGAGC | |
| | | TCCACCATCTGGACATCTCCTATAATCGCCTGCA | |
| | | TTTGGTGCCAAGAATGGGACCCTCAGGGGCTGC | |
| | | TCTGGGGGTCCTGATACTGCGAGGCAATGAGCT | |
| | | TCGGAGCCTGCATGGCCTAGAGCAGCTGAGGAA | |
| | | TCTGCGGCACCTGGATTTGGCATACAACCTGCTG | |
| | | GAAGGACACCGGGAGCTGTCACCACTGTGGCTG | |
| | | CTGGCTGAGCTCCGCAAGCTCTACCTGGAGGGG | |
| | | AACCCTCTTTGGTTCCACCCTGAGCACCGAGCAG | |
| | | CCACTGCCCAGTACTTGTCACCCCGGGCCAGGG | |
| | | ATGCTGCTACTGGCTTCCTTCTCGATGGCAAGGT | |
| | | CTTGTCACTGACAGATTTTCAGACTCACACATCC | |
| | | TTGGGGCTCAGCCCCATGGGCCCACCTTTGCCCT | |
| | | GGCCAGTGGGGAGTACTCCTGAAACCTCAGGTG | |
| | | GCCCTGACCTGAGTGACAGCCTCTCCTCAGGGG | |
| | | GTGTTGTGACCCAGCCCCTGCTTCATAAGGTTAA | |
| | | GAGCCGAGTCCGTGTGAGGCGGGCAAGCATCTC | |
| | | TGAACCCAGTGATACGGACCCGGAGCCCCGAAC | |
| | | TCTGAACCCCTCTCCGGCTGGATGGTTCGTGCAG | |
| | | CAGCACCCGGAGCTGGAGCTCATGAGCAGCTTC | |
| | | CGGGAACGGTTCGGCCGCAACTGGCTGCAGTAC | |
| | | AGGAGTCACCTGGAGCCCTCCGGAAACCCTCTG | |
| | | CCGGCCACCCCCACTACTTCTGCACCCAGTGCAC | |
| | | CTCCAGCCAGCTCCCAGGGCCCCGACACTGCAC | |
| | | CCAGACCTTCACCCCCGCAGGAGGAAGCCAGAG | |
| | | GCCCCCAGGAGTCACCACAGAAAATGTCAGAGG | |
| | | AGGTCAGGGCGGAGCCACAGGAGGAGGAAGAG | |
| | | GAGAAGGAGGGGAAGGAGGAGAAGGAGGAGGG | |
| | | GGAGATGGTGGAACAGGGAGAAGAGGAGGCAG | |
| | | GAGAGGAGGAAGAAGAGGAGCAGGACCAGAAG | |
| | | GAAGTGGAAGCGGAACTCTGTCGCCCCTTGTTG | |
| | | GTGTGTCCCCTGGAGGGGCCTGAGGGCGTACGG | |
| | | GGCAGGGAATGCTTTCTCAGGGTCACTTCTGCCC | |
| | | ACCTGTTTGAGGTGGAACTCCAAGCAGCTCGCA | |
| | | CCTTGGAGCGACTGGAGCTCCAGAGTCTGGAGG | |
| | | CAGCTGAGATAGAGCCGGAGGCCCAGGCCCAGA | |
| | | GGTCGCCCAGGCCCACGGGCTCAGATCTGCTCC | |
| | | CTGGAGCCCCCATCCTCAGTCTGCGCTTCTCCTA | |
| | | CATCTGCCCTGACCGGCAGTTGCGTCGCTATTTG | |
| | | GTGCTGGAGCCTGATGCCCACGCAGCTGTCCAG | |
| | | GAGCTGCTTGCCGTGTTGACCCCAGTCACCAATG | |
| | | TGGCTCGGGAACAGCTTGGGGAGGCCAGGGACC | |
| | | TCCTGCTGGGTAGATTCCAGTGTCTACGCTGTGG | |
| | | CCATGAGTTCAAGCCAGAGGAGCCCAGGATGGG | |
| | | ATTAGACAGTGAGGAAGGCTGGAGGCCTCTGTT | |
| | | CCAAAAGACAGAATCTCCTGCTGTGTGTCCTAAC | |
| | | TGTGGTAGTGACCACGTGGTTCTCCTCGCTGTGT | |
| | | CTCGGGGAACCCCCAACAGGGAGCGGAAACAG | |
| | | GGAGAGCAGTCTCTGGCTCCTTCTCCGTCTGCCA | |
| | | GCCCTGTCTGCCACCCTCCTGGCCATGGTGACCA | |
| | | CCTTGACAGGGCCAAGAACAGCCCACCTCAGGC | |
| | | ACCGAGCACCCGTGACCATGGTAGTTGGAGCCT | |
| | | CAGTCCCCCCCCTGAGCGCTGTGGCCTCCGCTCT | |
| | | GTGGACCACCGACTCCGGCTCTTCCTGGATGTTG | |
| | | AGGTGTTCAGCGATGCCCAGGAGGAGTTCCAGT | |
| | | GCTGCCTCAAGGTGCCAGTGGCATTGGCAGGCC | |
| | | ACACTGGGGAGTTCATGTGCCTTGTGGTTGTGTC | |
| | | TGACCGCAGGCTGTACCTGTTGAAGGTGACTGG | |
| | | GGAGATGCGTGAGCCTCCAGCTAGCTGGCTGCA | |
| | | GCTGACCCTGGCTGTTCCCCTGCAGGATCTGAGT | |
| | | GGCATAGAGCTGGGCCTGGCAGGCCAGAGCCTG | |
| | | CGGCTAGAGTGGGCAGCTGGGGGGGGCCGCTGT | |
| | | GTGCTGCTGCCCCGAGATGCCAGGCATTGCCGG | |
| | | GCCTTCCTAGAGGAGCTCCTTGATGTCTTGCAGT | |
| | | CTCTGCCCCCTGCCTGGAGGAACTGTGTCAGTGC | |
| | | CACAGAGGAGGAGGTCACCCCCCAGCACCGGCT | |
| | | CTGGCCATTGCTGGAAAAAGACTCATCCTTGGA | |
| | | GGCTCGCCAGTTCTTCTACCTTCGGGCGTTCCTG | |
| | | GTTGAAGGCCCTTCCACCTGCCTCGTATCCCTGT | |
| | | TGCTGACTCCGTCCACCCTGTTCCTGTTAGATGA | |
| | | GGATGCTGCAGGGTCCCCGGCAGAGCCCTCTCC | |
| | | TCCAGCAGCATCTGGCGAAGCCTCTGAGAAGGT | |
| | | GCCTCCCTCGGGGCCGGGCCCTGCTGTGCGTGTC | |
| | | AGGGAGCAGCAGCCACTCAGCAGCCTGAGCTCC | |
| | | GTGCTGCTCTACCGCTCAGCCCCTGAGGACTTGC | |
| | | GGCTGCTCTTCTACGATGAGGTGTCCCGGCTGGA | |
| | | GAGCTTTTGGGCACTCCGTGTGGTGTGTCAGGAG | |
| | | CAGCTGACAGCCCTGCTTGCCTGGATCCGGGAA | |
| | | CCATGGGAGGAGCTGTTTTCCATCGGACTCCGG | |
| | | ACAGTGATCCAAGAGGCGCTGGCCCTTGACCGA | |
| | | TGAGGGTCCCACGCTGACCTTGGCCCTGACCTCA | |
| | | GGAGCCACGCTGTAGACATTCCCTCTCCTGGTCT | |
| | | CTGGGTCTGGCTTCCAGGCTCTGGCTGTGGATGT | |
| | | CTTCAGCCTCTGGGTGCTGGCCAGTGAGGTCCCA | |
| | | AATGACCCAGGGCTTAAGGGAGAGGCGAGAGA | |
| | | ATGATCTGGCCTCAGGGGACAGGCCACCTGGTC | |
| | | AGGAGGAATATTTTTCCTGCACTTTTTCTCAGGT | |
| | | ATCAATAAAGTTGTTTCCAACTCATAA | |
| TBC1D10B | NM_015527.3 | GAGGGGCGGCCCGCGGCCATGGAGACGGGCAC | 40 |
| | | GGCGCCCCTGGTGGCCCCGCCGCGCCGTCATGG | |
| | | CGCCCCCGCGGCCCCCTCGCCGCCGCCCCGGGG | |
| | | TTCCCGGGCCGGGCCCGTCGTGGTGGTGGCTCCG | |
| | | GGACCTCCAGTGACTACGGCCACTTCGGCCCCC | |
| | | GTCACCCTGGTGGCCCCCGGGGAGGCGCGGCCC | |
| | | GCCTGGGTCCCGGGGTCGGCCGAGACCTCTGCT | |
| | | CCGGCCCCGGCCCCAGCCCCGGCCCCAGCCCCG | |
| | | GCTGTCACGGGCAGCACGGTGGTGGTGCTGACC | |
| | | CTGGAGGCCTCGCCCGAAGCCCCAAAGCCGCAG | |
| | | CTCCCCTCCGGCCCGGAATCCCCAGAGCCCGCG | |
| | | GCAGTGGCTGGAGTTGAGACATCGAGGGCTCTG | |
| | | GCCGCAGGGGCAGACTCGCCGAAGACAGAGGA | |
| | | GGCTCGACCCTCACCCGCCCCAGGACCAGGGAC | |
| | | CCCCACCGGGACCCCTACCAGGACCCCTTCCAG | |
| | | AACGGCTCCTGGTGCCCTGACCGCCAAACCCCC | |
| | | GCTTGCCCCCAAGCCGGGAACCACAGTGGCCTC | |
| | | AGGAGTGACTGCACGGAGTGCATCAGGACAAGT | |
| | | GACAGGTGGGCATGGAGCTGCCGCAGCAACATC | |
| | | AGCATCAGCAGGACAGGCTCCTGAGGACCCCTC | |
| | | AGGCCCTGGCACAGGCCCCTCTGGGACTTGTGA | |
| | | GGCTCCGGTAGCTGTCGTGACCGTGACCCCAGCT | |
| | | CCGGAGCCTGCTGAAAACTCTCAAGACCTGGGC | |
| | | TCCACGTCCAGCCTGGGACCTGGCATCTCTGGGC | |
| | | CTCGAGGGCAGGCCCCGGACACGCTGAGTTACT | |
| | | TGGACTCCGTGAGCCTCATGTCTGGGACCTTGGA | |
| | | GTCCTTGGCGGATGATGTGAGCTCCATGGGCTCA | |
| | | GATTCAGAGATAAACGGGCTGGCCCTGCGCAAG | |
| | | ACGGACAAGTATGGCTTCCTTGGGGGCAGCCAG | |
| | | TACTCGGGCAGCCTAGAGAGCTCCATTCCCGTG | |
| | | GACGTGGCTCGGCAGCGGGAGCTCAAATGGCTG | |
| | | GACATGTTCAGTAACTGGGATAAGTGGCTGTCA | |
| | | CGGCGATTCCAGAAGGTGAAGCTGCGCTGCCGG | |
| | | AAGGGGATCCCCTCCTCTCTCAGAGCCAAAGCC | |
| | | TGGCAGTACCTGTCTAATAGCAAGGAACTTCTG | |
| | | GAGCAGAACCCAGGAAAGTTTGAGGAGCTGGAA | |
| | | CGGGCTCCTGGGGACCCCAAGTGGCTGGATGTG | |
| | | ATTGAGAAGGACCTGCACCGCCAGTTCCCTTTCC | |
| | | ACGAGATGTTTGCTGCTCGAGGGGGGCATGGGC | |
| | | AACAGGACCTGTACCGAATCCTGAAGGCCTACA | |
| | | CCATCTACCGGCCTGACGAGGGTTACTGCCAGG | |
| | | CCCAGGCCCCCGTGGCTGCGGTCCTGCTCATGCA | |
| | | CATGCCTGCGGAGCAAGCCTTTTGGTGCCTGGTG | |
| | | CAGATCTGCGACAAGTACCTCCCAGGTTACTAC | |
| | | AGTGCAGGGCTGGAGGCCATTCAGCTGGACGGG | |
| | | GAGATCTTTTTTGCACTCCTGCGCCGGGCCTCCC | |
| | | CGCTGGCGCATCGCCACCTGCGGCGGCAGCGCA | |
| | | TTGACCCTGTGCTCTACATGACGGAGTGGTTCAT | |
| | | GTGCATCTTCGCCCGCACCCTGCCCTGGGCGTCG | |
| | | GTGCTGCGTGTCTGGGACATGTTTTTCTGTGAAG | |
| | | GCGTTAAGATCATCTTCCGGGTGGCCCTGGTCCT | |
| | | GCTGCGCCACACGCTGGGCTCAGTGGAGAAGCT | |
| | | GCGCTCCTGCCAAGGCATGTATGAGACCATGGA | |
| | | GCAGCTGCGTAACCTGCCCCAGCAGTGCATGCA | |
| | | GGAAGACTTCCTGGTGCATGAGGTGACCAATCT | |
| | | GCCGGTGACAGAAGCACTGATTGAGCGGGAGAA | |
| | | TGCAGCCCAGCTCAAGAAGTGGCGGGAAACGCG | |
| | | GGGGGAGCTGCAGTATCGGCCCTCACGGCGACT | |
| | | GCATGGGTCCCGGGCCATCCACGAGGAGCGCCG | |
| | | GCGGCAACAGCCACCCCTGGGCCCCTCCTCCAG | |
| | | CCTCCTCAGCCTCCCTGGCCTCAAGAGCCGAGGC | |
| | | TCCCGGGCAGCTGGAGGGGCCCCGTCCCCGCCG | |
| | | CCCCCCGTCCGCAGAGCCAGTGCTGGGCCTGCC | |
| | | CCAGGGCCTGTGGTCACTGCTGAGGGACTGCAT | |
| | | CCATCCCTTCCCTCACCCACTGGCAATAGCACCC | |
| | | CCTTGGGTTCCAGCAAGGAGACCCGGAAGCAGG | |
| | | AGAAGGAGCGGCAGAAACAGGAGAAGGAGCGG | |
| | | CAGAAACAGGAGAAGGAGCGGGAGAAGGAGCG | |
| | | GCAGAAGCAGGAGAAAGAGCGAGAGAAGCAGG | |
| | | AAAAGGAGCGAGAGAAGCAGGAGAAGGAGCGG | |
| | | CAGAAGCAGGAGAAGAAGGCTCAAGGCCGGAA | |
| | | GCTTTCGCTGCGTCGAAAGGCAGATGGGCCCCC | |
| | | AGGCCCCCATGATGGTGGGGACAGGCCCTCAGC | |
| | | CGAGGCCCGGCAGGACGCTTACTTCTGACCTCTG | |
| | | CCCTGGGGCTGGACTGCATGGCCCCCCTCTTTCC | |
| | | CTCAGCCAAGAACAGGCCTGGCCCAAGGTGCCA | |
| | | CCCCCTAGCACCTTGTCAGGCTGTCCCTTGCTGG | |
| | | GGAAAGTGGCTTGGTTCCCCATCTCCTCGCCAGC | |
| | | TGCTGATCCCTACACGGGCAGGACAGATGGGCA | |
| | | GCTGCAAATGAGTCTGGAGCCTCTCATCTCCCAT | |
| | | GAGGCTCAGCTGGGGTCTCTGTCGCTCCTGCCCC | |
| | | AGTTCCCTCTGGGTCCCCTCCTAGGTGCTGTCCT | |
| | | GAATGGCCCGTTGTCATCCCAGGGGTGACTCCTG | |
| | | GTGATGGGAGTCAGCAGTTTCAGATTCTTACACT | |
| | | CCATAGCTCCCCTTACCATGAGGTGGAGCTGGCT | |
| | | TCCTTTTCCCTGTCTTCAGCCCTCCCTGTCTCCCC | |
| | | CACTTCCTGGCCAGGGCTCTCATTCTGGACCTGT | |
| | | GTTGTAATTGTGTACAGAGGATGGCGTTGGCCTG | |
| | | GGGTGGGGGTGCTCGCTTTGTCTTCTGTCCTTTG | |
| | | GTTCTCCTTCCATAATGCTCCTGTACCCAGTTTAT | |
| | | TTAAGGGGACATGCACTGGAATAGGAAATGTCC | |
| | | CCCATCTCCCTTCCTGCACCCTGCTGTGCTCCCTC | |
| | | CAAACCCACCTTGCTCTGTGTTCTCAGGCCCCCC | |
| | | TGCTTTTGTCTCACCAGGACCCATACCTTTCACC | |
| | | TTGTTCCCTTCCACCCCTCCAGTTAGTCCCTATCT | |
| | | GGGTAAGGGTCTTCCCTTGAGCTCCAGGGGGTG | |
| | | GAACCCAATGTTTACATTCTCTTCTGTCTCTGCC | |
| | | CCCACCCCATGCAGCGCTTTGAGGAATTGGAAA | |
| | | AGAACCTGCTGTTGTACCTGGGAAAAAAAAAAA | |
| | | AAAAAAAAAAAAAAAAAAAAAAAAAAA | |
| TBP | NM_001172085.1 | GGCGGAAGTGACATTATCAACGCGCGCCAGGGG | 41 |
| | | TTCAGTGAGGTCGGGCAGGTTCGCTGTGGCGGG | |
| | | CGCCTGGGCCGCCGGCTGTTTAACTTCGCTTCCG | |
| | | CTGGCCCATAGTGATCTTTGCAGTGACCCAGGGT | |
| | | GCCATGACTCCCGGAATCCCTATCTTTAGTCCAA | |
| | | TGATGCCTTATGGCACTGGACTGACCCCACAGCC | |
| | | TATTCAGAACACCAATAGTCTGTCTATTTTGGAA | |
| | | GAGCAACAAAGGCAGCAGCAGCAACAACAACA | |
| | | GCAGCAGCAGCAGCAGCAGCAGCAACAGCAAC | |
| | | AGCAGCAGCAGCAGCAGCAGCAGCAGCAGCAG | |
| | | CAGCAGCAGCAGCAGCAGCAGCAGCAACAGGC | |
| | | AGTGGCAGCTGCAGCCGTTCAGCAGTCAACGTC | |
| | | CCAGCAGGCAACACAGGGAACCTCAGGCCAGGC | |
| | | ACCACAGCTCTTCCACTCACAGACTCTCACAACT | |
| | | GCACCCTTGCCGGGCACCACTCCACTGTATCCCT | |
| | | CCCCCATGACTCCCATGACCCCCATCACTCCTGC | |
| | | CACGCCAGCTTCGGAGAGTTCTGGGATTGTACC | |
| | | GCAGCTGCAAAATATTGTATCCACAGTGAATCTT | |
| | | GGTTGTAAACTTGACCTAAAGACCATTGCACTTC | |
| | | GTGCCCGAAACGCCGAATATAATCCCAAGCGGT | |
| | | TTGCTGCGGTAATCATGAGGATAAGAGAGCCAC | |
| | | GAACCACGGCACTGATTTTCAGTTCTGGGAAAA | |
| | | TGGTGTGCACAGGAGCCAAGAGTGAAGAACAGT | |
| | | CCAGACTGGCAGCAAGAAAATATGCTAGAGTTG | |
| | | TACAGAAGTTGGGTTTTCCAGCTAAGTTCTTGGA | |
| | | CTTCAAGATTCAGAATATGGTGGGGAGCTGTGA | |
| | | TGTGAAGTTTCCTATAAGGTTAGAAGGCCTTGTG | |
| | | CTCACCCACCAACAATTTAGTAGTTATGAGCCAG | |
| | | AGTTATTTCCTGGTTTAATCTACAGAATGATCAA | |
| | | ACCCAGAATTGTTCTCCTTATTTTTGTTTCTGGAA | |
| | | AAGTTGTATTAACAGGTGCTAAAGTCAGAGCAG | |
| | | AAATTTATGAAGCATTTGAAAACATCTACCCTAT | |
| | | TCTAAAGGGATTCAGGAAGACGACGTAATGGCT | |
| | | CTCATGTACCCTTGCCTCCCCCACCCCCTTCTTTT | |
| | | TTTTTTTTTAAACAAATCAGTTTGTTTTGGTACCT | |
| | | TTAAATGGTGGTGTTGTGAGAAGATGGATGTTG | |
| | | AGTTGCAGGGTGTGGCACCAGGTGATGCCCTTCT | |
| | | GTAAGTGCCCACCGCGGGATGCCGGGAAGGGGC | |
| | | ATTATTTGTGCACTGAGAACACCGCGCAGCGTG | |
| | | ACTGTGAGTTGCTCATACCGTGCTGCTATCTGGG | |
| | | CAGCGCTGCCCATTTATTTATATGTAGATTTTAA | |
| | | ACACTGCTGTTGACAAGTTGGTTTGAGGGAGAA | |
| | | AACTTTAAGTGTTAAAGCCACCTCTATAATTGAT | |
| | | TGGACTTTTTAATTTTAATGTTTTTCCCCATGAAC | |
| | | CACAGTTTTTATATTTCTACCAGAAAAGTAAAAA | |
| | | TCTTTTTTAAAAGTGTTGTTTTTCTAATTTATAAC | |
| | | TCCTAGGGGTTATTTCTGTGCCAGACACATTCCA | |
| | | CCTCTCCAGTATTGCAGGACAGAATATATGTGTT | |
| | | AATGAAAATGAATGGCTGTACATATTTTTTTCTT | |
| | | TCTTCAGAGTACTCTGTACAATAAATGCAGTTTA | |
| | | TAAAAGTGTTAGATTGTTGTTAAAAAAAAAAAA | |
| | | AAAAAA | |
| UBB | NM_018955.2 | CACTCGTTGCATAAATTTGCGCTCCGCCAGCCCG | 42 |
| | | GAGCATTTAGGGGCGGTTGGCTTTGTTGGGTGA | |
| | | GCTTGTTTGTGTCCCTGTGGGTGGACGTGGTTGG | |
| | | TGATTGGCAGGATCCTGGTATCCGCTAACAGGTC | |
| | | AAAATGCAGATCTTCGTGAAAACCCTTACCGGC | |
| | | AAGACCATCACCCTTGAGGTGGAGCCCAGTGAC | |
| | | ACCATCGAAAATGTGAAGGCCAAGATCCAGGAT | |
| | | AAGGAAGGCATTCCCCCCGACCAGCAGAGGCTC | |
| | | ATCTTTGCAGGCAAGCAGCTGGAAGATGGCCGT | |
| | | ACTCTTTCTGACTACAACATCCAGAAGGAGTCG | |
| | | ACCCTGCACCTGGTCCTGCGTCTGAGAGGTGGTA | |
| | | TGCAGATCTTCGTGAAGACCCTGACCGGCAAGA | |
| | | CCATCACCCTGGAAGTGGAGCCCAGTGACACCA | |
| | | TCGAAAATGTGAAGGCCAAGATCCAGGATAAAG | |
| | | AAGGCATCCCTCCCGACCAGCAGAGGCTCATCT | |
| | | TTGCAGGCAAGCAGCTGGAAGATGGCCGCACTC | |
| | | TTTCTGACTACAACATCCAGAAGGAGTCGACCCT | |
| | | GCACCTGGTCCTGCGTCTGAGAGGTGGTATGCA | |
| | | GATCTTCGTGAAGACCCTGACCGGCAAGACCAT | |
| | | CACTCTGGAGGTGGAGCCCAGTGACACCATCGA | |
| | | AAATGTGAAGGCCAAGATCCAAGATAAAGAAG | |
| | | GCATCCCCCCCGACCAGCAGAGGCTCATCTTTGC | |
| | | AGGCAAGCAGCTGGAAGATGGCCGCACTCTTTC | |
| | | TGACTACAACATCCAGAAAGAGTCGACCCTGCA | |
| | | CCTGGTCCTGCGCCTGAGGGGTGGCTGTTAATTC | |
| | | TTCAGTCATGGCATTCGCAGTGCCCAGTGATGGC | |
| | | ATTACTCTGCACTATAGCCATTTGCCCCAACTTA | |
| | | AGTTTAGAAATTACAAGTTTCAGTAATAGCTGA | |
| | | ACCTGTTCAAAATGTTAATAAAGGTTTCGTTGCA | |
| | | TGGTA | |
| ZBTB34 | NM_001099270.1 | CGGGGACTGGCCTGGCGCCGGCGGCGGCGGAGG | 43 |
| | | GGGCGCCGCGGGCGGGCGATGTGAGCGCGGCGC | |
| | | TCTGGACAGAGTACGCTTCATGTCAGTAGAAAT | |
| | | GGACAGCAGCAGTTTTATTCAGTTTGATGTGCCC | |
| | | GAGTACAGCAGCACCGTTCTGAGCCAGCTAAAC | |
| | | GAACTCCGCCTGCAGGGGAAACTATGTGACATC | |
| | | ATTGTACACATTCAGGGTCAGCCATTCCGAGCCC | |
| | | ACAAAGCAGTCCTTGCTGCCAGCTCCCCATATTT | |
| | | CCGGGACCATTCAGCGTTAAGTACCATGAGTGG | |
| | | CTTGTCAATATCAGTGATTAAAAATCCCAATGTG | |
| | | TTTGAGCAGTTGCTTTCTTTTTGTTACACTGGAA | |
| | | GAATGTCCTTGCAGCTGAAGGATGTTGTCAGTTT | |
| | | TCTGACTGCAGCCAGCTTTCTTCAGATGCAGTGT | |
| | | GTCATTGACAAGTGCACGCAGATCCTAGAGAGC | |
| | | ATCCATTCCAAAATCAGCGTTGGAGATGTTGACT | |
| | | CTGTTACCGTCGGTGCTGAAGAGAATCCCGAGA | |
| | | GTCGAAACGGAGTGAAAGACAGCAGCTTCTTTG | |
| | | CCAACCCAGTGGAGATCTCTCCTCCATATTGCTC | |
| | | TCAGGGACGGCAGCCCACCGCAAGCAGTGACCT | |
| | | CCGGATGGAGACGACCCCCAGCAAAGCTTTGCG | |
| | | CAGCCGCTTACAGGAGGAGGGGCACTCAGACCG | |
| | | CGGGAGCAGTGGGAGCGTTTCTGAATATGAGAT | |
| | | TCAGATAGAGGGAGACCATGAGCAAGGAGACCT | |
| | | ATTGGTGAGGGAGAGCCAGATCACCGAGGTGAA | |
| | | AGTGAAGATGGAGAAGTCCGACCGGCCCAGCTG | |
| | | TTCCGACAGCTCCTCCCTGGGTGACGATGGGTAC | |
| | | CACACCGAGATGGTTGATGGGGAACAAGTTGTG | |
| | | GCAGTGAATGTGGGCTCCTATGGTTCTGTGCTCC | |
| | | AGCACGCATACTCCTATTCCCAAGCAGCCTCACA | |
| | | GCCAACCAATGTATCAGAAGCTTTTGGAAGTTTG | |
| | | AGTAATTCCAGCCCATCCAGGTCCATGCTGAGCT | |
| | | GTTTCCGAGGAGGGCGTGCCCGCCAGAAGCGGG | |
| | | CTTTGTCTGTCCACCTGCACAGTGACCTGCAGGG | |
| | | CCTGGTGCAGGGCTCTGACAGTGAAGCCATGAT | |
| | | GAACAACCCCGGGTATGAGAGCAGTCCCCGGGA | |
| | | GAGGAGTGCGAGAGGGCATTGGTACCCGTACAA | |
| | | TGAGAGGTTGATCTGTATTTACTGTGGAAAGTCC | |
| | | TTCAACCAGAAAGGAAGCCTTGATAGGCACATG | |
| | | CGACTCCATATGGGAATCACCCCCTTTGTGTGCA | |
| | | AGTTCTGTGGGAAGAAGTACACACGGAAGGACC | |
| | | AACTGGAGTACCACATCCGGGGCCATACAGATG | |
| | | ATAAACCATTCCGCTGTGAGATCTGCGGGAAGT | |
| | | GCTTTCCATTCCAAGGTACCCTCAACCAGCACTT | |
| | | GCGGAAAAACCACCCAGGCGTTGCTGAAGTCAG | |
| | | GAGTCGCATTGAGTCCCCCGAGAGAACAGATGT | |
| | | GTACGTGGAACAGAAACTAGAAAATGACGCATC | |
| | | GGCCTCAGAGATGGGCCTAGATTCCCGGATGGA | |
| | | AATTCACACAGTGTCTGATGCTCCCGATTAAGAT | |
| | | GGTAAAGAAGTGCACCCAAACAAAGCACATTAA | |
| | | TCAATGCATATTTGTGATTTGCTTTGTTGTAATCT | |
| | | TTGGTTTTCCCAACCATCTGGAAATCTCTTGGTC | |
| | | TCTTGGCAGTTTTTCTAAAGTTTCTGGATGGAAC | |
| | | ACTTCGTTGTGTTTATCCTTTCCCCTGCCCTCCCT | |
| | | CCCCGAAGGAGCTCAAAGCATGAAGGGCAACGC | |
| | | ATCCAGGGAAAACACAGGCTGACAGTATTCCTC | |
| | | TTTGGCTGAACTCTTAATCCAAAATCTGCCAGTG | |
| | | ATTTAGCTATGCCAACTGGTTGACCCTCCATTCT | |
| | | CTGCCAAGAGGCATACTCTTTCTCATTGTGTGCG | |
| | | CTGGCAGCAGTGCACTTCCACGGAGGGAGATTA | |
| | | GGATGCCGTCAGCTGATACAAATGGGTAACCTT | |
| | | TTCTAATTTAAAATTCCTTTTAGGGGGTAGTTAG | |
| | | ACAATTTATATATATATATAATAAAACTATTATT | |
| | | ATATATATAGTATATATACATTTTCAAATTTGAT | |
| | | TTTATTCTGGTTGAGGTGAATGTAAGAGGAATAT | |
| | | ATAATTTAATACAATGTGAACAGGGCTTCTGAGT | |
| | | CTATCTCATCCCTACCTAATATGTTAGGGTTTTG | |
| | | CCCCTTCATTTCCCTTACAAAAGAATGTTAGTAG | |
| | | GTTTATATTAATCATTGTGTCCAAAAGCAAGCAA | |
| | | AGCAAATCACAGTGTTCACAGCTCTGCTTCATAA | |
| | | CAAATACATAAACCAAATGCCATAAAATTTCTTC | |
| | | AACTCTAGTTGGAAACCGTTTGGAATTTTTGTTA | |
| | | GTTGTCCAGCAGGTAAGCTGGATGACCTGTGGT | |
| | | GCTGACCTTTTTACATAGTGTAGTGTTATATTAG | |
| | | CCAACCCCAAAGGAGCAGTGGTTTTCAAGGTTTT | |
| | | TACTGGCCTACAAATCTACCTTCATTCCGTACTG | |
| | | TAGAAACATACATACCAGGTAACTAAATCGAAT | |
| | | CACTCTCTATCATGAGTTAGTACTCACTCGCACT | |
| | | TAAGGAAAGGGATTTGTAGTTCTGTCTACAAAA | |
| | | TTCTCCAAGCAGTGTTGTGGTTTTTTTTGTTTTTG | |
| | | TTTTTTTTCTTTCTCTTTTCAAACAGCCAGTTCAG | |
| | | GTGCACAGCAACTTTTTCTACATGCAGTTCCCAG | |
| | | GGAAACTGCAGAACTTAGAATTTGTACTTTTTGT | |
| | | AAAGCTATACTCTATGGGAATTGCAAGCAATAT | |
| | | ATCTATCTTAGTATTGTGTGTGCTAATGAGAGCC | |
| | | TCAGTGGCTCCCCCACTCTCTCAGTGTTTCCTGC | |
| | | TTAAAGAACCAACAGTTTAAAAGCCCTCTAAGA | |
| | | TACTCTGTGTGTCACCAAATCTGTGTGTCACCAT | |
| | | TTTTTGGTCATGTGGTGCTATTTTTGTTAAGTGTC | |
| | | TTTTTAGGTCAGTATAGTTGTAGAAAATGTGAAA | |
| | | TCTGATGGTAATAATGAATTATAATTGTTTTCCT | |
| | | CTCTTGAGTTCATAGCTTGAAAAGAGACCTCAA | |
| | | AAGCATGTGCTGGCAAACACGTTACTGTATGAA | |
| | | AACATACCTGAGTCCATTTGAATAATGTTTTATT | |
| | | AGTACTTTCGGAAATGTCTTCAGTTCTGTATTGT | |
| | | GTTCACATACACAAACAGGCTTTACAAGATTGCT | |
| | | TCGGTACTGTAAACTCTGGCAGAGAGTAATTTTG | |
| | | TAGGCAGTTTGGTGGTGAGTTTGTGCTGCAGGCT | |
| | | GCCTGTGGGATGTCAGCGTTCTGGTATCTGCCTG | |
| | | AGAACCTGGGCTCTGAGACGCACAACCAGTGCA | |
| | | CCTCCATAGGAGAACAGTGCAGCCACCTAAAAG | |
| | | AAAAACGAACGAAGGACCAGCCTCAGAGGCTA | |
| | | GAAGTTAAAGGAATACAGAATTAGATGTTTGCT | |
| | | GGTTTTCTGTGCTTTTTTGGCTCCTAAAATACCA | |
| | | ATGGTGGATTTGTTTTTGTTTTTGTTTTTTGTTTT | |
| | | GAGAAATAAAAAGTCATTCAAGCCCTTTGTGTG | |
| | | TAATAGCCCCCAGGGGTGGCAGCTGTGCAGTCG | |
| | | CATCTCTTTGGCACACAGGATCTGTTCACGTGTG | |
| | | AACTGCTGCGCTACACATCAGTGTTAACTCCCTA | |
| | | CAGATTACACTCTAATCCCGCTGCTCCCGAGGAG | |
| | | CGGCTTTGCTAAATCGGGTATATAGTATATGCCT | |
| | | TTTTCCTCGTCAAACTGCCTAAGTAGGGGTTCGT | |
| | | TCTCTCCCTGAAGCACTTGTTCAACTCCTGTTAA | |
| | | AGCCGCGTGCCTCAAGGGGAGGCTGGACCCCAA | |
| | | GTGTTTACCCACTTAAATATGTTCTGGGGTTTCA | |
| | | GGTAAATGTTTGTGGGTTTTTTTTTCCTTACATGA | |
| | | ATAAGTTTGGTTTTGATTTTTTTTTAATTGAATGC | |
| | | AAAAAATTTGTGTTGTGATACAAATTAAGTTTGT | |
| | | GACAAGAAATGCCCAAATCCAAGGACATAAGAG | |
| | | GTCAAGCTCAGGGAAGGAACCTCCTTTTCACTCA | |
| | | GGCTTGGGGCCTCCAGCGAGGTTTCCAGAGCAT | |
| | | TCCATGGTATGAGAGACAGTGAGGAGGGAGGGC | |
| | | ACCTGGCGCGGGCACTTCCAGCGTCCTGGCTCTT | |
| | | GGCATTGTCCGTCTTAACCTTATTTACATGGAGT | |
| | | TCTTTGTATTTGTGAATCTGTTTAACTGGTTTGAG | |
| | | TTTACCAAAGAGTGACTTATCCAAAATTGTCTTT | |
| | | GACAAAAATATCCATTGCTTTGATTGTACAGTTC | |
| | | AGGTTCAAACATTGTAATGGGACTGTTAAGGGG | |
| | | CAGAAAATTGATTGAGTTTCTCTCTAAGAATCAT | |
| | | GATTCCACATTTTGCAAGTTCCACTTGCTCCCAT | |
| | | TCGTGTTGCTAACACTTTACCCTTTCCACTGCTC | |
| | | GCAGTGTTAAGAATGAATTCTCAAGCCATAACA | |
| | | CAGTACTGTAAAGTTCCGCAGGGCTTCGAGGGA | |
| | | GGCAGCGCCTAGGCCAGCACGGAGCTGTGTAGC | |
| | | CTCTCTGAGCGTTCGCACTGTCATGCTTCCCAGG | |
| | | GGTGTGACTGGTGAGAGATTAACTCCATTCAGA | |
| | | TCGGGCAGCAGCAATTAATTGTGCCTTGCCGCAT | |
| | | GAGGATGTGTCAGGAGGATTAACATGACCACAG | |
| | | AACCGAAACATTCTCTCCCTGAAGTTCACTTCAC | |
| | | GTCTCCGCAGACGAAGTACGCTGTGTAACTCCTT | |
| | | AGAGCAACTCTTTTTGGAAAGCAAAGTCCCTATT | |
| | | TCTGTACAGTTTTAGGTTAGGTGTTTCATTTATA | |
| | | ACAGATGCAGAAATCAATTAAGATAAAGTGATA | |
| | | TGTGAAGAAATCTTTTACAGTAAAATATATCCTG | |
| | | AATTCATATAGGCTTGTTCATAATTGAGTCTCTT | |
| | | CTTGAGCTACCTTTTCAATATTAGACAATGTGAA | |
| | | GACAGTGACAGCGTCCTTTTCTAGAGATATTTAG | |
| | | CCTGTTATTACAAACTGTGAAGACAAAGAATTTT | |
| | | ATACTTTTACTAATGTTTGTGGTTTTAAACAGTT | |
| | | ATTTTCATTCTAATCAGTTCTCTACCCTCTAATTT | |
| | | CTACTAAAGCTGTAAATACATTTAGAAATTATAT | |
| | | TTGTAAATACAGTATATGGAGACAAGTTAATTTT | |
| | | TTGGTCAGTGGAAAAAGCCTCCCAACCAATTGG | |
| | | CCCTGCCTTGGCAGTTGTGTTTTTTGTTGTTGTTG | |
| | | TTGTTGTTTTAGTTTAGTTTTTTTTTTTAAACAGC | |
| | | AGAAAGGATACTGTCGGTTCACTGTTGAGCAGA | |
| | | ATATACTGTAGAACGAAAATGATAATTTTTAAAT | |
| | | CTTCCAGAGCATGAGTAAATGTCTTTTCTAATGA | |
| | | TAGCAAATATAACCAACTCTTTGTTTTTCCCTTA | |
| | | GCCCAGACCATATAGACCTGCGTATTTTGTGTGT | |
| | | GGTTTTGTTTTTATTTTTGTTCTTACAGCCTAGAC | |
| | | CCTAGGAAAAATTTGCAGGAACACGAAACAAGG | |
| | | GCTGGGGGGAAAATCATCTATGTGAATGAGCTT | |
| | | TACTTTAAAGAGATCAATGTATTTTATTTTATCA | |
| | | ACTTTTTCTCTTAGTTACTGTGATTTTTGTTGTTG | |
| | | TTGTCCTCGTTATTGTTAAATTCTGTAATGGTTTC | |
| | | CTGTGAAGCCTCCACTGAAAGGGACTCAAATAT | |
| | | GCAACACCTAAACTATTTTCCAAGGGCACATGC | |
| | | CCCTTGAATGGTGCTTCTAGACTGGTCAGGGTTA | |
| | | TTTATTAAATTTTATATATGAAAGTATTGGGGAA | |
| | | TTATGTAAATTCTTTATATGAAACTATCTAGTTC | |
| | | ATAAATCATAGATTTCATATTACTCAGTGCAACT | |
| | | GAACTAAAAGTTCAGAAAAGTCATTCACATTGT | |
| | | TCCAAATTTGTAATGGTTGTCACATGTCACATGC | |
| | | GTCTTTTTCAGTAAGTGCCAGAGTGTTCCCACTG | |
| | | TTTCTGCCCAGTGCTTGACTTCTCGGCCCGGAAG | |
| | | AGAACCTGCTTTCTCTGGTTTCCTTCCTGAGTCT | |
| | | GGCACAGACGGGGCTATTGTAGTTCTTGATCAA | |
| | | GTCCTGGAGTCAGCCTTGCCTGGCTCTCCTTGTA | |
| | | GCAGATTCAGTCCACAGACCTCTTGCTGCCCCTC | |
| | | AGTGACAAGTATGCTGTGAATTCAACCTTTGGAC | |
| | | TTGCTGCCCAAGCCTTTGGTTGCTGCCCTGACTA | |
| | | TTGTAAGAGGTAAACTTACCTGGTTTGTTTGAGA | |
| | | ATGACCATTTTCCTAATGTGAAAACCATCTCTCT | |
| | | CACCACTTTTATTAGTAGGGCTAACATTTTTTTC | |
| | | CGTTATAAATGGTTGAGCAATTTGAATGACTTAA | |
| | | CACAGTGTCATTATCTTGCAATATAAACTGGTAA | |
| | | CCTCACAACTCCACACTTCATCACCATATGAAGT | |
| | | AAATGAAGCTAGCTAAGCGGATGCTGTATCAAC | |
| | | TAGTAACTTGCCATTAAGGATTATTTTATAGCAT | |
| | | GAATTTAAGACTATTTATTCAAATGATATTTTAC | |
| | | TCTTGTATTCACTTTGTTTTAGATTTGTGACATGA | |
| | | ATATTTCAGTGCTGCTTAATTTTGTTCTGAATTCT | |
| | | TGTTTCTTGCTTGTAAATGGCTTTTTTATGGTATA | |
| | | AATAAAGTCAATGGACATTGCTGTTTGTAAATA | |
| | | AAAATGCTGCTAGAGCAAAAAAAAAAAAAAAA | |

In aspects of the methods of the present disclosure, gene expression is measured using methods known in the art that utilize probes targeting the genes of interest. The genes and exemplary target regions of those genes useful for determining gene expression in the methods of identifying mismatch repair deficiency in a subject disclosed herein are shown in Table 3.

TABLE 3
Exemplary Gene Targets for Determining Gene Expression

| | | Exemplary | | SEQ. |
|---|---|---|---|---|
| | GenBank | Target | | ID |
| Gene | Accession No. | Region | Target Sequence | NO. |
| MLH1 | NM_000249.2 | 1606-1705 | CAGGGACATGAGGTTCTCCGGGAGATGTT | 44 |
| | | | GCATAACCACTCCTTCGTGGGCTGTGTGA | |
| | | | ATCCTCAGTGGGCCTTGGCACAGCATCAA | |
| | | | ACCAAGTTATACC | |
| MSH2 | NM_000251.1 | 2515-2614 | AGGTGAAGAAAGGTGTCTGTGATCAAAGT | 45 |
| | | | TTTGGGATTCATGTTGCAGAGCTTGCTAAT | |
| | | | TTCCCTAAGCATGTAATAGAGTGTGCTAA | |
| | | | ACAGAAAGCCCT | |
| MSH6 | NM_000179.2 | 1016-1115 | AGGCCTGAACAGCCCTGTCAAAGTTGCTC | 46 |
| | | | GAAAGCGGAAGAGAATGGTGACTGGAAA | |
| | | | TGGCTCTCTTAAAAGGAAAAGCTCTAGGA | |
| | | | AGGAAACGCCCTCA | |
| PMS2 | NM_000535.6 | 895-994 | TCAGGTTTCATTTCACAATGCACGCATGGA | 47 |
| | | | GTTGGAAGGAGTTCAACAGACAGACAGTT | |
| | | | TTTCTTTATCAACCGGCGGCCTTGTGACCC | |
| | | | AGCAAAGGTCT | |
| EPM2AIP1 | NM_014805.3 | 1323-1422 | GGGGCAACAACAGTCCACTTCTCAGACAA | 48 |
| | | | ACAATGGCTTTGTGACTTTGGCTTCTTGGT | |
| | | | GGACATTATGGAACACCTTCGAGAACTCA | |
| | | | GTGAAGAATTAC | |
| TTC30A | NM_152275.3 | 2493-2592 | TGCCCTCAAGCAACAATTGCTAGAGTAAC | 49 |
| | | | ATCTTTGTATAAGCAAGTAACCCCAGATA | |
| | | | GAGTTGACGTTTCAGCTTTGGGCTGTCAAA | |
| | | | AGGGTATGTCAT | |
| SMAP1 | NM_001044305.2 | 824-923 | GAAAAGCTGCAGAAGAAAGATCAGCAAC | 50 |
| | | | TGGAGCCTAAAAAAAGTACCAGCCCTAAA | |
| | | | AAAGCTGCGGAGCCCACTGTGGATCTTTT | |
| | | | AGGACTTGATGGCC | |
| RNLS | NM_001031709.2 | 727-826 | CTCTTTTATGAAGCTGGTACGAAGATTGAT | 51 |
| | | | GTCCCTTGGGCTGGGCAGTACATCACCAG | |
| | | | TAATCCCTGCATACGCTTCGTCTCCATTGA | |
| | | | TAATAAGAAGC | |
| WNT11 | XM_011545241.2 | 1016-1115 | CTCTGCTTGTGAATTCCAGATGCCAGGCAT | 52 |
| | | | GGGAGGCGGCTTGTGCTTTGCCTTCACTTG | |
| | | | GAAGCCACCAGGAACAGAAGGTCTGGCCA | |
| | | | CCCTGGAAGGA | |
| SFXN1 | NM_001322977.1 | 192-291 | CTACCACCAAACATTAACATCAAGGAACC | 53 |
| | | | TCGATGGGATCAAAGCACTTTCATTGGAC | |
| | | | GAGCCAATCATTTCTTCACTGTAACTGACC | |
| | | | CCAGGAACATTC | |
| SREBF1 | NM_001005291.1 | 1393-1492 | TTCGCTTTCTGCAACACAGCAACCAGAAA | 54 |
| | | | CTCAAGCAGGAGAACCTAAGTCTGCGCAC | |
| | | | TGCTGTCCACAAAAGCAAATCTCTGAAGG | |
| | | | ATCTGGTGTCGGC | |
| TYMS | NM_001071.1 | 396-495 | TGCTAAAGAGCTGTCTTCCAAGGGAGTGA | 55 |
| | | | AAATCTGGGATGCCAATGATCCCGAGACT | |
| | | | TTTTGGACAGCCTGGGATTCTCCACCAGA | |
| | | | GAAGAAGGGGAC | |
| EIF5AL1 | NM_001099692.1 | 2211-2310 | AAAGGAAACACGAAGATTAATCAAGCAG | 56 |
| | | | GAAGGACAAGCTCAGTTTTGCACCCACTG | |
| | | | AATTTGCCACAAATATTGTGGAAAATATT | |
| | | | CTCGGGGACATTGC | |
| WDR76 | NM_024908.3 | 1876-1975 | CGTTTGGTGGAGAATACCTTGTCTCTGTGT | 57 |
| | | | GTTCCATCAATGCCATGCACCCAACTCGGT | |
| | | | ATATTTTGGCTGGAGGTAATTCCAGCGGG | |
| | | | AAGATACATGT | |

### Definitions

The terms “non-hypermutated” and “non-hypermutated samples” refer to tumor samples that have a mutation rate of less than 7 mutations in every 10⁶ bases, or have a mutation rate of less than 8 mutations in every 10⁶ bases, or have a mutation rate of less than 9 mutations in every 10⁶ bases, or have a mutation rate of less than 10 mutations in every 10⁶ bases, or have a mutation rate of less than 11 mutations in every 10⁶ bases, or have a mutation rate of less than 12 mutations in every 10⁶ bases.

The terms “hypermutated” and “hypermutated samples” refer to tumor samples that have a mutation rate of more than 12 mutations in every 10⁶ bases, or have a mutation rate of more than 13 mutations in every 10⁶ bases, or have a mutation rate of more than 14 mutations in every 10⁶ bases, or have a mutation rate of more than 15 mutations in every 10⁶ bases.

The term “mismatch repair deficiency” (MMRd), refers to the loss of function of at least one gene involved in DNA mismatch repair due to biallelic inactivation of the at least one gene. The biallelic inactivation can be caused by a variety of factors, including, but not limited to, somatic or germline mutations within the coding region of the at least one gene, methylation of the promoter of the at least one gene, leading to silencing of that promoter through a mechanism referred to as the CpG island methylator phenotype (CpG), and/or microRNA-induced downregulation of the expression of the at least one gene. The current state of the art for determining whether a sample displays mismatch repair deficiency is through the use of immunohistochemistry to visualize the expression of genes involved in DNA mismatch repair. The at least one gene involved in DNA mismatch repair can comprise MLH1, MSH2, MSH6 and PMS2. Mismatch repair deficiency causes hypermutation and microsatellite instability. Thus, determining that a tumor is mismatch repair deficient also indicates that the tumor is hypermutated and that the tumor is microsatellite instable.

The term “microsatellite instability” refers to length variations at short, repetitive DNA sequences, known as microsatellites (MS), within the genome. Tumors that are said to be microsatellite instable are tumors that display higher variations in the length of these short, repetitive DNA sequences as compared to normal, non-cancerous cells. Microsatellite instability can be caused by mismatch repair deficiency. In clinical settings, detection of MSI is customarily profiling the Bethesda markers, which often include two mononucleotide (BAT25 and BAT26) and three dinucleotide (D5S346, D2S123 and D17S250) MS loci. Colorectal tumors unstable at >40% of the Bethesda markers are considered high level microsatellite instable (MSI-H) and are known to have a better prognosis and to be less prone to metastasis than microsatellite stable (MSS) tumors. More recent guidelines suggest analyzing the length of four mononucleotide repeat loci comprising BAT25, BAT26, BAT40, and transforming growth factor receptor type II and three dinucleotide repeat loci comprising D2S123, D5S346 and D17S250 to determine the MSI status of a tumor sample. The length of these loci in a tumor sample is compared to the length of these loci in a non-tumor sample of the same tissue or mononuclear blood cells using multiplex-fluorescent labeled PCR and capillary electrophoresis. Tumors are classified as microsatellite stable (MSS) if none of the loci show a change in size in the tumor sample as compared to the non-tumor and blood cell sample. Tumors are classified as low level microsatellite instable (MSI-L) if one or two of the loci show a change in size in the tumor sample as compared to the non-tumor and blood cell sample. Tumors are classified as high level microsatellite instable (MSI-H) if three or more loci show a change in size in the tumor sample as compared to the non-tumor and blood cell sample.

As described in the preceding, the methods of the present disclosure can be used to identify mismatch repair deficiency in a subject using gene expression data in a tumor sample from a subject. The sample can be a biological sample. As will be appreciated by those in the art, the sample may comprise any number of things, including, but not limited to: cells (including both primary cells and cultured cell lines) and tissues (including cultured or explanted). In aspects, a tissue sample (fixed or unfixed) is embedded, serially sectioned, and immobilized onto a microscope slide. As is well known, a pair of serial sections will include at least one cell that is present in both serial sections. Structures and cell types, located on a first serial section will have a similar location on an adjacent serial section. The sample can be cultured cells or dissociated cells (fixed or unfixed) that have been immobilized onto a slide.

In aspects, a tissue sample is a biopsied tumor or a portion thereof, i.e., a clinically-relevant tissue sample. For example, the tumor may be from a breast cancer. The sample may be an excised lymph node.

The sample can be obtained from virtually any organism including multicellular organisms, e.g., of the plant, fungus, and animal kingdoms; preferably, the sample is obtained from an animal, e.g., a mammal. Human samples are particularly preferred.

In some aspects, the preceding methods are used in the diagnosis of a condition. As used herein the term diagnose or diagnosis of a condition includes predicting or diagnosing the condition, determining predisposition to the condition, monitoring treatment of the condition, diagnosing a therapeutic response of the disease, and prognosis of the condition, condition progression, and response to particular treatment of the condition. For example, a tissue sample can be assayed according to any of the methods described herein to determine the presence and/or quantity of markers of a disease or malignant cell type in the sample (relative to the non-diseased condition), thereby diagnosing or staging a disease or a cancer.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, and leukemia. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or cervical cancer.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

Any of the above aspects and embodiments can be combined with any other aspect or embodiment as disclosed here in the Summary and/or Detailed Description sections.

The term “immunotherapy” can refer to activating immunotherapy or suppressing immunotherapy. As will be appreciated by those in the art, activating immunotherapy refers to the use of a therapeutic agent that induces, enhances, or promotes an immune response, including, e.g., a T cell response while suppressing immunotherapy refers to the use of a therapeutic agent that interferes with, suppresses, or inhibits an immune response, including, e.g., a T cell response.

As will be appreciated by those in the art, activating immunotherapy may comprise the use of checkpoint inhibitors. Checkpoint inhibitors are readily available in the art and include, but are not limited to, a PD-1 inhibitor, PD-L1 inhibitor, PD-L2 inhibitor, or a combination thereof. Checkpoint inhibitors can comprise antibodies. These antibodies can include, but are not limited to anti-PD1 antibodies, anti-PDL1 antibodies, or anti-CTLA4 antibodies. Anti-PD1 antibodies and anti-PD-L1 antibodies can include, but are not limited to, pembrolizumab, nivolumab, atezolizumab, avelumab, durvalumab, pidilizumab, REGN2810, AMP-224, MEDI0680, PDR001 and CT-001. Anti-CTLA4 antibodies can include but are not limited to ipilimumab and tremelimumab.

Additionally, the immunotherapy that is provided to a patient in need thereof according to the methods of the present invention comprises providing a cytokine agonist or cytokine antagonist, that is an agonist or antagonist of interferon, IL-2, GMCSF, IL-17E, IL-6, IL-Ia, IL-12, TFGB2, IL-15, IL-3, IL-13, IL-2R, IL-21, IL-4R, IL-7, M-CSF, MIF, myostatin, Il-10, 11-24, CEA, IL-11, IL-9, IL-15, IL-2Ra, TNF or a combination thereof.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody that binds to a target refers to an antibody that is capable of binding the target with sufficient affinity such that the antibody is useful as a diagnostic and/or therapeutic agent in targeting the target. In one embodiment, the extent of binding of an anti-target antibody to an unrelated, non-target protein is less than about 10% of the binding of the antibody to target as measured, e.g., by a radioimmunoassay (RIA) or biacore assay. In certain embodiments, an antibody that binds to a target has a dissociation constant (Kd) of <1 μM, <100 nM, <10 nM, <1 nM, <0.1 nM, <0.01 nM, or <0.001 nM (e.g. 10⁸ M or less, e.g. from 10⁸ M to 10¹³ M, e.g., from 10⁹ M to 10¹³ M). In certain embodiments, an anti-target antibody binds to an epitope of a target that is conserved among different species.

A “blocking antibody” or an “antagonist antibody” is one that partially or fully blocks, inhibits, interferes, or neutralizes a normal biological activity of the antigen it binds. For example, an antagonist antibody may block signaling through an immune cell receptor (e.g., a T cell receptor) so as to restore a functional response by T cells (e.g., proliferation, cytokine production, target cell killing) from a dysfunctional state to antigen stimulation.

An “agonist antibody” or “activating antibody” is one that mimics, promotes, stimulates, or enhances a normal biological activity of the antigen it binds. Agonist antibodies can also enhance or initiate signaling by the antigen to which it binds. In some embodiments, agonist antibodies cause or activate signaling without the presence of the natural ligand. For example, an agonist antibody may increase memory T cell proliferation, increase cytokine production by memory T cells, inhibit regulatory T cell function, and/or inhibit regulatory T cell suppression of effector T cell function, such as effector T cell proliferation and/or cytokine production.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless defined otherwise, all technical and scientific terms used herein have the same meaning as commonly understood by one of ordinary skill in the art to which the invention pertains. Although other probes, compositions, methods, and kits similar, or equivalent, to those described herein can be used in the practice of the present disclosure, the preferred materials and methods are described herein. It is to be understood that the terminology used herein is for the purpose of describing particular aspects only, and is not intended to be limiting.

### EXAMPLES

### Example 1—Loss of Mismatch Repair Gene Expression Predicts Microsatellite Instability and Hypermutation

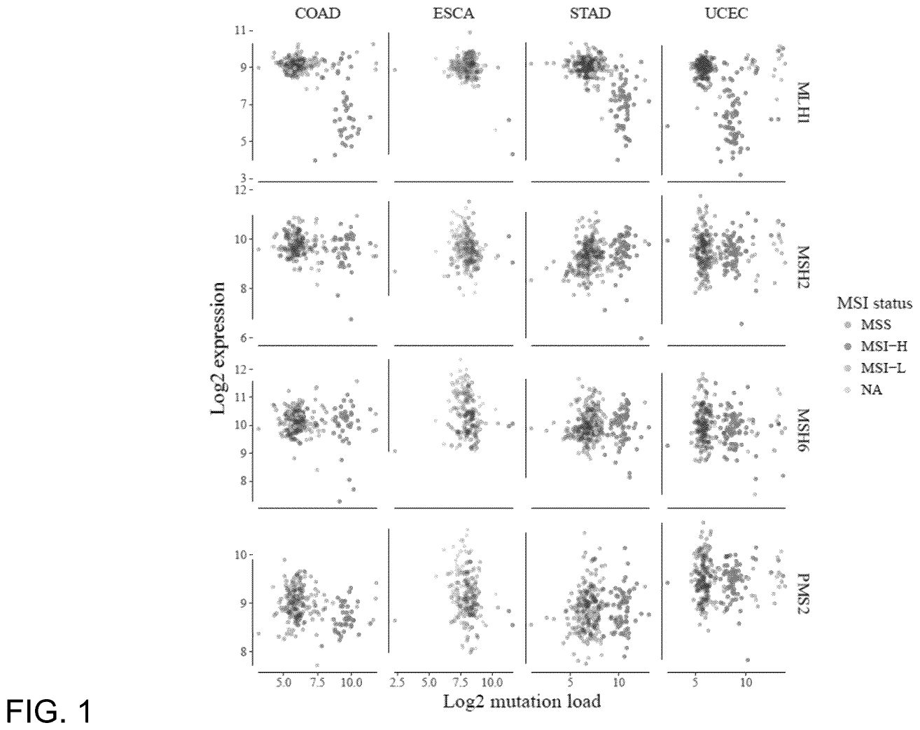

Because loss of protein expression for any of the mismatch repair (MMR) genes MLH1, MSH2, MSH6, or PMS2 is sufficient to identify tumors with microsatellite instability, it is plausible that loss of mRNA expression in these genes can provide a surrogate measurement of tumor microsatellite instability (MSI). FIG. 1 shows a series of graphs in which MMR gene expression is plotted against mutation burden and MSI status in colon adenocarcinoma (COAD), esophageal carcinoma (ESCA), stomach adenocarcinoma (STAD) and uterine corpus endometrial carcinoma (UCEC) tumors. FIG. 1 reveals the strong association between these three phenomena, and it shows that loss of MMR gene expression predicts MSI and hypermutation with high specificity.

In all 4 tumor types (colon, esophageal, stomach, and uterine), a cluster of hypermutated tumors is easily visible, with the subtype being relatively abundant in the colon, stomach, and uterine cancer The Cancer Genome Atlas (TCGA) data sets and rare in esophageal cancers. In all four datasets, these hypermutated tumors are strongly enriched for MSI. In colon, stomach, and uterine cancers, a small third cluster of tumors with an even higher mutation burden is apparent. These ultramutated tumors are often MSS or low-level MSI (MSI-L) in the TCGA datasets. Instead, these tumors have a mutation in one of the polymerase genes POLE or POLD1, consistent with a mechanism in which defective polymerase leads to widespread errors in DNA replication. A small fraction of each cancer type is minimally mutated. Furthermore, the average mutation burden within a given cluster is not preserved across tumor types; for example, non-hypermutated (typical) esophageal cancers have 3.8 times the mutation rate of non-hypermutated colon cancers.

MSI-H status as determined by PCR occurs in most (67%-86%) of the hypermutated tumors in these cancers types and in a smaller fraction of the ultramutated tumors. MSI-H occurs in less than 1.4% of non-hypermutated tumors in each dataset. MSI-L status occurs primarily (>92%) in non-hypermutated tumors in the colon, esophageal, and stomach datasets, while in the uterine dataset MSI-L status occurs with approximately equal frequency across non-hypermutated, hypermutated, and ultramutated tumors.

FIG. 1 also shows that loss of expression of the four MMR genes, observed as low-expression outliers, are also apparent within each cancer type. MLH1 is by far the most frequently under-expressed of these genes. In TCGA, MLH1 expression loss occurs in 16% of colon cancers, 3% of esophageal cancers, 20% of stomach cancers, and 29% of uterine cancers. MLH1 loss on its own is a surprisingly sensitive biomarker, detecting two thirds or more of the hypermutation cases in each of these cancer types. Expression loss in the other three MMR genes detects a small number of additional hypermutated/MSI samples not captured by MLH1: MSH2 loss detects 5 additional MSI-H tumors in these 4 datasets, MSH6 loss detects 2, and PMS2 loss detects none. These loss of expression events are highly specific predictors of both MSI and hypermutation, occurring almost exclusively within hypermutated and MSI-H tumors. However, a subset of less than 10% of MSI tumors display normal expression levels of these 4 genes, indicating MMR dysfunction arising from a cause other than loss of mRNA expression.

### Example 2—Hypermutated Tumors Share Common Transcriptional Patterns in Colon, Stomach, and Uterine Cancers

Approximately one third of hypermutation or ultramutation events as measured by next-generation sequencing cannot be detected by loss of MMR gene expression. In such cases, transcriptomic events downstream of mismatch repair deficiency (MMRd) might enable detection of hypermutation independent of the expression levels of the classic MMR genes. In cancers where hypermutation has a common origin in MMRd, and possibly in CpG island methylator phenotype (CIMP), it is plausible that hypermutated tumors will display common transcriptional patterns across tumor types. To evaluate whether broader expression patterns could predict MSI and hypermutation, univariate linear models testing the association of hypermutation status with each gene in the TCGA whole transcriptome RNA-Seq datasets were run. These models were fit separately within the colon, stomach, and uterine cancer datasets, omitting esophageal cancer because the presence of only 4 hypermutated tumors in that dataset limited statistical power.

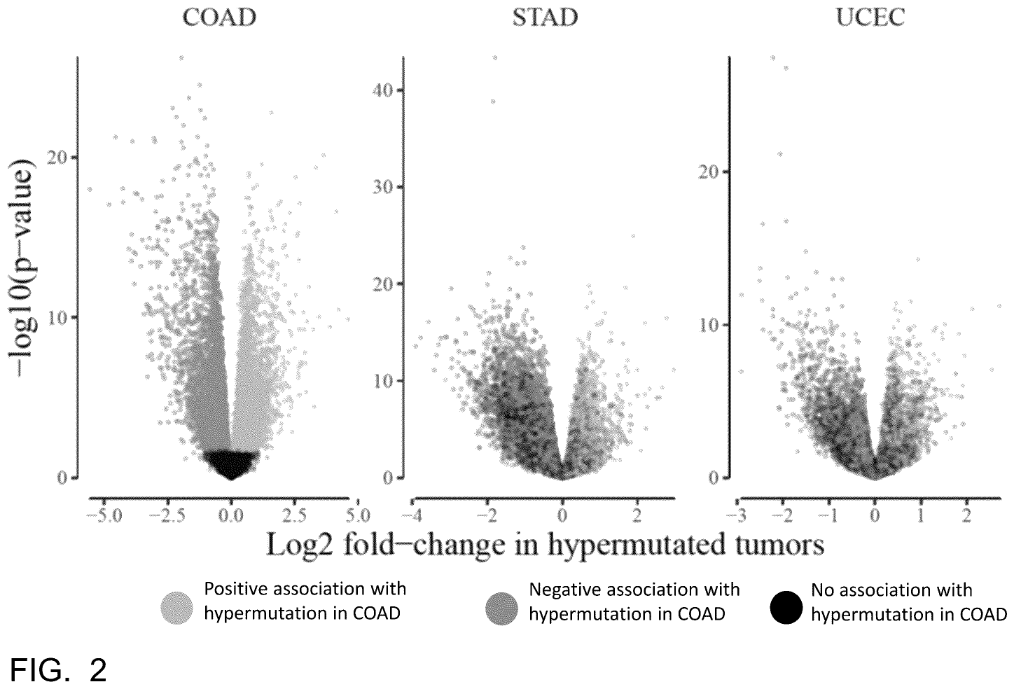

A great deal of the transcriptome had significant association with hypermutation status in these datasets: a Benjamini-Hochberg false discovery rate (FDR)<0.05 was achieved by 7800 genes in colon adenocarcinomas, 9337 genes in stomach adenocarcinomas, and 3848 genes in uterine carcinomas. FIG. 2 is a series of volcano plots that show genes' associations with hypermutation in COAD, STAD and UCEC tumors. FIG. 2 shows that a number of these genes behaved similarly across all 3 cancer types: 420 genes had a FDR<0.05 and a positive association with hypermutation in all 3 datasets, and 672 genes had a FDR<0.05 and a negative association with hypermutation in all 3 cancer types.

Some consistent biology emerges from this comparison, in that gene sets relating to DNA replication machinery and metabolism are highly enriched for genes with consistent positive associations with hypermutation. Table 4 shows the proportion of the genes in each gene set that are consistently down-regulated and consistently up-regulated with hypermutation across COAD, STAD and UCEC datasets, where “consistently up-regulated” is taken to mean “false discover rate<0.05 and a positive association with hypermutation in all 3 datasets. For Table 4, Kyoto Encyclopedia of Genes and Genomes (KEGG), Biocarta, and Reactome gene sets were downloaded from the Molecular Signatures Database (MSigDB).

TABLE 4
Genes down-regulated and up-regulated in cancer datasets

| | Proportion | Proportion |
|---|---|---|
| | down in | up in |
| | COAD, | COAD, |
| | STAD, | STAD, |
| | and | and |
| | UCEC | UCEC |
| BIOCARTA_KREB_PATHWAY | 0 | 0.38 |
| REACTOME_ACTIVATION_OF_THE_PRE_REPLICATIVE_COMPLEX | 0 | 0.33 |
| REACTOME_G1_S_SPECIFIC_TRANSCRIPTION | 0 | 0.31 |
| REACTOME_PROCESSIVE_SYNTHESIS_ON_THE_LAGGING_STRAND | 0 | 0.27 |
| REACTOME_UNWINDING_OF_DNA | 0 | 0.27 |
| REACTOME_E2F_MEDIATED_REGULATION_OF_DNA_REPLICATION | 0 | 0.26 |
| BIOCARTA_MCM_PATHWAY | 0 | 0.25 |
| REACTOME_ASSOCIATION_OF_LICENSING_FACTORS_WITH_THE_PRE_R | 0 | 0.25 |
| REACTOME_DNA_STRAND_ELONGATION | 0 | 0.23 |
| REACTOME_CITRIC_ACID_CYCLE_TCA_CYCLE | 0 | 0.21 |
| REACTOME_LAGGING_STRAND_SYNTHESIS | 0 | 0.21 |
| BIOCARTA_DNAFRAGMENT_PATHWAY | 0 | 0.2 |
| BIOCARTA_GLYCOLYSIS_PATHWAY | 0 | 0.2 |
| REACTOME_CDC6_ASSOCIATION_WITH_THE_ORC_ORIGIN_COMPLEX | 0 | 0.2 |
| REACTOME_REMOVAL_OF_THE_FLAP_INTERMEDIATE_FROM_THE_C_STRAND | 0 | 0.2 |
| KEGG_GLYOXYLATE_AND_DICARBOXYLATE_METABOLISM | 0 | 0.19 |
| KEGG_DNA_REPLICATION | 0 | 0.19 |
| REACTOME_HOMOLOGOUS_RECOMBINATION_REPAIR_OF_REPLS | 0.06 | 0.19 |

This study demonstrates that numerous genes display strong differential expression with hypermutation across all cancer types and suggests that a data-driven predictor of hypermutation could prove informative.

### Example 3—Gene Expression Algorithms for Predicting MMRd, Hypermutation, and MSI

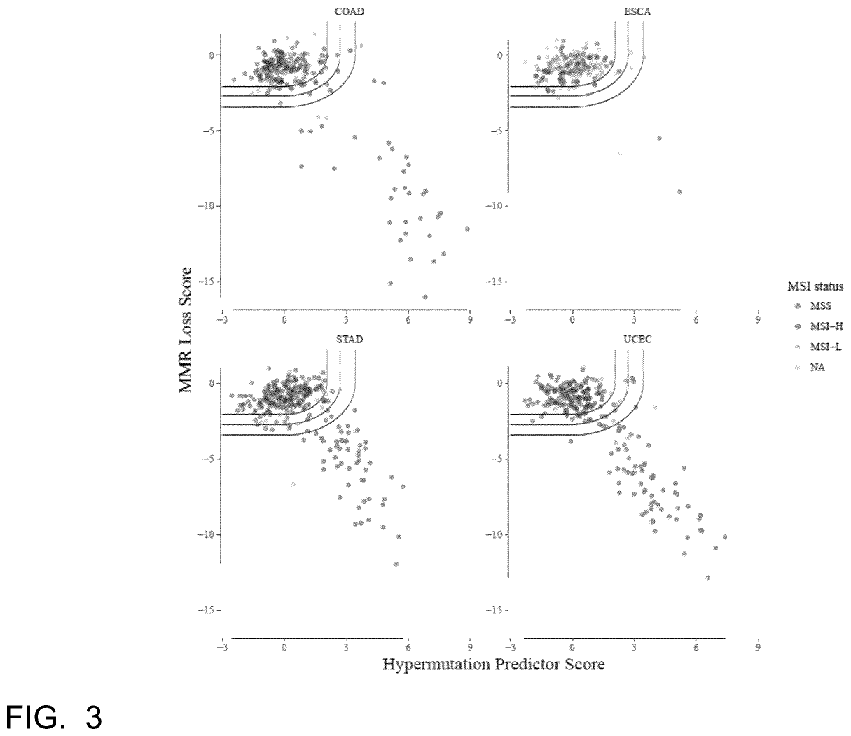

Based on the results from examples 1 and 2, three gene expression algorithms for predicting MMRd, hypermutation, and MSI were trained. The “MMR Loss” algorithm uses the results from FIG. 1 to measure loss of expression of the four MMR genes (MLH1, MSH2, MSH6, and PMS2). The “Hypermutation Predictor” algorithm relies on the results from FIG. 2, using genes differentially expressed in hypermutated tumors to predict a tumor's hypermutation status. Finally, to attain the most powerful prediction with all available information, the “MSI Predictor” algorithm combines the MMR Loss and Hypermutation Predictor algorithms in a single score designed to predict MSI status. FIG. 3 is a series of graphs that show how the three algorithms relate to each other. The curved lines in FIG. 3 show the show the decision boundaries corresponding, from top-left to bottom-right, to MSI predictor score p-value cutoffs of 0.05, 0.01, and 0.00. The derivations of these algorithms are described in the materials and methods section below.

### Results

The ability of the MSI Predictor algorithm and its 2 component algorithms to predict tumor MSI was evaluated. Table 5 shows that the MMR Loss (also referred to herein as MLS score) and Hypermutation Predictor (also referred to herein as HPS score) algorithms were each accurate predictors of MSI, with the MSI Predictor (also referred to herein as MPS score) algorithm showing higher accuracy as measured by True Positive Rate (TPR; the proportion of MSI-high cases detected by each algorithm) and False Positive Rate (FPR; the proportion of non-hypermutated cases falsely called hypermutated by the gene expression algorithms). A p-value threshold of 0.01 was used for all gene expression algorithms. Numbers in the parentheses in Table 5 give 95% confidence intervals calculated by the Wilson method.

TABLE 5
MMR loss and hypermutation predictor performance

| | COAD | | ESCA | | STAD | | UCEC | |
|---|---|---|---|---|---|---|---|---|
| TPR MMR | 0.9 | (0.76-0.96) | 1 | (0.34-1) | 0.92 | (0.82-0.96) | 0.94 | (0.86-0.98) |
| loss score | | | | | | | | |
| TPR | 0.74 | (0.59-0.85) | 1 | (0.34-1) | 0.8 | (0.68-0.88) | 0.94 | (0.86-0.98) |
| Hypermutation | | | | | | | | |
| Predictor score | | | | | | | | |
| TPR MSI | 0.9 | (0.76-0.96) | 1 | (0.34-1) | 0.9 | (0.8-0.95) | 0.93 | (0.84-0.97) |
| Predictor score | | | | | | | | |
| FPR MMR loss | 0.26 | (0.2-0.32) | 0.08 | (0.04-0.17) | 0.3 | (0.24-0.36) | 0.36 | (0.3-0.43) |
| score | | | | | | | | |
| FPR | 0.17 | (0.12-0.23) | 0.04 | (0.01-0.12) | 0.23 | (0.18-0.29) | 0.37 | (0.31-0.43) |
| Hypermutation | | | | | | | | |
| Predictor score | | | | | | | | |
| FPR MSI | 0.21 | (0.16-0.28) | 0.03 | (0.01-0.1) | 0.25 | (0.19-0.31) | 0.3 | (0.24-0.36) |
| Predictor score | | | | | | | | |

However, because the Hypermutation Predictor algorithm was trained from these samples it is subject to overfitting. Therefore, its performance, as well as the performance of the MSI Predictor algorithm, may be exaggerated in this data. In contrast, the MMR Loss algorithm was developed using a minimal training procedure that only required estimates of the mean and interquartile range of each gene in non-hypermutated samples; as such, this algorithm's performance is more likely to be reproduced in new datasets.

Table 6 shows that the gene expression algorithms predicted hypermutation in TCGA datasets almost as well as they predicted MSI. TCGA's PCR-based MSI assay was a slightly more powerful predictor of hypermutation, though this advantage was generally not statistically significant.

TABLE 6
Prediction of hypermutation using gene expression algorithms

| | COAD | | ESCA | | STAD | | UCEC | |
|---|---|---|---|---|---|---|---|---|
| TPR MMR loss | 0.77 | (0.62-0.87) | 0.75 | (0.3-0.95) | 0.8 | (0.69-0.88) | 0.73 | (0.63-0.81) |
| score | | | | | | | | |
| TPR | 0.65 | (0.5-0.78) | 0.75 | (0.3-0.95) | 0.74 | (0.63-0.83) | 0.83 | (0.74-0.9) |
| Hypermutation | | | | | | | | |
| Predictor score | | | | | | | | |
| TPR MSI | 0.79 | (0.65-0.89) | 0.75 | (0.3-0.95) | 0.79 | (0.67-0.87) | 0.74 | (0.65-0.82) |
| Predictor score | | | | | | | | |
| TPR MSI status | 0.86 | (0.73-0.93) | 0.67 | (0.21-0.94) | 0.88 | (0.78-0.94) | 0.74 | (0.65-0.82) |
| FPR MMR loss | 0.1 | (0.06-0.15) | 0.06 | (0.03-0.11) | 0.11 | (0.07-0.16) | 0.13 | (0.08-0.19) |
| score | | | | | | | | |
| FPR | 0.02 | (0.01-0.05) | 0.03 | (0.01-0.06) | 0.04 | (0.02-0.08) | 0.12 | (0.08-0.18) |
| Hypermutation | | | | | | | | |
| Predictor score | | | | | | | | |
| FPR MSI | 0.04 | (0.02-0.08) | 0.02 | (0.01-0.05) | 0.03 | (0.02-0.07) | 0.03 | (0.01-0.07) |
| Predictor score | | | | | | | | |
| FPR MSI status | 0.01 | (0-0.04) | 0 | (0-0.04) | 0 | (0-0.03) | 0.01 | (0-0.05) |

### Materials and Methods

Development and Validation of the MMR Loss Algorithm for Calling MSI Status from Loss of MMR Genes

FIG. 1 suggests that low gene expression values in MLH1, MSH2, MSH6, and PMS2 could be used to detect hypermutation and MSI. Therefore, an algorithm for predicting MSI by detecting loss of expression in these genes was developed. To do so, the uncharacteristically low expression of any one of these genes for a MSS tumor was investigated.

To quantify how atypically low a gene's expression is, knowledge of its mean expression and standard deviation in MSS samples was required. Both of these quantities will vary between cancer types, so the mean and standard deviation were estimated separately for each tumor dataset. A gene's mean expression in MSS samples will vary with platform and batch effects. Therefore, this parameter must be estimated anew when deploying this algorithm on a new platform. To ensure an unbiased procedure, this mean parameter was estimated without reference to known mutation or MSI status, either by taking each gene's median expression across a whole dataset (under the assumption that most cases are MSS) or by fitting a Gaussian mixture model with 2 clusters and taking the mean of the higher cluster. If this algorithm were to be applied in a locked assay, each gene's mean in non-hypermutated samples could be estimated directly and fixed.

The standard deviation of a gene's log-scale expression should be platform-agnostic, as platform effects are generally well-modelled as unique scaling factors applied to each gene, amounting to additive constants on the log-scale. Therefore, this parameter can be estimated in TCGA and applied it to future datasets without further calibration. In colon, stomach, and uterine cancers, each MMR gene's standard deviation in the MSS/non-hypermutated subtype was estimated using the cases where MSS status was known. In the esophageal dataset, in which many MSI calls were missing, samples with unknown MSI were included in this analysis, as MSI is rare in this indication, with only 4 cases in TCGA. These standard deviation estimates are reported Table 7.

TABLE 7
Standard deviations of each mismatch repair gene in microsatellite
stable samples in The Cancer Genome Atlas

| | MLH1 | MSH2 | MSH6 | PMS2 |
|---|---|---|---|---|
| COAD | 0.3241 | 0.4108 | 0.4198 | 0.3259 |
| ESCA | 0.5221 | 0.6602 | 0.7347 | 0.4927 |
| STAD | 0.4245 | 0.6020 | 0.4814 | 0.4314 |
| UCEC | 0.4543 | 0.7312 | 0.6158 | 0.4217 |

Upon calculation of means and standard deviations, the remainder of the algorithm was simple to execute. Each gene was Z-scored, and the minimum of the four Z-scores was taken for each sample. To place the score on a familiar scale, this minimum Z score was then rescaled by the theoretical mean and standard deviation of the minimum of four standard normal random variables, attaining a final “MMR Loss” score with a mean of 0 and standard deviation of 1 in non-hypermutated samples.

A concise description of the procedure for calculating MMR Loss score is as follows. The below algorithm is proposed for calling hypermutation events resulting from loss of expression of 1 of the 4 key MMR genes (MLH1, MSH2, MSH6, or PMS2).
1. Normalize the gene expression dataset using a sensible method.
2. For each gene, estimate μ, the gene's mean expression in non-hypermutated samples. If a low rate of hypermutation is expected in the dataset, each gene's median expression provides a good estimate. If hypermutation is expected to be common, a Gaussian mixture model with two clusters can be fit to each gene's expression data, and the mean of the higher expression cluster should be taken as μ. For single sample applications, μ must be pre-defined using a training dataset run on the same assay.
3. For each gene, look up its standard deviation (σ) in non-hypermutated tumors of the appropriate cancer type in TCGA. Examples of the 4 MMR genes' a values are provided in Table 7.
4. For each sample, score each gene relative to its expected value in non-hypermutated samples as [Z=(x−μ)/σ], where x is the gene's normalized log 2 expression value.
5. For each sample, call Zₘ the minimum Z score from the 4 genes. Calculate the final MMR Loss score, [MLS=(Zₘ+1.03)/0.69], where 1.03 and 0.69 are the theoretical expectation and standard deviation of the minimum of 4 standard normal random variables.
6. Calculate a p-value for each sample: [p=Φ(MLS)], where Φ is the standard normal distribution function. Choose a stringent p-value threshold for calling loss events, at least as strict as 0.01. Most loss of expression events are substantial enough that they are easily detected, so p-values between 0.05 and 0.01 will often result in false positives.
   Development and Validation of the Hypermutation Predictor Algorithm for Calling MSI Status from Genes Differentially Expressed in Hypermutated Tumors

Given an abundance of genes with consistent and highly significant associations with hypermutation, the derivation of a data-driven predictor of hypermutation was sought. 10 genes with good performance across all 3 datasets were selected. Selection was based on multiple considerations, including effect size in the linear models described above and effect size in models fit to subsets of the data (e.g. models excluding ultramutated tumors or hypermutated tumors without MMR gene expression loss). Table 8 shows the genes selected for this process.

TABLE 8
Genes used in the hypermutation predictor score and false
discovery rates (FDR) for various cancer types

| | | COAD | STAD | UCEC |
|---|---|---|---|---|
| Gene | Weight | FDR | FDR | FDR |
| EPM2AIP1 | −0.31218 | 2.13E−19 | 1.49E−35 | 6.80E−24 |
| TTC30A | −0.19894 | 1.54E−13 | 5.22E−17 | 2.59E−07 |
| SMAP1 | −0.1835 | 7.96E−18 | 2.57E−13 | 0.001251 |
| RNLS | −0.19023 | 2.23E−14 | 0.000156 | 4.52E−18 |
| WNT11 | −0.11515 | 1.52E−08 | 0.036791 | 7.02E−06 |
| SFXN1 | 0.214676 | 1.22E−15 | 1.11E−16 | 0.000229 |
| SREBF1 | 0.194835 | 8.58E−11 | 5.48E−14 | 8.62E−06 |
| TYMS | 0.206972 | 2.08E−17 | 2.73E−14 | 0.001611 |
| EIF5AL1 | 0.194935 | 5.99E−13 | 2.86E−13 | 9.06E−05 |
| WDR76 | 0.188582 | 4.26E−12 | 3.80E−09 | 2.67E−07 |

Using the 10 selected genes, a linear predictor score was derived. Each gene was given a weight equal to its mean t-statistic across the 3 datasets and each sample's score was calculated as the sum of its weighted log 2-transformed gene expression values. As the positive and negative weights were nearly balanced, weights were rescaled such that they summed to 0, achieving a score that is invariant to any normalization scheme that adjusts each sample by a scaling constant (i.e., a sample's score was the same under any housekeeping gene normalization regimen, or even in unnormalized data. As a final step, the score was centered and scaled by its mean and standard deviation in MSS samples. Similar to the MMR Loss algorithm, the mean score was estimated in MSS samples anew on each platform. Model-based clustering was again used to estimate this parameter without reference to known MSI status. Also similar to the MMR Loss algorithm, the score's standard deviation in MSS samples in each TCGA dataset was estimated and this parameter was fixed for all future datasets. In the TCGA data from which it was trained, the Hypermutation Predictor score predicts MSI and hypermutation almost as well as the MMR Loss score.

A concise description of the algorithm for calculating Hypermutation Predictor score is as follows. The below algorithm for calling hypermutation events from genes that are differentially expressed between hypermutated/tumors with microsatellite instability (MSI) and non-hypermutated/MSS tumors is proposed.
1. For a given sample, Log 2-transform the expression data for each of the genes in Table 8, multiply each gene by its given weight, and take the sum of these weighted expression values. Call this value x.
2. If applying the assay to a new platform, calibrate the mean parameter for the dataset: fit a Gaussian mixture model with two classes to the data, and take the lower of the two mean parameters. If the mean parameter for the platform has been previously estimated, use that value instead. Call the mean parameter p.
3. Look up the score's standard deviation (a) in non-hypermutated tumors of the appropriate cancer type in TCGA. The 4 datasets' a values are provided in Table 9.

TABLE 9
Standard deviations of the Hypermutation Predictor score in
microsatellite stable samples in The Cancer Genome Atlas

| Tumor | |
|---|---|
| Type | σ |
| COAD | 0.6604 |
| ESCA | 0.7617 |
| STAD | 0.8153 |
| UCEC | 0.7027 |

4. Z-transform the score to have a mean of 0 and standard deviation of 1 in non-hypermutated sample: calculate the Hypermutation Predictor score [HPS=(x−μ)/σ].
5. For each sample: [p=Φ(HPS)], where Φ is the standard normal distribution function. Choose a stringent p-value threshold for calling loss events, at least as strict as 0.01.
   Development and Validation of the MSI Predictor Algorithm for Calling MSI Status from Combined Information in the MMR Loss and Hypermutation Predictor Scores

Ultimately, a single procedure for calling tumors' MSI status was required. The MSI predictor algorithm described below combines the information in the MMR Loss and Hypermutation Predictor scores into a single score for predicting MSI status. First, it was observed that both the MMR Loss and Hypermutation Predictor scores were approximately Gaussian with a mean of 0 and standard deviation of 1 in MSS samples. Furthermore, they appeared uncorrelated in MSS samples. These observations suggested a test that rejects the null hypothesis of MSS/non-hypermutation in samples that fall in extreme values of the joint distribution of these two scores, which could be reasonably approximated as a bivariate normal distribution.

However, a one-sided test was desired and the rejection of the null hypothesis of MSS/non-hypermutation (e.g., when MLH1 expression was extremely high) was unwanted. Additionally, allowing a null score from one test to counteract the evidence from an impressive score from the other test was unwanted (e.g., if the Hypermutation Predictor score suggested hypermutation but all the MMR genes were unusually high, letting the MMR genes' results counteract the evidence from the Hypermutation Predictor score was unwanted). Thus, both the MMR Loss score and the Hypermutation Predictor score were truncated at 0.

This truncation and the assumption of approximate bivariate normality lead to the following test statistic: MSI predictor score=[(max(HPS,mean(HPS))²+min(MLS,0)²)^{1/2}], where HPS is the Hypermutation Predictor score and MLS is the MMR Loss score. Selected contours of this test score, or equivalently, decision boundaries it could delineate, are shown in FIG. 3. By assuming bivariate normality a p-value for the test statistic could be calculated, equal to the mass of a bivariate normal probability distribution falling above the decision boundary implied by the test statistic's value. Using numerical integration, it was found that p-values of 0.05, 0.01, 0.005, and 0.001 correspond to test statistics of 2.058, 2.699, 2.939, and 3.429, respectively.

A concise description of the algorithm for calculating MSI status from combined information in the MMR Loss and Hypermutation Predictor scores is as follows. The below algorithm for calling hypermutation events in a given sample is proposed:
1. Calculate the MMR Loss and Hypermutation Predictor scores as described above. Call MLS the Z-score from the MMR Loss algorithm, and call HPS the Z-score from the Hypermutation Predictor algorithm.

$$2. Calculate the final score : MSI Predictor Score = [ {( {max ( H P S , 0 )}^{2} + {min ( MLS , 0 )}^{2} )}^{1 / 2} ] .$$

3. Compare the score to a pre-specified cutoff. A cutoff of 7.287 is suggested, which corresponds to a p=0.01 threshold for rejecting the null hypothesis of MSS/non-hypermutation.

### Example 4—Validation of MSI Predictor Algorithm in Two Independent Sample Sets Using the NanoString nCounter System

To validate the algorithms trained in TCGA, the NanoString nCounter (NanoString Technologies, Inc., Seattle, Washington, USA) was used to profile two new sample sets for which results of the MMRd IHC assay were available (MSI assays were not run, but the MMRd IHC assay is commonly accepted as a surrogate for MSI). One sample set consisted of 30 MMR-proficient and 30 MMRd colorectal carcinoma samples. The other sample set was 5 MMR-proficient and 10 MMRd endometrial and neuroendocrine tumors, with MMRd status determined by IHC. Endometrial and neuroendocrine samples were combined in a single analysis because of the limited sample sizes.

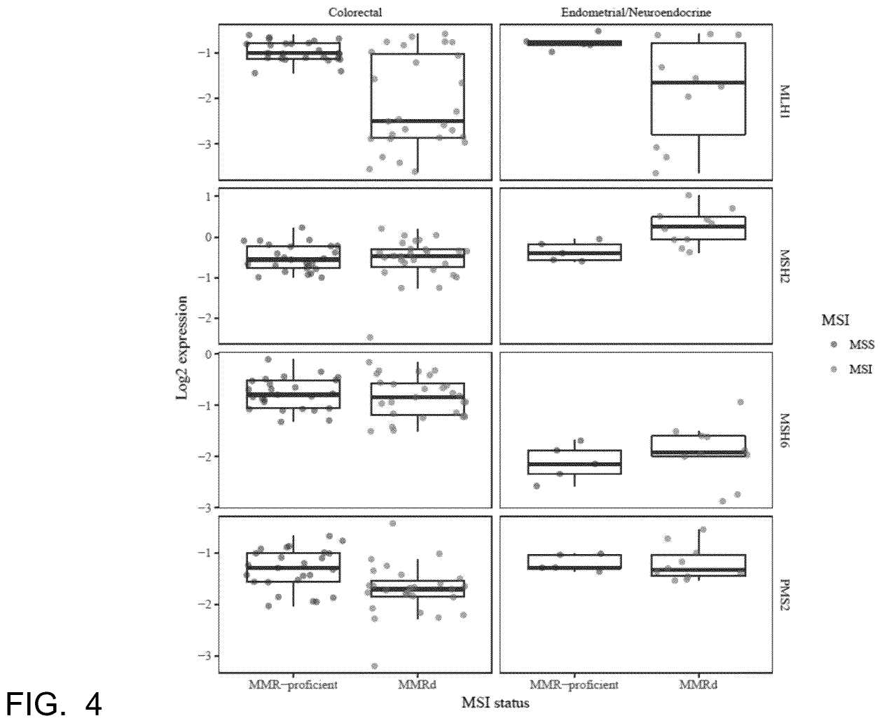

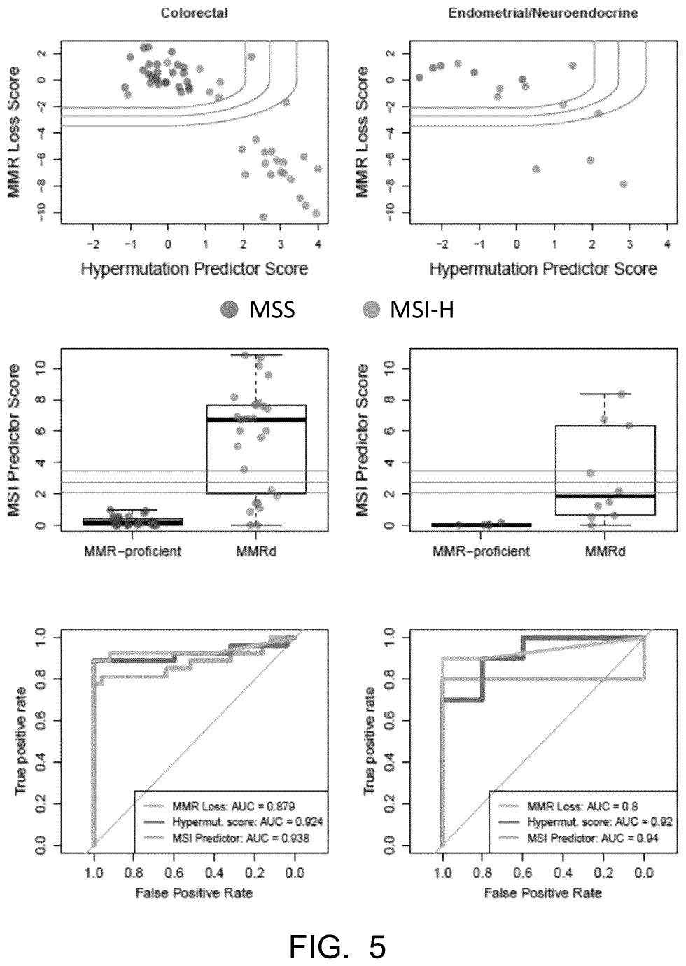

FIG. 4 is a series of box plots that show that, like the phenomenon seen in TCGA, the validation datasets revealed loss of expression events in a majority of MSI samples. In the endometrial and neuroendocrine samples, losses were only observed for MLH1. PMS2 expression was not noticeably suppressed in 2 tumors with mutations in that gene and in 2 tumors with loss of nuclear PMS2 expression seen in IHC. In the colorectal samples, frequent MLH1 loss of expression was apparent, as were a single instance each of MSH2 and PMS2 loss. Loss of expression events occurred exclusively in MMRd tumors. FIG. 5 shows that the MMR loss score, which measures the evidence for loss in any of the four MMR genes, attained an area under the ROC curve (AUC) of 0.80 in endometrial samples and 0.87 in colorectal samples.

FIG. 5 also shows that the Hypermutation Predictor score, a linear combination of 10 genes, retained strong predictive performance in these independent datasets and outperformed the MMR Loss score (area under curve [AUC]=0.902 in endometrial samples and 0.932 in colorectal samples). The MSI Predictor score added negligible predictive power to the Hypermutation Predictor score. The majority of MMRd cases are unambiguously detected by the MSI Predictor score, and the score's overall predictive power was very high (area under curve [AUC]=0.940 in endometrial samples and 0.963 in colorectal samples).

The TCGA training did not map perfectly to the validation datasets. Examining the top row of FIG. 5, it appears that moving the score contours/decision boundaries left would capture more MMRd samples while incurring no false positives. These suboptimal decision boundaries of the Hypermutation Predictor score appear to result from a lower standard deviation in the validation MSS samples than in TCGA MSS samples. If the Hypermutation Predictor score's standard deviation in MSS samples were to be estimated anew in these datasets, it would shift the score contours/decision boundaries left and thereby achieve even better prediction. By implementing the MSI Predictor score using the pre-defined standard deviation estimates from TCGA, the differential score in MSI calling is underutilized and the results are unnecessarily conservative. The reason for the narrower distribution of Hypermutation Predictor scores in MSS samples in NanoString data is unclear. It could result from more precise gene expression measurements or from some unknown difference in the studies' sample preparation methods or clinical populations.

### Materials and Methods

### Calculation of Gene Expression Algorithms in NanoString Validation Datasets

Before the algorithms could be applied to data from a new platform, an up-front calibration step was required: for each of the 4 MMR genes and for the Hypermutation Predictor score, the mean value in non-hypermutated samples (or the “center”) had to be estimated. This calibration was performed using unsupervised techniques blind to the samples' MSI status as described in the methods sections for the respective algorithms.

### MMRd Assay in Colorectal Carcinoma Samples

MSI-H and MSS/MSI-L colorectal cancer tumor samples in formalin-fixed paraffin-embedded (FFPE) blocks were purchased from iSpecimen (Lexington, Massachusetts, USA). MMR status was determined by the original clinical source using IHC for MLH1, MSH2, MSH6, and PMS2. Blocks were then sent to CellNetix (Seattle, Washington, USA) for pathology review and slide cutting.

### MMRd Assay in Endometrial Samples

MMR status was determined by IHC performed at PhenoPath Laboratories, PLLC (Seattle, Washington, USA). Antibody clones used were MSH2 (mouse monoclonal FE11, catalog #M3639; Dako), MSH6 (rabbit monoclonal EP49, catalog #M3646; Dako), MLH1 (mouse monoclonal ES05, catalog #M3640; Dako) and PMS2 (rabbit monoclonal EP51, catalog #M3647; Dako) (Agilent Technologies, Inc., Santa Clara, California, USA). All samples were stained with hematoxylin and eosin to allow for morphological evaluation. MMR status was reviewed by a board-certified pathologist and reported as “no loss of expression” or “loss of expression.”

### NanoString Assay and Normalization

Samples were run using the standard nCounter Gene Expression assay methodology (NanoString Technologies, Inc., Seattle, Washington, USA; see, e.g. Geiss G K et al. Nature biotechnology. 2008 Mar. 1; 26(3):317-25). Total RNA was extracted from each FFPE tumor sample using the Qiagen FFPE RNeasy kit (Qiagen, Inc., Hilden, Germany). A total of 100 ng of RNA was hybridized with the nCounter IO 360 gene expression panel (NanoString Technologies, Inc., Seattle, Washington, USA), with downstream processing and data collection following manufacturer's instructions.

Both NanoString datasets were normalized such that the mean log 2 expression of 10 housekeeping genes was constant across all samples. All analyses used log 2-transformed data.

### Calculation of MSI Algorithms in NanoString Data

Platform differences prevented us from directly applying the TCGA-trained algorithms to NanoString data. Because gene expression platforms differ in the efficiency with which they measure each target sequence, platform effects can be well-modelled by a constant shift in each gene's log-scale normalized expression. Therefore, to apply the algorithms to NanoString data, these constant factors were estimated for each MMR gene and for the Hypermutation Predictor score. To preserve the integrity of this dataset as an unbiased test set for the algorithms, all of these calibration parameters were estimated using unsupervised methods without reference to the known MSI calls. The R library Mclust was used to fit a two-component Gaussian mixture model to each MMR gene's log 2-transformed, normalized expression and to the Hypermutation Predictor score. For the MMR genes, the mean of the higher of the two clusters was taken as the estimate of the mean expression level in non-hypermutated samples; for the Hypermutation Predictor score, the mean in the lower of the two clusters was used. Apart from these mean estimates, all other parameters needed to calculate algorithm scores were calculated from TCGA data without reference to the validation dataset.

### Example 5—Association of MSI Status with Extent of Anti-Tumor Immunity as Measured by the Tumor Inflammation Signature

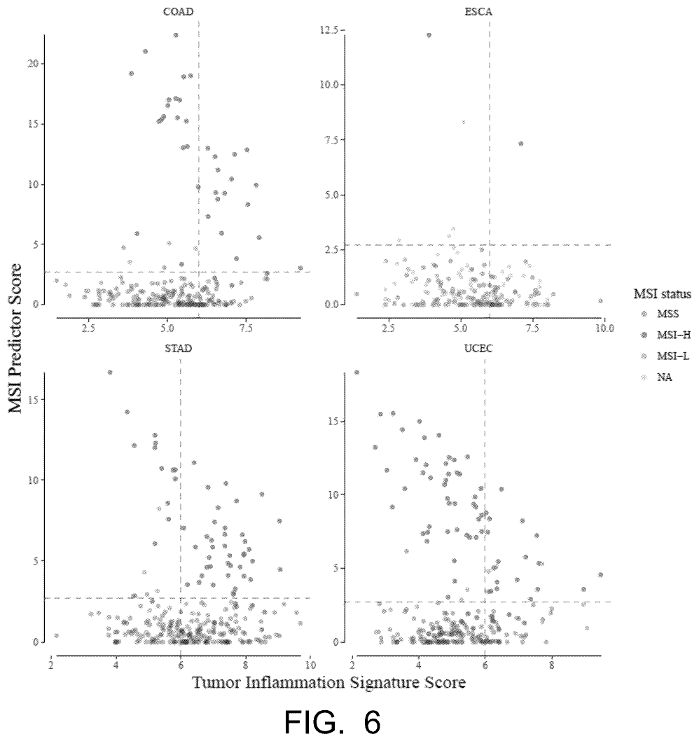

It is well-established that gene expression can predict immunotherapy response by measuring the inflamed microenvironment phenotype. In particular, the Tumor Inflammation Signature as disclosed in PCT/US2015/064445 (WO2016/094377), which is incorporated herein by reference in its entirety, uses 18 genes involved in adaptive anti-tumor immunity to predict response to the anti-PD-1 agent, pembrolizumab (also see e.g. Ayers M et al. The Journal of clinical investigation. 2017 Aug. 1; 127(8):2930-40). The motivation of this study was to enable gene expression to capture an additional, genotypic predictor of immunotherapy response: hypermutation. FIG. 6 compares these genotype and phenotype variables in TCGA, plotting the MSI Predictor score against the Tumor Inflammation Signature score. As a visual guide, thresholds for calling MSI or high immunity have been drawn.

Together, the Tumor Inflammation Signature and MSI scores measured in the same sample identify more potential responders than either test alone. Importantly, very few patients called MSI-H by standard techniques are missed by both the Tumor Inflammation Signature and MSI gene expression score. Interestingly, MSI scores in true MSI-H samples become attenuated in tumors with high Tumor Inflammation Signature scores. One explanation for this phenomenon is that in inflamed tumors, highly abundant immune cells contribute background expression of MLH1 and other MSI signature genes, clouding the otherwise clear signal of the tumor cells' mRNA. Importantly, nearly all MSI-H tumors missed by the MSI gene expression score have high Tumor Inflammation Signature scores, and their potential for anti-tumor immunity would be identified based on that variable alone.

### Summary of Examples

In summary, the examples described herein demonstrate here that RNA expression can be used to identify MSI-H tumors with both high sensitivity and specificity. This discovery opens the possibility of using RNA expression profiling to identify multiple orthogonal biomarkers of checkpoint inhibitor efficacy in a single assay, thereby improving the ability to identify the best treatment option for every patient. Additionally, there are benefits to measuring both anti-tumor immune activity and MSI status using a single test. Rather than using multiple tissue samples and potentially sending those out to multiple laboratories for analysis, combining these two measurements into a single assay allows for conservation of biological material and simplification of personalized treatment decisions.

These findings should have broad applicability in gene expression studies of cancer types where MSI occurs. It is reasonable to posit that outlier low expression values of MHL1, MSH2, MSH6, and PMS2 will nearly always occur in tandem with MSI, regardless of tumor type.

Based on these results, MSI and immune status should together form the foundation of any analysis of immunotherapy in solid tumors. Because these variables are non-redundant, they promise to offer superior prediction together than either can alone. Responders missed by one of these variables may often be identified by the other. To more optimally guide treatment choices, drug efficacy should be evaluated separately in MSI-H/immune-high, MSI-H/immune-low, MSI-L/immune-high, and MSI-L/immune-low subsets.

What is claimed is:

1. A method of identifying mismatch repair deficiency in a subject comprising
a) measuring the gene expression level of at least one gene comprising MLH1, MSH2, MSH6 or PMS2 in a tumor sample from the subject;
b) determining for each of the at least one gene a score Z, wherein Z=(x−μ₁)/σ₁, wherein x is the log-transformed normalized expression of the at least one gene, μ₁ is the mean of the log-transformed normalized expression of the at least one gene in non-hypermutated samples, and σ₁ is the standard deviation of the log-transformed normalized expression of the at least one gene in non-hypermutated samples;
c) determining a score MLS, wherein MLS=(Zₘ+c₁)/c₂, wherein Zₘ is the minimum Z score of the at least one gene, and wherein
c₁ is 0 and c₂ is 1 when one gene is used,
c₁ is 0.56 and c₂ is 0.83 when two genes are used,
c₁ is 0.85 and c₂ is 0.75 when three genes are used, or
c₁ is 1.03 and c₂ is 0.70 when four genes are used;
d) measuring the gene expression level of at least one gene comprising EPM2AIP1, TTC30A, SMAP1, RNLS, WNT11, SFXN1, SREBF1, TYMS, EIF5AL1, or WDR76 in a tumor sample from the subject;
e) determining a score HPS, wherein HPS=(y−μ₂)/σ₂, wherein y=Σᵢ₌₁¹⁰yᵢwᵢ, wherein yᵢ is the log-transformed normalized expression of the at least one gene i in the tumor sample and wᵢ is the prespecified weight for gene i, μ₂ is the mean of the linear combination of the log-transformed normalized expression of the at least one gene in non-hypermutated samples, and σ₂ is the standard deviation of the linear combination of the log-transformed normalized expression of the at least one gene in non-hypermutated samples;
$$f ) determining a score MPS wherein MPS = {( {max ( H P S , 0 )}^{2} + {min ( MLS , 0 )}^{2} )}^{1 / 2} ;$$
g) comparing the MPS score with a predetermined cutoff value, wherein the cutoff value identifies mismatch repair deficiency in a subject with at least 95% specificity; and
h) identifying the presence of mismatch repair deficiency in the subject when the MPS score is equal to or greater than the predetermined cutoff value or identifying the absence of mismatch repair deficiency in the subject when the MPS score is less than the predetermined cutoff value.

2. The method of claim 1, wherein the weight wᵢ for the at least one gene is

| Gene | Weight |
|---|---|
| EPM2AIP1 | −0.31218 |
| TTC30A | −0.19894 |
| SMAP1 | −0.1835 |
| RNLS | −0.19023 |
| WNT11 | −0.11515 |
| SFXN1 | 0.214676 |
| SREBF1 | 0.194835 |
| TYMS | 0.206972 |
| EIF5AL1 | 0.194935 |
| WDR76 | 0.188582 |

3. The method of claim 1, wherein the cutoff value identifies mismatch repair deficiency in a subject with at least 99% specificity.

4. The method of claim 1, wherein the cutoff value identifies mismatch repair deficiency in a subject with at least 99.5% specificity.

5. The method of claim 1, wherein the cutoff value is 2.058.

6. The method of claim 3, wherein the cutoff value is 2.699.

7. The method of claim 4, wherein the cutoff value is 2.939.

8. The method of claim 1, wherein the at least one gene in a) comprises MLH1.

9. The method of claim 1, wherein the at least one gene in a) comprises each of MLH1, MSH2, MSH6 and PMS2.

10. The method of claim 1, wherein the at least one gene in d) comprises each of EPM2AIP1, TTC30A, SMAP1, RNLS, WNT11, SFXN1, SREBF1, TYMS, EIF5AL1 and WDR76

11. The method of claim 1, wherein the at least one gene in a) comprises MLH1 and the at least one gene in d) comprises each of EPM2AIP1, TTC30A, SMAP1, RNLS, WNT11, SFXN1, SREBF1, TYMS, EIF5AL1 and WDR76.

12. The method of claim 1, wherein the at least one gene in a) comprises each of MLH1, MSH2, MSH6 and PMS2 and the at least one gene in d) comprises each of EPM2AIP1, TTC30A, SMAP1, RNLS, WNT11, SFXN1, SREBF1, TYMS, EIF5AL1 and WDR76.

13. The method of claim 1, wherein identifying the presence of mismatch repair deficiency further identifies the subject as having cancer.

14. The method of claim 13, wherein identifying the presence of mismatch repair deficiency further identifies the subject for treatment with an anti-cancer therapy.

15. The method of claim 14, further comprising administering a treatment to a subject identified as having mismatch repair deficiency.

16. The method of claim 14, wherein the treatment comprises administering to the subject an immunotherapy.

17. The method of claim 14, wherein the treatment comprises administering to the subject a checkpoint inhibitor therapy.

18. The method of claim 14, wherein the treatment comprises administering to the subject an anti-PD1 antibody, an anti-PDL1 antibody, or an anti-CTLA4 antibody.

19. The method of claim 18, wherein the anti-PD1 antibody or the anti-PDL1 antibody comprises pembrolizumab, nivolumab, atezolizumab, avelumab, durvalumab, pidilizumab, REGN2810, AMP-224, MEDI0680, PDR001, or CT-001.

20. The method of claim 18, wherein the CTLA4 antibody comprises ipilimumab, tremelimumab, or a combination thereof.


SEQUENCE LISTING (57 sequences)

SEQ ID NO: 1 - DNA, 2524 bp, Homo sapiens
  source 1..2524: mol_type = unassigned DNA
attggctgaaggcacttccgttgagcatctagacgtttccttggctcttctggcgccaaaatgtcgttcgtggcaggggttattcggcggctggacgagacagtggtgaaccgcatcgcggcgggggaagttatccagcggccagctaatgctatcaaagagatgattgagaactgtttagatgcaaaatccacaagtattcaagtgattgttaaagagggaggcctgaagttgattcagatccaagacaatggcaccgggatcaggaaagaagatctggatattgtatgtgaaaggttcactactagtaaactgcagtcctttgaggatttagccagtatttctacctatggctttcgaggtgaggctttggccagcataagccatgtggctcatgttactattacaacgaaaacagctgatggaaagtgtgcatacagagcaagttactcagatggaaaactgaaagcccctcctaaaccatgtgctggcaatcaagggacccagatcacggtggaggaccttttttacaacatagccacgaggagaaaagctttaaaaaatccaagtgaagaatatgggaaaattttggaagttgttggcaggtattcagtacacaatgcaggcattagtttctcagttaaaaaacaaggagagacagtagctgatgttaggacactacccaatgcctcaaccgtggacaatattcgctccatctttggaaatgctgttagtcgagaactgatagaaattggatgtgaggataaaaccctagccttcaaaatgaatggttacatatccaatgcaaactactcagtgaagaagtgcatcttcttactcttcatcaaccatcgtctggtagaatcaacttccttgagaaaagccatagaaacagtgtatgcagcctatttgcccaaaaacacacacccattcctgtacctcagtttagaaatcagtccccagaatgtggatgttaatgtgcaccccacaaagcatgaagttcacttcctgcacgaggagagcatcctggagcgggtgcagcagcacatcgagagcaagctcctgggctccaattcctccaggatgtacttcacccagactttgctaccaggacttgctggcccctctggggagatggttaaatccacaacaagtctgacctcgtcttctacttctggaagtagtgataaggtctatgcccaccagatggttcgtacagattcccgggaacagaagcttgatgcatttctgcagcctctgagcaaacccctgtccagtcagccccaggccattgtcacagaggataagacagatatttctagtggcagggctaggcagcaagatgaggagatgcttgaactcccagcccctgctgaagtggctgccaaaaatcagagcttggagggggatacaacaaaggggacttcagaaatgtcagagaagagaggacctacttccagcaaccccagaaagagacatcgggaagattctgatgtggaaatggtggaagatgattcccgaaaggaaatgactgcagcttgtaccccccggagaaggatcattaacctcactagtgttttgagtctccaggaagaaattaatgagcagggacatgaggttctccgggagatgttgcataaccactccttcgtgggctgtgtgaatcctcagtgggccttggcacagcatcaaaccaagttataccttctcaacaccaccaagcttagtgaagaactgttctaccagatactcatttatgattttgccaattttggtgttctcaggttatcggagccagcaccgctctttgaccttgccatgcttgccttagatagtccagagagtggctggacagaggaagatggtcccaaagaaggacttgctgaatacattgttgagtttctgaagaagaaggctgagatgcttgcagactatttctctttggaaattgatgaggaagggaacctgattggattaccccttctgattgacaactatgtgccccctttggagggactgcctatcttcattcttcgactagccactgaggtgaattgggacgaagaaaaggaatgttttgaaagcctcagtaaagaatgcgctatgttctattccatccggaagcagtacatatctgaggagtcgaccctctcaggccagcagagtgaagtgcctggctccattccaaactcctggaagtggactgtggaacacattgtctataaagccttgcgctcacacattctgcctcctaaacatttcacagaagatggaaatatcctgcagcttgctaacctgcctgatctatacaaagtctttgagaggtgttaaatatggttatttatgcactgtgggatgtgttcttctttctctgtattccgatacaaagtgttgtatcaaagtgtgatatacaaagtgtaccaacataagtgttggtagcacttaagacttatacttgccttctgatagtattcctttatacacagtggattgattataaataaatagatgtgtcttaacataa

SEQ ID NO: 2 - DNA, 3145 bp, Homo sapiens
  source 1..3145: mol_type = unassigned DNA
ggcgggaaacagcttagtgggtgtggggtcgcgcattttcttcaaccaggaggtgaggaggtttcgacatggcggtgcagccgaaggagacgctgcagttggagagcgcggccgaggtcggcttcgtgcgcttctttcagggcatgccggagaagccgaccaccacagtgcgccttttcgaccggggcgacttctatacggcgcacggcgaggacgcgctgctggccgcccgggaggtgttcaagacccagggggtgatcaagtacatggggccggcaggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactgagacagataattcaaagaggaggaattctgatcacagaaagaaaaaaagctgacttttccacaaaagacatttatcaggacctcaaccggttgttgaaaggcaaaaagggagagcagatgaatagtgctgtattgccagaaatggagaatcaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcagggttctgttgaagataccactggctctcagtctctggctgccttgctgaataagtgtaaaacccctcaaggacaaagacttgttaaccagtggattaagcagcctctcatggataagaacagaatagaggagagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggaaaacaccagaaattattgttggcagtttttgtgactcctcttactgatcttcgttctgacttctccaagtttcaggaaatgatagaaacaactttagatatggatcaggtggaaaaccatgaattccttgtaaaaccttcatttgatcctaatctcagtgaattaagagaaataatgaatgacttggaaaagaagatgcagtcaacattaataagtgcagccagagatcttggcttggaccctggcaaacagattaaactggattccagtgcacagtttggatattactttcgtgtaacctgtaaggaagaaaaagtccttcgtaacaataaaaactttagtactgtagatatccagaagaatggtgttaaatttaccaacagcaaattgacttctttaaatgaagagtataccaaaaataaaacagaatatgaagaagcccaggatgccattgttaaagaaattgtcaatatttcttcaggctatgtagaaccaatgcagacactcaatgatgtgttagctcagctagatgctgttgtcagctttgctcacgtgtcaaatggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggccccaatatgggaggtaaatcaacatatattcgacaaactggggtgatagtactcatggcccaaattgggtgttttgtgccatgtgagtcagcagaagtgtccattgtggactgcatcttagcccgagtaggggctggtgacagtcaattgaaaggagtctccacgttcatggctgaaatgttggaaactgcttctatcctcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaactatgctttatcaggtgaagaaaggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagagcaaggtgaaaaaattattcaggagttcctgtccaaggtgaaacaaatgccctttactgaaatgtcagaagaaaacatcacaataaagttaaaacagctaaaagctgaagtaatagcaaagaataatagctttgtaaatgaaatcatttcacgaataaaagttactacgtgaaaaatcccagtaatggaatgaaggtaatattgataagctattgtctgtaatagttttatattgttttatattaaccctttttccatagtgttaactgtcagtgcccatgggctatcaacttaataagatatttagtaatattttactttgaggacattttcaaagatttttattttgaaaaatgagagctgtaactgaggactgtttgcaattgacataggcaataataagtgatgtgctgaattttataaataaaatcatgtagtttgtgg

SEQ ID NO: 3 - DNA, 4435 bp, Homo sapiens
  source 1..4435: mol_type = unassigned DNA
ggcgaggcgcctgttgattggccactggggcccgggttcctccggcggagcgcgcctccccccagatttcccgccagcaggagccgcgcggtagatgcggtgcttttaggagctccgtccgacagaacggttgggccttgccggctgtcggtatgtcgcgacagagcaccctgtacagcttcttccccaagtctccggcgctgagtgatgccaacaaggcctcggccagggcctcacgcgaaggcggccgtgccgccgctgcccccggggcctctccttccccaggcggggatgcggcctggagcgaggctgggcctgggcccaggcccttggcgcgctccgcgtcaccgcccaaggcgaagaacctcaacggagggctgcggagatcggtagcgcctgctgcccccaccagttgtgacttctcaccaggagatttggtttgggccaagatggagggttacccctggtggccttgtctggtttacaaccacccctttgatggaacattcatccgcgagaaagggaaatcagtccgtgttcatgtacagttttttgatgacagcccaacaaggggctgggttagcaaaaggcttttaaagccatatacaggttcaaaatcaaaggaagcccagaagggaggtcatttttacagtgcaaagcctgaaatactgagagcaatgcaacgtgcagatgaagccttaaataaagacaagattaagaggcttgaattggcagtttgtgatgagccctcagagccagaagaggaagaagagatggaggtaggcacaacttacgtaacagataagagtgaagaagataatgaaattgagagtgaagaggaagtacagcctaagacacaaggatctaggcgaagtagccgccaaataaaaaaacgaagggtcatatcagattctgagagtgacattggtggctctgatgtggaatttaagccagacactaaggaggaaggaagcagtgatgaaataagcagtggagtgggggatagtgagagtgaaggcctgaacagccctgtcaaagttgctcgaaagcggaagagaatggtgactggaaatggctctcttaaaaggaaaagctctaggaaggaaacgccctcagccaccaaacaagcaactagcatttcatcagaaaccaagaatactttgagagctttctctgcccctcaaaattctgaatcccaagcccacgttagtggaggtggtgatgacagtagtcgccctactgtttggtatcatgaaactttagaatggcttaaggaggaaaagagaagagatgagcacaggaggaggcctgatcaccccgattttgatgcatctacactctatgtgcctgaggatttcctcaattcttgtactcctgggatgaggaagtggtggcagattaagtctcagaactttgatcttgtcatctgttacaaggtggggaaattttatgagctgtaccacatggatgctcttattggagtcagtgaactggggctggtattcatgaaaggcaactgggcccattctggctttcctgaaattgcatttggccgttattcagattccctggtgcagaagggctataaagtagcacgagtggaacagactgagactccagaaatgatggaggcacgatgtagaaagatggcacatatatccaagtatgatagagtggtgaggagggagatctgtaggatcattaccaagggtacacagacttacagtgtgctggaaggtgatccctctgagaactacagtaagtatcttcttagcctcaaagaaaaagaggaagattcttctggccatactcgtgcatatggtgtgtgctttgttgatacttcactgggaaagtttttcataggtcagttttcagatgatcgccattgttcgagatttaggactctagtggcacactatcccccagtacaagttttatttgaaaaaggaaatctctcaaaggaaactaaaacaattctaaagagttcattgtcctgttctcttcaggaaggtctgatacccggctcccagttttgggatgcatccaaaactttgagaactctccttgaggaagaatattttagggaaaagctaagtgatggcattggggtgatgttaccccaggtgcttaaaggtatgacttcagagtctgattccattgggttgacaccaggagagaaaagtgaattggccctctctgctctaggtggttgtgtcttctacctcaaaaaatgccttattgatcaggagcttttatcaatggctaattttgaagaatatattcccttggattctgacacagtcagcactacaagatctggtgctatcttcaccaaagcctatcaacgaatggtgctagatgcagtgacattaaacaacttggagatttttctgaatggaacaaatggttctactgaaggaaccctactagagagggttgatacttgccatactccttttggtaagcggctcctaaagcaatggctttgtgccccactctgtaaccattatgctattaatgatcgtctagatgccatagaagacctcatggttgtgcctgacaaaatctccgaagttgtagagcttctaaagaagcttccagatcttgagaggctactcagtaaaattcataatgttgggtctcccctgaagagtcagaaccacccagacagcagggctataatgtatgaagaaactacatacagcaagaagaagattattgattttctttctgctctggaaggattcaaagtaatgtgtaaaattatagggatcatggaagaagttgctgatggttttaagtctaaaatccttaagcaggtcatctctctgcagacaaaaaatcctgaaggtcgttttcctgatttgactgtagaattgaaccgatgggatacagcctttgaccatgaaaaggctcgaaagactggacttattactcccaaagcaggctttgactctgattatgaccaagctcttgctgacataagagaaaatgaacagagcctcctggaatacctagagaaacagcgcaacagaattggctgtaggaccatagtctattgggggattggtaggaaccgttaccagctggaaattcctgagaatttcaccactcgcaatttgccagaagaatacgagttgaaatctaccaagaagggctgtaaacgatactggaccaaaactattgaaaagaagttggctaatctcataaatgctgaagaacggagggatgtatcattgaaggactgcatgcggcgactgttctataactttgataaaaattacaaggactggcagtctgctgtagagtgtatcgcagtgttggatgttttactgtgcctggctaactatagtcgagggggtgatggtcctatgtgtcgcccagtaattctgttgccggaagataccccccccttcttagagcttaaaggatcacgccatccttgcattacgaagactttttttggagatgattttattcctaatgacattctaataggctgtgaggaagaggagcaggaaaatggcaaagcctattgtgtgcttgttactggaccaaatatggggggcaagtctacgcttatgagacaggctggcttattagctgtaatggcccagatgggttgttacgtccctgctgaagtgtgcaggctcacaccaattgatagagtgtttactagacttggtgcctcagacagaataatgtcaggtgaaagtacattttttgttgaattaagtgaaactgccagcatactcatgcatgcaacagcacattctctggtgcttgtggatgaattaggaagaggtactgcaacatttgatgggacggcaatagcaaatgcagttgttaaagaacttgctgagactataaaatgtcgtacattattttcaactcactaccattcattagtagaagattattctcaaaatgttgctgtgcgcctaggacatatggcatgcatggtagaaaatgaatgtgaagaccccagccaggagactattacgttcctctataaattcattaagggagcttgtcctaaaagctatggctttaatgcagcaaggcttgctaatctcccagaggaagttattcaaaagggacatagaaaagcaagagaatttgagaagatgaatcagtcactacgattatttcgggaagtttgcctggctagtgaaaggtcaactgtagatgctgaagctgtccataaattgctgactttgattaaggaattatagactgactacattggaagctttgagttgacttctgacaaaggtggtaaattcagacaacattatgatctaataaactttattttttaaaaatgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 5156 bp, Homo sapiens
  source 1..5156: mol_type = unassigned DNA
agccaatgggagttcaggaggcggagcgcctgtgggagccctggagggaactttcccagtccccgaggcggatcgggtgttgcatccatggagcgagctgagagctcgagtacagaacctgctaaggccatcaaacctattgatcggaagtcagtccatcagatttgctctgggcaggtggtactgagtctaagcactgcggtaaaggagttagtagaaaacagtctggatgctggtgccactaatattgatctaaagcttaaggactatggagtggatcttattgaagtttcagacaatggatgtggggtagaagaagaaaacttcgaaggcttaactctgaaacatcacacatctaagattcaagagtttgccgacctaactcaggttgaaacttttggctttcggggggaagctctgagctcactttgtgcactgagcgatgtcaccatttctacctgccacgcatcggcgaaggttggaactcgactgatgtttgatcacaatgggaaaattatccagaaaaccccctacccccgccccagagggaccacagtcagcgtgcagcagttattttccacactacctgtgcgccataaggaatttcaaaggaatattaagaaggagtatgccaaaatggtccaggtcttacatgcatactgtatcatttcagcaggcatccgtgtaagttgcaccaatcagcttggacaaggaaaacgacagcctgtggtatgcacaggtggaagccccagcataaaggaaaatatcggctctgtgtttgggcagaagcagttgcaaagcctcattccttttgttcagctgccccctagtgactccgtgtgtgaagagtacggtttgagctgttccgatgctctgcataatcttttttacatctcaggtttcatttcacaatgcacgcatggagttggaaggagttcaacagacagacagtttttctttatcaaccggcggccttgtgacccagcaaaggtctgcagactcgtgaatgaggtctaccacatgtataatcgacaccagtatccatttgttgttcttaacatttctgttgattcagaatgcgttgatatcaatgttactccagataaaaggcaaattttgctacaagaggaaaagcttttgttggcagttttaaagacctctttgataggaatgtttgatagtgatgtcaacaagctaaatgtcagtcagcagccactgctggatgttgaaggtaacttaataaaaatgcatgcagcggatttggaaaagcccatggtagaaaagcaggatcaatccccttcattaaggactggagaagaaaaaaaagacgtgtccatttccagactgcgagaggccttttctcttcgtcacacaacagagaacaagcctcacagcccaaagactccagaaccaagaaggagccctctaggacagaaaaggggtatgctgtcttctagcacttcaggtgccatctctgacaaaggcgtcctgagacctcagaaagaggcagtgagttccagtcacggacccagtgaccctacggacagagcggaggtggagaaggactcggggcacggcagcacttccgtggattctgaggggttcagcatcccagacacgggcagtcactgcagcagcgagtatgcggccagctccccaggggacaggggctcgcaggaacatgtggactctcaggagaaagcgcctaaaactgacgactctttttcagatgtggactgccattcaaaccaggaagataccggatgtaaatttcgagttttgcctcagccaactaatctcgcaaccccaaacacaaagcgttttaaaaaagaagaaattctttccagttctgacatttgtcaaaagttagtaaatactcaggacatgtcagcctctcaggttgatgtagctgtgaaaattaataagaaagttgtgcccctggacttttctatgagttctttagctaaacgaataaagcagttacatcatgaagcacagcaaagtgaaggggaacagaattacaggaagtttagggcaaagatttgtcctggagaaaatcaagcagccgaagatgaactaagaaaagagataagtaaaacgatgtttgcagaaatggaaatcattggtcagtttaacctgggatttataataaccaaactgaatgaggatatcttcatagtggaccagcatgccacggacgagaagtataacttcgagatgctgcagcagcacaccgtgctccaggggcagaggctcatagcacctcagactctcaacttaactgctgttaatgaagctgttctgatagaaaatctggaaatatttagaaagaatggctttgattttgttatcgatgaaaatgctccagtcactgaaagggctaaactgatttccttgccaactagtaaaaactggaccttcggaccccaggacgtcgatgaactgatcttcatgctgagcgacagccctggggtcatgtgccggccttcccgagtcaagcagatgtttgcctccagagcctgccggaagtcggtgatgattgggactgctcttaacacaagcgagatgaagaaactgatcacccacatgggggagatggaccacccctggaactgtccccatggaaggccaaccatgagacacatcgccaacctgggtgtcatttctcagaactgaccgtagtcactgtatggaataattggttttatcgcagatttttatgttttgaaagacagagtcttcactaaccttttttgttttaaaatgaacctgctacttaaaaaaaatacacatcacacccatttaaaagtgatcttgagaaccttttcaaaccagatggagcattgcttgcaaattttttttctctatgtttgcatgcgctcgtgtgtgtgtgtccaggcaagaacacattttataaaaataagaacacttgggctgggcatggtggctcatgcctgtgatcgcagcactttgggaggccgaggccggcggatcacctgagatcagaagttcgagaccagcctgaccaacatggagaaaccctgcctctactaaaaatacaaaattagccaggtgtgctggcgcatgcctgtaatccccgctacccaggaggctgaggcaggagaatcgcttgaacccgggagacggaggttgcagtgaaccgagattgcgccactgcgctccagcctgggtgagatagaacaagactgtgtctcaaaaaacaaaacaaaacaaaacaaaaaaaaaaaaaccaaaccactttggaagttactcaggcctctgctctggctggacatagtttagtctataactttcaacccttaatgataattaaattcatctttgtttaatttcataaatttaaaagtagggtccttttcagttagtgattctcagccctgattcacattaaatttttaaacacgggggattctctgcccggctggaagaaaatgactggatgggacaggggtcactatttgaaacattcctctgtgcggccaaggtcgcaaaatgctgtcctcgcaggggaacaaaaagagtttgatttcccataatttgatgctgtgatttggtttcctcaggatgtgaactgtagaacattccagttactggccttgaatggttctgggaatataagaatccctgtctgtcttttcaaatagttttcatggaaccttgtcctgtttgaacttggctgaaaatggaagtaaagatgccctcttgggggcccagagatgacagatgtggctccccctgctgcccccaccccttctccagactgtgggcggctccccttcctgctttagaatccctcagatggaggaggcagtacagtagtcactgtgccatcgtgtctggcactgtgctggcgtggtctgcaggatcccacttatgaactctccagattgggagctgtggcaggataacagcccccaagacagctgtgtcctaatccccagaacctgtgaccacgctgcctcacgtggcagaagggactcggcaggtgtgattgagtgaaggatcttttttttttttttctttgagatgaagtttcgctcttgttgcccaggctggagttcaatagcatgatctcagctcactgcagcctctgcctcccaggttcaagtgattctcccacctcagcctcccgagtagctgggattacaggtgtccagaaccatactggctaatttttgtatttttagtagagacagggtttcaccatgttgaccaggctggtctcgaactcctgacctcaggtgatccgaccgcctcggcctcccaaagtgctgggattacaggtgtgagccatcatgcctggctgagttaaggatcttgcaacagagagattatcctggattgtctgggtgggcccagtccattgggtgagtccttcaaaggtggagacctttccctgctggccagagagaggctgtcttgctggttttggagatggaaggaggtaccactagtcaaggattgcaagcagtctctagaacagggattccaacactccggacacagaccagtagtggtccatggcctattaggaagtggggtgcacagcaggttaggggccggcaagccagcgaagcttcatctgtatttatagccactccccgtcgctggcgttaccacccgagctccgcctcctgtcacatcagcggtgggcattagattctcatagcagcacgagccctattgtgaactgcacacacgagggatgtaggttgcacgctccttatgagaatctgatgcctgatgatctgtcactgtctcccgtcacccccagatggggctgtctagttgcaggaaaacaagctcagggctcccactgagtctctgtgatggtgagttgtagaattatttaattatatgttacaatgtaataatagtagaaataaagtgcacaataaatgcaatgcacttgaatcgtcctgaaaccatccctccccgaccccaatccatggaaaaattgtgttccgcgaaaccggtctctggtgccaaaaaggttggggaccgcttctggaaaagctggaaaaggcaagaaaacgcattctctccctcagcctctggaaggaaccagcactgtgggactaatttacatactgtagggtaataaatttgtgttgcttcgaaccactaaaaaaaaa

SEQ ID NO: 5 - DNA, 7439 bp, Homo sapiens
  source 1..7439: mol_type = unassigned DNA
gcttgcgcgttagagatcgctgtccgctcttcctattggttcgtttttaggagctcggggaatacgaaatatccagccaataggagcagagatgccggaaccgggcttgtgtgcctctgctgaggtgatctggcgcagagcggaggaggtgcttggcgcttctcaggctcctcctctccccttgcggcctttctaacgttggccctgctcttgtggcctcccgcagaatgtggatgacgcccaaaagaagcaagatggaagtcgacgaggctctagttttccggcccgagtggacccagcgttatttggtggtggagcctccggagggcgatggggccctgtgcctggtctgtcgccgcctcatcgtagctacccgcgaacgcgacgtcaggcgccactacgaggctgagcacgaatactacgagcggtatgtggcggacggcgagcgcgcggccctggtggagcgtctgcgtcagggcgacttgcccgtggcctccttcactcctgaagagagagctgctcgtgcaggcctcgggctctgccgcctcttggccttgaagggtcgcggctggggtgagggggactttgtataccagtgcatggaggtgttgctgagagaggtactgcccgagcatgtaagcgtcctgcaaggcgttgacttatctccagatatcacaaggcagaggatcctgagcattgacaggaatctacgcaaccagctttttaaccgagccagggactttaaagcctattctcttgccttggacgaccaggcttttgtggcctatgagaactacctcctggtctttatccgcggtgtaggccctgagttggaggtgcaagaagatcttctgaccataatcaacctgactcatcatttcagtgttggtgcgctcatgtcggcaatcctagagtccctgcagacagcagggcttagcttgcagagaatggttggactgaccacgacccatactttgaggatgattggtgagaactcaggactcgtctcatacatgagagaaaaggccgtaagccccaactgttggaatgtcattcattattcaggatttcttcacttggaactgttgagctcctatgatgtagatgttaatcagatcataaataccatatccgaatggatagttttgattaagaccagaggcgttaggcgacctgaatttcagactttactaacggaatctgaatcagagcatggtgaaagggttaatggacgatgtctgaacaattggcttaggagagggaaaactttaaaactaatattctctctaagaaaagaaatggaagcgttcttggtttcagtaggggcaacaacagtccacttctcagacaaacaatggctttgtgactttggcttcttggtggacattatggaacaccttcgagaactcagtgaagaattacgagttagtaaagtctttgctgctgctgcctttgaccatatttgtactttcgaagttaagctgaatttatttcaaagacatattgaggaaaaaaatctaacagactttcctgccctcagagaagttgttgatgagctaaaacagcaaaataaggaagatgaaaaaatatttgatcctgataggtatcaaatggtgatctgtcgtctccaaaaagaatttgagagacattttaaggacctcaggttcattaaaaaggacttagaacttttttcaaatccatttaactttaaacctgaatatgcacctatttcagtgagggtggagctaacaaaacttcaggcaaacactaatctttggaatgaatacagaatcaaagacttggggcagttttatgctggattgtctgctgaatcctacccaattatcaaaggggttgcctgtaaggtggcatccttgtttgatagtaaccaaatctgtgaaaaggctttttcatatttgactcgaaaccaacacactttgagtcagccattaacagatgagcatctccaagccctgtttcgggttgccacaactgaaatggagcccggttgggatgaccttgtgagagaaagaaatgaatctaatccataaggctttgtagtacaagattgaaaaactcaacaagaatttaattctaaaagcaaaaattggtttgagttttcaagtttactaatttggattgtgagaaagtaccaagtaccagccgtccaaactgatcacaattaaaattctgacagttgccttttttttcatctcaaatggcagcatgggactgaaacatgagaatgccaccttttttaaaacttagtttagtgacaaagtcattgtcttttatgatatagttaattttaaagagatttagtattaatgtgagttgaatttgcagtctgttttttaggtgttctgaagataaatgccaaaaatttcagctcttattttaatggagtgttaaaattctgattcatatagtcttaaattatcaactccttaaatgtgcttttgaaccaatttgcagaagctcacatagcaagttcataagtttccaaaaaggaagcccatacataacagtggaggtgttttgtctaaccatcaaaatgtttgagacttttttttaaacatttctgagttcgaaggtaatactgacagatttcttccctcttccctccccatcacccacctcagtgataacacattactgatagaggaagtcattagaatcatttttaagtttcagatataggagacttcatgcaatttggagataagactaattattgggggttttccttggattttttttttaataactgggggctattttatcagcttgcctattaaaggactatggtaagtatagaatcttaatggttgccagttagtaattctttttttttttttttttactgtagacacaagtttggccctatcaaaaacgatgaggaaaaaagattgcactccaggattaggaggtgtgagatattttagcttttttgtcttatctgcgtgggtattgctgctttattttaaaaaatcctgcctaaagtaaacactttgttttaaaatgatacagtatcagattttgttagatgctagaaatggatttattctaaaatttggaactgtcgtacacattctatatgtaagatagcacacaagtagaaatatttaaaagcagtcttattcacagattgcagtaattctgtatttctactaagataatctgctttgtgccaaaacagtaatttccaaacttctgttcaccatgaaaaggcaatcttaaagttcattatgtaaaactaattataaacaggacccaatttatattcatagatcctctcaagtattatacaatttaaaaactcttgttccaaagtcctgtcttaactattgaaacaccttaatctgtggttactaatccagcaaattcaaggaaccaggctatgactaagaatttaggtggaattgatgtctgggcaattaaaataaatggcataagagcttaaaaaccaaagttgtgccagtggctttcaactagaggcagtaacctgtcattccagaggatgctgagaaatgtgtaggggcacttttttggttgtcatatttactaggggcttctgttggcatttaagcctaaagacactcacccctgcagtgcatgggacagcctggcacaatgaagaattagccctcccaaaatgtagattattttatttcaagggatagggcagattaccattagaagcaaaattaaaagtacaagctgggcaaactgacagaatactagataggagagactaattccaaccttctaaatttggctagtaaagtgcaataaaggcattgataagttctgttagctcaccatagcacttgtaaatcaggaattaataattgaatcagatttaagggctctgtcctgttatacatatttaaggcagaaaaaaagttacatgtcgattaggtacttatcaagaatggtcaagctgagattttggttaatagagtaagcttacatatctagagaaacaacatagtggaaaaccgaaaaaaaaaaacagaaaaatctaccggtaatttcccaatagctttgaatattcacagcagagctttattacttgagagaaagactggaagacctgaaagccacttctgctttctaaccccagttccttaaatattgaaatcttgtacattttgtgaaattccagtatgttttgcttaaggtgttaataaaattagtttgcatcatgtagtcattgagtgagggggagatataagccaaggattttaaattgacccttagctatagagaatttgctataagctagtcttgtttgtaaaaaaaaaaaaaaaaaaagaaaaagaaaaaagtgtattttactgttttctgtattaagtaattctgtaactgcatggcagtctttttttttttaaataaatatagttgttactggtcctgttgtagcagtgaatatagttaaaatacgtacattaaaaaaaaaattattaggtccttaccagttactgtcctatagctcattcctactagttttcttgacagatttgtattcccagtgtcccgtattgccactcaaattgctctactatgctaagtccttgttaatagtcttaccctccttgaaacacttgaacacttgatgactttagctttgaggagataccatctccaggtgtgctttcttagtctttgcaggcacctcttcccttcaatatctgttcttcgtatttttaaaaaaatttgttttagactgccttgttctgtgtcagctcgctagctgatctcatttccttccatggtttccttaccatttatatgcaaatgactgtcagattcatatctcctttctagatcttccctaattgatgtatctaattgctaacaaatgctctttgctgtctcaggcactacatgtcattgatcttgcccccaatcctgctcctcctctcatgtttcctctttgactaaatggcattaccactaccaaccattcatttgtcctttttaccaattctccaatgctgccattttaattcaggccatcaacctacctaaattatagcaacagcctccttattagtctccctgttttttatttttattcctttctacactacaaccaaattgctccaaaagacttactgatcatgtcactgcattgctttcaccattgctcttagggtacaatacaaatttatcttcatctttaaggtctcagtatgccacttcatctaggaaaccttcattgatgccctctagattaggtgcccttactatccattccctatacaccctgttctttcccagacatacacttggcacactttattgttactgcttattgatcactgctagactgtaagctttgtaagggcagggaccatataagccttgttcactgttatatctctagtgcttagcacaatgcctggcatttcaataaatgtttggacaaacgaatatttgtgtagtgttttacaatttttgaagctctttcacagtcttatttgaccttcacagtcattctgccttagactgtccattgggtaacttttatccacatattactaattgaaaaatgaagacaagttctttgtaactagggacctcgttgtattctcagaatttagtgtagtgcttagcatgtgacttaaatatgtattatgtgactgttaaacaaattgtggttttctctgttgtatgaaaggagagaaggataacaaattgcggttttccctggtaaacacagtaagtagtaaactcaggattcaaaaccaaatatacacaccaaatccactatgtaatattaagtttgcatatccatgtatagaatcttatttttttttaccctttgtaaacagtgtcatatatatatatatattttttttttttttttaaatttccaaaggaacctacatatagagggaaaagattagacaactacttagtgaactaaaacaatatgtttttactaaatgttacatttagtattggaaaaagataatgccgcctaagagttaataatcatttttccttttgtaggcatcaacactaggagaaaatggcatgctatttacttgctactttcctttacagatgatttttggctcttctgggatttaaaagtaagtaaatttaacaaagtagaagactgactcagcccttctggtcactatatattcagttcacttgtttttacacctgcagaatgtccttatcacccaaagggagatgacccaaaagtgacatctagttaatgtatacttctaaagtttgctgtattcctttgccttcttgttcccatgcctctctgaacttaatttctgggtaactgaggcttttcaggcttaggtgggaaagccacacccttagtctgtttccttaagccattttgaccaatttatgggattaactagtataatcttagttggagttttagtctgaggcatattaagtcattcagagatcttaacagtaggtgtcatagtcatccagtgatttggtgcttgctgcaaaactggcttttttttttttttttttttttgaggcggggtctcactttgtcacccaggctggagtgcagaggtacaatctcagctcaatgcaatctctgccttcctggctcaagcaattctcccactgcagcctcctaagtagctgggaatacaggtatacacgagtacacccagctaatttttgtatttttatgtggagacagggtcttgctgtattccccaggctagtctcgaattcctggactcaagcagtccgcccgcctcgggctcccaaaatgttggtgttatacgtgtgagcctctgcacccggcggcaaaactggcttttaatcaaccttttggctaaaggatttctctttttatttatttgtaaaaggatttcccatttttatctttctttttgatattaaaatgttgcctcatcctacccagtaagtacttgaatttgaattctcttccttttcatttttgcctgcaaactgaccagtcttttctgagttcatctcttctgtacgttttgtcaagtgcagtgaacagcaactacaaaatattttgtttttctgtctttttctttagtaaagggtagatgatctgcctttcaggttatctcaaggggcagtttcacctttccataatataaattacccttgtgtaagttatttcttccatcttctgatagcaatttcctgaatgcctgccagctaaccattaagccagtgttcagtattttagcattttaaaaaacaagggaccaatttctgtgtcagcatgggctagcttgccattgaataacaaaggcaaaatctcactgtctcacacaacttttctattgcaacttgcctagggactttggtttagatcataggttggccatgatcaaactatggtccatgggcaaaatctgtctagctccttatttatctaaataaagttttactggaatata

SEQ ID NO: 6 - DNA, 4686 bp, Homo sapiens
  source 1..4686: mol_type = unassigned DNA
gcggcgccacaggaacgatgcatgccgggaccgggaagattcagtctctgaacggcccggagtagtcgtctttccccttctgactgccgccacgctgcagtccagaatatttgaagatcaaaccgaacttgagagactaacgagaacggtccctttttattcctaacagattccttccgtggcaaagtaacccgtcgtcttccgtttccggttgcccggttgccctgttgccgtggtaaccgcacgcataacagccgtggtggttatggctggtctgagcggcgcgcagatccccgacggggagtttaccgcgctagtgtaccggctcatccgcgatgcccgctacgccgaggcggtgcagctgctgggccgagaactgcagcggagccccaggagccgtgccggcctgtcgctgctaggctactgctactaccgcctgcaggagttcgcgctggcggccgagtgctatgagcagctgggccagctgcacccggaactggagcagtaccgcctgtaccaggcccaggccctgtacaaggcctgcctttatccggaggccactcgggtcgccttccttctcctggataaccccgcctaccacagccgggtcctccgcctgcaagctgccatcaagtatagcgagggcgatctgccagggtccaggagcctggtggagcagctgctgagtggggaagggggagaagaaagtggaggcgacaatgagaccgatggccaggtcaacctgggttgtttgctctacaaggagggacagtatgaagctgcatgctccaagttttctgccacactgcaggcctcgggctaccagcctgacctttcctacaacctggctttggcctattacagcagccgacagtatgcctcagcactgaagcatatcgctgagattattgagcgtggcatccgccagcatcctgagctaggtgtgggcatgaccaccgagggctttgatgttcgcagtgttggcaacaccttagttctccatcagactgctctggtggaagccttcaaccttaaggcagccatagaataccaactgagaaactatgaggtagctcaagaaaccctcaccgacatgccacccagggcagaggaagagttggaccctgtgaccctgcacaaccaggcactaatgaacatggatgccaggcctacagaagggtttgaaaagctacagtttttgctccaacagaatccctttcctccagagacttttggcaacctgttgctgctctactgtaaatatgagtattttgacctggcagcagatgtcctggcagaaaatgcccatttgacgtataagttcctcacaccctatctctatgacttcttagatgccctgatcacttgccagacagctcctgaagaggctttcattaagcttgatgggctagcagggatgctgactgagcagcttcggagactcaccaagcaagtacaggaagcaagacacaacagagatgatgaagctatcaaaaaggcagtgaatgaatatgatgaaaccatggagaaatacattcctgtgttgatggctcaggcaaaaatctactggaatcttgaaaattatccaatggtggaaaaggtcttccgcaaatctgtggaattctgtaacgaccatgatgtgtggaagttgaatgtggctcatgttctgttcatgcaggaaaacaaatacaaagaagccattggtttctatgaacccatagtcaagaagcattatgataacatcctgaatgtcagtgctattgtactggctaatctctgtgtttcctatattatgacaagtcaaaatgaagaagcagaggagttgatgaggaagattgaaaaggaggaagagcagctctcttatgatgacccaaatcggaaaatgtaccatctctgcattgtgaatttggtgataggaactctttattgtgccaaaggaaactatgagtttggtatttctcgagttatcaaaagcttggagccttataataaaaagctgggaacagatacctggtattatgccaaaagatgcttcctgtccttgttagaaaacatgtcaaaacacatgatagtcattcatgacagtgttattcaagaatgtgtccagtttttaggacactgtgaactttatggcacaaacatacctgctgttattgaacaacccctcgaagaagaaagaatgcatgttgggaagaatacagtcacagatgagtccagacaattgaaagctttgatttatgagattataggatggaataagtagttatgactgatagtggcttttttcaaaatggctttcttacgtaccacacttttttttatctgtatttagccttggcatctttatatttgtcttattttgaatcttatccactttgtaagaacaagtttatgtttgagcaactttttcatttaatccagaagggtagggactatgcagtgtaagctgcatcacttctgctttcttcctactagtgacaatcatctggtcttgccctcaagcaacaattgctagagtaacatctttgtataagcaagtaaccccagatagagttgacgtttcagctttgggctgtcaaaagggtatgtcatggaccaaagcactgttagtacgggtatgtttgcatttggtcactgatatgtaaatgactgctagcccacggctggaccacttctcaatcagcaaataaagccatgtctattttgctatctcagcatagactatgctgtctgataaatctaattcttaactctatttctccagttttttagtcctttaactttctggattgcaacgaagtctagtttagacctctaagcccttttagaagtacaagtataatgggaatttcttttcttggttcttttcaggttatgaggtttggtcagtgacaaaattttttttcataatttggttgattggttgcttcttaagttttataataaacgtttttcttcatgttctatttttgattttacagaaatgattttgcctccttgtggatactgacatatattaagtgtggaagcttattaatatttttggttttttaaaaactgaaatttttaatttttactttttaattttttaggaaaaaataagcactgaactgagaatgagaagaataaaagtatgagttccataccttctaattttaggctgtcagaaattcctttattctttgggatttcacaatcatttgaactatcagaagcctttacaattacttttagctgtaacatccgattctgtataagccacatagaaaaaagttgcctttctttttttatgacctggatatataagcaaatcagctaggaaatatataattgtattttatattaatgttttctaggattttggcttacagtaaatgttaacccctatggtaagtgattgttattgttggatgttatactgattattaataagaaatttggatttttgcctttttacctggaatttttgcttacagccgtagctatgaatatatatagggtggtccccagtctctgttatggttgcgcataaattaataattttataagtatttagaaatggtataattctcttaacttcctctttcagtttttgtactaatgtttgtttttgttcgggaagaggagatttgcttttaatccttccaaaaaatgatgaaccaccgttccattcagtagtttgacaagctgttataatgtgtattttttcctcaattattcttgaaatatttagagcctctcctgcttctaacatgaaggcctttagatgccagtctgccagaaatctggaaacagaggaaccggtgaagtgaagatgtaatggagatttagctaatgatgtacttcacaatccaccttggatctcctgcatgtccaaatctcagtagttaatcaagtgtctgctgccattaacagaacagaagtaatggataacagaatggaaataagagatgccagaactacttccataactaactcaccaaatcaaatcatcagtcctcatattcttgttttatttaatacaaggagaagaggccatgcactttccaaaaggtcaaagccacatagaataggaaggcaatctctagtttaaagctttctcttggagtgttttctccccctgtcttcaaagggtctacttgagagatagtggtgtttactgctgcagcatgtatcacaagataagaaatgaaaaatcaatctttcttaccaccctgttctctttcccttttttatcttttcccttttgtcaattatagaattatagggacatttttctctgatagctggaagttgaacctcaaccaggtataaaagatgcataacaaccttttagcagtaagtgtcaagtgagtgagcactatgattatcaaggtgactttggaaaccttttaaaaatgcatttttgcaaaacaagataacatatattgataaaaagtgactctcagattggtaatgccagaaaaaattttaagaggactcaccaaaagtactagatctatgtaagttgtagaatagagtgaagtttttttatatatttgtggtagcctccatcttttaaactttttgaactcagtagaaaaacagactgaaattttaaagacatgcagtatttgtatcattttaaattctgtaacactgggaattaaatatactcaactttagaggaaaaaaaaaaaaaaaaaa

SEQ ID NO: 7 - DNA, 3349 bp, Homo sapiens
  source 1..3349: mol_type = unassigned DNA
gacccagtccccctccccctcccctcgccggctagggtggtgcgtgccggcaggccggtcaaggaggcgggacacgtcggcgctaccaccgccaccgccgccgccgcccctcctcccgttccagctgccgctgccgcttcctgggctgagtccgcccgcggtcccggcggcgccaggtgcgttcactctgcccggctccagccagcgtccgccgccgccgtagctgccccaggctccccgccccgctgccgagatggcgacgcgctcctgtcgggagaaggctcagaagctgaacgagcagcaccagctcatcctatccaagcttctgagggaggaggacaacaagtactgcgccgactgcgaggccaaaggtcctcgatgggcttcctggaatattggtgtgtttatttgcatcagatgtgctggaattcatagaaatcttggggttcatatatccagggtcaaatcagtcaacctagaccaatggacagcagaacagatacagtgcatgcaagatatgggaaatactaaagcaagactactctatgaagccaatcttccagagaactttcgaagaccacagacagatcaagcagtggaatttttcatcagagataaatatgaaaagaagaaatactacgataaaaatgccatagctattacaaatatttcctcctctgatgctcctcttcagcctttggtatcctctccttctctgcaagctgctgttgacaaaaataaattggagaaagaaaaggaaaaaaaaaaggaagagaaaaagagagaaaaggagccagaaaagccggcaaaaccacttacagctgaaaagctgcagaagaaagatcagcaactggagcctaaaaaaagtaccagccctaaaaaagctgcggagcccactgtggatcttttaggacttgatggccctgctgtggcaccagtgaccaacgggaacacaacggtgccacccctgaacgatgatctggacatctttggaccgatgatttctaatcccttacctgcaactgtcatgcccccagctcaggggacaccctctgcaccagcagctgcaaccctgtctacagtaacatctggggatctagatttattcactgagcaaactacaaaatcagaagaagtggcaaagaaacaactttccaaagactccatcttatctctgtatggcacaggaaccattcaacagcaaagtactcctggtgtatttatgggacccacaaatataccatttacctcacaagcaccagctgcatttcagggctttccatcgatgggcgtgcctgtgcctgcagctcctggccttataggaaatgtgatgggacagagtccaagcatgatggtgggcatgcccatgcccaatgggtttatgggaaatgcacaaactggtgtgatgccacttcctcagaacgttgttggcccccaaggaggaatggtgggacaaatgggtgcaccccagagtaagtttggcctgccgcaagctcagcagccccagtggagcctctcacagatgaatcagcagatggctggcatgagtatcagtagtgcaacccctactgcaggttttggccagccctccagcacaacagcaggatggtctggaagctcatcaggtcagactctcagcacacaactgtggaaatgaaaactgcaatacaagtttcatccagaactaccacctgacattccttgctgaaacgcatctagttcccctgtttattcatatgcatattttttttctttttacccatttgttcatattaagaatgatctgattgaccgtgttggtctgtactgattcaatttgatgtggtgaaaagcaggttgataaatcattttatgtcaagggcagctttgctcatatttcccatgatttcatgtactgcattatttgagaagctgctcaacttgcaaaatcagttttcctctcaataaaattatagctctaatgtttgcatataagggaagtagttatcatgttagtaatacctctaatagtataaaccccaccccaaaattagccagtaatcctgtaggaaggtactgtatgatcaaatgtttaatcatataaatagaatgtaaatgtctcactgagcactgttttctagtgtatcaaaatgctcttatttcatcattcacttcactgtgctgttgttatgatgtgcttaacagggaacgtgattagtgaaaggaagataaacgtggatgttactccaaaacttcgtttaatgaatgcttaaagaattcaaattttatctgcctctcttgtaatttggatctcttcttaatgtacatagtgctaacatgaagacctttttctgcactatatgcaaacagggtaactaactaaaacaaagccactttcaatcttcaatccttgaaggtatatctaggtttatgacagtaattgtgtttacattttatggtgcctagtattgacaaaatgttatttccctacattaaacatgactccatagaccttttcatttgtgggtttttatttcctatgatgtatactgccactaaccttccaaaaattacttagtattgcaaagtcaggaatcatcaggaacgtttagctgacaaaatacttgtctgttttaaaaacctgttcaagtctaccaacctgttcaagtctaccaattataagggcaaattggagaaaaagaaaaaatatatactcaagagtggtatcttgcagtatcggcactgtacaaaaaaatcttccaatttagttgttgtagagaaaacatgcagaacaaatgaagacaaaacatacattttgtaccaaccatccaattagcttatgttaactgacaagctccatttaaacagatgtccatcagatgacaagaaaggctgctgtactgaagtaaaacaaacaatacctgaatgctctgtagcctaaactccaaacatcctcttccatatggatccactggctggacaaactgcaccagttgctgcttcaatttatacctcaattttcactgtgtccaggtggtactttggctcgttggctagattaaccttctctgtccgagtgtgccacacgagaacctgaaggggaaggaaatagcttgggtagcgcactcttcatggtgacactcgaggtcgggcagcacaagtgtaatgaataccttagtgcagttatttgctttcggttccagttcttcgactgttgttatctgtttgagaaagtcagattcttgcatccctggctgggatccacgacgcttaaatacagcttttggattggacaaaatgacttgaagacttacagcaaatcctttgtgaaaaataaaaaaaaaaaagagactttaaaaaaaaaaaaaaa

SEQ ID NO: 8 - DNA, 2420 bp, Homo sapiens
  source 1..2420: mol_type = unassigned DNA
aaagctcagggcccaggtcggcccagggagcacggaaccaaagagcgctagcgccggttcggccgcctttccagaaagcccgggccgaacggccccgccgcagagactcagcgcggatcgctgctccctctcgccatggcgcaggtgctgatcgtgggcgccgggatgacaggaagcttgtgcgctgcgctgctgaggaggcagacgtccggtcccttgtaccttgctgtgtgggacaaggctgaggactcagggggaagaatgactacagcctgcagtcctcataatcctcagtgcacagctgacttgggtgctcagtacatcacctgcactcctcattatgccaaaaaacaccaacgtttttatgatgaactgttagcctatggcgttttgaggcctctaagctcgcctattgaaggaatggtgatgaaagaaggagactgtaactttgtggcacctcaaggaatttcttcaattattaagcattacttgaaagaatcaggtgcagaagtctacttcagacatcgtgtgacacagatcaacctaagagatgacaaatgggaagtatccaaacaaacaggctcccctgagcagtttgatcttattgttctcacaatgccagttcctgagattctgcagcttcaaggtgacatcaccaccttaattagtgaatgccaaaggcagcaactggaggctgtgagctactcctctcgatatgctctgggcctcttttatgaagctggtacgaagattgatgtcccttgggctgggcagtacatcaccagtaatccctgcatacgcttcgtctccattgataataagaagcgcaatatagagtcatcagaaattgggccttccctcgtgattcacaccactgtcccatttggagttacatacttggaacacagcattgaggatgtgcaagagttagtcttccagcagctggaaaacattttgccgggtttgcctcagccaattgctaccaaatgccaaaaatggagacattcacaggttacaaatgctgctgccaactgtcctggccaaatgactctgcatcacaaacctttccttgcatgtggaggggatggatttactcagtccaactttgatggctgcatcacttctgccctatgtgttctggaagctttaaagaattatatttagtgcctatatccttattctctacatgtgtattgggtttttattttcacaattttctgttattgattattttgttttctattttgctaagaaaaattactggaaaattgttcttcacttattatcatttttcatgtggagtataaaatcaattttgtaattttgatagttacaacccatgctagaatggaaattcctcacaccttgcaccttccctacttttctgaattgctatgactactccttgttggaggaaaagtggtacttaaaaaataacaaacgactctctcaaaaaaattacattaaatcacaataacagtttgtgtgccaaaaacttgattatccttatgaaaatttcaattctgaataaagaataatcacattatcaaagccccatcttaagtcttcggatgtgtccttgaatcaatatttttgcaaattatacaaaacaagatttttccaaaatgtaggtaacagagtgtaattcttatttctcatttatcccccaagttattaagtgatcctgaattgtaggtcatatatgtcatcatcttagtgtggagggcaacttgactgataaagagaccttccttcagattttcagaaagtataagattccacatgattttcccagccacacagtactttttaactttcaaacaaattccagtcctaatatgaaagataaaaattaaatagaaacagagagaaagtatatcgatccttaccttttgctatattttatagctgttgctgttactttatgggttctccagtatgtgctgtggcatttagactgtgtcgagtttaatgaatttaacacaacaaaaaatttactgaaccagaaaatagatgcacttaaaatagttcaatatttgccaagttggtggttcagcatatcacccacatgcttcagtgacctgaccccacgacttgctagctggagagaaatcaatctccagccttccaaaccagctacctgttgctaatttgaaaagcaaaatgatgagttctatttcagcattttgaaaggagaaaaatcattgcagcctctcaaactaacaaaagttcaacaaaagacttcttactgtaatagtgtttaaagtttcacacttacatgtccactgtcatacatacacatacacaggcacaggcagaacttgcttctatagctgcaaagtgggttttatgaccctatagcatattattatatgtttcctcttagcaataaattggtgaaaaacttaaatgccaaaaaa

SEQ ID NO: 9 - DNA, 1595 bp, Homo sapiens
  source 1..1595: mol_type = unassigned DNA
ccgggcctttgccgacatgcgctggaactgctcctccattgagctcgcccccaactatttgcttgacctggagagaggacaccagccactggcctagggcccaccctgatccggtatgacctcgtctcagccccattacatctgcaaagaccccacttcgtcataagattatgctcacagggacccgggagtcggccttcgtgtatgcgctgtcggccgccgccatcagccacgccatcgcccgggcctgcacctccggcgacctgcccggctgctcctgcggccccgtcccaggtgagccacccgggcccgggaaccgctggggaggatgtgcggacaacctcagctacgggctcctcatgggggccaagttttccgatgctcctatgaaggtgaaaaaaacaggatcccaagccaataaactgatgcgtctacacaacagtgaagtggggagacaggctctgcgcgcctctctggaaatgaagtgtaagtgccatggggtgtctggctcctgctccatccgcacctgctggaaggggctgcaggagctgcaggatgtggctgctgacctcaagacccgatacctgtcggccaccaaggtagtgcaccgacccatgggcacccgcaagcacctggtgcccaaggacctggatatccggcctgtgaaggactcggaactcgtctatctgcagagctcacctgacttctgcatgaagaatgagaaggtgggctcccacgggacacaagacaggcagtgcaacaagacatccaacggaagcgacagctgcgaccttatgtgctgcgggcgtggctacaacccctacacagaccgcgtggtcgagcggtgccactgtaagtaccactggtgctgctacgtcacctgccgcaggtgtgagcgtaccgtggagcgctatgtctgcaagtgaggccctgccctccgccccacgcaggagcgaggactctgctcaaggaccctcagcaactggggccaggggcctggagacactccatggagctctgcttgtgaattccagatgccaggcatgggaggcggcttgtgctttgccttcacttggaagccaccaggaacagaaggtctggccaccctggaaggagggcaggacatcaaaggaaaccgacaagattaaaaataacttggcagcctgaggctctggagtgcccacaggctggtgtaaggagcggggcttgggatcggtgagactgatacagacttgacctttcagggccacagagaccagcctccgggaaggggtctgcccgccttcttcagaatgttctgcgggaccccctggcccaccctggggtctgagcctgctgggcccaccacatggaatcactagcttgggttgtaaatgttttcttttgttttttgctttttcttcctttgggatgtggaagctacagaaatatttataaaacatagctttttctttggggtggcacttctcaattcctctttatatattttatatatataaatatatatgtatatatataatgatctctattttaaaactagctttttaagcagctgtatgaaataaatgctgagtgagccccagcccgcccctgca

SEQ ID NO: 10 - DNA, 4156 bp, Homo sapiens
  source 1..4156: mol_type = unassigned DNA
cggacgcgcgctcacaggcgcgcgcgaggacgcgctccggggacgcgcgaggacgccgtggcgggagaagcgtttccggtggcggcggaggctgcactgagcgggacctgcgagcagcgcgggcggcagcccgggggaagcggtgagtcgcgggcggcaggcccagccagtccgggaccatgtctggagaactaccaccaaacattaacatcaaggaacctcgatgggatcaaagcactttcattggacgagccaatcatttcttcactgtaactgaccccaggaacattctgttaaccaacgaacaactcgagagtgcgagaaaaatagtacatgattacaggcaaggaattgttcctcctggtcttacagaaaatgaattgtggagagcaaagtacatctatgattcagcttttcatcctgacactggtgagaagatgattttgataggaagaatgtcagcccaggttcccatgaacatgaccatcacaggttgtatgatgacgttttacaggactacgccggctgtgctgttctggcagtggattaaccagtccttcaatgccgtcgtcaattacaccaacagaagtggagacgcacccctcactgtcaatgagttgggaacagcttacgtttctgcaacaactggtgccgtagcaacagctctaggactcaatgcattgaccaagcatgtctcaccactgataggacgttttgttccctttgctgccgtagctgctgctaattgcattaatattccattaatgaggcaaagggaactcaaagttggcattcccgtcacggatgagaatgggaaccgcttgggggagtcggcgaacgctgcgaaacaagccatcacgcaagttgtcgtgtccaggattctcatggcagcccctggcatggccatccctccattcattatgaacactttggaaaagaaagcctttttgaagaggttcccatggatgagtgcacccattcaagttgggttagttggcttctgtttggtgtttgctacacccctgtgttgtgccctgtttcctcagaaaagttccatgtctgtgacaagcttggaggccgagttgcaagctaagatccaagagagccatcctgaattgcgacgcgtgtacttcaataagggattgtaaagcagggaggaaacctctgcagctcattctgccactgcaaagctggtgtagccatgctggtgagaaaaatcctgttcaacctgggttctcccagttacggaaaccttttaaagatccacattagccttttagaataaagctgctactttaacagagcacctggcgtgggccaagtgcctgatactcccttacactgaatcatgttatgatttatagaaatacctttcctgtagcttttatagtcattgtttttcaaagacgatataccagccctcacccaggttttaaaaaagcactggtaggcatagaataggtgctcagtatatggtcagtaaatgttctattgattatcaatcagtgaaaaaagaaatctgtttaaaatactgaattttcatctcactcccattgcaaatcaaggagatctcagcagtgaactgggaaaatacaaaagctctgggctaatctataaaaacttaccctgaaatattaagggcagtttgcttctagtttggggattgcgctagcccaatgaaggtgatgaagcttttggatttggagggtaaaagctccttcacaccccttccaaaagtcagtcacagaccactgcaacatgccttccctgctggatcattatatacattcagattgtgagtggattgccttggttgacttttaatttattgttttttgttcttataaagatgataatcttaccttgcagttattgactttatattcaattatttacatcaaataatgaaataactgaaatgtacaaatgtcaaattttggaagtatattcaataccaatgctgtatgagtgggctgaatccagttcattgttttttttttggtaagaagtgagactacagttccagctacctacatgtcttttcttgtcatccttatagatctctttggctttcagaaagatacagtgataatgtgtgtatgaatcagtcacaatgaattttacttgaatattgtatgttgcattccacttcatttgaaaataatgaaaccatgtaccactgtttacatcatctgtagtgatttcatagataatatatttaatatgacagattatgtttcaactctgtagatgtttaacgtcatagacagttggccctctgtatccgtgagctctatatctgtgaattcaaccaagtttggatggaaaattttttttttttttttttttttgagacggagtctcgctctgtcacccaggctggagtgcagtggcgtagtctcggctcactgcaagctccgcctcccgggttcacgccgttctcctgcctcagcctctctgagaagctgggactacaggcgcccgccaccacgcccggctaatttttttgtatttttagtagagacggggtttcactgtggtctcgatctcctgacctcgtgatccgcccgccttggcctcccaaggtgctgggattacaagcgtgagccaccgcacccggcctgaaaatattttctaaaaagataaaaaatatacataacgatgaaaaataatacaaatttaaaaaccaatacagtataacaactatttacatagtgcttacattgtattaggtgttataagcaatctagagatgatttagcaagtatacaggaggatgtgcctaggttatatgcaaatactgtgccattttatatcaggaacttgagcatctgcagatattggtatcggagggcggtcctggaaccaagcatccacggatactgaggggtgacatttcatgaagtgtagatcattgtattcagagattgtaaatgaaaaaaatatagaaactatttagttttggtagattttttttctgacaatgtgaccagactgaatttcctcataaagaaaaaatggcgtgccttgtgtctgtgtttctcttttctctgaaaggattaatagatctgaagctttgggccactcagagccttccttgatgctgccagagtcttcttatttagattttctgtcttaaaccattggaagcaaaacggttttcccatgacattctggccttggacagattctgttgtcctcgacgcgtctctttataaagtggtaaaagcctgaaattcagggcagctctccatgaggtgctgaagggctcttttcataagaagctaaggcactgctgcctgccccaggtgtcccgctcctctcagagtcctccccctaccaggtagtgtgtagctccatttcagaatgttaacctccagtgaagagctaatgactggttagaagattgacaaactaaccaaaattttacacactccggttatgtgtgtgaaaggttataaaaggaatggccgggtgcggtggctcacccctgtaatcccagcactttgggaggccgaggcgggtggatcacctgaggtcaggagtttgagaccagcctggccaacatggagaaaccccgcctctactaaaaatacaaaaaattagccaggcatggaggcacatgcctataatcccagctactcgggaggctgaggtaggagaatcgcttgaatccgggagctggaggttgcagtgagccaagatcgcaccattgcactccagcctgggcaacaagagcgaaactccatctcaaaaaaaaaaaaaagagattataaaagggatgatgaacatggagctgcatctttttaaacgttgttttttgatgcttcagactcttaatgcttttatataaagctatcaactgtatgttgatcacagtttataagaaagaacaaatcaagattggcaatccttgccgatcttttagaaataccttttctggagaaaaaaaaatccacatgaagtgcaataagcttataaagctaagtagttattaatatttctattaacatgatacaaaggatgatgattgtaagtgtttactgactggcagcttttatttcagtattagcacagcgtcttgccagtgttggaggccatgtattatttcagttcaactggatgaaatgttaaataaactcagaatgaaaataaa

SEQ ID NO: 11 - DNA, 4284 bp, Homo sapiens
  source 1..4284: mol_type = unassigned DNA
agcagagctgcggccgggggaacccagtttccgaggaacttttcgccggcgccgggccgcctctgaggccagggcaggacacgaacgcgcggagcggcggcggcgactgagagccggggccgcggcggcgctccctaggaagggccgtacgaggcggcgggcccggcgggcctcccggaggaggcggctgcgccatggacgagccacccttcagcgaggcggctttggagcaggcgctgggcgagccgtgcgatctggacgcggcgctgctgaccgacatcgaaggtgaagtcggcgcggggaggggtagggccaacggcctggacgccccaagggcgggcgcagatcgcggagccatggattgcactttcgaagacatgcttcagcttatcaacaaccaagacagtgacttccctggcctatttgacccaccctatgctgggagtggggcagggggcacagaccctgccagccccgataccagctccccaggcagcttgtctccacctcctgccacattgagctcctctcttgaagccttcctgagcgggccgcaggcagcgccctcacccctgtcccctccccagcctgcacccactccattgaagatgtacccgtccatgcccgctttctcccctgggcctggtatcaaggaagagtcagtgccactgagcatcctgcagacccccaccccacagcccctgccaggggccctcctgccacagagcttcccagccccagccccaccgcagttcagctccacccctgtgttaggctaccccagccctccgggaggcttctctacaggaagccctcccgggaacacccagcagccgctgcctggcctgccactggcttccccgccaggggtcccgcccgtctccttgcacacccaggtccagagtgtggtcccccagcagctactgacagtcacagctgcccccacggcagcccctgtaacgaccactgtgacctcgcagatccagcaggtcccggtcctgctgcagccccacttcatcaaggcagactcgctgcttctgacagccatgaagacagacggagccactgtgaaggcggcaggtctcagtcccctggtctctggcaccactgtgcagacagggcctttgccgaccctggtgagtggcggaaccatcttggcaacagtcccactggtcgtagatgcggagaagctgcctatcaaccggctcgcagctggcagcaaggccccggcctctgcccagagccgtggagagaagcgcacagcccacaacgccattgagaagcgctaccgctcctccatcaatgacaaaatcattgagctcaaggatctggtggtgggcactgaggcaaagctgaataaatctgctgtcttgcgcaaggccatcgactacattcgctttctgcaacacagcaaccagaaactcaagcaggagaacctaagtctgcgcactgctgtccacaaaagcaaatctctgaaggatctggtgtcggcctgtggcagtggagggaacacagacgtgctcatggagggcgtgaagactgaggtggaggacacactgaccccacccccctcggatgctggctcacctttccagagcagccccttgtcccttggcagcaggggcagtggcagcggtggcagtggcagtgactcggagcctgacagcccagtctttgaggacagcaaggcaaagccagagcagcggccgtctctgcacagccggggcatgctggaccgctcccgcctggccctgtgcacgctcgtcttcctctgcctgtcctgcaaccccttggcctccttgctgggggcccgggggcttcccagcccctcagataccaccagcgtctaccatagccctgggcgcaacgtgctgggcaccgagagcagagatggccctggctgggcccagtggctgctgcccccagtggtctggctgctcaatgggctgttggtgctcgtctccttggtgcttctctttgtctacggtgagccagtcacacggccccactcaggccccgccgtgtacttctggaggcatcgcaagcaggctgacctggacctggcccggggagactttgcccaggctgcccagcagctgtggctggccctgcgggcactgggccggcccctgcccacctcccacctggacctggcttgtagcctcctctggaacctcatccgtcacctgctgcagcgtctctgggtgggccgctggctggcaggccgggcagggggcctgcagcaggactgtgctctgcgagtggatgctagcgccagcgcccgagacgcagccctggtctaccataagctgcaccagctgcacaccatggggaagcacacaggcgggcacctcactgccaccaacctggcgctgagtgccctgaacctggcagagtgtgcaggggatgccgtgtctgtggcgacgctggccgagatctatgtggcggctgcattgagagtgaagaccagtctcccacgggccttgcattttctgacacgcttcttcctgagcagtgcccgccaggcctgcctggcacagagtggctcagtgcctcctgccatgcagtggctctgccaccccgtgggccaccgtttcttcgtggatggggactggtccgtgctcagtaccccatgggagagcctgtacagcttggccgggaacccagtggaccccctggcccaggtgactcagctattccgggaacatctcttagagcgagcactgaactgtgtgacccagcccaaccccagccctgggtcagctgatggggacaaggaattctcggatgccctcgggtacctgcagctgctgaacagctgttctgatgctgcgggggctcctgcctacagcttctccatcagttccagcatggccaccaccaccggcgtagacccggtggccaagtggtgggcctctctgacagctgtggtgatccactggctgcggcgggatgaggaggcggctgagcggctgtgcccgctggtggagcacctgccccgggtgctgcaggagtctgagagacccctgcccagggcagctctgcactccttcaaggctgcccgggccctgctgggctgtgccaaggcagagtctggtccagccagcctgaccatctgtgagaaggccagtgggtacctgcaggacagcctggctaccacaccagccagcagctccattgacaaggccgtgcagctgttcctgtgtgacctgcttcttgtggtgcgcaccagcctgtggcggcagcagcagcccccggccccggccccagcagcccagggcaccagcagcaggccccaggcttccgcccttgagctgcgtggcttccaacgggacctgagcagcctgaggcggctggcacagagcttccggcccgccatgcggagggtgttcctacatgaggccacggcccggctgatggcgggggccagccccacacggacacaccagctcctcgaccgcagtctgaggcggcgggcaggccccggtggcaaaggaggcgcggtggcggagctggagccgcggcccacgcggcgggagcacgcggaggccttgctgctggcctcctgctacctgccccccggcttcctgtcggcgcccgggcagcgcgtgggcatgctggctgaggcggcgcgcacactcgagaagcttggcgatcgccggctgctgcacgactgtcagcagatgctcatgcgcctgggcggtgggaccactgtcacttccagctagaccccgtgtccccggcctcagcacccctgtctctagccactttggtcccgtgcagcttctgtcctgcgtcgaagctttgaaggccgaaggcagtgcaagagactctggcctccacagttcgacctgcggctgctgtgtgccttcgcggtggaaggcccgaggggcgcgatcttgaccctaagaccggcggccatgatggtgctgacctctggtggccgatcggggcactgcaggggccgagccattttggggggcccccctccttgctctgcaggcaccttagtggcttttttcctcctgtgtacagggaagagaggggtacatttccctgtgctgacggaagccaacttggctttcccggactgcaagcagggctctgccccagaggcctctctctccgtcgtgggagagagacgtgtacatagtgtaggtcagcgtgcttagcctcctgacctgaggctcctgtgctactttgccttttgcaaactttattttcatagattgagaagttttgtacagagaattaaaaatgaaattatttataatctggaaaaaa

SEQ ID NO: 12 - DNA, 1536 bp, Homo sapiens
  source 1..1536: mol_type = unassigned DNA
ggggggggggggaccacttggcctgcctccgtcccgccgcgccacttggcctgcctccgtcccgccgcgccacttcgcctgcctccgtcccccgcccgccgcgccatgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgaccgcacgggcaccggcaccctgtcggtattcggcatgcaggcgcgctacagcctgagagatgaattccctctgctgacaaccaaacgtgtgttctggaagggtgttttggaggagttgctgtggtttatcaagggatccacaaatgctaaagagctgtcttccaagggagtgaaaatctgggatgccaatggatcccgagactttttggacagcctgggattctccaccagagaagaaggggacttgggcccagtttatggcttccagtggaggcattttggggcagaatacagagatatggaatcagattattcaggacagggagttgaccaactgcaaagagtgattgacaccatcaaaaccaaccctgacgacagaagaatcatcatgtgcgcttggaatccaagagatcttcctctgatggcgctgcctccatgccatgccctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaagccaggtgactttatacacactttgggagatgcacatatttacctgaatcacatcgagccactgaaaattcagcttcagcgagaacccagacctttcccaaagctcaggattcttcgaaaagttgagaaaattgatgacttcaaagctgaagactttcagattgaagggtacaatccgcatccaactattaaaatggaaatggctgtttagggtgctttcaaaggagcttgaaggatattgtcagtctttaggggttgggctggatgccgaggtaaaagttctttttgctctaaaagaaaaaggaactaggtcaaaaatctgtccgtgacctatcagttattaatttttaaggatgttgccactggcaaatgtaactgtgccagttctttccataataaaaggctttgagttaactcactgagggtatctgacaatgctgaggttatgaacaaagtgaggagaatgaaatgtatgtgctcttagcaaaaacatgtatgtgcatttcaatcccacgtacttataaagaaggttggtgaatttcacaagctatttttggaatatttttagaatattttaagaatttcacaagctattccctcaaatctgagggagctgagtaacaccatcgatcatgatgtagagtgtggttatgaactttatagttgttttatatgttgctataataaagaagtgttctgc

SEQ ID NO: 13 - DNA, 3849 bp, Homo sapiens
  source 1..3849: mol_type = unassigned DNA
ggggtcgagtcagtgccgtttgcgccagttggaatcgaagcctcttaaaatggcagatgatttggacttcgagacaggagatgcaggggcctcagccaccttcccaatgcagtgctcagcattacgtaagaatggctttgtggtgctcaaaggctggccatgtaagatcgtggagatgtctgcttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtaccagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccaaggtggcagtggtggcagcagtgatcctccgaacctgcagaggccccctcccccagcctggcctggctctggcctggtcctaggttggactcctcctacacaatttatttgacgttttattttggttttccccaccccctcaatctgtcagggagcccctgcccttcacctagctcccttggccaggagcgagcgaagccatggccttggtgaagctgccctcctcttctcccctcacactacagccctggtgggggagaagggggtgggtgctgcttgtggtttagtcttttttttttttttaaattcaatctggaatcagaaagcggtggattctggcaaatggtccttgtgccctccccactcatccttggtctggtcccctgttgcccatagccctttaccctgagcaccacccaacagactggggaccagccccctcgcctgcctgtgtctctccccaaacccctttagatggggagggaagaagaggagaggggaggggacctgccccctcctcaggcatctgggaagggcctgcccccatgggctttacccttccctgcgggctctctccccgacacatttgttaaaatcaaacctgaataaaactacaagtttaatatgaaaaaaaaaaaaaaagaaagaaagacgtgtaaaatgccaagaactctaggaaacagggacaaaaacacttcaaagagaaagttcatgcacttgtttctgaccacccagggcacccttcagcacacgctgtctggagtggcctgaagcaaggagtgtcttgtgaggtgcagaggatgcaatgggagcagggtcctgtccccaccctaaaggagttcacagtttaacgcaaatgagaagccagtgaggacatcactactcctgctgtgaacttgggaactagaaacacaaaacctgagtctggagggaagctaaggaagcattctgctctggagtagacatgagtgcgtgtgaagcttctgatctcccatgagagcaatggggacatggggcagaatctaaaacccatgactgaaagcaccaaattgctaaaatggcaataaagagacatgaggccaagatggagaagaaggaacccaggacgagggtcagcctcacatttggggctcatttccctcagtttcctcactgaatttcagaagggactaactgagatgcaaagaagcagagcagcttttgcaccatgtggaggactagatggaaaacaagtagactgagggtctgctagtgaaggtgacccctactgaagtccactggctttggttgggacccagaagagtcacacgccaggaatagaggtggacaggaaacaccctgacttttgtagggactgaacctcactgataacctcaattgcggatggtatggagggtgtctaggtgtgctaggacccctgcccattccccagaaatagactcccatcttttctacagcaagataacgtgctagtaggcctcaattcattgctaaatatttttaacgagtgtcttacatttagccaaaaagactagtcatgtggcaggaaaaatacaatgtcatatgaccaaaagctaaaagactgtgaaaatgaatccagaggtgacccaagcattgaatttaacaatgccagtacctggacctccgcttgcccctaaaacattacaatcaagaatgtaggaagggaaaggaaacacgaagattaatcaagcaggaaggacaagctcagttttgcacccactgaatttgccacaaatattgtggaaaatattctcggggacattgcagttgtctactttggttggcacatggttcatacaacagtgtttgtgtcagtgaacatcttactcttcctcggcagtctttctttgcccagagatttcgcaatgactgttgaccttcatcatcaccttttggactttggcttgcactttagcttctgtagatctccatgatgtaaagaagtattttaggtccattttaattcctgcaaaggataaaatccttctatttgtgtgcatataagtggacctgagcccttggttagggtgtagagaggagaaggggagaaacctgagggccagaagctgttctttcccttaaaagggcaaactcatttccacactatggggactctgacagatagcataccttcctgtctatggctattggacctgcaggctttcccctgtaaatccgtgttctgtcattgacattttgtgactgtaagacagacttgagataagacatctagaaaacaataattgaacaatgatgtgaatatatttcacacaactgaactgtacatttcaacaaggttaagatggtaattatcacgttatacattttttaccgcaggttaaaatgtttcacaggttgaaaggaaagcaactaccttcagttctctgagttcaagaatttgtaacatttcaccccctgctccttcctgatcttctgtggagcatcttttttccatccatgctctactcagagcccactttcccttccctgacaccagcttcactgaggctggttggaacctaacacaaaacattctcagtaatgactgaattcccacaaagaattccatatagactgcatatgagttgaatcttctaagacatgaaatatttgttctcttcttggctaatatgcaatgcaaatcctgttgcagatgtacgtcatatacctctgaaattcctgatgtattcaatgaaataacatctttaaagttctgtgtagaatgttttttttctgatttcttcacatacgatagaaaaaaaaacccaaaaaaacatgtactaggatttcaatagaagcaatgggtgatctaaaaagatgaaagagcaaccgcatgcgccctacagctaccgctagattttatggggaaagcagctggcccagtttgcagctaggagaaatgtcaaacacatgaagaaatgagaagcaaagaaaaaccatgaggcatgaacatttcatggcaatcacgatgtcctggtttgtgagataatgggatagaggagtagaaaacaaggagaaagatgagaaggtacaaagtggttcaagtcaaacagctcaactgaacttttcttaatggaatatttaaaaagtggtacattaaaaaacttcccccagttcacatcaaaaattctctcttcaggactaagttgggtagagactgttcaatgtgcctagatatcttcagaacttatatattttctgttttctacgtatgttgaagggcagtgccaaatgatgtgtaattatctaggttgtaaaaataaaacatactcccccttcccttgaggataaaaaaaaaaaaaaa

SEQ ID NO: 14 - DNA, 4037 bp, Homo sapiens
  source 1..4037: mol_type = unassigned DNA
ctgctctggcgctgcggccgctggggatctgagtgggctccgccccgcctcggacccgcccctcccggcctcccgccgcaatcttggcgggaaggcgccggccgctaagaagccgaaagatgtccaggtcgggcgcggcggctgagaaggcggactccagacagcgaccccagatgaaggtaaatgaatataaagaaaatcaaaacatcgcttatgtgtctctgagaccagcacagactacagttttaataaaaacagctaaggtctatcttgcccccttttcactcagtaattaccagctagaccagcttatgtgccccaaatccctatcagaaaagaattctaacaatgaagtggcgtgtaagaagactaaaataaagaaaacttgcagaaggattatacctccaaagatgaaaaacacatcttccaaggcagaatccacgctgcaaaattcatcctcagctgttcatactgaaagtaacaagctacaacccaagagaacggcagatgcgatgaatctcagtgttgatgtggaaagtagtcaggatggagacagtgatgaagataccacaccatccctggatttttcgggattgtcaccctacgaaaggaagagactgaagaacatatcagaaaacgcagacttttttgcttctcttcagttgtctgagtctgctgcaagactccgtgaaatgatagagaagagacagcctcctaaatccaaaagaaagaagcctaagagagaaaatgggattggatgtagaaggtcaatgcgattactaaaagttgatccttcgggagtttcattaccagcagctccaacaccgccgacattagtagcagatgaaactcctttgttacctcctgggcctttagaaatgacttctgaaaatcaagaagacaacaatgaacgatttaaaggatttctgcacacatgggcaggaatgagcaagccaagtagtaagaacactgagaagggattatctagcattaaaagctacaaagccaatttaaatggcatggtcattagtgaagataccgtttacaaagttaccacaggcccaatattctctatggctctccatccatcagaaactagaactttggtagcagttggggccaaatttgggcaagttggactttgtgatttgacccagcaacctaaagaagatggagtttatgtttttcatccccatagtcagccagttagctgtctttacttctcacccgccaatccggcccacatactgtcactgagctatgatggcacgttacgctgtggggatttttccagggctatttttgaagaggtgtatagaaatgaaagaagtagcttttcctccttcgacttcttggcagaagatgcctccactttaatagtaggacactgggatggaaatatgtcactggtggatagacggacacctggaacttcttatgagaaacttaccagttcttctatgggaaaaataagaactgttcatgtccacccagtgcatagacagtattttatcactgccggattgagggatactcatatttatgatgcaaggcgattgaattccaggagaagtcagcctttgatttctttgactgaacatacaaagagcattgcttccgcctatttttcacctcttactggtaacagagtggtgaccacatgtgctgattgtaatctgagaatttttgacagcagctgtatatcttctaagattccgctcctcaccaccatcaggcacaacactttcactgggcgatggctgaccaggttccaagccatgtgggatcctaaacaagaagactgtgtcatagttggcagcatggcccatccacgacgggtagaaatcttccatgagacaggaaagagggtgcattcgtttggtggagaataccttgtctctgtgtgttccatcaatgccatgcacccaactcggtatattttggctggaggtaattccagcgggaagatacatgtttttatgaatgaaaaaagctgctgagtttttggtttaggaacatcaatttgttcaaattgaccactgtctaaggagcctagtaatcggcgtgccttagtgtgtttatgtggtaatgtgttacatttagcaattataacattgttttattaataagactataagaagagtgtacttttagtaagggagaagtcttggagggttgcttctgcaggacggggagggaatttgaggggaggctgaggtgccgtcaggactttttttttttttttttttttgagatggagttttgctcttgttgcccaggctggagtgcaatagcgcgatcttggctcaccgcaacctccgcctcccaggttcaagcgattctcctgcctcagactcctaagtagctgggattacaggcacctgccaccacgcctggctatttttttgtatttttagtagagatggggtttcatcatgttggccaggctggtctcgagctcctgacctcaggtgatctgcccgcctcggcctccaaaagtgctggaattacaggcgtgagccaccatgcctggccatcagaacttgtaatcaagacagtatgttgagaaattctaacattataaattacaaagctttgactattaaagtttttgtgatctaatgatacagttttgattctatagtaatttgtggcttattttatagtttataatgaatacttatttctagactcatacactggaaggggacccggaaaggtaatgtaactcagtgattttaaaacttgatttttttaactgagaactttttttgccccctgcctgtaggttaagtcttacgtgaaatgccaagataattgctgagcagctttggttacccagggcggggtctgggtctgtctgtactttgcctttactctagatggctcctgagacacaggcaggactcccaagcaccgggttgggatctgccctggtcccggcattccagtataagattgcctcagacctgtgtttttcagactgggtttttgctcttcacatgaaatcaagttagatgacaatgactggtgttgaaaaaaatgaaaaggaaagaatttgtaaagaacagaaaatatatttgagtaagtattgtttggtaaaacttagttacatatgcatatatatttgttaggtatatatgtttatgtgtattctgatgtaaaatatatatatatatatattttattactatagtaccatgggtaatggataaagaagttaaagctactgcttagaatgaagaaggccccaggcttacctgtcccgatctttaaactgtccgaaggaaattcaatagcctgttaagtgaataccttcattcttacttgtatttgggggaatattatgaaatactcaccacttttggtattttatgaaaatgttttcttttcagaagttatggtaatttcaatgtgtttgttgttgggaggggagctgccaaatcagttactaatattactgtgtgacatctatccaacttttttcattattcttcattgccaaatactgaaagacttgtaaatggctttggcaatatgtttgaattctaagaggaaatattttcccataattgtatatcagagaaatatagtgatatacaatttccttgaaaaccaatttctaaataattttcttctctgtaatctaagtgtaaaaaggtttagttttttaataggtttaggtgtttataagcaatagttctctattttctagttgatataagtagaagaattgacaagtgagatggaaatgttaatttataaagggaaagaaaagctaggtgaggttgagttataattaaactgttcaggaaacatcgtaaaggctttaggctccctttttcatttctataccaattaatctcatgggttctagagtggttagttctacgggaattgtttttgtttttgtttttaaagatgctgaaaactactctcaatcaaattagtaccatcatttaagctttgaatacttggcagtaattgcctgggctcgtcaataaatgttagcaaattcttgatgttcaaaaaaaaaa

SEQ ID NO: 15 - DNA, 1237 bp, Homo sapiens
  source 1..1237: mol_type = unassigned DNA
gctgcagaggattcctgcagaggatcaagacagcacgtggacctcgcacagcctctcccacaggtaccatgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttgaagggcccagattctaccacacagcagcagttacaaaaaccttccccaggctggacgtggtggctcacgcctgtaatcccagcactttgggaggccaaggtgggtggatcacttgaggtcaggagttcgagaccagcctggccaacatgatgaaaccccatctctactaaaaatacaaaaaattagccgggcgtggtagcgggcgcctgtagtcccagctactcgggaggctgaggcaggagaatggcgtgaacccgggaggcggagcttgcagtgagccgagatcgcgccactgcactccagcctgggcgacagagcgagactccgtctcaaaaaaaaaaaaaaaaaaaaaaatacaaaaattagccgggcgtggtggcccacgcctgtaatcccagctactcgggaggctaaggcaggaaaattgtttgaacccaggaggtggaggctgcagtgagctgagattgtgccacttcactccagcctgggtgacaaagtgagactccgtcacaacaacaacaacaaaaagcttccccaactaaagcctagaagagcttctgaggcgctgctttgtcaaaaggaagtctctaggttctgagctctggctttgccttggctttgccagggctctgtgaccaggaaggaagtcagcatgcctctagaggcaaggaggggaggaacactgcactcttaagcttccgccgtctcaacccctcacaggagcttactggcaaacatgaaaaatcggcttaccattaaagttctcaatgcaaccataaaaaaaaaa

SEQ ID NO: 16 - DNA, 1320 bp, Homo sapiens
  source 1..1320: mol_type = unassigned DNA
cggaaggggaagggggtggaggttgctgctatgagagagaaaaaaaaaacagccacaatagagattctgccttcaaaggttggcttgccacctgaagcagccactgcccagggggtgcaaagaagagacagcagcgcccagcttggaggtgctaactccagaggccagcatcagcaactgggcacagaaaggagccgcctgggcagggaccatggcacggccacatccctggtggctgtgcgttctggggaccctggtggggctctcagctactccagcccccaagagctgcccagagaggcactactgggctcagggaaagctgtgctgccagatgtgtgagccaggaacattcctcgtgaaggactgtgaccagcatagaaaggctgctcagtgtgatccttgcataccgggggtctccttctctcctgaccaccacacccggccccactgtgagagctgtcggcactgtaactctggtcttctcgttcgcaactgcaccatcactgccaatgctgagtgtgcctgtcgcaatggctggcagtgcagggacaaggagtgcaccgagtgtgatcctcttccaaacccttcgctgaccgctcggtcgtctcaggccctgagcccacaccctcagcccacccacttaccttatgtcagtgagatgctggaggccaggacagctgggcacatgcagactctggctgacttcaggcagctgcctgcccggactctctctacccactggccaccccaaagatccctgtgcagctccgattttattcgcatccttgtgatcttctctggaatgttccttgttttcaccctggccggggccctgttcctccatcaacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccctgagccagcacctgcgggagctgcactacagccctggcctccacccccaccccgccgaccatccaagggagagtgagacctggcagccacaactgcagtcccatcctcttgtcagggccctttcctgtgtacacgtgacagagtgccttttcgagactggcagggacgaggacaaatatggatgaggtggagagtgggaagcaggagcccagccagctgcgcctgcgctgcaggagggcgggggctctggttgtaaaacacacttcctgctgcgaaagacccacatgctacaagacgggcaaaataaagtgacagatgaccaccctgca

SEQ ID NO: 17 - DNA, 3691 bp, Homo sapiens
  source 1..3691: mol_type = unassigned DNA
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa

SEQ ID NO: 18 - DNA, 3419 bp, Homo sapiens
  source 1..3419: mol_type = unassigned DNA
ccggcctcagggacgcaccggagccgcctttccgggcctcaggcggattctccggcgcggcccgccccgcccctcggactccccgggccgcccccggcccccattcgggccgggcctcgctgcggcggcgactgagccaggctgggccgcgtccctgagtcccagagtcggcgcggcgcggcaggggcagccttccaccacggggagcccagctgtcagccgcctcacaggaagatgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggccctgtgggtgaccgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctgaccatgaggaccagggagctgctacccctccctacagctcctaccctctggctgcaatggggctgcactgtgagccctgcccccaacagatgcatcctgctctgacaggtgggctccttctccaaaggatgcgatacacagaccactgtgcagccttatttctccaatggacatgattcccaagtcatcctgctgccttttttcttatagacacaatgaacagaccacccacaaccttagttctctaagtcatcctgcctgctgccttatttcacagtacatacatttcttagggacacagtacactgaccacatcaccaccctcttcttccagtgctgcgtggaccatctggctgccttttttctccaaaagatgcaatattcagactgactgaccccctgccttatttcaccaaagacacgatgcatagtcaccccggccttgtttctccaatggccgtgatacactagtgatcatgttcagccctgcttccacctgcatagaatcttttcttctcagacagggacagtgcggcctcaacatctcctggagtctagaagctgtttcctttcccctccttcctcctcttgctctagccttaatactggccttttccctccctgccccaagtgaagacagggcactctgcgcccaccacatgcacagctgtgcatggagacctgcaggtgcacgtgctggaacacgtgtggttcccccctggcccagcctcctctgcagtgcccctctcccctgcccatcctccccacggaagcatgtgctggtcacactggttctccaggggtctgtgatggggcccctgggggtcagcttctgtccctctgccttctcacctctttgttcctttcttttcatgtatccattcagttgatgtttattgagcaactacagatgtcagcactgtgttaggtgctgggggccctgcgtgggaagataaagttcctccctcaaggactccccatccagctgggagacagacaactaactacactgcaccctgcggtttgcagggggctcctgcctggctccctgctccacacctcctctgtggctcaaggcttcctggatacctcacccccatcccacccataattcttacccagagcatggggttggggcggaaacctggagagagggacatagcccctcgccacggctagagaatctggtggtgtccaaaatgtctgtccaggtgtgggcaggtgggcaggcaccaaggccctctggacctttcatagcagcagaaaaggcagagcctggggcagggcagggccaggaatgctttggggacaccgaggggactgccccccacccccaccatggtgctattctggggctggggcagtcttttcctggcttgcctctggccagctcctggcctctggtagagtgagacttcagacgttctgatgccttccggatgtcatctctccctgccccaggaatggaagatgtgaggacttctaatttaaatgtgggactcggagggattttgtaaactgggggtatattttggggaaaataaatgtctttgtaaaaagcttaaaaaaaaaaaaaaaaaa

SEQ ID NO: 19 - DNA, 2325 bp, Homo sapiens
  source 1..2325: mol_type = unassigned DNA
cgaaaaggagggtgactctcctcggcgggggcttcgggtgacatcacatcctccaaatgcgaaatcaggctccgggccggccgaagggcgcaactttcccccctcggcgccccaccggctcccgcgcgcctcccctcgcgcccgagcttcgagccaagcagcgtcctggggagcgcgtcatggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaaccctgtgcaacagccactacattacttcaaactgagatccttccttttgagggagcaagtccttccctttcattttttccagtcttcctccctgtgtattcattctcatgattattattttagtgggggcggggtgggaaagattactttttctttatgtgtttgacgggaaacaaaactaggtaaaatctacagtacaccacaagggtcacaatactgttgtgcgcacatcgcggtagggcgtggaaaggggcaggccagagctacccgcagagttctcagaatcatgctgagagagctggaggcacccatgccatctcaacctcttccccgcccgttttacaaagggggaggctaaagcccagagacagcttgatcaaaggcacacagcaagtcagggttggagcagtagctggagggaccttgtctcccagctcagggctctttcctccacaccattcaggtctttctttccgaggcccctgtctcagggtgaggtgcttgagtctccaacggcaagggaacaagtacttcttgatacctgggatactgtgcccagagcctcgaggaggtaatgaattaaagaagagaactgcctttggcagagttctataatgtaaacaatatcagactttttttttttataatcaagcctaaaattgtatagacctaaaataaaatgaagtggtgagcttaaccctggaaaatgaatccctctatctctaaagaaaatctctgtgaaacccctatgtggaggcggaattgctctcccagcccttgcattgcagaggggcccatgaaagaggacaggctacccctttacaaatagaatttgagcatcagtgaggttaaactaaggccctcttgaatctctgaatttgagatacaaacatgttcctgggatcactgatgactttttatactttgtaaagacaattgttggagagcccctcacacagccctggcctctgctcaactagcagatacagggatgaggcagacctgactctcttaaggaggctgagagcccaaactgctgtcccaaacatgcacttccttgcttaaggtatggtacaagcaatgcctgcccattggagagaaaaaacttaagtagataaggaaataagaaccactcataattcttcaccttaggaataatctcctgttaatatggtgtacattcttcctgattattttctacacatacatgtaaaatatgtctttcttttttaaatagggttgtactatgctgttatgagtggctttaatgaataaacatttgtagcatcctctttaatgggtaaacagcatccgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 20 - DNA, 1900 bp, Homo sapiens
  source 1..1900: mol_type = unassigned DNA
gaattcggcacgagtcagggaagcagccccggcggccagcagggagctcaggacagagcaggctccctgggaagcctccgggtgataggggtgttccagctgcggcgctctgggggttcagagggggatcttgaatgaacaaatgaatgaactgctttctgggcaaacagccacagccagaggagcctgtgattggcagaaagaagccagggtgtgcaagtctccccaacagcctcgagtggcctgcagtcacagggaaccctcaggaagaccttccgggcagagaccagagggaagcccatctctccagcagaactgcttggatttttctaccaggaggctcagggctctgcaacaatgatagcagaagctgatggcatctagagatctaggctgggactagcacagcatcacttctaccactttctgttggtcacagcaactcaccatgccagtgcagattcaaggggaggagaaatagagtccacttcttgatgggaggcgtgacatagaatggaggatgaagattacaacacttccatcagttacggtgatgaataccctgattatttagactccattgtggttttggaggacttatcccccttggaagccagggtgaccaggatcttcctggtggtggtctacagcatcgtctgcttcctcgggattctgggcaatggtctggtgatcatcattgccaccttcaagatgaagaagacagtgaacatggtctggttcctcaacctggcagtggcagatttcctgttcaacgtcttcctcccaatccatatcacctatgccgccatggactaccactgggttttcgggacagccatgtgcaagatcagcaacttccttctcatccacaacatgttcaccagcgtcttcctgctgaccatcatcagctctgaccgctgcatctctgtgctcctccctgtctggtcccagaaccaccgcagcgttcgcctggcttacatggcctgcatggtcatctgggtcctggctttcttcttgagttccccatctctcgtcttccgggacacagccaacctgcatgggaaaatatcctgcttcaacaacttcagcctgtccacacctgggtcttcctcgtggcccactcactcccaaatggaccctgtggggtatagccggcacatggtggtgactgtcacccgcttcctctgtggcttcctggtcccagtcctcatcatcacagcttgctacctcaccatcgtgtgcaaactgcagcgcaaccgcctggccaagaccaagaagcccttcaagattattgtgaccatcatcattaccttcttcctctgctggtgcccctaccacacactcaacctcctagagctccaccacactgccatgcctggctctgtcttcagcctgggtttgcccctggccactgcccttgccattgccaacagctgcatgaaccccattctgtatgttttcatgggtcaggacttcaagaagttcaaggtggccctcttctctcgcctggtcaatgctctaagtgaagatacaggccactcttcctaccccagccatagaagctttaccaagatgtcatcaatgaatgagaggacttctatgaatgagagggagaccggcatgctttgatcctcactgtggaacccctcaatggactctctcaacccagggacacccaaggatatgtcttctgaagatcaaggcaagaacctctttagcatccaccaattttcactgcattttgcatgggatgaacagtgttttatgctgggaatctagggcctggaacccctttcttctagtggacagaacatgctgtgttccatacagccttggactagcaatttatgcttcttgggaggccagccttgactgactcaaagcaaaaaaggaagaattc

SEQ ID NO: 21 - DNA, 2545 bp, Homo sapiens
  source 1..2545: mol_type = unassigned DNA
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 22 - DNA, 1953 bp, Homo sapiens
  source 1..1953: mol_type = unassigned DNA
gcagaccttgcttcatgagcaagctcatctctggaacaaactggcaaagcatctctgctggtgttcatcagaacagacaccatggcagagcatgattaccatgaagactatgggttcagcagtttcaatgacagcagccaggaggagcatcaagacttcctgcagttcagcaaggtctttctgccctgcatgtacctggtggtgtttgtctgtggtctggtggggaactctctggtgctggtcatatccatcttctaccataagttgcagagcctgacggatgtgttcctggtgaacctacccctggctgacctggtgtttgtctgcactctgcccttctgggcctatgcaggcatccatgaatgggtgtttggccaggtcatgtgcaagagcctactgggcatctacactattaacttctacacgtccatgctcatcctcacctgcatcactgtggatcgtttcattgtagtggttaaggccaccaaggcctacaaccagcaagccaagaggatgacctggggcaaggtcaccagcttgctcatctgggtgatatccctgctggtttccttgccccaaattatctatggcaatgtctttaatctcgacaagctcatatgtggttaccatgacgaggcaatttccactgtggttcttgccacccagatgacactggggttcttcttgccactgctcaccatgattgtctgctattcagtcataatcaaaacactgcttcatgctggaggcttccagaagcacagatctctaaagatcatcttcctggtgatggctgtgttcctgctgacccagatgcccttcaacctcatgaagttcatccgcagcacacactgggaatactatgccatgaccagctttcactacaccatcatggtgacagaggccatcgcatacctgagggcctgccttaaccctgtgctctatgcctttgtcagcctgaagtttcgaaagaacttctggaaacttgtgaaggacattggttgcctcccttaccttggggtctcacatcaatggaaatcttctgaggacaattccaagactttttctgcctcccacaatgtggaggccaccagcatgttccagttataggccttgccagggtttcgagaagctgctctggaatttgcaagtcatggctgtgccctcttgatgtggtgaggcaggctttgtttatagcttgcgcattctcatggagaagttatcagacactctggctggtttggaatgcttcttctcaggcatgaacatgtactgttctcttcttgaacactcatgctgaaagcccaagtagggggtctaaaatttttaaggactttccttcctccatctccaagaatgctgaaaccaagggggatgacatgtgactcctatgatctcaggttctccttgattgggactggggctgaaggttgaagaggtgagcacggccaacaaagctgttgatggtaggtggcacactgggtgcccaagctcagaaggctcttctgactactgggcaaagagtgtagatcagagcagcagtgaaaacaagtgctggcaccaccaggcacctcacagaaatgagatcaggctctgcctcaccttggggcttgacttttgtataggtagatgttcagattgctttgattaatccagaataactagcaccagggactatgaatgggcaaaactgaattataagaggctgataattccagtggtccatggaatgcttgaaaaatgtgcaaaacagcgtttaagactgtaatgaatctaagcagcatttctgaagtggactctttggtggctttgcattttaaaaatgaaattttccaatgtctgccacacaaacgtatgtaaatgtatatacccacacacatacacacatatgtcatatattactagcatatgagtttcatagctaagaaataaaactgttaaagtctccaaact

SEQ ID NO: 23 - DNA, 1542 bp, Homo sapiens
  source 1..1542: mol_type = unassigned DNA
acaattactctacagctcagaacaccaactgctgaggctgccttgggaagaggatgatcctaaacaaagctctgctgctgggggccctcgctctgaccaccgtgatgagcccctgtggaggtgaagacattgtggctgaccacgttgcctcttgtggtgtaaacttgtaccagttttacggtccctctggccagtacacccatgaatttgatggagatgagcagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatgaggttcctgaggtcacagtgttttccaagtctcccgtgacactgggtcagcccaacaccctcatttgtcttgtggacaacatctttcctcctgtggtcaacatcacatggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactgggagcctgagattccagcccctatgtcagagctcacagagactgtggtctgtgccctggggttgtctgtgggcctcatgggcattgtggtgggcactgtcttcatcatccaaggcctgcgttcagttggtgcttccagacaccaagggccattgtgaatcccatcctggaagggaaggtgcatcgccatctacaggagcagaagaatggacttgctaaatgacctagcactattctctggcccgatttatcatatcccttttctcctccaaatatttctcctctcaccttttctctgggacttaagctgctatatcccctcagagctcacaaatgcctttacattctttccctgacctcctgattttttttttcttttctcaaatgttacctacaaagacatgcctggggtaagccacccggctacctaattcctcagtaacctccatctaaaatctccaaggaagcaataaattccttttatgagatctatgtcaaatttttccatctttcatccagggctgactgaaactatggctaataattggggtactcttatgtttcaatccaatttaacctcatttcccagatcatttttcatgtccagtaacacagaagccaccaagtacagtatagcctgataatatgttgatttcttagctgacattaatatttcttgcttccttgtgttcccacccttggcactgccacccacccctcaattcaggcaacaatgaaattaatggataccgtctgcccttggcccagaattgttatagcaaaaattttagaaccaaaaaataagtctgtactaatttcaatgtggcttttaaaagtatgacagagaaataagttaggataaaggaaatttgaatctca

SEQ ID NO: 24 - DNA, 1182 bp, Homo sapiens
  source 1..1182: mol_type = unassigned DNA
tagttctccctgagtgagacttgcctgcttctctggcccctggtcctgtcctgttctccagcatggtgtgtctgaagctccctggaggctcctgcatgacagcgctgacagtgacactgatggtgctgagctccccactggctttggctggggacacccgaccacgtttcttgtggcagcttaagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggcgagttgagcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgctgagtggagtcgggggcttcgtgctgggcctgctcttccttggggccgggctgttcatctacttcaggaatcagaaaggacactctggacttcagccaacaggattcctgagctgaaatgcagatgaccacattcaaggaagaaccttctgtcccagctttgcagaatgaaaagctttcctgcttggcagttattcttccacaagagagggctttctcaggacctggttgctactggttcggcaactgcagaaaatgtcctcccttgtggcttcctcagctcctgcccttggcctgaagtcccagcattgatgacagcgcctcatcttcaacttttgtgctcccctttgcctaaaccgtatggcctcccgtgcatctgtactcaccctgtacgacaaacacattacattattaaatgtttctcaaagatggagtt

SEQ ID NO: 25 - DNA, 1701 bp, Homo sapiens
  source 1..1701: mol_type = unassigned DNA
cggactcaagaagttctcaggactcagaggctgggatcatggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaaagcctgagacagctgccttgtgtgcgactgagatgcacagctgccttgtgtgcgactgagatgcaggatttcctcacgcctcccctatgtgtcttaggggactctggcttctctttttgcaagggcctctgaatctgtctgtgtccctgttagcacaatgtgaggaggtagagaaacagtccacctctgtgtctaccatgacccccttcctcacactgacctgtgttccttccctgttctcttttctattaaaaataagaacctgggcagagtgcggcagctcatgcctgtaatcccagcacttagggaggccgaggagggcagatcacgaggtcaggagatcgaaaccatcctggctaacacggtgaaaccccgtctctactaaaaaatacaaaaaattagctgggcgcagaggcacgggcctgtagtcccagctactcaggaggcggaggcaggagaatggcgtcaacccgggaggcggaggttgcagtgagccaggattgtgcgactgcactccagcctgggtgacagggtgaaacgccatctcaaaaaataaaaattgaaaaataaaaaaaaaaaaaaaaaa

SEQ ID NO: 26 - DNA, 1655 bp, Homo sapiens
  source 1..1655: mol_type = unassigned DNA
aatttctcactgcccctgtgataaactgtggtcactggctgtggcagcaactattataagatgctctgaaaactcttcagacactgaggggcaccagaggagcagactacaagaatggcacacgctatggaaaactcctggacaatcagtaaagagtaccatattgatgaagaagtgggctttgctctgccaaatccacaggaaaatctacctgatttttataatgactggatgttcattgctaaacatctgcctgatctcatagagtctggccagcttcgagaaagagttgagaagttaaacatgctcagcattgatcatctcacagaccacaagtcacagcgccttgcacgtctagttctgggatgcatcaccatggcatatgtgtggggcaaaggtcatggagatgtccgtaaggtcttgccaagaaatattgctgttccttactgccaactctccaagaaactggaactgcctcctattttggtttatgcagactgtgtcttggcaaactggaagaaaaaggatcctaataagcccctgacttatgagaacatggacgttttgttctcatttcgtgatggagactgcagtaaaggattcttcctggtctctctattggtggaaatagcagctgcttctgcaatcaaagtaattcctactgtattcaaggcaatgcaaatgcaagaacgggacactttgctaaaggcgctgttggaaatagcttcttgcttggagaaagcccttcaagtgtttcaccaaatccacgatcatgtgaacccaaaagcatttttcagtgttcttcgcatatatttgtctggctggaaaggcaacccccagctatcagacggtctggtgtatgaagggttctgggaagacccaaaggagtttgcagggggcagtgcaggccaaagcagcgtctttcagtgctttgacgtcctgctgggcatccagcagactgctggtggaggacatgctgctcagttcctccaggacatgagaagatatatgccaccagctcacaggaacttcctgtgctcattagagtcaaatccctcagtccgtgagtttgtcctttcaaaaggtgatgctggcctgcgggaagcttatgacgcctgtgtgaaagctctggtctccctgaggagctaccatctgcaaatcgtgactaagtacatcctgattcctgcaagccagcagccaaaggagaataagacctctgaagacccttcaaaactggaagccaaaggaactggaggcactgatttaatgaatttcctgaagactgtaagaagtacaactgagaaatcccttttgaaggaaggttaatgtaacccaacaagagcacattttatcatagcagagacatctgtatgcattcctgtcattacccattgtaacagagccacaaactaatactatgcaatgttttaccaataatgcaatacaaaagacctcaaaatacctgtgcatttcttgtaggaaaacaacaaaaggtaattatgtgtaattatactagaagttttgtaatctgtatcttatcattggaataaaatgacattcaataaataaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27 - DNA, 1995 bp, Homo sapiens
  source 1..1995: mol_type = unassigned DNA
acaggggtgaaggcccagagaccagcagaacggcatcccagccacgacggccactttgctctgtctgctctccgccacggccctgctctgttccctgggacacccccgcccccacctcctcaggctgcctgatctgcccagctttccagctttcctctggattccggcctctggtcatccctccccaccctctctccaaggccctctcctggtctcccttcttctagaaccccttcctccacctccctctctgcagaacttctcctttaccccccaccccccaccactgccccctttccttttctgacctccttttggagggctcagcgctgcccagaccataggagagatgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcctgctgtttctcatccttggtgtcctttctctgctccttttggtgactggagcctttggctttcacctttggagaagacagtggcgaccaagacgattttctgccttagagcaagggattcaccctccgcaggctcagagcaagatagaggagctggagcaagaaccggagccggagccggagccggaaccggagcccgagcccgagcccgagccggagcagctctgacctggagctgaggcagccagcagatctcagcagcccagtccaaataaactccctgtcagcagcaaaaa

SEQ ID NO: 28 - DNA, 826 bp, Homo sapiens
  source 1..826: mol_type = unassigned DNA
tcatgtgacaaagcgcaggacccctcactgccccaactgcttgctgttctctctttcttgggctctaaggacccaggagtctgggtgcacagcctccttctctctgagattcaagagtctgatcagcagcctcttcctcctccaggacccagaagccctgagcttatccccatggagctctgccggtccctggccctgctggggggctccctgggcctgatgttctgcctgattgctttgagcaccgatttctggtttgaggctgtgggtcccacccactcagctcactcgggcctctggccaacagggcatggggacatcatatcaggctacatccacgtgacgcagaccttcagcattatggctgttctgtgggccctggtgtccgtgagcttcctggtcctgtcctgcttcccctcactgttccccccaggccacggcccgcttgtctcaaccaccgcagcctttgctgcagccatctccatggtggtggccatggcggtgtacaccagcgagcggtgggaccagcctccacacccccagatccagaccttcttctcctggtccttctacctgggctgggtctcagctatcctcttgctctgtacaggtgccctgagcctgggtgctcactgtggcggtccccgtcctggctatgaaaccttgtgagcagaaggcaagagcggcaagatgagttttgagcgttgtattccaaaggcctcatctggagcctcgggaaagtctggtcccacatctgcccgcccttccagcccttccccagcccctcctcttgtttcttcattcattcaacaaaatttggctggaa

SEQ ID NO: 29 - DNA, 2418 bp, Homo sapiens
  source 1..2418: mol_type = unassigned DNA
gcaaaccttaagctgaatgaacaacttttcttctcttgaatatatcttaacgccaaattttgagtgcttttttgttacccatcctcatatgtcccagctagaaagaatcctgggttggagctactgcatgttgattgttttgtttttccttttggctgttcattttggtggctactataaggaaatctaacacaaacagcaactgttttttgttgtttacttttgcatctttacttgtggagctgtggcaagtcctcatatcaaatacagaacatgatcttcctcctgctaatgttgagcctggaattgcagcttcaccagatagcagctttattcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaagtcaaagcttcctacaggaaaataaacactcacatcctaaaggttccagaaacagatgaggtagagctcacctgccaggctacaggttatcctctggcagaagtatcctggccaaacgtcagcgttcctgccaacaccagccactccaggacccctgaaggcctctaccaggtcaccagtgttctgcgcctaaagccaccccctggcagaaacttcagctgtgtgttctggaatactcacgtgagggaacttactttggccagcattgaccttcaaagtcagatggaacccaggacccatccaacttggctgcttcacattttcatccccttctgcatcattgctttcattttcatagccacagtgatagccctaagaaaacaactctgtcaaaagctgtattcttcaaaagacacaacaaaaagacctgtcaccacaacaaagagggaagtgaacagtgctatctgaacctgtggtcttgggagccagggtgacctgatatgacatctaaagaagcttctggactctgaacaagaattcggtggcctgcagagcttgccatttgcacttttcaaatgcctttggatgacccagcactttaatctgaaacctgcaacaagactagccaacacctggccatgaaacttgccccttcactgatctggactcacctctggagcctatggctttaagcaagcactactgcactttacagaattaccccactggatcctggacccacagaattccttcaggatccttcttgctgccagactgaaagcaaaaggaattatttcccctcaagttttctaagtgatttccaaaagcagaggtgtgtggaaatttccagtaacagaaacagatgggttgccaatagagttattttttatctatagcttcctctgggtactagaagaggctattgagactatgagctcacagacagggcttcgcacaaactcaaatcataattgacatgttttatggattactggaatcttgatagcataatgaagttgttctaattaacagagagcatttaaatatacactaagtgcacaaattgtggagtaaagtcatcaagctctgtttttgaggtctaagtcacaaagcatttgttttaacctgtaatggcaccatgtttaatggtggttttttttttgaactacatctttcctttaaaaattattggtttctttttatttgtttttaccttagaaatcaattatatacagtcaaaaatatttgatatgctcatacgttgtatctgcagcaatttcagataagtagctaaaatggccaaagccccaaactaagcctccttttctggccctcaatatgactttaaatttgacttttcagtgcctcagtttgcacatctgtaatacagcaatgctaagtagtcaaggcctttgataattggcactatggaaatcctgcaagatcccactacatatgtgtggagcagaagggtaactcggctacagtaacagcttaattttgttaaatttgttctttatactggagccatgaagctcagagcattagctgacccttgaactattcaaatgggcacattagctagtataacagacttacataggtgggcctaaagcaagctccttaactgagcaaaatttggggcttatgagaatgaaagggtgtgaaattgactaacagacaaatcatacatctcagtttctcaattctcatgtaaatcagagaatgcctttaaagaataaaactcaattgttattcttcaacgttctttatatattctacttttgggta

SEQ ID NO: 30 - DNA, 1009 bp, Homo sapiens
  source 1..1009: mol_type = unassigned DNA
agacgtgaagcctagcagaggactttttagctgctcactggccccgcttgtctggccgactcatccgcccgcgacccctaatcccctctgcctgccccaagatgctgaagccagccctggagccccgagggggcttctccttcgagaactgccaaagaaatgcatcattggaacgcgtcctcccggggctcaaggtccctcacgcacgcaagaccgggaccaccatcgcgggcctggtgttccaagacggggtcattctgggcgccgatacgcgagccactaacgattcggtcgtggcggacaagagctgcgagaagatccacttcatcgcccccaaaatctactgctgtggggctggagtagccgcggacgccgagatgaccacacggatggtggcgtccaagatggagctacacgcgttatctacgggccgcgagccccgcgtggccacggtcactcgcatcctgcgccagacgctcttcaggtaccagggccacgtgggtgcatcgctgatcgtgggcggcgtagacctgactggaccgcagctctacggcgtgcatccccatggctcctacagccgtctgcccttcacagccctgggctctggtcaggacgcggccctggcggtgctagaagaccggttccagccgaacatgacgctggaggctgctcaggggctgctggtggaagccgtcaccgccgggatcttgggtgacctgggctccgggggcaatgtggacgcatgtgtgatcacaaagactggcgccaagctgctgcggacactgagctcacccacagagcccgtgaagaggtctggccgctaccactttgtgcctggaaccacagctgtcctgacccagacagtgaagccactaaccctggagctagtggaggaaactgtgcaggctatggaggtggagtaagctgaggcttagagcttggaacaagggggaataaacccagaaaatacagttaaacaaaaaaaaaaaaaaaaaaaaaaaaaaaaaaa

SEQ ID NO: 31 - DNA, 4157 bp, Homo sapiens
  source 1..4157: mol_type = unassigned DNA
agcggggcggggcgccagcgctgccttttctcctgccgggtagtttcgctttcctgcgcagagtctgcggaggggctcggctgcaccggggggatcgcgcctggcagaccccagaccgagcagaggcgacccagcgcgctcgggagaggctgcaccgccgcgcccccgcctagcccttccggatcctgcgcgcagaaaagtttcatttgctgtatgccatcctcgagagctgtctaggttaacgttcgcactctgtgtatataacctcgacagtcttggcacctaacgtgctgtgcgtagctgctcctttggttgaatccccaggcccttgttggggcacaaggtggcaggatgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagttcacccttctagacttcagaccacagacaacctgctccccatgtctcctgaggagtttgacgaggtgtctcggatagtgggctctgtagaattcgacagtatgatgaacacagtatagagcatgaatttttttcatcttctctggcgacagttttccttctcatctgtgattccctcctgctactctgttccttcacatcctgtgtttctagggaaatgaaagaaaggccagcaaattcgctgcaacctgttgatagcaagtgaatttttctctaactcagaaacatcagttactctgaagggcatcatgcatcttactgaaggtaaaattgaaaggcattctctgaagagtgggtttcacaagtgaaaaacatccagatacacccaaagtatcaggacgagaatgagggtcctttgggaaaggagaagttaagcaacatctagcaaatgttatgcataaagtcagtgcccaactgttataggttgttggataaatcagtggttatttagggaactgcttgacgtaggaacggtaaatttctgtgggagaattcttacatgttttctttgctttaagtgtaactggcagttttccattggtttacctgtgaaatagttcaaagccaagtttatatacaattatatcagtcctctttcaaaggtagccatcatggatctggtagggggaaaatgtgtattttattacatctttcacattggctatttaaagacaaagacaaattctgtttcttgagaagagaatattagctttactgtttgttatggcttaatgacactagctaatatcaatagaaggatgtacatttccaaattcacaagttgtgtttgatatccaaagctgaatacattctgctttcatcttggtcacatacaattatttttacagttctcccaagggagttaggctattcacaaccactcattcaaaagttgaaattaaccatagatgtagataaactcagaaatttaattcatgtttcttaaatgggctactttgtcctttttgttattagggtggtatttagtctattagccacaaaattgggaaaggagtagaaaaagcagtaactgacaacttgaataatacaccagagataatatgagaatcagatcatttcaaaactcatttcctatgtaactgcattgagaactgcatatgtttcgctgatatatgtgtttttcacatttgcgaatggttccattctctctcctgtactttttccagacacttttttgagtggatgatgtttcgtgaagtatactgtatttttacctttttccttccttatcactgacacaaaaagtagattaagagatgggtttgacaaggttcttcccttttacatactgctgtctatgtggctgtatcttgtttttccactactgctaccacaactatattatcatgcaaatgctgtattcttctttggtggagataaagatttcttgagttttgttttaaaattaaagctaaagtatctgtattgcattaaatataatatgcacacagtgctttccgtggcactgcatacaatctgaggcctcctctctcagtttttatatagatggcgagaacctaagtttcagttgattttacaattgaaatgactaaaaaacaaagaagacaacattaaaacaatattgtttcta

SEQ ID NO: 32 - DNA, 2139 bp, Homo sapiens
  source 1..2139: mol_type = unassigned DNA
acatctgcttcctgtaggccctctgggcagaagcatgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggccttaccctccagtcgctgaccgtgaacgatacaggggagtacttctgcatctatcacacctaccctgatggggcgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttagcaaccagaggcatcttctggaagatacacttttgtctttgctattatagatgaatatataagcagctgcactctccatcagtgctgcgtgtgtgtgtgtgtgtgtatgtgtgtgtgtgttcagttgagtgaataaatgtcatcctcttctccatcttcatttccttggccttttcgttctattccattttgcattatggcaggcctagggtgagtaacgtggatcttgatcataaatgcaaaattaaaaaatatcttgacctggttttaaatctggcagtttgagcagatcctatgtctctgagagacacattcctcataatggccagcattttgggctacaaggttttgtggttgatgatgaggatggcatgactgcagagccatcctcatctcattttttcacgtcattttcagtaactttcactcattcaaaggcaggttataagtaagtcctggtagcagcctctatggggagatttgagagtgactaaatcttggtatctgccctcaagaacttacagttaaatggggagacaatgttgtcatgaaaaggtattatagtaaggagagaaggagacatacacaggccttcaggaagagacgacagtttggggtgaggtagttggcataggcttatctgtgatgaagtggcctgggagcaccaaggggatgttgaggctagtctgggaggagcaggagttttgtctagggaacttgtaggaaattcttggagctgaaagtcccacaaagaaggccctggcaccaagggagtcagcaaacttcagattttattctctgggcaggcatttcaagtttccttttgctgtgacatactcatccattagacagcctgatacaggcctgtagcctcttccggccgtgtgtgctggggaagccccaggaaacgcacatgcccacacagggagccaagtcgtagcatttgggccttgatctaccttttctgcatcaatacactcttgagcctttgaaaaaagaacgtttcccactaaaaagaaaatgtggatttttaaaatagggactcttcctaggggaaaaaggggggctgggagtgatagagggtttaaaaaataaacaccttcaaactaacttcttcgaacccttttattcactccctgacgactttgtgctggggttggggtaactgaactgcttatttctgtttaattgcattcaggctggatcttagaagacttttatccttccaccatctctctcagaggaatgagcggggaggttggatttactggtgactgattttctttcatgggccaaggaactgaaagagaatgtgaagcaaggttgtgtcttgcgcatggttaaaaataaagcattgtcctgcttcctaag

SEQ ID NO: 33 - DNA, 3360 bp, Homo sapiens
  source 1..3360: mol_type = unassigned DNA
gcgccagcttggagagccagccccatcggggttccccgccgccggaagcggaaatagcaccgggcgccgccacagtagctgtaactgccaccgcgatgccgaaggcgcccaagcagcagccgccggagcccgagtggatcggggacggagagagcacgagcccatcagacaaagtggtgaagaaagggaagaaggacaagaagatcaaaaaaacgttctttgaagagctggcagtagaagataaacaggctggggaagaagagaaagtgctcaaggagaaggagcagcagcagcagcaacagcaacagcagcaaaaaaaaaagcgagatacccgaaaaggcaggcggaagaaggatgtggatgatgatggagaagagaaagagctcatggagcgtcttaagaagctctcagtgccaaccagtgatgaggaggatgaagtacccgccccaaaaccccgcggagggaagaaaaccaagggtggtaatgtttttgcagccctgattcaggatcagagtgaggaagaggaggaggaagaaaaacatcctcctaagcctgccaagccggagaagaatcggatcaataaggccgtatctgaggaacagcagcctgcactcaagggcaaaaagggaaaggaagagaagtcaaaagggaaggctaagcctcaaaataaattcgctgctctggacaatgaagaggaggataaagaagaagaaattataaaggaaaaggagcctcccaaacaagggaaggagaaggccaagaaggcagagcagatggagtatgagcgccaagtggcttcattaaaagcagccaatgcagctgaaaatgacttctccgtgtcccaggcggagatgtcctcccgccaagccatgttagaaaatgcatctgacatcaagctggagaagttcagcatctccgctcatggcaaggagctgttcgtcaatgcagacctgtacattgtagccggccgccgctacgggctggtaggacccaatggcaagggcaagaccacactcctcaagcacattgccaaccgagccctgagcatccctcccaacattgatgtgttgctgtgtgagcaggaggtggtagcagatgagacaccagcagtccaggctgttcttcgagctgacaccaagcgattgaagctgctggaagaggagcggcggcttcagggacagctggaacaaggggatgacacagctgctgagaggctagagaaggtgtatgaggaattgcgggccactggggcggcagctgcagaggccaaagcacggcggatcctggctggcctgggctttgaccctgaaatgcagaatcgacccacacagaagttctcagggggctggcgcatgcgtgtctccctggccagggcactgttcatggagcccacactgctgatgctggatgagcccaccaaccacctggacctcaacgctgtcatctggcttaataactacctccagggctggcggaagaccttgctgatcgtctcccatgaccagggcttcttggatgatgtctgcactgatatcatccacctcgatgcccagcggctccactactataggggcaattacatgaccttcaaaaagatgtaccagcagaagcagaaagaactgctgaaacagtatgagaagcaagagaaaaagctgaaggagctgaaggcaggcgggaagtccaccaagcaggcggaaaaacaaacgaaggaagccctgactcggaagcagcagaaatgccgacggaaaaaccaagatgaggaatcccaggaggcccctgagctcctgaagcgccctaaggagtacactgtgcgcttcacttttccagaccccccaccactcagccctccagtgctgggtctgcatggtgtgacattcggctaccagggacagaaaccactctttaagaacttggattttggcatcgacatggattcaaggatttgcattgtgggccctaatggtgtggggaagagtacgctactcctgctgctgactggcaagctgacaccgacccatggggaaatgagaaagaaccaccggctgaaaattggcttcttcaaccagcagtatgcagagcagctgcgcatggaggagacgcccactgagtacctgcagcggggcttcaacctgccctaccaggatgcccgcaagtgcctgggccgcttcggcctggagagtcacgcccacaccatccagatctgcaaactctctggtggtcagaaggcgcgagttgtgtttgctgagctggcctgtcgggaacctgatgtcctcatcttggacgagccaaccaataacctggacatagagtctattgatgctctaggggaggccatcaatgaatacaagggtgctgtgatcgttgtcagccatgatgcccgactcatcacagaaaccaattgccagctgtgggtggtggaggagcagagtgttagccaaatcgatggtgactttgaagactacaagcgggaggtgttggaggccctgggtgaagtcatggtcagccggccccgagagtgagctttccttcccagaagtctcccgagagacatatttgtgtggcctagaagtcctctgtggtctcccctcctctgaagactgcctctggcctgcagctgacctggcaaccattcaggcacatgaaggtggagtgtgaccttgatgtgaccgggatcccactctgattgcatccatttctctgaaagacttgtttgttctgcttctcttcatataactgagctggccttatccttggcatccccctaaacaaacaagaggtgaccaccttattgtgaggttccatccagccaagtttatgtggcctattgtctcaggactctcatcactcagaagcctgcctctgatttaccctacagcttcaggcccagctgccccccagtctttgggtggtgctgttcttttctggtggatttaatgctgactcactggtacaaacagctgttgaagctcagagctggaggtgagcttctgaggcctttgccattatccagcccaagatttggtgcctgcagcctcttgtctggttgaggacttggggcaggaaaggaatgctgctgaacttgaatttccctttacaaggggaagaaataaaggaaaggagttgctgccgacctgtcactgtttggagattgatgggagttggaactgttctcagtcttgatttgctttattcagttttctagcagcttttaatagtcccctcttccccactaaatggatcttgtttgcagtcttgctgacagtgtttgctgtttaaggatcataggattcctttcccccaacccttcacgcaaggaaaaagcaaagtgattcataccttctatcttggaaaaaaaaaaaa

SEQ ID NO: 34 - DNA, 4026 bp, Homo sapiens
  source 1..4026: mol_type = unassigned DNA
ccccttggccccgccccaccctgctttgccctgcctctccctgccccgccgcgccccagtcccttgacgaccctcctctctgggccccgcccctcccgcttcggggtcaagccccagagagcgccgcgaaaaccacatttcccagagtgcaccgcgacggcaggggtcctcagaccggcgctcgctcgccggcgccatccctatagagaagaacggaggtacggcctgtggtcatggcgctgttcccagcctttgcggggcttagtgaggctcccgatggcgggagctccaggaaagagttagactggctgagcaacccaagcttttgtgttggatccataacgtccctgagccaacaaactgaagcagctccagcccatgtttctgaagggttaccgctgacaaggagtcatctgaaatcagagtcttcagatgaaagtgacactaacaaaaagctcaaacaaacaagtagaaaaaagaagaaagagaaaaagaaaaaaaggaagcatcagcatcataagaaaacaaagaggaagcatgggccgtcgagtagcagcaggtctgagacagacaccgattctgaaaaggacaaaccttccagaggcgttggaggcagtaaaaaggaatctgaggaaccgaatcaaggaaataatgctgcagctgatactggacatcgctttgtttggcttgaggacattcaggctgtgacgggagaaaccttcagaacagataagaaaccagatcctgcgaactgggagtacaagtctctctaccgaggggatatagcaagatacaagaggaaaggagactcctgccttggcattaaccctaagaagcagtgcatatcttgggaagggacttccacagagaagaagcattcacgcaagcaggttgaacgctattttactaagaagagtgtgggattaatgaacatcgatggagttgccattagcagtaaaactgaacctccctcatctgagcccatctcctttataccagtgaaggacttggaagatgcggctcctgttacaacctggttgaatcctctggggatttatgatcagtcaaccacacattggctacaaggacagggtcctccagagcaggaatcaaagcagccagacgcacagccagacagcgagagtgcggctctcaaggccaaggtggaggagtttaacaggagggtgcgggagaatcctcgggatacgcagctgtggatggcatttgttgcttttcaggacgaggtcatgaaaagtcctggcctgtatgccatcgaggaaggagagcaggaaaagcgaaagaggtccctgaagctcattctggagaagaagctggccattctggagcgggccattgagagcaaccagagcagtgtggatctgaaactggccaagctgaagctctgcacagagttctgggagccctccactctggtcaaagagtggcagaaactgatatttttgcatcccaataatacagccctttggcagaaataccttttattttgccagagccagtttagtaccttttcgatatcaaaaattcacagtctttatggaaaatgcttgagcactttgtctgctgttaaggacggcagcatcttatctcaccctgcgttgcctggcacggaagaggccatgtttgcactctttcttcagcagtgccactttctgcggcaggctggccactctgagaaggccatctcattgttccaggccatggtggacttcaccttcttcaaacccgacagcgtgaaagatctgcctaccaaaggacaggtggaattctttgaacccttttgggacagtggagagccccgggctggggagaagggagcccgaggctggaaggcgtggatgcaccagcaggaacgaggtggctgggtggtcatcaacccagatgaggatgacgatgaaccagaagaggatgaccaggaaataaaagataagactctgcccaggtggcagatctggcttgctgctgagcgttcccgtgaccagaggcactggcggccctggcgccctgataagaccaagaagcaaaccgaggaagactgtgaggatcccgagagacaggtgttgtttgatgatattgggcaatctttgatcagactttccagccatgatcttcagttccagctggtggaggccttcctgcagttcttgggtgtgccttctggctttactcctccagcctcctgtctttatctggccatggatgagaacagcatctttgataatggactttatgatgaaaagcccttgacttttttcaaccctttgttttctggggctagctgtgttggccgcatggataggttgggctatcctcgctggaccaggggtcagaaccgagagggcgaggagttcatccgcaatgtcttccaccttgtcatgcctttattttcaggcaaagagaagtcccagctctgcttctcctggttacagtatgagattgcaaaggtcatttggtgcctgcacactaaaaacaagaagagattaaagtctcaagggaagaactgcaaaaaactagccaagaatctccttaaggagccagaaaactgcaacaacttttgcctgtggaagcagtatgcacatctggagtggttgcttggcaacacggaggatgccagaaaagtttttgacacagcacttggcatggcaggaagcagagaactgaaagactctgacctctgtgagctcagtctgctctatgctgagctggaggtggagctgtcgccagaagtgagaagggctgccacagctcgagctgttcacatattaaccaagctgactgagagcagcccctatgggccctacactggacaggtgttggctgttcacattttgaaagcgcgaaaggcttatgagcacgcactgcaggactgtttgggtgacagctgtgtctccaatccagctcccaccgattcctgtagccgcctaattagcctggctaaatgcttcatgctcttccagtatttgaccatagggattgatgctgctgtgcagatatacgaacaggtgtttgcaaaactgaacagttctgttttcccagaaggctctggcgagggggacagtgccagctcccagagttggaccagtgttctcgaagccatcacactgatgcacacgagcctgctgagattccacatgaaagtgagtgtttacccgctggcccctctgcgagaggcactctcacaggctttaaagttgtatccaggcaaccaggttctttggaggtcctatgtacagattcagaataagtcccacagtgccagcaaaaccaggagattttttgacacaatcaccaggtctgccaaacccttggagccttggttgtttgcaattgaagctgagaaactgaggaagagactggtggaaactgtccagaggttagacggtagagagatccacgccacaattcctgagaccggcttaatgcatcggatccaagccctgtttgaaaatgccatgcgcagcgacagtggcagccagtgccccttgctgtggaggatgtatttgaactttctggtttccttaggaaataaagaaagaagcaaaggtgtattctacaaagcacttcagaattgcccttgggcaaaggtgttgtacctggacgccgtggagtatttccccgatgagatgcaggagatcctggacctgatgactgagaaggagctccgggtgcgcctgccgctggaggagctggagctgctgctggaggattagagagcagcgggaaaacgggctgtgcctgcgaggccaagttgcccaccctgcggagctaggaggcgcgagcagagaacgtgtgtgttaggagaactcggcttttgaaatgttctttctcgatagtaataatgtgggctgccagcctctcacatcttgcacactttttgggtgtgtaaatgacacaaaagttatttacatattatatatgtgaatatgtgtatatatgtacatagccagagagtcatgccacgtggtcattaaaccgatgatgattgaggcgtgaaaaaaaaaaaaaaaa

SEQ ID NO: 35 - DNA, 2650 bp, Homo sapiens
  source 1..2650: mol_type = unassigned DNA
agggacagcccagaggaggcgtggccacgctgccggcggaagtggagccctccgcgagcgcgcgaggccgccggggcaggcggggaaaccggacagtaggggcggggccgggccggcgatggggatgcgggagcactacgcggagctgcacccgtgcccgccggaattggggatgcagagcagcggcagcgggtatggcaggcagccggcgggccggcctccagcgcaggtgcccgagaggcaggggctggcctgggatgcgcgcgcacctgccctcgccccgccccgcccgcacgaggggtggtggccgaggccccgccccgcacgcctcgcctgaggcgggtccgctcagcccaggcgcccgcccccgcccccgccgattaaatgggccggcggggctcagcccccggaaacggtcgtaacttcggggctgcgagcgcggagggcgacgacgacgaagcgcagacagcgtcatggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatggatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctacgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctgagccctgggcacccacctccacccccgccacggccaccctccttcccgccgcccgaccccgagtcgggaggactccgggaccattgacctcagctgcacattcctggccccgggctctggccaccctggcccgcccctcgctgctgctactacccgagcccagctacattcctcagctgccaagcactcgagaccatcctggcccctccagaccctgcctgagcccaggagctgagtcacctcctccactcactccagcccaacagaaggaaggaggagggcgcccattcgtctgtcccagagcttattggccactgggtctcactcctgagtggggccagggtgggagggagggacaagggggaggaaaggggcgagcacccacgtgagagaatctgcctgtggccttgcccgccagcctcagtgccacttgacattccttgtcaccagcaacatctcgagccccctggatgtcccctgtcccaccaactctgcactccatggccaccccgtgccacccgtaggcagcctctctgctataagaaaagcagacgcagcagctgggacccctcccaacctcaatgccctgccattaaatccgcaaacagccaaaaaaaaaaaaaaaaaaaa

SEQ ID NO: 36 - DNA, 1146 bp, Homo sapiens
  source 1..1146: mol_type = unassigned DNA
ttttgcgaacggcgagcagcggcggcggcgcggagagacgcagcggaggttttcctggtttcggaccccagcggccggatggtgaaatcctccctgcagcggatcctcaatagccactgcttcgccagagagaaggaaggggataaacccagcgccaccatccacgccagccgcaccatgccgctcctaagcctgcacagccgcggcggcagcagcagtgagagttccagggtctccctccactgctgtagtaacccgggtccggggcctcggtggtgctcctgatgcccctcacccacccctgaagatcccaggtgggcgagggaatagtcagagggatcacaatctttcagctaacttattctactccgatgatcggctgaatgtaacagaggaactaacgtccaacgacaagacgaggattctcaacgtccagtccaggctcacagacgccaaacgcattaactggcgaacagtgctgagtggcggcagcctctacatcgagatcccgggcggcgcgctgcccgaggggagcaaggacagctttgcagttctcctggagttcgctgaggagcagctgcgagccgaccatgtcttcatttgcttccacaagaaccgcgaggacagagccgccttgctccgaaccttcagctttttgggctttgagattgtgagaccggggcatccccttgtccccaagagacccgacgcttgcttcatggcctacacgttcgagagagagtcttcgggagaggaggaggagtagggccgcctcggggctgggcatccggcccctggggccaccccttgtcagccgggtgggtaggaaccgtagactcgctcatctcgcctgggtttgtccgcatgttgtaatcgtgcaaataaacgctcactccgaattagcggtgtatttcttgaagtttaatattgtgtttgtgatactgaagtatttgctttaattctaaataaaaatttatattttacttttttattgctggtttaagatgattcagattatccttgtactttgaggagaagtttcttatttggagtcttttggaaacagtcttagtcttttaacttggaaagatgaggtattaatcccctccattgctctccaaaagccaataaagtgattacacccga

SEQ ID NO: 37 - DNA, 6732 bp, Homo sapiens
  source 1..6732: mol_type = unassigned DNA
gagagcgcggccgggacggttggagaagaaggcggctccccggaagggggagagacaaactgccgtaacctctgccgttcaggaacccggttacttatttattcgttaccctttttcttcttcctcccccaaaaaccttttccttttcccttctttttttttcctttttgggagctgaaaaatttccggtaagggaaagaagggctcctttcgctccttatttcgccgcctccttccctccgccaccttcccctcctccggctttttcctcccaactcggggaggtccttcccggtggccgccctgacgaggtctgagcacctaggcggaggcggcgcaggctttttgtagtgaggtttgcgcctgcgcaggcgcctgcctccgccatgcacgggggtggccccccctcgggggacagcgcatgcccgctgcgcaccatcaagagagtccagttcggagtcctgagtccggatgaactgaagcgaatgtctgtgacggagggtggcatcaaatacccagagacgactgagggaggccgccccaagcttggggggctgatggacccgaggcagggggtgattgagcggactggccgctgccaaacatgtgcaggaaacatgacagagtgtcctggccactttggccacattgaactggccaagcctgtgtttcacgtgggcttcctggtgaagacaatgaaagttttgcgctgtgtctgcttcttctgctccaaactgcttgtggactctaacaacccaaagatcaaggatatcctggctaagtccaagggacagcccaagaagcggctcacacatgtctacgacctttgcaagggcaaaaacatatgcgagggtggggaggagatggacaacaagttcggtgtggaacaacctgagggtgacgaggatctgaccaaagaaaagggccatggtggctgtgggcggtaccagcccaggatccggcgttctggcctagagctgtatgcggaatggaagcacgttaatgaggactctcaggagaagaagatcctgctgagtccagagcgagtgcatgagatcttcaaacgcatctcagatgaggagtgttttgtgctgggcatggagccccgctatgcacggccagagtggatgattgtcacagtgctgcctgtgcccccgctctccgtgcggcctgctgttgtgatgcagggctctgcccgtaaccaggatgacctgactcacaaactggctgacatcgtgaagatcaacaatcagctgcggcgcaatgagcagaacggcgcagcggcccatgtcattgcagaggatgtgaagctcctccagttccatgtggccaccatggtggacaatgagctgcctggcttgccccgtgccatgcagaagtctgggcgtcccctcaagtccctgaagcagcggttgaagggcaaggaaggccgggtgcgagggaacctgatgggcaaaagagtggacttctcggcccgtactgtcatcacccccgaccccaacctctccattgaccaggttggcgtgccccgctccattgctgccaacatgacctttgcggagattgtcacccccttcaacattgacagacttcaagaactagtgcgcagggggaacagtcagtacccaggcgccaagtacatcatccgagacaatggtgatcgcattgacttgcgtttccaccccaagcccagtgaccttcacctgcagaccggctataaggtggaacggcacatgtgtgatggggacattgttatcttcaaccggcagccaactctgcacaaaatgtccatgatggggcatcgggtccgcattctcccatggtctacctttcgcttgaatcttagcgtgacaactccgtacaatgcagactttgacggggatgagatgaacttgcacctgccacagtctctggagacgcgagcagagatccaggagctggccatggttcctcgcatgattgtcaccccccagagcaatcggcctgtcatgggtattgtgcaggacacactcacagcagtgcgcaaattcaccaagagagacgtcttcctggagcggggtgaagtgatgaacctcctgatgttcctgtcgacgtgggatgggaaggtcccacagccggccatcctaaagccccggcccctgtggacaggcaagcaaatcttctccctcatcatacctggtcacatcaattgtatccgtacccacagcacccatcccgatgatgaagacagtggcccttacaagcacatctctcctggggacaccaaggtggtggtggagaatggggagctgatcatgggcatcctgtgtaagaagtctctgggcacgtcagctggctccctggtccacatctcctacctagagatgggtcatgacatcactcgcctcttctactccaacattcagactgtcattaacaactggctcctcatcgagggtcatactattggcattggggactccattgctgattctaagacttaccaggacattcagaacactattaagaaggccaagcaggacgtaatagaggtcatcgagaaggcacacaacaatgagctggagcccaccccagggaacactctgcggcagacgtttgagaatcaggtgaaccgcattcttaacgatgcccgagacaagactggctcctctgctcagaaatccctgtctgaatacaacaacttcaagtctatggtcgtgtccggagctaaaggttccaagattaacatctcccaggtcattgctgtcgttggacagcagaacgtcgagggcaagcggattccatttggcttcaagcaccggactctgcctcacttcatcaaggatgactacgggcctgagagccgtggctttgtggagaactcctacctagccggcctcacacccactgagttctttttccacgccatggggggtcgtgaggggctcattgacacggctgtcaagactgctgagactggatacatccagcggcggctgatcaagtccatggagtcagtgatggtgaagtacgacgcgactgtgcggaactccatcaaccaggtggtgcagctgcgctacggcgaagacggcctggcaggcgagagcgttgagttccagaacctggctacgcttaagccttccaacaaggcttttgagaagaagttccgctttgattataccaatgagagggccctgcggcgcactctgcaggaggacctggtgaaggacgtgctgagcaacgcacacatccagaacgagttggagcgggaatttgagcggatgcgggaggatcgggaggtgctcagggtcatcttcccaactggagacagcaaggtcgtcctcccctgtaacctgctgcggatgatctggaatgctcagaaaatcttccacatcaacccacgccttccctccgacctgcaccccatcaaagtggtggagggagtcaaggaattgagcaagaagctggtgattgtgaatggggatgacccactaagtcgacaggcccaggaaaatgccacgctgctcttcaacatccacctgcggtccacgttgtgttcccgccgcatggcagaggagtttcggctcagtggggaggccttcgactggctgcttggggagattgagtccaagttcaaccaagccattgcgcatcccggggaaatggtgggggctctggctgcgcagtcccttggagaacctgccacccagatgaccttgaataccttccactatgctggtgtgtctgccaagaatgtgacgctgggtgtgccccgacttaaggagctcatcaacatttccaagaagccaaagactccttcgcttactgtcttcctgttgggccagtccgctcgagatgctgagagagccaaggatattctgtgccgtctggagcatacaacgttgaggaaggtgactgccaacacagccatctactatgaccccaacccccagagcacggtggtggcagaggatcaggaatgggtgaatgtctactatgaaatgcctgactttgatgtggcccgaatctccccctggctgttgcgggtggagctggatcggaagcacatgactgaccggaagctcaccatggagcagattgctgaaaagatcaatgctggttttggtgacgacttgaactgcatctttaatgatgacaatgcagagaagctggtgctccgtattcgcatcatgaacagcgatgagaacaagatgcaagaggaggaagaggtggtggacaagatggatgatgatgtcttcctgcgctgcatcgagtccaacatgctgacagatatgaccctgcagggcatcgagcagatcagcaaggtgtacatgcacttgccacagacagacaacaagaagaagatcatcatcacggaggatggggaattcaaggccctgcaggagtggatcctggagacggacggcgtgagcttgatgcgggtgctgagtgagaaggacgtggaccccgtacgcaccacgtccaatgacattgtggagatcttcacggtgctgggcattgaagccgtgcggaaggccctggagcgggagctgtaccacgtcatctcctttgatggctcctatgtcaattaccgacacttggctctcttgtgtgataccatgacctgtcgtggccacttgatggccatcacccgacacggagtcaaccgccaggacacaggaccactcatgaagtgttcctttgaggaaacggtggacgtgcttatggaagcagccgcacacggtgagagtgaccccatgaagggggtctctgagaatatcatgctgggccagctggctccggccggcactggctgctttgacctcctgcttgatgcagagaagtgcaagtatggcatggagatccccaccaatatccccggcctgggggctgctggacccaccggcatgttctttggttcagcacccagtcccatgggtggaatctctcctgccatgacaccttggaaccagggtgcaacccctgcctatggcgcctggtcccccagtgttgggagtggaatgaccccaggggcagccggcttctctcccagtgctgcgtcagatgccagcggcttcagcccaggttactcccctgcctggtctcccacaccgggctccccggggtccccaggtccctcaagcccctacatcccttcaccaggtggtgccatgtctcccagctactcgccaacgtcacctgcctacgagccccgctctcctgggggctacacaccccagagtccctcttattcccccacttcaccctcctactcccctacctctccatcctattctccaaccagtcccaactatagtcccacatcacccagctattcgccaacgtcacccagctactcaccgacctctcccagctactcacccacctctcccagctactcgcccacctctcccagctattcgcccacctctcccagctactcacccacttcccctagctattcgcccacttcccctagctactcgccaacgtctcccagctactcgccgacatctcccagctactcgccaacttcacccagctattctcccacttctcccagctactcacctacctctccaagctattcacccacctcccccagctactcacccacttccccaagttactcacccaccagcccgaactattctccaaccagtcccaattacaccccaacatcacccagctacagcccgacatcacccagctattcccctactagtcccaactacacacctaccagccctaactacagcccaacctctccaagctactctccaacatcacccagctattccccgacctcaccaagttactccccttccagcccacgatacacaccacagtctccaacctataccccaagctcacccagctacagccccagttcgcccagctacagcccaacctcacccaagtacaccccaaccagtccttcttatagtcccagctccccagagtataccccaacctctcccaagtactcacctaccagtcccaaatattcacccacctctcccaagtactcgcctaccagtcccacctattcacccaccaccccaaaatactccccaacatctcctacttattccccaacctctccagtctacaccccaacctctcccaagtactcacctactagccccacttactcgcccacttcccccaagtactcgcccaccagccccacctactcgcccacctcccccaaaggctcaacctactctcccacttcccctggttactcgcccaccagccccacctacagtctcacaagcccggctatcagcccggatgacagtgacgaggagaactgagggcacgtggggtgcggcagcgggctagggcccagggcagcttgcccgtgctgccgtgcagttcttgcctccctcacggggcgtcacccccagcccagctccgttgtacataaataccttgtgacagagctcccggtgaacttctggatcccgtttctgatgcagattcttgtcttgttctccacttgtgctgttagaactcactggcccagtggtgttctacctcctaccccacccaccccctgcctgtccccaaattgaagatccttccttgcctgtggcttgatgcggggcgggtaaagggtattttaacttaggggtagttcctgctgtgagtggttacagctgatcctcgggaagaacaaagctaaagctgccttttgtctgttattttatttttttgaagtttaaataaagtttactaattttgacc

SEQ ID NO: 38 - DNA, 2277 bp, Homo sapiens
  source 1..2277: mol_type = unassigned DNA
gactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcgcagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggacggtctctgcgatatgataccagctattttgtggagtattttgccttggatctcctgatggagaacggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccccacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtcgatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacagacctggtggagaccctggagctgcagaacctgatgctgtgtgcgctgcagaccatctacggagcagaggcgcggaaggagtcacggggcgcgcatgccagggaagactacaaggtgcggattgatgagtacgattactccaagcccatccaggggcaacagaagaagccctttgaggagcactggaggaagcacaccctgtcctttgtggacgttggcactgggaaggtcactctggaatatagacccgtaatcgacaaaactttgaacgaggctgactgtgccaccatcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgag

SEQ ID NO: 39 - DNA, 3602 bp, Homo sapiens
  source 1..3602: mol_type = unassigned DNA
gataggcgccgggcagctgagctggtaggaggaccagacggggatgttcggctccgccccccagcgtcccgtggccatgacgaccgctcagagggactccctgttgtggaagctcgcggggttgctgcgggagtccggggatgtggtcctgtctggctgtagcaccctgagcctgctgactcccacactgcaacagctgaaccacgtatttgagctgcacctggggccatggggccctggccagacaggctttgtggctctgccctcccatcctgccgactcccctgttattcttcagcttcagtttctcttcgatgtgctgcagaaaacactttcactcaagctggtccatgttgctggtcctggccccacagggcccatcaagattttccccttcaaatcccttcggcacctggagctccgaggtgttcccctccactgtctgcatggcctccgaggcatctactcccagctggagaccctgatttgcagcaggagcctccaggcattagaggagctcctctcagcctgcggcggcgacttctgctctgccctcccttggctggctctgctttctgccaacttcagctacaatgcactgaccgccttagacagctccctgcgcctcttgtcagctctgcgtttcttgaacctaagccacaatcaagtccaggactgtcagggattcctgatggatttgtgtgagctccaccatctggacatctcctataatcgcctgcatttggtgccaagaatgggaccctcaggggctgctctgggggtcctgatactgcgaggcaatgagcttcggagcctgcatggcctagagcagctgaggaatctgcggcacctggatttggcatacaacctgctggaaggacaccgggagctgtcaccactgtggctgctggctgagctccgcaagctctacctggaggggaaccctctttggttccaccctgagcaccgagcagccactgcccagtacttgtcaccccgggccagggatgctgctactggcttccttctcgatggcaaggtcttgtcactgacagattttcagactcacacatccttggggctcagccccatgggcccacctttgccctggccagtggggagtactcctgaaacctcaggtggccctgacctgagtgacagcctctcctcagggggtgttgtgacccagcccctgcttcataaggttaagagccgagtccgtgtgaggcgggcaagcatctctgaacccagtgatacggacccggagccccgaactctgaacccctctccggctggatggttcgtgcagcagcacccggagctggagctcatgagcagcttccgggaacggttcggccgcaactggctgcagtacaggagtcacctggagccctccggaaaccctctgccggccacccccactacttctgcacccagtgcacctccagccagctcccagggccccgacactgcacccagaccttcacccccgcaggaggaagccagaggcccccaggagtcaccacagaaaatgtcagaggaggtcagggcggagccacaggaggaggaagaggagaaggaggggaaggaggagaaggaggagggggagatggtggaacagggagaagaggaggcaggagaggaggaagaagaggagcaggaccagaaggaagtggaagcggaactctgtcgccccttgttggtgtgtcccctggaggggcctgagggcgtacggggcagggaatgctttctcagggtcacttctgcccacctgtttgaggtggaactccaagcagctcgcaccttggagcgactggagctccagagtctggaggcagctgagatagagccggaggcccaggcccagaggtcgcccaggcccacgggctcagatctgctccctggagcccccatcctcagtctgcgcttctcctacatctgccctgaccggcagttgcgtcgctatttggtgctggagcctgatgcccacgcagctgtccaggagctgcttgccgtgttgaccccagtcaccaatgtggctcgggaacagcttggggaggccagggacctcctgctgggtagattccagtgtctacgctgtggccatgagttcaagccagaggagcccaggatgggattagacagtgaggaaggctggaggcctctgttccaaaagacagaatctcctgctgtgtgtcctaactgtggtagtgaccacgtggttctcctcgctgtgtctcggggaacccccaacagggagcggaaacagggagagcagtctctggctccttctccgtctgccagccctgtctgccaccctcctggccatggtgaccaccttgacagggccaagaacagcccacctcaggcaccgagcacccgtgaccatggtagttggagcctcagtcccccccctgagcgctgtggcctccgctctgtggaccaccgactccggctcttcctggatgttgaggtgttcagcgatgcccaggaggagttccagtgctgcctcaaggtgccagtggcattggcaggccacactggggagttcatgtgccttgtggttgtgtctgaccgcaggctgtacctgttgaaggtgactggggagatgcgtgagcctccagctagctggctgcagctgaccctggctgttcccctgcaggatctgagtggcatagagctgggcctggcaggccagagcctgcggctagagtgggcagctggggcgggccgctgtgtgctgctgccccgagatgccaggcattgccgggccttcctagaggagctccttgatgtcttgcagtctctgccccctgcctggaggaactgtgtcagtgccacagaggaggaggtcaccccccagcaccggctctggccattgctggaaaaagactcatccttggaggctcgccagttcttctaccttcgggcgttcctggttgaaggcccttccacctgcctcgtatccctgttgctgactccgtccaccctgttcctgttagatgaggatgctgcagggtccccggcagagccctctcctccagcagcatctggcgaagcctctgagaaggtgcctccctcggggccgggccctgctgtgcgtgtcagggagcagcagccactcagcagcctgagctccgtgctgctctaccgctcagcccctgaggacttgcggctgctcttctacgatgaggtgtcccggctggagagcttttgggcactccgtgtggtgtgtcaggagcagctgacagccctgcttgcctggatccgggaaccatgggaggagctgttttccatcggactccggacagtgatccaagaggcgctggcccttgaccgatgagggtcccacgctgaccttggccctgacctcaggagccacgctgtagacattccctctcctggtctctgggtctggcttccaggctctggctgtggatgtcttcagcctctgggtgctggccagtgaggtcccaaatgacccagggcttaagggagaggcgagagaatgatctggcctcaggggacaggccacctggtcaggaggaatatttttcctgcactttttctcaggtatcaataaagttgtttccaactcataa

SEQ ID NO: 40 - DNA, 3326 bp, Homo sapiens
  source 1..3326: mol_type = unassigned DNA
gaggggcggcccgcggccatggagacgggcacggcgcccctggtggccccgccgcgccgtcatggcgcccccgcggccccctcgccgccgccccggggttcccgggccgggcccgtcgtggtggtggctccgggacctccagtgactacggccacttcggcccccgtcaccctggtggcccccggggaggcgcggcccgcctgggtcccggggtcggccgagacctctgctccggccccggccccagccccggccccagccccggctgtcacgggcagcacggtggtggtgctgaccctggaggcctcgcccgaagccccaaagccgcagctcccctccggcccggaatccccagagcccgcggcagtggctggagttgagacatcgagggctctggccgcaggggcagactcgccgaagacagaggaggctcgaccctcacccgccccaggaccagggacccccaccgggacccctaccaggaccccttccagaacggctcctggtgccctgaccgccaaacccccgcttgcccccaagccgggaaccacagtggcctcaggagtgactgcacggagtgcatcaggacaagtgacaggtgggcatggagctgccgcagcaacatcagcatcagcaggacaggctcctgaggacccctcaggccctggcacaggcccctctgggacttgtgaggctccggtagctgtcgtgaccgtgaccccagctccggagcctgctgaaaactctcaagacctgggctccacgtccagcctgggacctggcatctctgggcctcgagggcaggccccggacacgctgagttacttggactccgtgagcctcatgtctgggaccttggagtccttggcggatgatgtgagctccatgggctcagattcagagataaacgggctggccctgcgcaagacggacaagtatggcttccttgggggcagccagtactcgggcagcctagagagctccattcccgtggacgtggctcggcagcgggagctcaaatggctggacatgttcagtaactgggataagtggctgtcacggcgattccagaaggtgaagctgcgctgccggaaggggatcccctcctctctcagagccaaagcctggcagtacctgtctaatagcaaggaacttctggagcagaacccaggaaagtttgaggagctggaacgggctcctggggaccccaagtggctggatgtgattgagaaggacctgcaccgccagttccctttccacgagatgtttgctgctcgaggggggcatgggcaacaggacctgtaccgaatcctgaaggcctacaccatctaccggcctgacgagggttactgccaggcccaggcccccgtggctgcggtcctgctcatgcacatgcctgcggagcaagccttttggtgcctggtgcagatctgcgacaagtacctcccaggttactacagtgcagggctggaggccattcagctggacggggagatcttttttgcactcctgcgccgggcctccccgctggcgcatcgccacctgcggcggcagcgcattgaccctgtgctctacatgacggagtggttcatgtgcatcttcgcccgcaccctgccctgggcgtcggtgctgcgtgtctgggacatgtttttctgtgaaggcgttaagatcatcttccgggtggccctggtcctgctgcgccacacgctgggctcagtggagaagctgcgctcctgccaaggcatgtatgagaccatggagcagctgcgtaacctgccccagcagtgcatgcaggaagacttcctggtgcatgaggtgaccaatctgccggtgacagaagcactgattgagcgggagaatgcagcccagctcaagaagtggcgggaaacgcggggggagctgcagtatcggccctcacggcgactgcatgggtcccgggccatccacgaggagcgccggcggcaacagccacccctgggcccctcctccagcctcctcagcctccctggcctcaagagccgaggctcccgggcagctggaggggccccgtccccgccgccccccgtccgcagagccagtgctgggcctgccccagggcctgtggtcactgctgagggactgcatccatcccttccctcacccactggcaatagcacccccttgggttccagcaaggagacccggaagcaggagaaggagcggcagaaacaggagaaggagcggcagaaacaggagaaggagcgggagaaggagcggcagaagcaggagaaagagcgagagaagcaggaaaaggagcgagagaagcaggagaaggagcggcagaagcaggagaagaaggctcaaggccggaagctttcgctgcgtcgaaaggcagatgggcccccaggcccccatgatggtggggacaggccctcagccgaggcccggcaggacgcttacttctgacctctgccctggggctggactgcatggcccccctctttccctcagccaagaacaggcctggcccaaggtgccaccccctagcaccttgtcaggctgtcccttgctggggaaagtggcttggttccccatctcctcgccagctgctgatccctacacgggcaggacagatgggcagctgcaaatgagtctggagcctctcatctcccatgaggctcagctggggtctctgtcgctcctgccccagttccctctgggtcccctcctaggtgctgtcctgaatggcccgttgtcatcccaggggtgactcctggtgatgggagtcagcagtttcagattcttacactccatagctccccttaccatgaggtggagctggcttccttttccctgtcttcagccctccctgtctcccccacttcctggccagggctctcattctggacctgtgttgtaattgtgtacagaggatggcgttggcctggggtgggggtgctcgctttgtcttctgtcctttggttctccttccataatgctcctgtacccagtttatttaaggggacatgcactggaataggaaatgtcccccatctcccttcctgcaccctgctgtgctccctccaaacccaccttgctctgtgttctcaggcccccctgcttttgtctcaccaggacccatacctttcaccttgttcccttccacccctccagttagtccctatctgggtaagggtcttcccttgagctccagggggtggaacccaatgtttacattctcttctgtctctgcccccaccccatgcagcgctttgaggaattggaaaagaacctgctgttgtacctgggaaaaaaaaaaaaaaaaaaaaaaaaaaaaaaaaaaaaaa

SEQ ID NO: 41 - DNA, 1719 bp, Homo sapiens
  source 1..1719: mol_type = unassigned DNA
ggcggaagtgacattatcaacgcgcgccaggggttcagtgaggtcgggcaggttcgctgtggcgggcgcctgggccgccggctgtttaacttcgcttccgctggcccatagtgatctttgcagtgacccagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttagattgttgttaaaaaaaaaaaaaaaaaa

SEQ ID NO: 42 - DNA, 971 bp, Homo sapiens
  source 1..971: mol_type = unassigned DNA
cactcgttgcataaatttgcgctccgccagcccggagcatttaggggcggttggctttgttgggtgagcttgtttgtgtccctgtgggtggacgtggttggtgattggcaggatcctggtatccgctaacaggtcaaaatgcagatcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggtatgcagatcttcgtgaagaccctgaccggcaagaccatcaccctggaagtggagcccagtgacaccatcgaaaatgtgaaggccaagatccaggataaagaaggcatccctcccgaccagcagaggctcatctttgcaggcaagcagctggaagatggccgcactctttctgactacaacatccagaaggagtcgaccctgcacctggtcctgcgtctgagaggtggtatgcagatcttcgtgaagaccctgaccggcaagaccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgcgcctgaggggtggctgttaattcttcagtcatggcattcgcagtgcccagtgatggcattactctgcactatagccatttgccccaacttaagtttagaaattacaagtttcagtaatagctgaacctgttcaaaatgttaataaaggtttcgttgcatggta

SEQ ID NO: 43 - DNA, 6577 bp, Homo sapiens
  source 1..6577: mol_type = unassigned DNA
cggggactggcctggcgccggcggcggcggagggggcgccgcgggcgggcgatgtgagcgcggcgctctggacagagtacgcttcatgtcagtagaaatggacagcagcagttttattcagtttgatgtgcccgagtacagcagcaccgttctgagccagctaaacgaactccgcctgcaggggaaactatgtgacatcattgtacacattcagggtcagccattccgagcccacaaagcagtccttgctgccagctccccatatttccgggaccattcagcgttaagtaccatgagtggcttgtcaatatcagtgattaaaaatcccaatgtgtttgagcagttgctttctttttgttacactggaagaatgtccttgcagctgaaggatgttgtcagttttctgactgcagccagctttcttcagatgcagtgtgtcattgacaagtgcacgcagatcctagagagcatccattccaaaatcagcgttggagatgttgactctgttaccgtcggtgctgaagagaatcccgagagtcgaaacggagtgaaagacagcagcttctttgccaacccagtggagatctctcctccatattgctctcagggacggcagcccaccgcaagcagtgacctccggatggagacgacccccagcaaagctttgcgcagccgcttacaggaggaggggcactcagaccgcgggagcagtgggagcgtttctgaatatgagattcagatagagggagaccatgagcaaggagacctattggtgagggagagccagatcaccgaggtgaaagtgaagatggagaagtccgaccggcccagctgttccgacagctcctccctgggtgacgatgggtaccacaccgagatggttgatggggaacaagttgtggcagtgaatgtgggctcctatggttctgtgctccagcacgcatactcctattcccaagcagcctcacagccaaccaatgtatcagaagcttttggaagtttgagtaattccagcccatccaggtccatgctgagctgtttccgaggagggcgtgcccgccagaagcgggctttgtctgtccacctgcacagtgacctgcagggcctggtgcagggctctgacagtgaagccatgatgaacaaccccgggtatgagagcagtccccgggagaggagtgcgagagggcattggtacccgtacaatgagaggttgatctgtatttactgtggaaagtccttcaaccagaaaggaagccttgataggcacatgcgactccatatgggaatcaccccctttgtgtgcaagttctgtgggaagaagtacacacggaaggaccaactggagtaccacatccggggccatacagatgataaaccattccgctgtgagatctgcgggaagtgctttccattccaaggtaccctcaaccagcacttgcggaaaaaccacccaggcgttgctgaagtcaggagtcgcattgagtcccccgagagaacagatgtgtacgtggaacagaaactagaaaatgacgcatcggcctcagagatgggcctagattcccggatggaaattcacacagtgtctgatgctcccgattaagatggtaaagaagtgcacccaaacaaagcacattaatcaatgcatatttgtgatttgctttgttgtaatctttggttttcccaaccatctggaaatctcttggtctcttggcagtttttctaaagtttctggatggaacacttcgttgtgtttatcctttcccctgccctccctccccgaaggagctcaaagcatgaagggcaacgcatccagggaaaacacaggctgacagtattcctctttggctgaactcttaatccaaaatctgccagtgatttagctatgccaactggttgaccctccattctctgccaagaggcatactctttctcattgtgtgcgctggcagcagtgcacttccacggagggagattaggatgccgtcagctgatacaaatgggtaaccttttctaatttaaaattccttttagggggtagttagacaatttatatatatatataataaaactattattatatatatagtatatatacattttcaaatttgattttattctggttgaggtgaatgtaagaggaatatataatttaatacaatgtgaacagggcttctgagtctatctcatccctacctaatatgttagggttttgccccttcatttcccttacaaaagaatgttagtaggtttatattaatcattgtgtccaaaagcaagcaaagcaaatcacagtgttcacagctctgcttcataacaaatacataaaccaaatgccataaaatttcttcaactctagttggaaaccgtttggaatttttgttagttgtccagcaggtaagctggatgacctgtggtgctgacctttttacatagtgtagtgttatattagccaaccccaaaggagcagtggttttcaaggtttttactggcctacaaatctaccttcattccgtactgtagaaacatacataccaggtaactaaatcgaatcactctctatcatgagttagtactcactcgcacttaaggaaagggatttgtagttctgtctacaaaattctccaagcagtgttgtggttttttttgtttttgttttttttctttctcttttcaaacagccagttcaggtgcacagcaactttttctacatgcagttcccagggaaactgcagaacttagaatttgtactttttgtaaagctatactctatgggaattgcaagcaatatatctatcttagtattgtgtgtgctaatgagagcctcagtggctcccccactctctcagtgtttcctgcttaaagaaccaacagtttaaaagccctctaagatactctgtgtgtcaccaaatctgtgtgtcaccattttttggtcatgtggtgctatttttgttaagtgtctttttaggtcagtatagttgtagaaaatgtgaaatctgatggtaataatgaattataattgttttcctctcttgagttcatagcttgaaaagagacctcaaaagcatgtgctggcaaacacgttactgtatgaaaacatacctgagtccatttgaataatgttttattagtactttcggaaatgtcttcagttctgtattgtgttcacatacacaaacaggctttacaagattgcttcggtactgtaaactctggcagagagtaattttgtaggcagtttggtggtgagtttgtgctgcaggctgcctgtgggatgtcagcgttctggtatctgcctgagaacctgggctctgagacgcacaaccagtgcacctccataggagaacagtgcagccacctaaaagaaaaacgaacgaaggaccagcctcagaggctagaagttaaaggaatacagaattagatgtttgctggttttctgtgcttttttggctcctaaaataccaatggtggatttgtttttgtttttgttttttgttttgagaaataaaaagtcattcaagccctttgtgtgtaatagcccccaggggtggcagctgtgcagtcgcatctctttggcacacaggatctgttcacgtgtgaactgctgcgctacacatcagtgttaactccctacagattacactctaatcccgctgctcccgaggagcggctttgctaaatcgggtatatagtatatgcctttttcctcgtcaaactgcctaagtaggggttcgttctctccctgaagcacttgttcaactcctgttaaagccgcgtgcctcaaggggaggctggaccccaagtgtttacccacttaaatatgttctggggtttcaggtaaatgtttgtgggtttttttttccttacatgaataagtttggttttgatttttttttaattgaatgcaaaaaatttgtgttgtgatacaaattaagtttgtgacaagaaatgcccaaatccaaggacataagaggtcaagctcagggaaggaacctccttttcactcaggcttggggcctccagcgaggtttccagagcattccatggtatgagagacagtgaggagggagggcacctggcgcgggcacttccagcgtcctggctcttggcattgtccgtcttaaccttatttacatggagttctttgtatttgtgaatctgtttaactggtttgagtttaccaaagagtgacttatccaaaattgtctttgacaaaaatatccattgctttgattgtacagttcaggttcaaacattgtaatgggactgttaaggggcagaaaattgattgagtttctctctaagaatcatgattccacattttgcaagttccacttgctcccattcgtgttgctaacactttaccctttccactgctcgcagtgttaagaatgaattctcaagccataacacagtactgtaaagttccgcagggcttcgagggaggcagcgcctaggccagcacggagctgtgtagcctctctgagcgttcgcactgtcatgcttcccaggggtgtgactggtgagagattaactccattcagatcgggcagcagcaattaattgtgccttgccgcatgaggatgtgtcaggaggattaacatgaccacagaaccgaaacattctctccctgaagttcacttcacgtctccgcagacgaagtacgctgtgtaactccttagagcaactctttttggaaagcaaagtccctatttctgtacagttttaggttaggtgtttcatttataacagatgcagaaatcaattaagataaagtgatatgtgaagaaatcttttacagtaaaatatatcctgaattcatataggcttgttcataattgagtctcttcttgagctaccttttcaatattagacaatgtgaagacagtgacagcgtccttttctagagatatttagcctgttattacaaactgtgaagacaaagaattttatacttttactaatgtttgtggttttaaacagttattttcattctaatcagttctctaccctctaatttctactaaagctgtaaatacatttagaaattatatttgtaaatacagtatatggagacaagttaattttttggtcagtggaaaaagcctcccaaccaattggccctgccttggcagttgtgttttttgttgttgttgttgttgttttagtttagtttttttttttaaacagcagaaaggatactgtcggttcactgttgagcagaatatactgtagaacgaaaatgataatttttaaatcttccagagcatgagtaaatgtcttttctaatgatagcaaatataaccaactctttgtttttcccttagcccagaccatatagacctgcgtattttgtgtgtggttttgtttttatttttgttcttacagcctagaccctaggaaaaatttgcaggaacacgaaacaagggctggggggaaaatcatctatgtgaatgagctttactttaaagagatcaatgtattttattttatcaactttttctcttagttactgtgatttttgttgttgttgtcctcgttattgttaaattctgtaatggtttcctgtgaagcctccactgaaagggactcaaatatgcaacacctaaactattttccaagggcacatgccccttgaatggtgcttctagactggtcagggttatttattaaattttatatatgaaagtattggggaattatgtaaattctttatatgaaactatctagttcataaatcatagatttcatattactcagtgcaactgaactaaaagttcagaaaagtcattcacattgttccaaatttgtaatggttgtcacatgtcacatgcgtctttttcagtaagtgccagagtgttcccactgtttctgcccagtgcttgacttctcggcccggaagagaacctgctttctctggtttccttcctgagtctggcacagacggggctattgtagttcttgatcaagtcctggagtcagccttgcctggctctccttgtagcagattcagtccacagacctcttgctgcccctcagtgacaagtatgctgtgaattcaacctttggacttgctgcccaagcctttggttgctgccctgactattgtaagaggtaaacttacctggtttgtttgagaatgaccattttcctaatgtgaaaaccatctctctcaccacttttattagtagggctaacatttttttccgttataaatggttgagcaatttgaatgacttaacacagtgtcattatcttgcaatataaactggtaacctcacaactccacacttcatcaccatatgaagtaaatgaagctagctaagcggatgctgtatcaactagtaacttgccattaaggattattttatagcatgaatttaagactatttattcaaatgatattttactcttgtattcactttgttttagatttgtgacatgaatatttcagtgctgcttaattttgttctgaattcttgtttcttgcttgtaaatggcttttttatggtataaataaagtcaatggacattgctgtttgtaaataaaaatgctgctagagcaaaaaaaaaaaaaaaa

SEQ ID NO: 44 - DNA, 100 bp, Homo sapiens
  source 1..100: mol_type = unassigned DNA
cagggacatgaggttctccgggagatgttgcataaccactccttcgtgggctgtgtgaatcctcagtgggccttggcacagcatcaaaccaagttatacc

SEQ ID NO: 45 - DNA, 100 bp, Homo sapiens
  source 1..100: mol_type = unassigned DNA
aggtgaagaaaggtgtctgtgatcaaagttttgggattcatgttgcagagcttgctaatttccctaagcatgtaatagagtgtgctaaacagaaagccct

SEQ ID NO: 46 - DNA, 100 bp, Homo sapiens
  source 1..100: mol_type = unassigned DNA
aggcctgaacagccctgtcaaagttgctcgaaagcggaagagaatggtgactggaaatggctctcttaaaaggaaaagctctaggaaggaaacgccctca

SEQ ID NO: 47 - DNA, 100 bp, Homo sapiens
  source 1..100: mol_type = unassigned DNA
tcaggtttcatttcacaatgcacgcatggagttggaaggagttcaacagacagacagtttttctttatcaaccggcggccttgtgacccagcaaaggtct

SEQ ID NO: 48 - DNA, 100 bp, Homo sapiens
  source 1..100: mol_type = unassigned DNA
ggggcaacaacagtccacttctcagacaaacaatggctttgtgactttggcttcttggtggacattatggaacaccttcgagaactcagtgaagaattac

SEQ ID NO: 49 - DNA, 100 bp, Homo sapiens
  source 1..100: mol_type = unassigned DNA
tgccctcaagcaacaattgctagagtaacatctttgtataagcaagtaaccccagatagagttgacgtttcagctttgggctgtcaaaagggtatgtcat

SEQ ID NO: 50 - DNA, 100 bp, Homo sapiens
  source 1..100: mol_type = unassigned DNA
gaaaagctgcagaagaaagatcagcaactggagcctaaaaaaagtaccagccctaaaaaagctgcggagcccactgtggatcttttaggacttgatggcc

SEQ ID NO: 51 - DNA, 100 bp, Homo sapiens
  source 1..100: mol_type = unassigned DNA
ctcttttatgaagctggtacgaagattgatgtcccttgggctgggcagtacatcaccagtaatccctgcatacgcttcgtctccattgataataagaagc

SEQ ID NO: 52 - DNA, 100 bp, Homo sapiens
  source 1..100: mol_type = unassigned DNA
ctctgcttgtgaattccagatgccaggcatgggaggcggcttgtgctttgccttcacttggaagccaccaggaacagaaggtctggccaccctggaagga

SEQ ID NO: 53 - DNA, 100 bp, Homo sapiens
  source 1..100: mol_type = unassigned DNA
ctaccaccaaacattaacatcaaggaacctcgatgggatcaaagcactttcattggacgagccaatcatttcttcactgtaactgaccccaggaacattc

SEQ ID NO: 54 - DNA, 100 bp, Homo sapiens
  source 1..100: mol_type = unassigned DNA
ttcgctttctgcaacacagcaaccagaaactcaagcaggagaacctaagtctgcgcactgctgtccacaaaagcaaatctctgaaggatctggtgtcggc

SEQ ID NO: 55 - DNA, 99 bp, Homo sapiens
  source 1..99: mol_type = unassigned DNA
tgctaaagagctgtcttccaagggagtgaaaatctgggatgccaatgatcccgagactttttggacagcctgggattctccaccagagaagaaggggac

SEQ ID NO: 56 - DNA, 100 bp, Homo sapiens
  source 1..100: mol_type = unassigned DNA
aaaggaaacacgaagattaatcaagcaggaaggacaagctcagttttgcacccactgaatttgccacaaatattgtggaaaatattctcggggacattgc

SEQ ID NO: 57 - DNA, 100 bp, Homo sapiens
  source 1..100: mol_type = unassigned DNA
cgtttggtggagaataccttgtctctgtgtgttccatcaatgccatgcacccaactcggtatattttggctggaggtaattccagcgggaagatacatgt